(19) United States
(12) Patent Application Publication
(10) Pub. No.: US 2022/0348590 A1
(43) Pub. Date: Nov. 3, 2022

(54) CYCLOOXYGENASE-2 INHIBITORS AND USES THEREOF

(71) Applicants: The Board Institute, Inc., Cambridge, MA (US); The General Hospital Corporation, Boston, MA (US); Children's Medical Center Corporation, Boston, MA (US); President and Fellows of Harvard College, Cambridge, MA (US)

(72) Inventors: Florence Fevrier Wagner, Cambridge, MA (US); Michel Weiwer, Cambridge, MA (US); Arthur J. Campbell, Cambridge, MA (US); Joshua R. Sacher, Cambridge, MA (US); Antoine Bigot, Cambridge, MA (US); Agathe Fayet, Cambridge, MA (US); Besnik Bajrami, Cambridge, MA (US); Jacob Hooker, Boston, MA (US); Michael Placzek, Boston, MA (US); Beth A. Stevens, Boston, MA (US); Daniel Wilton, Boston, MA (US); Steven McCarroll, Cambridge, MA (US)

(73) Assignees: The Board Institute, Inc., Cambridge, MA (US); The General Hospital Corporation, Boston, MA (US); Children's Medical Center Corporation, Boston, MA (US); President and Fellows of Harvard College, Cambridge, MA (US)

(21) Appl. No.: 17/642,662

(22) PCT Filed: Sep. 10, 2020
(86) PCT No.: PCT/US2020/050163

§ 371 Date: Mar. 11, 2022

Related U.S. Application Data
(60) Provisional application No. 63/063,833, filed on Aug. 10, 2020.
(60) Provisional application No. 62/900,457, filed on Sep. 13, 2019.

(51) Int. Cl. C07D 491/048; C07D 409/12; C07D 405/12; C07D 213/34; C07D 405/06; C07D 409/14; C07D 241/20; C07D 409/06; C07D 213/64; C07D 401/12; C07D 417/12; C07D 405/08; C07D 241/18; C07D 413/12; C07D 213/74; C07D 241/26; C07D 471/04; C07D 473/00; C07D 333/20; C07D 239/34; C07D 239/42; C07D 487/04; C07D 295/00

(52) U.S. Cl. CPC C07D 491/048; C07D 409/12; C07D 405/12; C07D 213/34; C07D 405/06; C07D 409/14; C07D 241/20; C07D 409/06; C07D 213/64; C07D 401/12; C07D 417/12; C07D 405/08; C07D 241/18; C07D 413/12; C07D 213/74; C07D 241/26; C07D 471/04; C07D 473/00; C07D 333/20; C07D 239/34; C07D 239/42; C07D 487/04; C07D 295/00

(57) ABSTRACT

The present disclosure describes compounds of the formula: (I), (II), (III), (IV), (V). The compounds described herein may be cyclooxygenase (COX) (e.g., cyclooxygenase 2 (COX2)) inhibitors. The compounds may be radiolabeled. The compounds (e.g., radiolabeled compounds) may be useful (e.g., as positron emission tomography (PET) imaging agents) for diagnosing a disease. The compounds may also be useful for treating or preventing a disease. The present disclosure also describes pharmaceutical compositions and kits including the compounds; and methods of using the compounds.

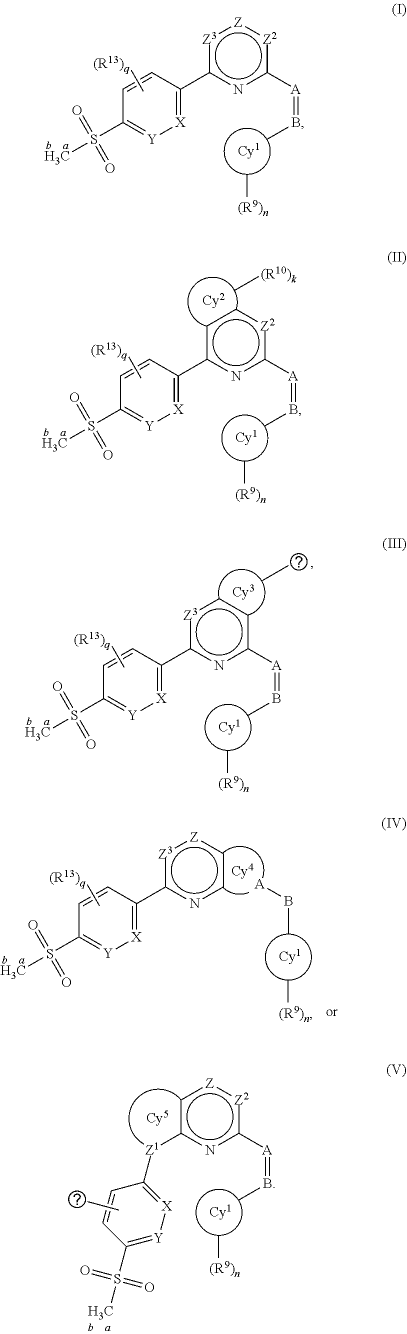

### RELATED APPLICATIONS

The present application claims priority under 35 U.S.C. § 119(e) to U.S. Provisional Application Nos. 62/900,457, filed Sep. 13, 2019, and 63/063,833, filed Aug. 10, 2020, each of which is incorporated herein by reference.

### BACKGROUND OF THE INVENTION

Positron emission tomography (PET) imaging enables in vivo longitudinal measurements of molecular changes in healthy and diseased brains, facilitating the study of disease progression and the identification of molecular markers associated with disease. Due to the discovery that the complement system and activated microglia may be involved in the pathogenesis of schizophrenia (SCZ), the therapeutics team is pursuing the development of a microglia specific PET radiotracer as a potential diagnostic tool for patients in the prodromal state of the disease. There is a need for improved imaging agents.

### SUMMARY OF THE INVENTION

In one aspect, the present disclosure describes compounds of the formula:

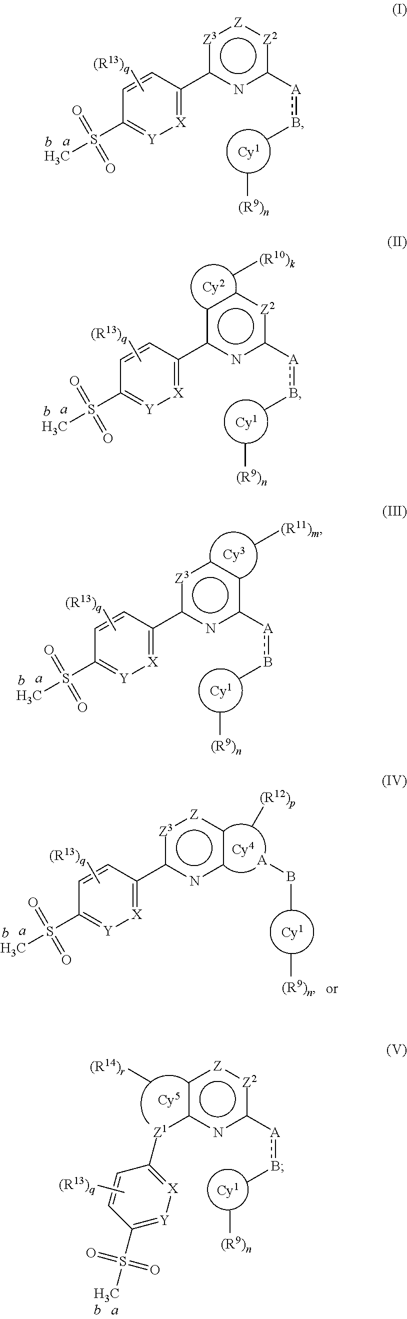

or a salt, solvate, hydrate, polymorph, co-crystal, tautomer, stereoisomer, isotopically labeled derivative, or prodrug thereof.

The compounds described herein may be cyclooxygenase (COX) (e.g., cyclooxygenase 2 (COX2)) inhibitors. The compounds may be radiolabeled. The compounds (e.g., radiolabeled compounds) may be useful for diagnosing a disease. For example, the compounds may be useful as positron emission tomography (PET) imaging agents. The compounds may also be useful for treating or preventing a disease.

The present disclosure also describes pharmaceutical compositions and kits including the compounds; and methods of using the compounds.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC), supercritical fluid chromatography (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

In a formula, the bond [symbol] is a single bond, the dashed line - - - is a single bond or absent, and the bond [symbol] or [symbol] is a single or double bond.

Unless otherwise provided, a formula depicted herein includes compounds that do not include isotopically enriched atoms and also compounds that include isotopically enriched atoms. Compounds that include isotopically enriched atoms may be useful as, for example, analytical tools, and/or probes in biological assays.

The term “aliphatic” includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups (e.g., halo, such as fluorine). As will be appreciated by one of ordinary skill in the art, “aliphatic” is intended herein to include alkyl, alkenyl, alkynyl, cycloalkyl, cycloalkenyl, and cycloalkynyl moieties.

When a range of values (“range”) is listed, it is intended to encompass each value and sub-range within the range. A range is inclusive of the values at the two ends of the range unless otherwise provided. For example, “an integer between 1 and 4” refers to 1, 2, 3, and 4. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₆, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₈), 3-methyl-2-butanyl (C₈), tertiary amyl (C₈), and n-hexyl (C₆). Additional examples of alkyl groups include n-heptyl (C₇), n-octyl (C₈) and the like. Unless otherwise specified, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₂ alkyl (e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)). The attachment point of alkyl may be a single bond (e.g., as in —CH₃), double bond (e.g., as in ═CH₂), or triple bond (e.g., as in ═CH). The moieties ═CH₂ and ═CH are also alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂C₁, and the like.

“Alkenyl” refers to a radical of a straight-chain or branched hydrocarbon group having from 2 to 20 carbon atoms, one or more (e.g., two, three, or four, as valency permits) carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₈ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

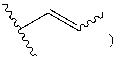

may be in the (E)- or (Z)-configuration.

“Alkynyl” refers to a radical of a straight-chain or branched hydrocarbon group having from 2 to 20 carbon atoms, one or more (e.g., two, three, or four, as valency permits) carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₈ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 13 ring carbon atoms (“C₃₋₁₃ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged, or spiro ring system such as a bicyclic system (“bicyclic carbocyclyl”). Carbocyclyl can be saturated, and saturated carbocyclyl is referred to as “cycloalkyl.” In some embodiments, carbocyclyl is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅_cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. Carbocyclyl can be partially unsaturated. Carbocyclyl may include zero, one, or more (e.g., two, three, or four, as valency permits) C═C double bonds in all the rings of the carbocyclic ring system that are not aromatic or heteroaromatic. Carbocyclyl including one or more (e.g., two or three, as valency permits) C═C double bonds in the carbocyclic ring is referred to as “cycloalkenyl.” Carbocyclyl including one or more (e.g., two or three, as valency permits) C° C. triple bonds in the carbocyclic ring is referred to as “cycloalkynyl.” Carbocyclyl includes aryl. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl. In certain embodiments, the carbocyclyl is substituted or unsubstituted, 3- to 7-membered, and monocyclic. In certain embodiments, the carbocyclyl is substituted or unsubstituted, 5- to 13-membered, and bicyclic. In certain embodiments, the carbocyclyl is substituted or unsubstituted, 6- to 13-membered, and tricyclic.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₁₀ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₃₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 13-membered non-aromatic ring system having ring carbon atoms and 1 to 4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system such as a bicyclic system (“bicyclic heterocyclyl”). A heterocyclyl group can be saturated or can be partially unsaturated. Heterocyclyl may include zero, one, or more (e.g., two, three, or four, as valency permits) double bonds in all the rings of the heterocyclic ring system that are not aromatic or heteroaromatic. Partially unsaturated heterocyclyl groups includes heteroaryl. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl. In certain embodiments, the heterocyclyl is substituted or unsubstituted, 3- to 7-membered, and monocyclic. In certain embodiments, the heterocyclyl is substituted or unsubstituted, 5- to 13-membered, and bicyclic. In certain embodiments, the heterocyclyl is substituted or unsubstituted, 6- to 13-membered, and tricyclic.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include azirdinyl, oxiranyl, or thiiranyl. Exemplary 4-membered heterocyclyl groups containing one heteroatom include azetidinyl, oxetanyl and thietanyl. Exemplary 5-membered heterocyclyl groups containing one heteroatom include tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include dioxolanyl, oxasulfuranyl, disulfuranyl, and oxazolidin-2-one. Exemplary 5-membered heterocyclyl groups containing three heteroatoms include triazolinyl, oxadiazolinyl, and thiadiazolinyl. Exemplary 6-membered heterocyclyl groups containing one heteroatom include piperidinyl, tetrahydropyranyl, dihydropyridinyl, and thianyl. Exemplary 6-membered heterocyclyl groups containing two heteroatoms include piperazinyl, morpholinyl, dithianyl, and dioxanyl. Exemplary 6-membered heterocyclyl groups containing two heteroatoms include triazinanyl. Exemplary 7-membered heterocyclyl groups containing one heteroatom include azepanyl, oxepanyl and thiepanyl. Exemplary 8-membered heterocyclyl groups containing one heteroatom include azocanyl, oxecanyl, and thiocanyl. Exemplary 5-membered heterocyclyl groups fused to a C₆ aryl ring (also referred to herein as a 5,6-bicyclic heterocyclic ring) include indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 n electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include pyrrolyl, furanyl and thiophenyl. Exemplary 5-membered heteroaryl groups containing two heteroatoms include imidazolyl, pyrazolyl, oxazolyl, isoxazolyl, thiazolyl, and isothiazolyl. Exemplary 5-membered heteroaryl groups containing three heteroatoms include triazolyl, oxadiazolyl, and thiadiazolyl. Exemplary 5-membered heteroaryl groups containing four heteroatoms include tetrazolyl. Exemplary 6-membered heteroaryl groups containing one heteroatom include pyridinyl. Exemplary 6-membered heteroaryl groups containing two heteroatoms include pyridazinyl, pyrimidinyl, and pyrazinyl. Exemplary 6-membered heteroaryl groups containing three or four heteroatoms include triazinyl and tetrazinyl, respectively. Exemplary 7-membered heteroaryl groups containing one heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, aliphatic, alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include halogen, —CN, —NO₂, —N₃, —SO₂H, —SO₃H, —OH, —OR—, —ON(R^{bb})₂, —N(R^{bb})₂, —N(R^{bb})₃⁺X⁻, —N(OR^{cc})R^{bb}, —SH, —SR—, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂-6 alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(Rb)₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group. In certain embodiments, the carbon atom substituents are independently halogen, substituted (e.g., substituted with one or more halogen) or unsubstituted C₁₋₆ alkyl, —OR^{aa}, —SR—, —N(R^{bb})₂, —CN, —SCN, or —NO₂. In certain embodiments, the carbon atom substituents are independently halogen, substituted (e.g., substituted with one or more halogen moieties) or unsubstituted C₁₋₆ alkyl, —OR^{aa}, —SR^{aa}, —N(R^{bb})₂, —CN, —SCN, or —NO₂, wherein R^{a} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO4⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{cc}, —C(═NR^{cc})N(R^{cc})₂, —SO₂N(R^{cc})₂, —SO₂R^{cc}, —SO₂R^{cc}, —SOR^{aa}, —C(═S)N(RC)₂, —C(═O)SR^{cc}, —C(═S)SR^{cc}, —P(═O)(OR^{cc})₂, —P(═O)(R^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups, and wherein R^{aa}, R^{bb}, R^{cc} and R^{dd} are as defined above.

In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group, wherein R¹¹ is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the nitrogen atom substituents are independently substituted (e.g., substituted with one or more halogen) or unsubstituted C₁₋₆ alkyl or a nitrogen protecting group.

In certain embodiments, the substituent present on a nitrogen atom is a nitrogen protecting group (also referred to as an amino protecting group). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{cc}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{aa}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, a nitrogen protecting group is Bn, Boc, Cbz, Fmoc, trifluoroacetyl, triphenylmethyl, acetyl, or Ts.

In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group. In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂ or an oxygen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the oxygen atom substituents are independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{aa})₃⁺X⁻, —P(OR^{cc})₂, —P(OR^{aa})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, an oxygen protecting group is silyl, TBDPS, TBDMS, TIPS, TES, TMS, MOM, THP, t-Bu, Bn, allyl, acetyl, pivaloyl, or benzoyl.

In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group. In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the sulfur atom substituents are independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). Sulfur protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R′)₂, —P(═O)(OR^{cc})₂, and —P(═O)(N(R^{bb}) 2)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

The “molecular weight” of -R, wherein -R is any monovalent moiety, is calculated by substracting the atomic weight of a hydrogen atom from the molecular weight of the molecule R—H. The “molecular weight” of -L-, wherein -L- is any divalent moiety, is calculated by substracting the combined atomic weight of two hydrogen atoms from the molecular weight of the molecule H-L-H.

In certain embodiments, the molecular weight of a substituent is lower than 200, lower than 150, lower than 100, lower than 50, or lower than 25 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, and/or iodine atoms. In certain embodiments, a substituent consists of carbon, hydrogen, and/or fluorine atoms. In certain embodiments, a substituent does not comprise one or more, two or more, or three or more hydrogen bond donors. In certain embodiments, a substituent does not comprise one or more, two or more, or three or more hydrogen bond acceptors.

These and other exemplary substituents are described in more detail in the Detailed Description, Examples, Figures, and Claims. The present disclosure is not intended to be limited in any manner by the above exemplary listing of substituents.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences (1977) 66:1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

The term “tautomers” refer to compounds that are interchangeable forms of a particular compound structure, and that vary in the displacement of hydrogen atoms and electrons. Thus, two structures may be in equilibrium through the movement of 7 electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refer to compounds, including derivatives of the compounds of Formula (I), which have cleavable groups and become by solvolysis or under physiological conditions the compounds of Formula (I) which are pharmaceutically active in vivo. Such examples include, but are not limited to, ester derivatives and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. C₁ to C₈ alkyl, C₂-C₈ alkenyl, C₂₋₈ alkynyl, aryl, C₇-C₁₂ substituted aryl, and C₇-C₁₂ arylalkyl esters of the compounds of Formula (I) may be preferred.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The terms “administer,” “administering,” or “administration,” refers to implanting, absorbing, ingesting, injecting, inhaling, or otherwise introducing a compound, or a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The terms “condition,” “disease,” and “disorder” are used interchangeably.

An “effective amount” of a compound of Formula (I) refers to an amount sufficient to elicit the desired biological response, i.e., treating the condition. As will be appreciated by those of ordinary skill in this art, the effective amount of a compound of Formula (I) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of a compound may reduce the tumor burden or stop the growth or spread of a tumor.

A “therapeutically effective amount” of a compound of Formula (I) is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, autoinflammatory diseases, and autoimmune diseases.

The terms “neoplasm” and “tumor” are used interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “angiogenesis” refers to the formation and the growth of new blood vessels. Normal angiogenesis occurs in the healthy body of a subject for healing wounds and for restoring blood flow to tissues after injury. The healthy body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal or pathological angiogenesis refers to angiogenesis greater than that in a normal body, especially angiogenesis in an adult not related to normal angiogenesis (e.g., menstruation or wound healing). Abnormal angiogenesis can provide new blood vessels that feed diseased tissues and/or destroy normal tissues, and in the case of cancer, the new vessels can allow tumor cells to escape into the circulation and lodge in other organs (tumor metastases). In certain embodiments, the angiogenesis is pathological angiogenesis.

An “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid, arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme arthritis, Guillain-Barré syndrome, Hashimoto's thyroiditis, and cardiomyopathy.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including Alzheimer's disease, Parkinson's disease, amyotrophic lateral sclerosis, tauopathies (including frontotemporal dementia), and Huntington's disease. Examples of neurological diseases include headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D. C. (1994). Psychiatric disorders include anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include the endocrine (hormonal) control system (e.g., the insulin pathway, the enteroendocrine hormones including GLP-1, PYY or the like), the neural control system (e.g., GLP-1 in the brain), or the like. Examples of metabolic disorders include diabetes (e.g., Type I diabetes, Type II diabetes, gestational diabetes), hyperglycemia, hyperinsulinemia, insulin resistance, and obesity.

A “protein” or “peptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, or synthetic, or any combination of these.

### BRIEF DESCRIPTION OF THE DRAWINGS

FIGS. 1A to 1F show that COX2 is specifically and significantly increased in microglia of HD patients and HD mouse models. FIGS. 1A and 1B show quantification of western blot band intensity of COX2 (FIG. 1A) and TSPO (FIG. 1B) in human post-mortem globus pallidus tissue from healthy controls (n=8) and HD patients (n=12). Both normalized to GAPDH. FIG. 1C shows fold change of C. COX2 (% COX2+ cells) and

FIG. 1D shows TSPO (mean intensity) expressed in microglia, neurons and astrocytes as denoted by colocalization with RCA-1, NeuN and Sox9 respectively in post-mortem tissue from healthy controls and HD patients (Vonsattel grade 4, HD4). FIGS. 1E and 1F show that COX2 transcript are elevated in the caudate nucleus of manifest (FIG. 1E) and pre-symptomatic (FIG. 1F) HD patients. FIG. 1E shows qPCR of mRNA from caudate nucleus (CdN) and cerebellum (Cer) of manifest HD patients and control (clinically normal) individuals. (n=4-5) FIG. 1F shows qPCR of mRNA from caudate nucleus of 2 pre-symptomatic HD patients and 2 age-matched control (clinically normal) individuals. Only COX2, and not other microglia enriched genes or TSPO, is elevated in the pre-symptomatic samples.

FIG. 2 shows that COX2 ligand optimization leads to increase in potency and free fraction, and translate into increase in specific binding in in-vitro autoradiography. Ligand structures and key properties are shown.

FIG. 3 shows exemplary properties for radiotracer development and corresponding data on selected compounds.

FIG. 4 shows radiosynthesis of [¹¹C]47 illustrating our thioester precursor strategy.

FIG. 5 shows that increasing binding affinity and fraction unbound led to improved COX2 radioligands, showing increased specific binding in rat in vitro autoradiography using self-block experiments.

FIG. 6 shows that [¹¹C]36 specific binding is COX2 dependent in a human brain tissue region known to be affected in Huntington disease.

FIG. 7 illustrates that [¹¹C]47 and [¹¹C]36 show high, rapid and homogeneous brain uptake followed by fast clearance in baboon.

### DETAILED DESCRIPTION OF CERTAIN EMBODIMENTS

It has been identified that COX2 is a PET ligand target. Due to the lack of animal models of SCZ, a strategic choice was made to first focus on the development of a biomarker of early onset and progression of pathology in Huntington's disease (HD), in which microglial activation is present early in pathology. Neuropathological studies indicate that there is an immune activation in the HD brain, or neuroinflammation, which is evident at early stages of disease progression [Rosenblatt, 2000]. Monitoring key proteins essential to this cascade could provide an opportunity to identify imbalances critical to neuronal and synapse loss. Thus far, exploration of neuroinflammation in neurodegenerative disease pathology in the living human brain with positron emission tomography (PET) has been limited to the translocator protein (TSPO), which has not yet been linked mechanistically to disease pathology. TSPO has several drawbacks, most importantly, nonspecific cellular expression in the brain, confounding mechanistic interpretation of the signal [Albrecht, 2016]. Alternatively, cyclooxygenase (COX) is a central enzyme in the neuroinflammatory cascade, which through generation of prostaglandins, triggers an increase in the expression of inflammatory cytokines in microglia [Farooqui, 2007]. In the brain, the inducible COX isoform, COX2, is specifically upregulated in response to inflammatory events, including certain degenerative diseases [Minghetti, 2004].

In one aspect, the present disclosure describes compounds of any one of Formula (I) to (V), or a salt, solvate, hydrate, polymorph, co-crystal, tautomer, stereoisomer, isotopically labeled derivative, or prodrug thereof. The compounds described herein may be cyclooxygenase (COX) (e.g., cyclooxygenase 2 (COX2)) inhibitors. The compounds may be radiolabeled. The compounds (e.g., radiolabeled compounds) may be useful for diagnosing a disease (e.g., as imaging agents (e.g., PET imaging agents). The compounds may also be useful for treating or preventing a disease. The present disclosure also describes pharmaceutical compositions and kits including the compounds; and methods of using the compounds.

The compounds described herein may be advantageous over known compounds because the compounds described herein are more selective in inhibiting COX2 over COX1, more potent in inhibiting COX2, or result in more free fraction, or a combination thereof.

### Compounds

In one aspect, the present disclosure describes compounds of the formula:

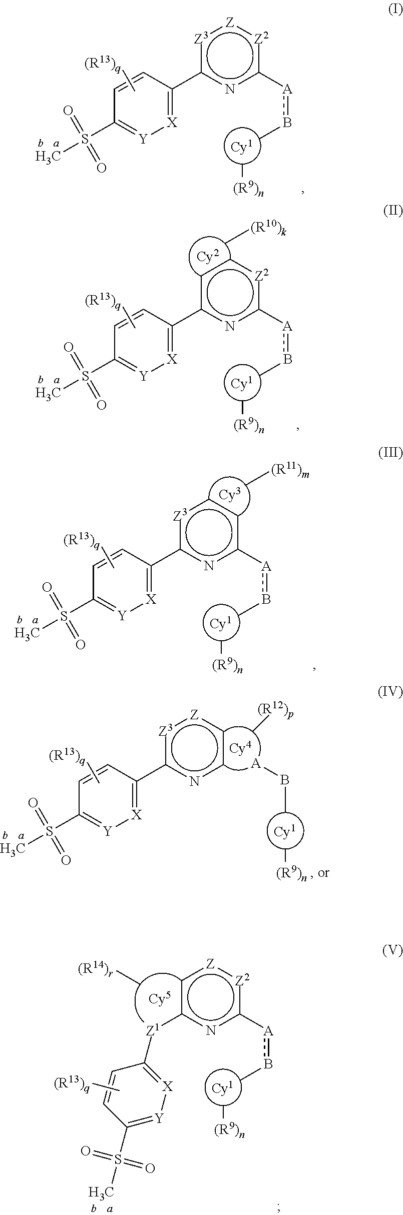

or a salt, solvate, hydrate, polymorph, co-crystal, tautomer, stereoisomer, isotopically labeled derivative, or prodrug thereof, wherein:
Y is CR¹³ or N;
each instance of R¹³ is independently hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
each instance of R^{a} is independently hydrogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form substituted or unsubstituted heterocyclyl or substituted or unsubstituted heteroaryl;
q is 0, 1, or 2;
X is CR¹³ or N;
Z³ is CR¹ or N;
R¹ is hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
Z is CH, CF, or N;
Cy² is monocyclic heteroaryl, monocyclic carbocyclyl, monocyclic heterocyclyl, or phenyl;
each instance of R¹⁰ is independently hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂, or two instances of R¹⁰ on the same carbon atom taken together with the carbon atom form C(═O);
k is 0, 1, 2, 3, 4, 5, 6, 7, or 8, as valency permits;
Z² is CR² or N;
R² is hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
Cy³ is monocyclic heteroaryl, monocyclic carbocyclyl, monocyclic heterocyclyl, or phenyl;
each instance of R¹¹ is independently hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂, or two instances of R¹¹ on the same carbon atom taken together with the carbon atom form C(═O);
m is 0, 1, 2, 3, 4, 5, 6, 7, or 8, as valency permits;
Cy⁴ is monocyclic heteroaryl, monocyclic carbocyclyl, monocyclic heterocyclyl, or phenyl;
each instance of R¹² is independently hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂, or two instances of R¹² on the same carbon atom taken together with the carbon atom form C(═O);
p is 0, 1, 2, 3, 4, 5, or 6, as valency permits;
Z¹ is C, CR¹⁵, or N;
R¹⁵ is hydrogen, halogen, or substituted or unsubstituted alkyl;
Cy⁵ is monocyclic heteroaryl, monocyclic carbocyclyl, monocyclic heterocyclyl, or phenyl;
each instance of R¹⁴ is independently hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂, or two instances of R¹⁴ on the same carbon atom taken together with the carbon atom form C(═O);
r is 0, 1, 2, 3, 4, 5, or 6, as valency permits;
[symbol] is a single bond or double bond;
in any one of Formulae (I) to (III) and (V), A is O, NR¹⁶, S, S(═O), C(═O), or C(R¹⁹)₂ or A and B are joined to form substituted or unsubstituted, monocyclic carbocyclyl, or

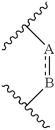

is

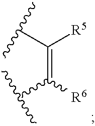

R¹⁶ is hydrogen, substituted or unsubstituted acyl, substituted or unsubstituted alkyl, or a nitrogen protecting group;
each instance of R¹⁹ is independently hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
in Formula (IV), A is N, C, or CR¹⁷;
R¹⁷ is hydrogen, halogen, or substituted or unsubstituted alkyl;
B is C(R³)(R⁴), C(═O), NR¹⁸, or a single bond;
R³ is hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
R⁴ is hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
R¹⁸ is hydrogen, substituted or unsubstituted acyl, substituted or unsubstituted alkyl, or a nitrogen protecting group;
R⁵ is hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
R⁶ is hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
Cy¹ is heteroaryl, aryl, carbocyclyl, or heterocyclyl;
each instance of R⁹ is independently hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂, or two instances of R⁹ on the same carbon atom taken together with the carbon atom form C(═O); and
n is 0, 1, 2, 3, 4, 5, 6, 7,8, 9, 10, or 11, as valency permits;
   provided that:
the compound is not of the formula:

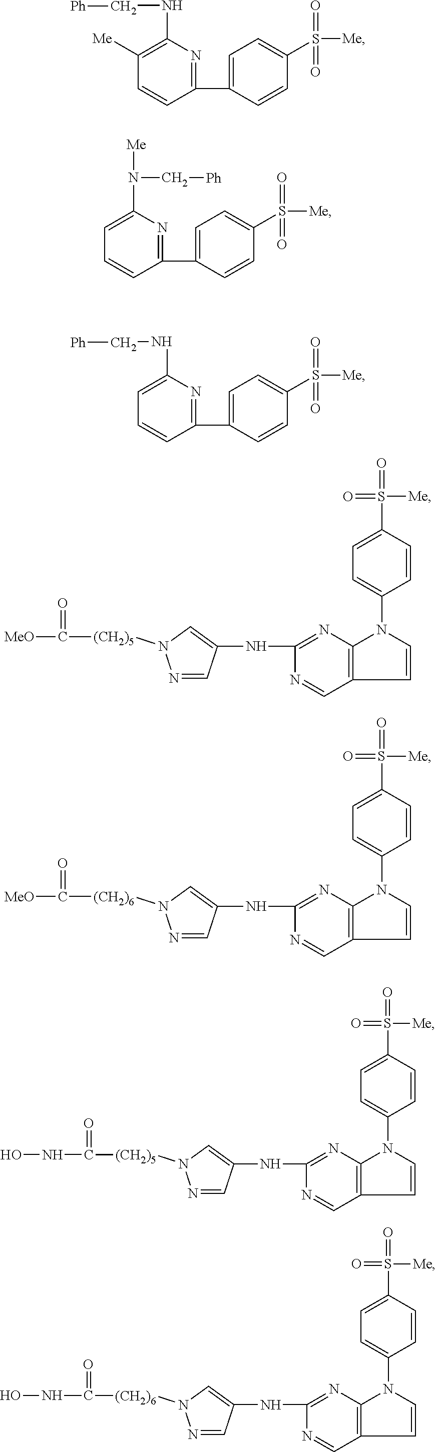

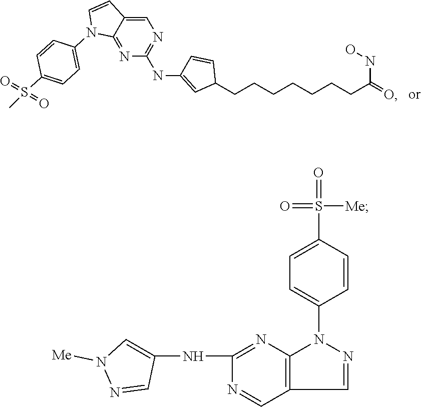 [SMILES: CS(=O)(=O)c1ccc(-n2ccc3cnc(NC4=CC(CCCCCCCC(=O)NO)C=C4)nc32)cc1 ; Cn1cc(Nc2ncc3cnn(-c4ccc(S(C)(=O)=O)cc4)c3n2)cn1]

and
the molecular weight of the compound is not more than 500 g/mol.

In certain embodiments, the compound is of Formula (I), or a salt, solvate, hydrate, polymorph, co-crystal, tautomer, stereoisomer, isotopically labeled derivative, or prodrug thereof.

In certain embodiments, the compound is of Formula (II), or a salt, solvate, hydrate, polymorph, co-crystal, tautomer, stereoisomer, isotopically labeled derivative, or prodrug thereof.

In certain embodiments, the compound is of Formula (III), or a salt, solvate, hydrate, polymorph, co-crystal, tautomer, stereoisomer, isotopically labeled derivative, or prodrug thereof.

In certain embodiments, the compound is of Formula (IV), or a salt, solvate, hydrate, polymorph, co-crystal, tautomer, stereoisomer, isotopically labeled derivative, or prodrug thereof.

In certain embodiments, the compound is of Formula (V), or a salt, solvate, hydrate, polymorph, co-crystal, tautomer, stereoisomer, isotopically labeled derivative, or prodrug thereof.

In certain embodiments, the compound is of the formula:

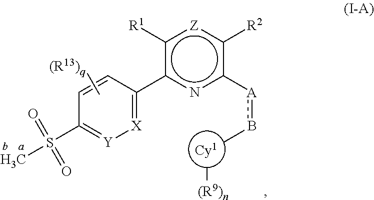

or a salt, solvate, hydrate, polymorph, co-crystal, tautomer, stereoisomer, isotopically labeled derivative, or prodrug thereof.

In certain embodiments, the compound is of the formula:

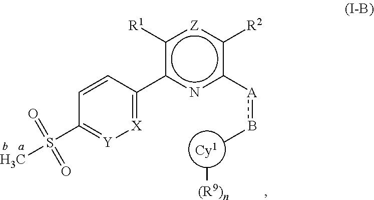

or a salt, solvate, hydrate, polymorph, co-crystal, tautomer, stereoisomer, isotopically labeled derivative, or prodrug thereof, wherein:
Y is CH or N;
X is CH or N;
R¹ is hydrogen, —OCH₃, —CH₃, F, or C₁;
Z is CH, CF, or N;
R² is hydrogen, F, Cl, —OCH₃, or —CH₃;
A is O, NH, NCH₃, or S;
B is C(R³)(R⁴) or C(═O);
R³ is hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
each instance of R^{a} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, or an oxygen protecting group when attached to an oxygen atom, or two instances of R^{a} are joined to form substituted or unsubstituted heterocyclyl or substituted or unsubstituted heteroaryl;
R⁴ is hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
or

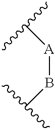

is

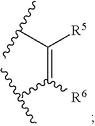

R⁵ is hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
R⁶ is hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂; and
Cy¹ is 5- or 6-membered, monocyclic heteroaryl, 3- to 6-membered, monocyclic, partially unsaturated heterocyclyl, or phenyl;
each instance of R⁹ is independently halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂, or two instances of R⁹ on the same carbon atom taken together with the carbon atom form C(═O); and
n is 0, 1, 2, 3, 4, 5, 6, 7, 8, 9, 10, or 11, as valency permits.

In certain embodiments, the compound is of the formula:

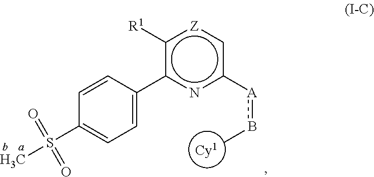

or a salt, solvate, hydrate, polymorph, co-crystal, tautomer, stereoisomer, isotopically labeled derivative, or prodrug thereof.

In certain embodiments, the compound is of the formula:

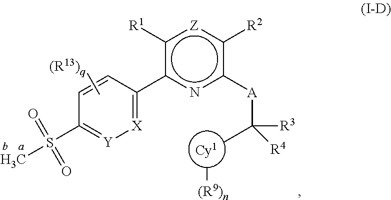

or a salt, solvate, hydrate, polymorph, co-crystal, tautomer, stereoisomer, isotopically labeled derivative, or prodrug thereof.

In certain embodiments, the compound is of the formula:

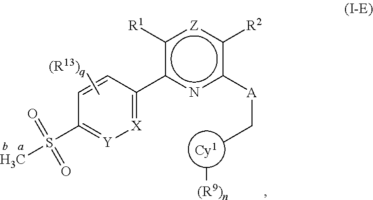

or a salt, solvate, hydrate, polymorph, co-crystal, tautomer, stereoisomer, isotopically labeled derivative, or prodrug thereof.

In certain embodiments, the compound is of the formula:

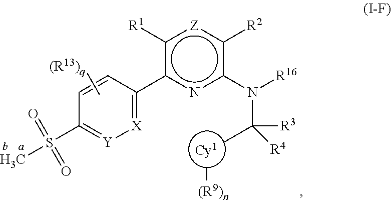

or a salt, solvate, hydrate, polymorph, co-crystal, tautomer, stereoisomer, isotopically labeled derivative, or prodrug thereof.

In certain embodiments, the compound is of the formula:

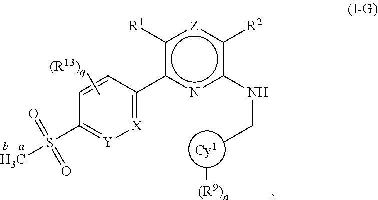

or a salt, solvate, hydrate, polymorph, co-crystal, tautomer, stereoisomer, isotopically labeled derivative, or prodrug thereof.

In certain embodiments, the compound is of the formula:

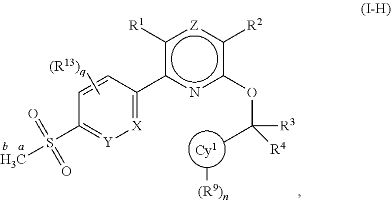

or a salt, solvate, hydrate, polymorph, co-crystal, tautomer, stereoisomer, isotopically labeled derivative, or prodrug thereof.

In certain embodiments, the compound is of the formula:

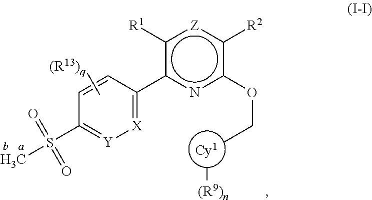

or a salt, solvate, hydrate, polymorph, co-crystal, tautomer, stereoisomer, isotopically labeled derivative, or prodrug thereof.

In certain embodiments, the compound is of the formula:

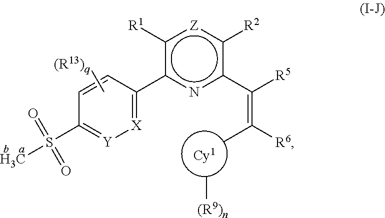

or a salt, solvate, hydrate, polymorph, co-crystal, tautomer, stereoisomer, isotopically labeled derivative, or prodrug thereof.

In certain embodiments, the compound is of the formula:

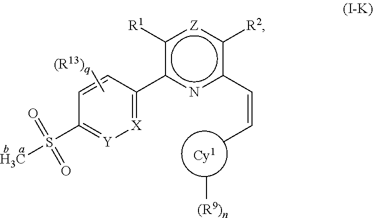

or a salt, solvate, hydrate, polymorph, co-crystal, tautomer, stereoisomer, isotopically labeled derivative, or prodrug thereof.

In certain embodiments, the compound is not of the formula:

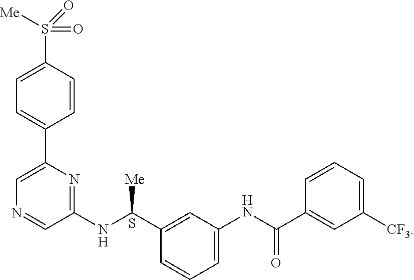

In certain embodiments, the compound is not of the formula:

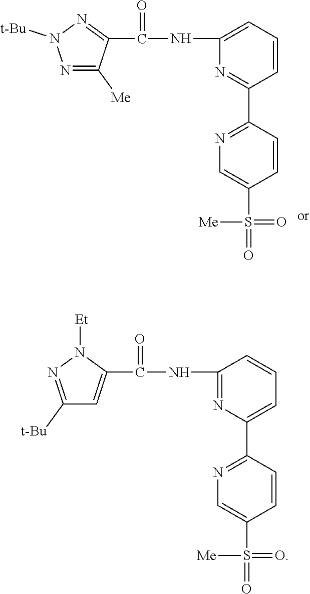 [SMILES: Cc1nn(C(C)(C)C)nc1C(=O)Nc1cccc(-c2ccc(S(C)(=O)=O)cn2)n1 ; CCn1nc(C(C)(C)C)cc1C(=O)Nc1cccc(-c2ccc(S(C)(=O)=O)cn2)n1]

In certain embodiments, the compound is of the formula:

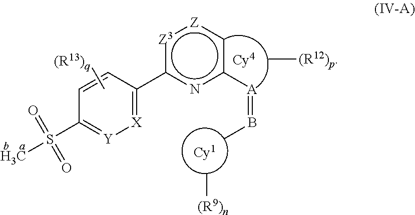

In certain embodiments, the compound is of the formula:

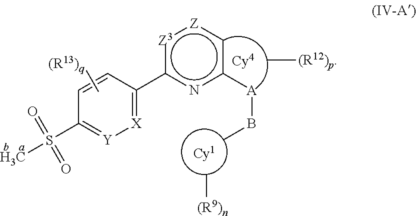

When a formula includes two or more instances of a moiety, unless otherwise provided, any two instances of the moiety may be the same or different from each other.

In certain embodiments, X is CR¹³. In certain embodiments, X is CH, C(halogen), C(substituted or unsubstituted alkyl), or C(O(substituted or unsubstituted alkyl)). In certain embodiments, X is CH, C(halogen), C(unsubstituted C₁₋₆ alkyl), or C(O(unsubstituted C₁₋₆ alkyl)). In certain embodiments, X is CH, C(halogen), CMe, CEt, C(OMe), or C(OEt). In certain embodiments, X is C(OH). In certain embodiments, X is CH. In certain embodiments, X is N.

In certain embodiments, Y is CR¹³. In certain embodiments, Y is CH, C(halogen), C(substituted or unsubstituted alkyl), or C(O(substituted or unsubstituted alkyl)). In certain embodiments, Y is CH, C(halogen), C(unsubstituted C₁₋₆ alkyl), or C(O(unsubstituted C₁₋₆ alkyl)). In certain embodiments, Y is CH, C(halogen), CMe, CEt, C(OMe), or C(OEt). In certain embodiments, Y is C(OH). In certain embodiments, Y is CH. In certain embodiments, Y is N.

In certain embodiments, each of Y and X is CH. In certain embodiments, Y is CH, and X is N. In certain embodiments, Y is N, and X is CH.

In certain embodiments, each of Y and X is CR¹³. In certain embodiments, each of Y and X is independently CH, C(halogen), C(substituted or unsubstituted alkyl), or C(O(substituted or unsubstituted alkyl)). In certain embodiments, each of Y and X is independently CH, C(halogen), C(unsubstituted C₁₋₆ alkyl), or C(O(unsubstituted C₁₋₆ alkyl)). In certain embodiments, each of Y and X is independently CH, C(halogen), CMe, CEt, C(OMe), or C(OEt). In certain embodiments, Y is CR¹³, and X is N. In certain embodiments, Y is CH, C(halogen), C(substituted or unsubstituted alkyl), or C(O(substituted or unsubstituted alkyl)), and X is N. In certain embodiments, Y is CH, C(halogen), C(unsubstituted C₁₋₆ alkyl), or C(O(unsubstituted C₁₋₆ alkyl)), and X is N. In certain embodiments, Y is CH, C(halogen), CMe, CEt, C(OMe), or C(OEt), and X is N. In certain embodiments, X is CR¹³, and Y is N. In certain embodiments, X is CH, C(halogen), C(substituted or unsubstituted alkyl), or C(O(substituted or unsubstituted alkyl)), and Y is N. In certain embodiments, X is CH, C(halogen), C(unsubstituted C₁₋₆ alkyl), or C(O(unsubstituted C₁₋₆ alkyl)), and Y is N. In certain embodiments, X is CH, C(halogen), CMe, CEt, C(OMe), or C(OEt), and Y is N. In certain embodiments, each of Y and X is N.

In certain embodiments, each instance of R¹³ is hydrogen. In certain embodiments, at least one R¹³ is hydrogen. In certain embodiments, at least one R¹³ is not hydrogen. In certain embodiments, at least one instance of R¹³ is halogen (e.g., F, Cl, or Br). In certain embodiments, at least one instance of R¹³ is substituted alkyl (e.g., alkyl substituted with one or more instances of halogen (e.g., F)). In certain embodiments, at least one instance of R¹³ is —CF₃. In certain embodiments, at least one instance of R¹³ is unsubstituted alkyl. In certain embodiments, at least one instance of R¹³ is unsubstituted, C₁₋₆ alkyl. In certain embodiments, at least one instance of R¹³ is Me. In certain embodiments, at least one instance of R¹³ is Et, Pr, or Bu. In certain embodiments, at least one instance of R¹³ is substituted C₁₋₆ alkyl. In certain embodiments, at least one instance of R¹³ is substituted methyl. In certain embodiments, at least one instance of R¹³ is substituted ethyl, substituted propyl, or substituted butyl. In certain embodiments, at least one instance of R¹³ is substituted or unsubstituted alkenyl. In certain embodiments, at least one instance of R¹³ is substituted or unsubstituted, C₂₋₆ alkenyl (e.g., substituted or unsubstituted vinyl or substituted or unsubstituted allyl). In certain embodiments, at least one instance of R¹³ is substituted or unsubstituted alkynyl. In certain embodiments, at least one instance of R¹³ is substituted or unsubstituted, C₂₋₆ alkynyl (substituted or unsubstituted ethynyl). In certain embodiments, at least one instance of R¹³ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, at least one instance of R¹³ is —OH. In certain embodiments, at least one instance of R¹³ is —OMe. In certain embodiments, at least one instance of R¹³ is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, at least one instance of R¹³ is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe2)). In certain embodiments, at least one instance of R¹³ is —CN or —SCN. In certain embodiments, at least one instance of R¹³ is —NO₂. In certain embodiments, at least one instance of R¹³ is —C(═NR^{a})R^{a}, —C(═NR^{a})OR^{a}, or —C(═NR^{a})N(R^{a})₂. In certain embodiments, at least one instance of R¹³ is —C(═O)R^{a} (e.g., —C(═O)(substituted or unsubstituted alkyl) (e.g., —C(═O)Me) or —C(═O)(substituted or unsubstituted phenyl)). In certain embodiments, at least one instance of R¹³ is —C(═O)OR^{a}(e.g., —C(═O)OH, —C(═O)O(substituted or unsubstituted alkyl) (e.g., —C(═O)OMe), or —C(═O)O(substituted or unsubstituted phenyl)). In certain embodiments, at least one instance of R¹³ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, at least one instance of R¹³ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, at least one instance of R¹³ is —NR^{a}C(═O)OR^{a}. In certain embodiments, at least one instance of R¹³ is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, at least one instance of R¹³ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, at least one instance of R¹³ is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)).

In certain embodiments, at least one instance of R^{a} is hydrogen. In certain embodiments, each instance of R^{a} is hydrogen. In certain embodiments, at least one instance of R^{a} is not hydrogen. In certain embodiments, no instance of R^{a} is hydrogen. In certain embodiments, at least one instance of R^{a} is substituted alkyl (e.g., alkyl substituted with one or more instances of halogen (e.g., F)). In certain embodiments, at least one instance of R^{a} is unsubstituted alkyl. In certain embodiments, at least one instance of R^{a} is unsubstituted, C₁₋₆ alkyl. In certain embodiments, at least one instance of R^{a} is Me. In certain embodiments, at least one instance of R^{a} is Et, Pr, or Bu. In certain embodiments, at least one instance of R^{a} is substituted C₁₋₆ alkyl. In certain embodiments, at least one instance of R^{a} is substituted methyl. In certain embodiments, at least one instance of R^{a} is substituted ethyl, substituted propyl, or substituted butyl. In certain embodiments, at least one instance of R^{a} is substituted or unsubstituted alkenyl. In certain embodiments, at least one instance of R^{a} is substituted or unsubstituted, C₂₋₆ alkenyl (e.g., substituted or unsubstituted vinyl or substituted or unsubstituted allyl). In certain embodiments, at least one instance of R^{a} is substituted or unsubstituted alkynyl. In certain embodiments, at least one instance of R^{a} is substituted or unsubstituted, C₂₋₆ alkynyl (substituted or unsubstituted ethynyl). In certain embodiments, at least one instance of R^{a} is substituted or unsubstituted carbocyclyl (e.g., substituted or unsubstituted, monocyclic, 3- to 7-membered carbocyclyl comprising 0, 1, or 2 double bonds in the carbocyclic ring system, as valency permits). In certain embodiments, at least one instance of R^{a} is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R^{a} is substituted or unsubstituted heterocyclyl (e.g., substituted or unsubstituted, 3- to 7-membered, monocyclic heterocyclyl). In certain embodiments, at least one instance of R^{a} is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R^{a} is substituted or unsubstituted aryl. In certain embodiments, at least one instance of R^{a} is substituted or unsubstituted phenyl. In certain embodiments, at least one instance of R^{a} is substituted or unsubstituted naphthyl. In certain embodiments, at least one instance of R^{a} is substituted or unsubstituted heteroaryl. In certain embodiments, at least one instance of R^{a} is substituted or unsubstituted, 5- to 6-membered, monocyclic heteroaryl. In certain embodiments, at least one instance of R^{a} is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R^{a} is substituted or unsubstituted pyridinyl, substituted or unsubstituted pyrazinyl, substituted or unsubstituted pyrimidinyl, or substituted or unsubstituted pyridazinyl. In certain embodiments, at least one instance of R^{a} is substituted or unsubstituted, 9- to 10-membered, bicyclic heteroaryl. In certain embodiments, at least one instance of R^{a} is a nitrogen protecting group (e.g., Bn, Boc, Cbz, Fmoc, trifluoroacetyl, triphenylmethyl, acetyl, or Ts) when attached to a nitrogen atom. In certain embodiments, at least one instance of R^{a} is an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom. In certain embodiments, two instances of R^{a} are joined to form substituted or unsubstituted heterocyclyl (e.g., substituted or unsubstituted, 3- to 7-membered, monocyclic heterocyclyl). In certain embodiments, two instances of R^{a} are joined to form substituted or unsubstituted heteroaryl (e.g., substituted or unsubstituted, 5- to 6-membered, monocyclic heteroaryl).

In certain embodiments, q is 0. In certain embodiments, q is 1. In certain embodiments, q is 2.

In certain embodiments, Z³ is CR¹. In certain embodiments, Z³ is CH. In certain embodiments, Z³ is C(halogen) (e.g., CF). In certain embodiments, Z³ is C(substituted or unsubstituted, C₁₋₆ alkyl) (e.g., C(CH₃), C(CF₃), C(CH₂OCH₃)). In certain embodiments, Z³ is C(O-substituted or unsubstituted, C₁₋₆ alkyl) (e.g., C(OCH₃), C(OC₂H₅), C(OC₃H₇), C(OCH₂CH₂OCH₃)). In certain embodiments, Z³ is N.

In certain embodiments, R¹ is hydrogen. In certain embodiments, R¹ is halogen (e.g., F, Cl, or Br). In certain embodiments, R¹ is substituted alkyl (e.g., alkyl substituted with one or more instances of halogen (e.g., F)). In certain embodiments, R¹ is —CF₃. In certain embodiments, R¹ is unsubstituted alkyl. In certain embodiments, R¹ is unsubstituted, C₁₋₆ alkyl. In certain embodiments, R¹ is Me. In certain embodiments, R¹ is Et, Pr, or Bu. In certain embodiments, R¹ is substituted C₁₋₆ alkyl. In certain embodiments, R¹ is substituted methyl. In certain embodiments, R¹ is substituted ethyl, substituted propyl, or substituted butyl. In certain embodiments, R¹ is substituted or unsubstituted alkenyl. In certain embodiments, R¹ is substituted or unsubstituted, C₂₋₆ alkenyl (e.g., substituted or unsubstituted vinyl or substituted or unsubstituted allyl). In certain embodiments, R¹ is substituted or unsubstituted alkynyl. In certain embodiments, R¹ is substituted or unsubstituted, C₂₋₆ alkynyl (substituted or unsubstituted ethynyl). In certain embodiments, R¹ is substituted or unsubstituted, monocyclic carbocyclyl (e.g., substituted or unsubstituted, monocyclic, 3- to 7-membered carbocyclyl comprising 0, 1, or 2 double bonds in the carbocyclic ring system, as valency permits). In certain embodiments, R¹ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, R¹ is substituted or unsubstituted, monocyclic heterocyclyl (e.g., substituted or unsubstituted, 3- to 7-membered, monocyclic heterocyclyl). In certain embodiments, R¹ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, R¹ is substituted or unsubstituted phenyl. In certain embodiments, R¹ is substituted or unsubstituted, 5- to 6-membered, monocyclic heteroaryl. In certain embodiments, R¹ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, R¹ is substituted or unsubstituted pyridinyl, substituted or unsubstituted pyrazinyl, substituted or unsubstituted pyrimidinyl, or substituted or unsubstituted pyridazinyl. In certain embodiments, R¹ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, R¹ is —OMe. In certain embodiments, R¹ is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, R¹ is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, R¹ is —CN or —SCN. In certain embodiments, R¹ is —NO₂. In certain embodiments, R¹ is —C(═NR^{a})R^{a}, —C(═NR^{a})OR^{a}, or —C(═NR^{a})N(R^{a})₂. In certain embodiments, R¹ is —C(═O)R^{a} (e.g., —C(═O)(substituted or unsubstituted alkyl) (e.g., —C(═O)Me) or —C(═O)(substituted or unsubstituted phenyl)). In certain embodiments, R¹ is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, R¹ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R¹ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, R¹ is —NR^{a}C(═O)OR^{a}. In certain embodiments, R¹ is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, R¹ is —OC(═O)R^{a} (e.g., —OC(═O)(substituted or unsubstituted alkyl) or —OC(═O)(substituted or unsubstituted phenyl)), —OC(═O)OR^{a}(e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, R¹ is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)).

In certain embodiments, R¹ is hydrogen, —OCH₃, —CH₃, F, Cl, —CN, —CF₃, —OCF₃, —OC₂H₅, —OC₃H₇, —NH₂, —NHCH₃, —N(CH₃)₂, —C(═O)CH₃, —C(═O)NH₂, —SCH₃, —S(═O)CH₃, —S(═O)₂CH₃, —C(═O)OH, —CH₂OCH₃, or —OCH₂CH₂OCH₃. In certain embodiments, R¹ is hydrogen, —OCH₃, —CH₃, F, or C₁.

In certain embodiments, Z is CH. In certain embodiments, Z is CF. In certain embodiments, Z is N.

In certain embodiments, Cy² is monocyclic heteroaryl. In certain embodiments, Cy² is 5- or 6-membered, monocyclic heteroaryl. In certain embodiments, Cy² is furanyl, thienyl, pyrrolyl, imidazolyl, oxazolyl, isoxazolyl, thiazolyl, or isothiazolyl. In certain embodiments, Cy² is pyrazolyl. In certain embodiments, Cy² is pyridinyl, pyrazinyl, pyrimidinyl, or pyridazinyl. In certain embodiments, Cy² is furanyl or pyridinyl. In certain embodiments,

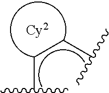 [SMILES: *CC*]

is

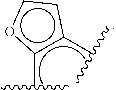

In certain embodiments,

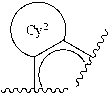 [SMILES: *CC*]

is

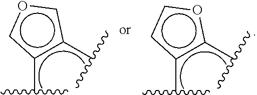

In certain embodiments,

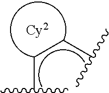 [SMILES: *CC*]

is

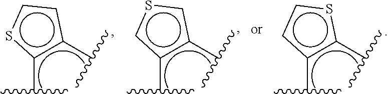

In certain embodiments,

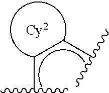 [SMILES: *CC*]

is

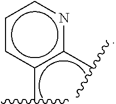

In certain embodiments,

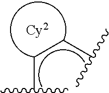 [SMILES: *CC*]

is

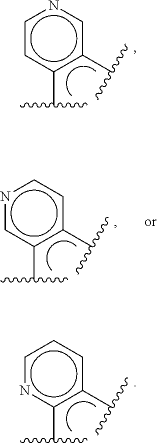

In certain embodiments, Cy² is monocyclic carbocyclyl (e.g., monocyclic, 3- to 7-membered carbocyclyl comprising 0, 1, or 2 double bonds in the carbocyclic ring system, as valency permits). In certain embodiments, Cy² is cyclopropyl, cyclobutyl, cyclopentyl, cyclohexyl, or cycloheptyl. In certain embodiments, Cy² is monocyclic heterocyclyl (e.g., 3- to 7-membered, monocyclic heterocyclyl). In certain embodiments, Cy² is oxetanyl, tetrahydrofuranyl, tetrahydropyranyl, azetidinyl, pyrrolidinyl, piperidinyl, morpholinyl, or piperazinyl. In certain embodiments, Cy² is phenyl.

In certain embodiments, each instance of R¹⁰ is hydrogen. In certain embodiments, at least one R¹⁰ is hydrogen. In certain embodiments, at least one R¹⁰ is not hydrogen. In certain embodiments, at least one instance of R¹⁰ is halogen (e.g., F, Cl, or Br). In certain embodiments, at least one instance of R¹⁰ is substituted alkyl (e.g., alkyl substituted with one or more instances of halogen (e.g., F)). In certain embodiments, at least one instance of R¹⁰ is —CF₃. In certain embodiments, at least one instance of R¹⁰ is unsubstituted alkyl. In certain embodiments, at least one instance of R¹⁰ is unsubstituted, C₁₋₆ alkyl. In certain embodiments, at least one instance of R¹⁰ is Me. In certain embodiments, at least one instance of R¹⁰ is Et, Pr, or Bu. In certain embodiments, at least one instance of R¹⁰ is substituted C₁₋₆ alkyl. In certain embodiments, at least one instance of R¹⁰ is substituted methyl. In certain embodiments, at least one instance of R¹⁰ is substituted ethyl, substituted propyl, or substituted butyl. In certain embodiments, at least one instance of R¹⁰ is substituted or unsubstituted alkenyl. In certain embodiments, at least one instance of R¹⁰ is substituted or unsubstituted, C₂₋₆ alkenyl (e.g., substituted or unsubstituted vinyl or substituted or unsubstituted allyl). In certain embodiments, at least one instance of R¹⁰ is substituted or unsubstituted alkynyl. In certain embodiments, at least one instance of R¹⁰ is substituted or unsubstituted, C₂₋₆ alkynyl (substituted or unsubstituted ethynyl). In certain embodiments, at least one instance of R¹⁰ is substituted or unsubstituted, monocyclic carbocyclyl (e.g., substituted or unsubstituted, monocyclic, 3- to 7-membered carbocyclyl comprising 0, 1, or 2 double bonds in the carbocyclic ring system, as valency permits). In certain embodiments, at least one instance of R¹⁰ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R¹⁰ is substituted or unsubstituted, monocyclic heterocyclyl (e.g., substituted or unsubstituted, 3- to 7-membered, monocyclic heterocyclyl). In certain embodiments, at least one instance of R¹⁰ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R¹⁰ is substituted or unsubstituted phenyl. In certain embodiments, at least one instance of R¹⁰ is substituted or unsubstituted, monocyclic heteroaryl. In certain embodiments, at least one instance of R¹⁰ is substituted or unsubstituted, 5- to 6-membered, monocyclic heteroaryl. In certain embodiments, at least one instance of R¹⁰ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R¹⁰ is substituted or unsubstituted pyridinyl, substituted or unsubstituted pyrazinyl, substituted or unsubstituted pyrimidinyl, or substituted or unsubstituted pyridazinyl. In certain embodiments, at least one instance of R¹⁰ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, at least one instance of R¹⁰ is —OMe. In certain embodiments, at least one instance of R¹⁰ is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, at least one instance of R¹⁰ is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe2)). In certain embodiments, at least one instance of R¹⁰ is —CN or —SCN. In certain embodiments, at least one instance of R¹⁰ is —NO₂. In certain embodiments, at least one instance of R¹⁰ is —C(═NR^{a})R^{a}, —C(═NR^{a})OR^{a}, or —C(═NR^{a})N(R^{a})₂. In certain embodiments, at least one instance of R¹⁰ is —C(═O)R^{a} (e.g., —C(═O)(substituted or unsubstituted alkyl) (e.g., —C(═O)Me) or —C(═O)(substituted or unsubstituted phenyl)). In certain embodiments, at least one instance of R¹⁰ is —C(═O)OR^{a}(e.g., —C(═O)OH, —C(═O)O(substituted or unsubstituted alkyl) (e.g., —C(═O)OMe), or —C(═O)O(substituted or unsubstituted phenyl)). In certain embodiments, at least one instance of R¹⁰ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, at least one instance of R¹⁰ is —NR^{a}C(═O)R^{a} (e.g., —NHC(═O)(substituted or unsubstituted, C₁₋₈ alkyl) (e.g., —NHC(═O)Me) or —NHC(═O)(substituted or unsubstituted phenyl)). In certain embodiments, at least one instance of R¹⁰ is —NR^{a}C(═O)OR^{a}. In certain embodiments, at least one instance of R¹⁰ is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, at least one instance of R¹⁰ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, at least one instance of R¹⁰ is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)). In certain embodiments, two instances of R¹⁰ on the same carbon atom taken together with the carbon atom form C(═O).

In certain embodiments, k is 0. In certain embodiments, k is 1. In certain embodiments, k is 2. In certain embodiments, k is 3. In certain embodiments, k is 4. In certain embodiments, k is 5. In certain embodiments, k is 6. In certain embodiments, k is 7. In certain embodiments, k is 8.

In certain embodiments, Z² is CR². In certain embodiments, Z² is CH. In certain embodiments, Z² is N.

In certain embodiments, R² is hydrogen. In certain embodiments, R² is halogen (e.g., F, Cl, or Br). In certain embodiments, R² is substituted alkyl (e.g., alkyl substituted with one or more instances of halogen (e.g., F)). In certain embodiments, R² is —CF₃. In certain embodiments, R² is unsubstituted alkyl. In certain embodiments, R² is unsubstituted, C₁₋₆ alkyl. In certain embodiments, R² is Me. In certain embodiments, R² is Et, Pr, or Bu. In certain embodiments, R² is substituted C₁₋₆ alkyl. In certain embodiments, R² is substituted methyl. In certain embodiments, R² is substituted ethyl, substituted propyl, or substituted butyl. In certain embodiments, R² is substituted or unsubstituted alkenyl. In certain embodiments, R² is substituted or unsubstituted, C₂₋₆ alkenyl (e.g., substituted or unsubstituted vinyl or substituted or unsubstituted allyl). In certain embodiments, R² is substituted or unsubstituted alkynyl. In certain embodiments, R² is substituted or unsubstituted, C₂₋₆ alkynyl (substituted or unsubstituted ethynyl). In certain embodiments, R² is substituted or unsubstituted, monocyclic carbocyclyl (e.g., substituted or unsubstituted, monocyclic, 3- to 7-membered carbocyclyl comprising 0, 1, or 2 double bonds in the carbocyclic ring system, as valency permits). In certain embodiments, R² is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, R² is substituted or unsubstituted, monocyclic heterocyclyl (e.g., substituted or unsubstituted, 3- to 7-membered, monocyclic heterocyclyl). In certain embodiments, R² is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, R² is substituted or unsubstituted phenyl. In certain embodiments, R² is substituted or unsubstituted, 5- to 6-membered, monocyclic heteroaryl. In certain embodiments, R² is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, R² is substituted or unsubstituted pyridinyl, substituted or unsubstituted pyrazinyl, substituted or unsubstituted pyrimidinyl, or substituted or unsubstituted pyridazinyl. In certain embodiments, R² is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, R² is —OMe. In certain embodiments, R² is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, R² is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, R² is —CN or —SCN. In certain embodiments, R² is —NO₂. In certain embodiments, R² is —C(═NR^{a})R^{a}, —C(═NR^{a})OR^{a}, or —C(═NR^{a})N(R^{a})₂. In certain embodiments, R² is —C(═O)R^{a} (e.g., —C(═O)(substituted or unsubstituted alkyl) (e.g., —C(═O)Me) or —C(═O)(substituted or unsubstituted phenyl)). In certain embodiments, R² is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, R² is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R² is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, R² is —NR^{a}C(═O)OR^{a}. In certain embodiments, R² is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, R² is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, R² is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)).

In certain embodiments, R² is hydrogen, —OCH₃, —CH₃, F, Cl, —CN, —CF₃, —OCF₃, —OC₂H₅, —OC₃H₇, —NH₂, —NHCH₃, —N(CH₃)₂, —C(═O)CH₃, —C(═O)NH₂, —SCH₃, —S(═O)CH₃, —S(═O)₂CH₃, —C(═O)OH, —CH₂OCH₃, or —OCH₂CH₂OCH₃. In certain embodiments, R² is hydrogen, —OCH₃, —CH₃, F, or C₁.

In certain embodiments, Cy³ is monocyclic heteroaryl. In certain embodiments, Cy³ is 5- or 6-membered, monocyclic heteroaryl. In certain embodiments, Cy³ is furanyl, thienyl, pyrrolyl, imidazolyl, oxazolyl, isoxazolyl, thiazolyl, or isothiazolyl. In certain embodiments, Cy³ is pyrazolyl. In certain embodiments, Cy³ is furanyl. In certain embodiments,

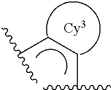 [SMILES: *CC*]

is

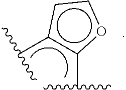

In certain embodiments,

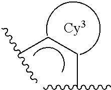 [SMILES: *CC*]

is

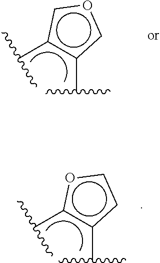

In certain embodiments,

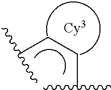 [SMILES: *CC*]

is

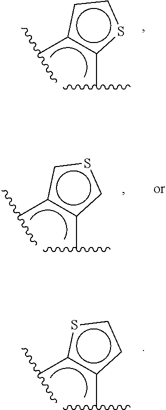

In certain embodiments, Cy³ is pyridinyl, pyrazinyl, pyrimidinyl, or pyridazinyl. In certain embodiments, Cy³ is pyridinyl. In certain embodiments,

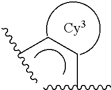 [SMILES: *CC*]

is

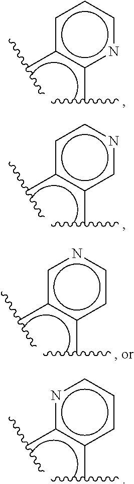

In certain embodiments, Cy³ is monocyclic carbocyclyl (e.g., monocyclic, 3- to 7-membered carbocyclyl comprising 0, 1, or 2 double bonds in the carbocyclic ring system, as valency permits). In certain embodiments, Cy³ is cyclopropyl, cyclobutyl, cyclopentyl, cyclohexyl, or cycloheptyl. In certain embodiments, Cy³ is monocyclic heterocyclyl (e.g., 3- to 7-membered, monocyclic heterocyclyl). In certain embodiments, Cy³ is oxetanyl, tetrahydrofuranyl, tetrahydropyranyl, azetidinyl, pyrrolidinyl, piperidinyl, morpholinyl, or piperazinyl. In certain embodiments, Cy³ is phenyl.

In certain embodiments, each instance of R¹¹ is hydrogen. In certain embodiments, at least one R¹¹ is hydrogen. In certain embodiments, at least one R¹¹ is not hydrogen. In certain embodiments, at least one instance of R¹¹ is halogen (e.g., F, Cl, or Br). In certain embodiments, at least one instance of R¹¹ is substituted alkyl (e.g., alkyl substituted with one or more instances of halogen (e.g., F)). In certain embodiments, at least one instance of R¹¹ is —CF₃. In certain embodiments, at least one instance of R¹¹ is unsubstituted alkyl. In certain embodiments, at least one instance of R¹¹ is unsubstituted, C₁₋₆ alkyl. In certain embodiments, at least one instance of R¹¹ is Me. In certain embodiments, at least one instance of R¹¹ is Et, Pr, or Bu. In certain embodiments, at least one instance of R¹¹ is substituted C₁₋₆ alkyl. In certain embodiments, at least one instance of R¹¹ is substituted methyl. In certain embodiments, at least one instance of R¹¹ is substituted ethyl, substituted propyl, or substituted butyl. In certain embodiments, at least one instance of R¹¹ is substituted or unsubstituted alkenyl. In certain embodiments, at least one instance of R¹¹ is substituted or unsubstituted, C₂₋₆ alkenyl (e.g., substituted or unsubstituted vinyl or substituted or unsubstituted allyl). In certain embodiments, at least one instance of R¹¹ is substituted or unsubstituted alkynyl. In certain embodiments, at least one instance of R¹¹ is substituted or unsubstituted, C₂₋₆ alkynyl (substituted or unsubstituted ethynyl). In certain embodiments, at least one instance of R¹¹ is substituted or unsubstituted, monocyclic carbocyclyl (e.g., substituted or unsubstituted, monocyclic, 3- to 7-membered carbocyclyl comprising 0, 1, or 2 double bonds in the carbocyclic ring system, as valency permits). In certain embodiments, at least one instance of R¹¹ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R¹¹ is substituted or unsubstituted, monocyclic heterocyclyl (e.g., substituted or unsubstituted, 3- to 7-membered, monocyclic heterocyclyl). In certain embodiments, at least one instance of R¹¹ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R¹¹ is substituted or unsubstituted phenyl. In certain embodiments, at least one instance of R¹¹ is substituted or unsubstituted, monocyclic heteroaryl. In certain embodiments, at least one instance of R¹¹ is substituted or unsubstituted, 5- to 6-membered, monocyclic heteroaryl. In certain embodiments, at least one instance of R¹¹ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R¹¹ is substituted or unsubstituted pyridinyl, substituted or unsubstituted pyrazinyl, substituted or unsubstituted pyrimidinyl, or substituted or unsubstituted pyridazinyl. In certain embodiments, at least one instance of R¹¹ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, at least one instance of R¹¹ is —OMe. In certain embodiments, at least one instance of R¹¹ is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, at least one instance of R¹¹ is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, at least one instance of R¹¹ is —CN or —SCN. In certain embodiments, at least one instance of R¹¹ is —NO₂. In certain embodiments, at least one instance of R¹¹ is —C(═NR^{a})R^{a}, —C(═NR^{a})OR^{a}, or —C(═NR^{a})N(R^{a})₂. In certain embodiments, at least one instance of R¹¹ is —C(═O)R^{a} (e.g., —C(═O)(substituted or unsubstituted alkyl) (e.g., —C(═O)Me) or —C(═O)(substituted or unsubstituted phenyl)). In certain embodiments, at least one instance of R¹¹ is —C(═O)OR^{a}(e.g., —C(═O)OH, —C(═O)O(substituted or unsubstituted alkyl) (e.g., —C(═O)OMe), or —C(═O)O(substituted or unsubstituted phenyl)). In certain embodiments, at least one instance of R¹¹ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, at least one instance of R¹¹ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, at least one instance of R¹¹ is —NR^{a}C(═O)OR^{a}. In certain embodiments, at least one instance of R¹¹ is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, at least one instance of R¹¹ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, at least one instance of R¹¹ is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)). In certain embodiments, two instances of R¹¹ on the same carbon atom taken together with the carbon atom form C(═O).

In certain embodiments, m is 0. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8.

In certain embodiments,

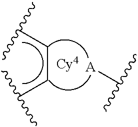

is

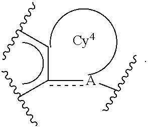

In certain embodiments, Cy⁴ is monocyclic heteroaryl. In certain embodiments, Cy⁴ is 5- or 6-membered, monocyclic heteroaryl. In certain embodiments, Cy⁴ is furanyl, thienyl, pyrrolyl, imidazolyl, oxazolyl, isoxazolyl, thiazolyl, or isothiazolyl. In certain embodiments, Cy⁴ is pyrrolyl. In certain embodiments, Cy⁴ is pyrazolyl. In certain embodiments, Cy⁴ is imidazolyl. In certain embodiments,

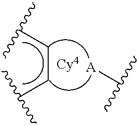

is

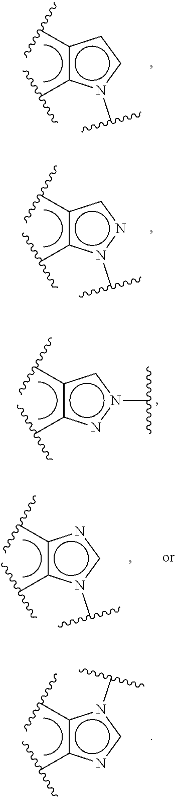

In certain embodiments,

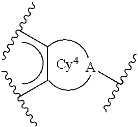

is

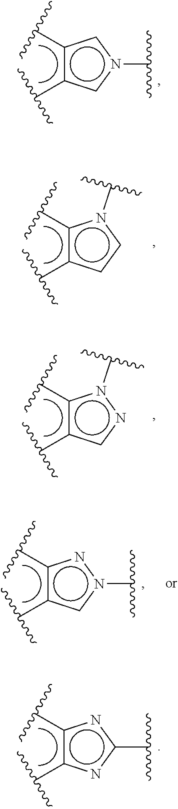

In certain embodiments, is N

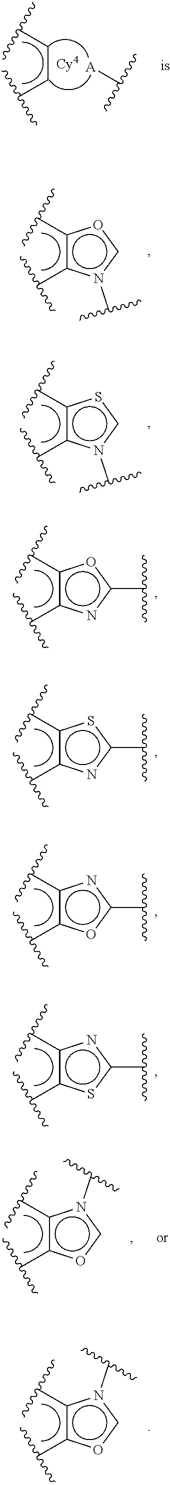

In certain embodiments, Cy⁴ is pyridinyl, pyrazinyl, pyrimidinyl, or pyridazinyl. In certain embodiments,

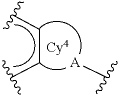

is

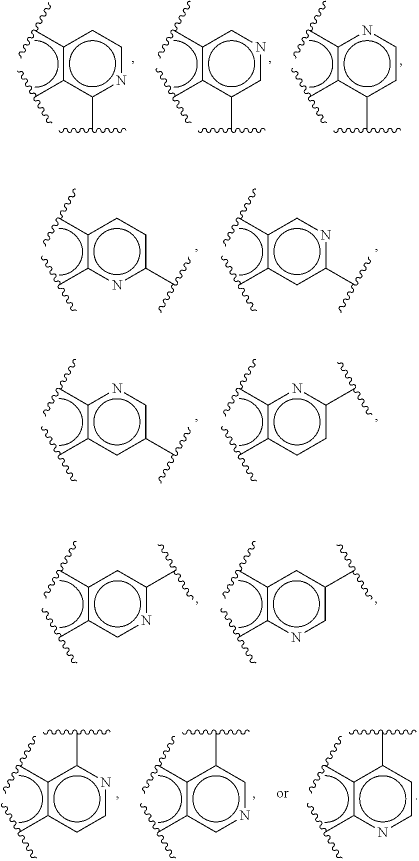

In certain embodiments, Cy⁴ is monocyclic carbocyclyl (e.g., monocyclic, 3- to 7-membered carbocyclyl comprising 0, 1, or 2 double bonds in the carbocyclic ring system, as valency permits). In certain embodiments, Cy⁴ is cyclopropyl, cyclobutyl, cyclopentyl, cyclohexyl, or cycloheptyl. In certain embodiments, Cy⁴ is monocyclic heterocyclyl (e.g., 3- to 7-membered, monocyclic heterocyclyl). In certain embodiments, Cy⁴ is oxetanyl, tetrahydrofuranyl, tetrahydropyranyl, azetidinyl, pyrrolidinyl, piperidinyl, morpholinyl, or piperazinyl. In certain embodiments, Cy⁴ is pyrrolidinyl. In certain embodiments,

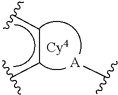

is

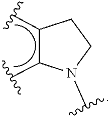 [SMILES: *C1CCN(*)C1*]

In certain embodiments,

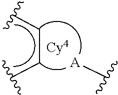

is

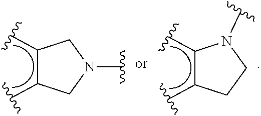 [SMILES: *C1CN(*)CC1* ; *C1CCN(*)C1*]

In certain embodiments,

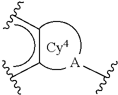

is

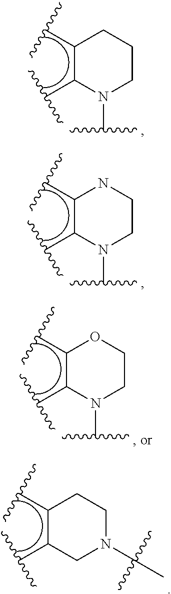

In certain embodiments,

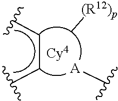

is

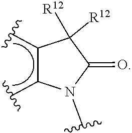

In certain embodiments

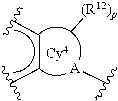

is

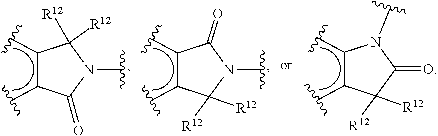

In certain embodiments,

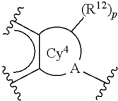

is

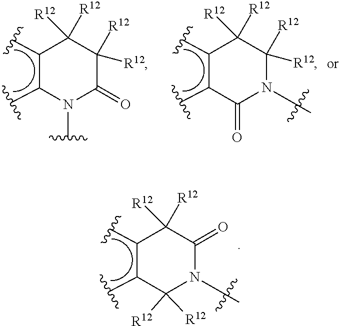

In certain embodiments, Cy⁴ is phenyl. In certain embodiments,

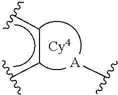

is

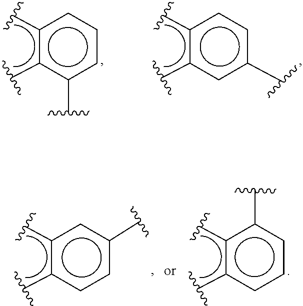

In certain embodiments, Cy⁴ is 5- or 6-membered, monocyclic heteroaryl or 5- or 6-membered, monocyclic heterocyclyl.

In certain embodiments, each instance of R¹² is hydrogen. In certain embodiments, at least one R¹² is hydrogen. In certain embodiments, at least one R¹² is not hydrogen. In certain embodiments, at least one instance of R¹² is halogen (e.g., F, Cl, or Br). In certain embodiments, at least one instance of R¹² is substituted alkyl (e.g., alkyl substituted with one or more instances of halogen (e.g., F)). In certain embodiments, at least one instance of R¹² is —CF₃. In certain embodiments, at least one instance of R¹² is unsubstituted alkyl. In certain embodiments, at least one instance of R¹² is unsubstituted, C₁₋₆ alkyl. In certain embodiments, at least one instance of R¹² is Me. In certain embodiments, at least one instance of R¹² is Et, Pr, or Bu. In certain embodiments, at least one instance of R¹² is substituted C₁₋₆ alkyl. In certain embodiments, at least one instance of R¹² is substituted methyl. In certain embodiments, at least one instance of R¹² is substituted ethyl, substituted propyl, or substituted butyl. In certain embodiments, at least one instance of R¹² is substituted or unsubstituted alkenyl. In certain embodiments, at least one instance of R¹² is substituted or unsubstituted, C₂₋₆ alkenyl (e.g., substituted or unsubstituted vinyl or substituted or unsubstituted allyl). In certain embodiments, at least one instance of R¹² is substituted or unsubstituted alkynyl. In certain embodiments, at least one instance of R¹² is substituted or unsubstituted, C₂₋₆ alkynyl (substituted or unsubstituted ethynyl). In certain embodiments, at least one instance of R¹² is substituted or unsubstituted, monocyclic carbocyclyl (e.g., substituted or unsubstituted, monocyclic, 3- to 7-membered carbocyclyl comprising 0, 1, or 2 double bonds in the carbocyclic ring system, as valency permits). In certain embodiments, at least one instance of R¹² is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R¹² is substituted or unsubstituted, monocyclic heterocyclyl (e.g., substituted or unsubstituted, 3- to 7-membered, monocyclic heterocyclyl). In certain embodiments, at least one instance of R¹² is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R¹² is substituted or unsubstituted phenyl. In certain embodiments, at least one instance of R¹² is substituted or unsubstituted, monocyclic heteroaryl. In certain embodiments, at least one instance of R¹² is substituted or unsubstituted, 5- to 6-membered, monocyclic heteroaryl. In certain embodiments, at least one instance of R¹² is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R¹² is substituted or unsubstituted pyridinyl, substituted or unsubstituted pyrazinyl, substituted or unsubstituted pyrimidinyl, or substituted or unsubstituted pyridazinyl. In certain embodiments, at least one instance of R¹² is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, at least one instance of R¹² is —OMe. In certain embodiments, at least one instance of R¹² is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, at least one instance of R¹² is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, at least one instance of R¹² is —CN or —SCN. In certain embodiments, at least one instance of R¹² is —NO₂. In certain embodiments, at least one instance of R¹² is —C(═NR^{a})R^{a}, —C(═NR^{a})OR^{a}, or —C(═NR^{a})N(R^{a})₂. In certain embodiments, at least one instance of R¹² is —C(═O)R^{a} (e.g., —C(═O)(substituted or unsubstituted alkyl) (e.g., —C(═O)Me) or —C(═O)(substituted or unsubstituted phenyl)). In certain embodiments, at least one instance of R¹² is —C(═O)OR^{a}(e.g., —C(═O)OH, —C(═O)O(substituted or unsubstituted alkyl) (e.g., —C(═O)OMe), or —C(═O)O(substituted or unsubstituted phenyl)). In certain embodiments, at least one instance of R¹² is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, at least one instance of R¹² is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, at least one instance of R¹² is —NR^{a}C(═O)OR^{a}. In certain embodiments, at least one instance of R¹² is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, at least one instance of R¹² is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, at least one instance of R¹² is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)). In certain embodiments, two instances of R¹² on the same carbon atom taken together with the carbon atom form C(═O).

In certain embodiments, p is 0. In certain embodiments, p is 1. In certain embodiments, p is 2. In certain embodiments, p is 3. In certain embodiments, p is 4. In certain embodiments, p is 5. In certain embodiments, p is 6. In certain embodiments, p is 0, 1, or 2.

In certain embodiments, Z¹ is C. In certain embodiments, Z¹ is CR¹⁵. In certain embodiments, Z¹ is CH. In certain embodiments, Z¹ is N. In certain embodiments, Z¹ is C or N.

In certain embodiments, R¹⁵ is hydrogen. In certain embodiments, R¹⁵ is halogen (e.g., F, Cl, or Br). In certain embodiments, R¹⁵ is substituted alkyl (e.g., alkyl substituted with one or more instances of halogen (e.g., F)). In certain embodiments, R¹⁵ is unsubstituted alkyl. In certain embodiments, R¹⁵ is unsubstituted, C₁₋₆ alkyl. In certain embodiments, R¹⁵ is Me. In certain embodiments, R¹⁵ is Et, Pr, or Bu. In certain embodiments, R¹⁵ is substituted C₁₋₆ alkyl. In certain embodiments, R¹⁵ is substituted methyl. In certain embodiments, R¹⁵ is substituted ethyl, substituted propyl, or substituted butyl.

In certain embodiments, Cy⁵ is monocyclic heteroaryl. In certain embodiments, Cy⁵ is 5- or 6-membered, monocyclic heteroaryl. In certain embodiments, Cy⁵ is furanyl, thienyl, pyrrolyl, imidazolyl, oxazolyl, isoxazolyl, thiazolyl, or isothiazolyl. In certain embodiments, Cy⁵ is pyridinyl, pyrazinyl, pyrimidinyl, or pyridazinyl. In certain embodiments, Cy⁵ is monocyclic carbocyclyl (e.g., monocyclic, 3- to 7-membered carbocyclyl comprising 0, 1, or 2 double bonds in the carbocyclic ring system, as valency permits). In certain embodiments, Cy⁵ is cyclopropyl, cyclobutyl, cyclopentyl, cyclohexyl, or cycloheptyl. In certain embodiments, Cy⁵ is monocyclic heterocyclyl (e.g., 3- to 7-membered, monocyclic heterocyclyl). In certain embodiments, Cy⁵ is oxetanyl, tetrahydrofuranyl, tetrahydropyranyl, azetidinyl, pyrrolidinyl, piperidinyl, morpholinyl, or piperazinyl. In certain embodiments, Cy⁵ is phenyl. In certain embodiments, Cy⁵ is 5- or 6-membered, monocyclic heteroaryl or 5- or 6-membered, monocyclic heterocyclyl. In certain embodiments,

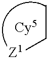

is

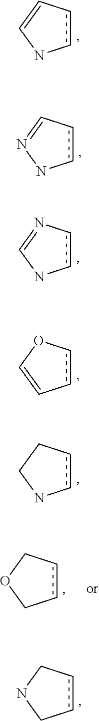

wherein [symbol] is a single bond or double bond. In certain embodiments

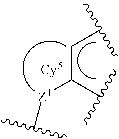

is

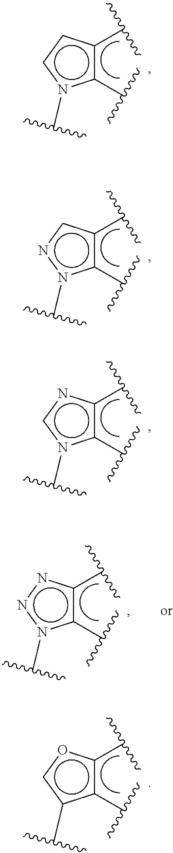

In certain embodiments is

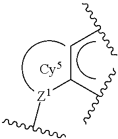

is

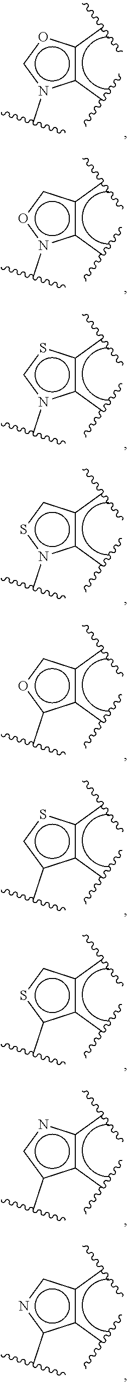

In certain embodiments,

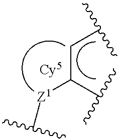

is

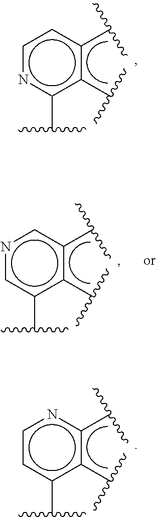

In certain embodiments,

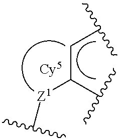

is

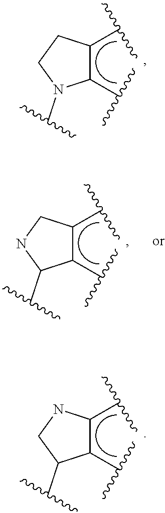 [SMILES: *C1CCN(*)C1* ; *C1CNC(*)C1* ; *C1CNC(*)C1*]

In certain embodiments,

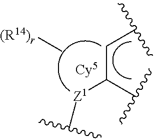

is

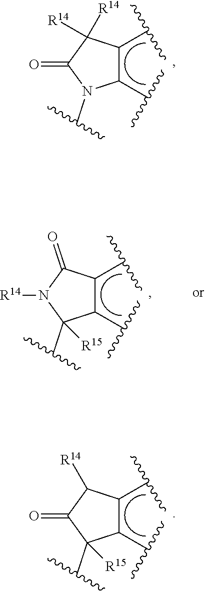

In certain embodiments,

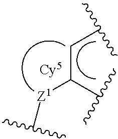

is

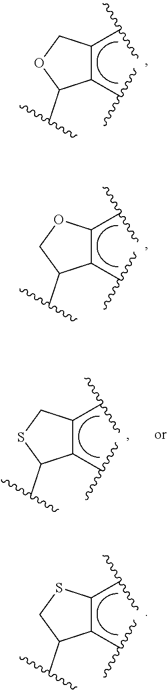 [SMILES: *C1COC(*)C1* ; *C1COC(*)C1* ; *C1CSC(*)C1* ; *C1CSC(*)C1*]

In certain embodiments, each instance of R¹⁴ is hydrogen. In certain embodiments, at least one R¹⁴ is hydrogen. In certain embodiments, at least one R¹⁴ is not hydrogen. In certain embodiments, at least one instance of R¹⁴ is halogen (e.g., F, Cl, or Br). In certain embodiments, at least one instance of R¹⁴ is substituted alkyl (e.g., alkyl substituted with one or more instances of halogen (e.g., F)). In certain embodiments, at least one instance of R¹⁴ is —CF₃. In certain embodiments, at least one instance of R¹⁴ is unsubstituted alkyl. In certain embodiments, at least one instance of R¹⁴ is unsubstituted, C₁₋₆ alkyl. In certain embodiments, at least one instance of R¹⁴ is Me. In certain embodiments, at least one instance of R¹⁴ is Et, Pr, or Bu. In certain embodiments, at least one instance of R¹⁴ is substituted C₁₋₆ alkyl. In certain embodiments, at least one instance of R¹⁴ is substituted methyl. In certain embodiments, at least one instance of R¹⁴ is substituted ethyl, substituted propyl, or substituted butyl. In certain embodiments, at least one instance of R¹⁴ is substituted or unsubstituted alkenyl. In certain embodiments, at least one instance of R¹⁴ is substituted or unsubstituted, C₂₋₆ alkenyl (e.g., substituted or unsubstituted vinyl or substituted or unsubstituted allyl). In certain embodiments, at least one instance of R¹⁴ is substituted or unsubstituted alkynyl. In certain embodiments, at least one instance of R¹⁴ is substituted or unsubstituted, C₂₋₆ alkynyl (substituted or unsubstituted ethynyl). In certain embodiments, at least one instance of R¹⁴ is substituted or unsubstituted, monocyclic carbocyclyl (e.g., substituted or unsubstituted, monocyclic, 3- to 7-membered carbocyclyl comprising 0, 1, or 2 double bonds in the carbocyclic ring system, as valency permits). In certain embodiments, at least one instance of R¹⁴ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R¹⁴ is substituted or unsubstituted, monocyclic heterocyclyl (e.g., substituted or unsubstituted, 3- to 7-membered, monocyclic heterocyclyl). In certain embodiments, at least one instance of R¹⁴ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R¹⁴ is substituted or unsubstituted phenyl. In certain embodiments, at least one instance of R¹⁴ is substituted or unsubstituted, monocyclic heteroaryl. In certain embodiments, at least one instance of R¹⁴ is substituted or unsubstituted, 5- to 6-membered, monocyclic heteroaryl. In certain embodiments, at least one instance of R¹⁴ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R¹⁴ is substituted or unsubstituted pyridinyl, substituted or unsubstituted pyrazinyl, substituted or unsubstituted pyrimidinyl, or substituted or unsubstituted pyridazinyl. In certain embodiments, at least one instance of R¹⁴ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, at least one instance of R¹⁴ is —OMe. In certain embodiments, at least one instance of R¹⁴ is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, at least one instance of R¹⁴ is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, at least one instance of R¹⁴ is —CN or —SCN. In certain embodiments, at least one instance of R¹⁴ is —NO₂. In certain embodiments, at least one instance of R¹⁴ is —C(═NR^{a})R^{a}, —C(═NR^{a})OR^{a}, or —C(═NR^{a})N(R^{a})₂. In certain embodiments, at least one instance of R¹⁴ is —C(═O)R^{a} (e.g., —C(═O)(substituted or unsubstituted alkyl) (e.g., —C(═O)Me) or —C(═O)(substituted or unsubstituted phenyl)). In certain embodiments, at least one instance of R¹⁴ is —C(═O)OR^{a}(e.g., —C(═O)OH, —C(═O)O(substituted or unsubstituted alkyl) (e.g., —C(═O)OMe), or —C(═O)O(substituted or unsubstituted phenyl)). In certain embodiments, at least one instance of R¹⁴ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, at least one instance of R¹⁴ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, at least one instance of R¹⁴ is —NR^{a}C(═O)OR^{a}. In certain embodiments, at least one instance of R¹⁴ is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, at least one instance of R¹⁴ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, at least one instance of R¹⁴ is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)). In certain embodiments, two instances of R¹⁴ on the same carbon atom taken together with the carbon atom form C(═O).

In certain embodiments, r is 0. In certain embodiments, r is 1. In certain embodiments, r is 2. In certain embodiments, r is 3. In certain embodiments, r is 4. In certain embodiments, r is 5. In certain embodiments, r is 6.

In certain embodiments, [symbol] is a single bond. In certain embodiments, [symbol] is a double bond.

In certain embodiments, A is O. In certain embodiments, A is NR¹⁶. In certain embodiments, A is NH. In certain embodiments, A is N(substituted or unsubstituted alkyl). In certain embodiments, A is N(unsubstituted C₁₋₆ alkyl) (e.g., NMe)). In certain embodiments, A is S. In certain embodiments, A is S(═O). In certain embodiments, A is C(═O). In certain embodiments, A is or C(R¹⁹)₂. In certain embodiments, A is CH₂. In certain embodiments, A is CH(substituted or unsubstituted alkyl). In certain embodiments, A is CH(unsubstituted C₁₋₆ alkyl). In certain embodiments, A is CHMe. In certain embodiments, A is C(substituted or unsubstituted alkyl)₂. In certain embodiments, A is C(unsubstituted C₁₋₆ alkyl)₂. In certain embodiments, A is C(Me)₂. In certain embodiments, A is O or NH.

In certain embodiments, A is N. In certain embodiments, A is C. In certain embodiments, A is CR¹⁷. In certain embodiments, A is CH. In certain embodiments, A is C(substituted or unsubstituted alkyl). In certain embodiments, A is C(unsubstituted C₁₋₆ alkyl). In certain embodiments, A is CMe.

In certain embodiments, R¹⁶ is hydrogen. In certain embodiments, R¹⁶ is substituted or unsubstituted acyl. In certain embodiments, R¹⁶ is —C(═O)R^{a} (e.g., —C(═O)(substituted or unsubstituted alkyl) (e.g., —C(═O)Me) or —C(═O)(substituted or unsubstituted phenyl)). In certain embodiments, R¹⁶ is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, R¹⁶ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R¹⁶ is unsubstituted alkyl. In certain embodiments, R¹⁶ is unsubstituted, C₁₋₆ alkyl. In certain embodiments, R¹⁶ is Me. In certain embodiments, R¹⁶ is Et, Pr, or Bu. In certain embodiments, R¹⁶ is substituted C₁₋₆ alkyl. In certain embodiments, R¹⁶ is substituted methyl. In certain embodiments, R¹⁶ is substituted ethyl, substituted propyl, or substituted butyl. In certain embodiments, R¹⁶ is a nitrogen protecting group (e.g., Bn, Boc, Cbz, Fmoc, trifluoroacetyl, triphenylmethyl, acetyl, or Ts).

In certain embodiments, each instance of R¹⁹ is hydrogen. In certain embodiments, at least one R¹⁹ is hydrogen. In certain embodiments, at least one R¹⁹ is not hydrogen. In certain embodiments, at least one instance of R¹⁹ is halogen (e.g., F, Cl, or Br). In certain embodiments, at least one instance of R¹⁹ is substituted alkyl (e.g., alkyl substituted with one or more instances of halogen (e.g., F)). In certain embodiments, at least one instance of R¹⁹ is —CF₃. In certain embodiments, at least one instance of R¹⁹ is unsubstituted alkyl. In certain embodiments, at least one instance of R¹⁹ is unsubstituted, C₁₋₆ alkyl. In certain embodiments, at least one instance of R¹⁹ is Me. In certain embodiments, at least one instance of R¹⁹ is Et, Pr, or Bu. In certain embodiments, at least one instance of R¹⁹ is substituted C₁₋₆ alkyl. In certain embodiments, at least one instance of R¹⁹ is substituted methyl. In certain embodiments, at least one instance of R¹⁹ is substituted ethyl, substituted propyl, or substituted butyl. In certain embodiments, at least one instance of R¹⁹ is substituted or unsubstituted alkenyl. In certain embodiments, at least one instance of R¹⁹ is substituted or unsubstituted, C₂₋₆ alkenyl (e.g., substituted or unsubstituted vinyl or substituted or unsubstituted allyl). In certain embodiments, at least one instance of R¹⁹ is substituted or unsubstituted alkynyl. In certain embodiments, at least one instance of R¹⁹ is substituted or unsubstituted, C₂₋₆ alkynyl (substituted or unsubstituted ethynyl). In certain embodiments, at least one instance of R¹⁹ is substituted or unsubstituted, monocyclic carbocyclyl (e.g., substituted or unsubstituted, monocyclic, 3- to 7-membered carbocyclyl comprising 0, 1, or 2 double bonds in the carbocyclic ring system, as valency permits). In certain embodiments, at least one instance of R¹⁹ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R¹⁹ is substituted or unsubstituted, monocyclic heterocyclyl (e.g., substituted or unsubstituted, 3- to 7-membered, monocyclic heterocyclyl). In certain embodiments, at least one instance of R¹⁹ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R¹⁹ is substituted or unsubstituted phenyl. In certain embodiments, at least one instance of R¹⁹ is substituted or unsubstituted, monocyclic heteroaryl. In certain embodiments, at least one instance of R¹⁹ is substituted or unsubstituted, 5- to 6-membered, monocyclic heteroaryl. In certain embodiments, at least one instance of R¹⁹ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R¹⁹ is substituted or unsubstituted pyridinyl, substituted or unsubstituted pyrazinyl, substituted or unsubstituted pyrimidinyl, or substituted or unsubstituted pyridazinyl. In certain embodiments, at least one instance of R¹⁹ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, at least one instance of R¹⁹ is —OMe. In certain embodiments, at least one instance of R¹⁹ is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, at least one instance of R¹⁹ is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, at least one instance of R¹⁹ is —CN or —SCN. In certain embodiments, at least one instance of R¹⁹ is —NO₂. In certain embodiments, at least one instance of R¹⁹ is —C(═NR^{a})R^{a}, —C(═NR^{a})OR^{a}, or —C(═NR^{a})N(R^{a})₂. In certain embodiments, at least one instance of R¹⁹ is —C(═O)R^{a} (e.g., —C(═O)(substituted or unsubstituted alkyl) (e.g., —C(═O)Me) or —C(═O)(substituted or unsubstituted phenyl)). In certain embodiments, at least one instance of R¹⁹ is —C(═O)OR^{a}(e.g., —C(═O)OH, —C(═O)O(substituted or unsubstituted alkyl) (e.g., —C(═O)OMe), or —C(═O)O(substituted or unsubstituted phenyl)). In certain embodiments, at least one instance of R¹⁹ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, at least one instance of R¹⁹ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, at least one instance of R¹⁹ is —NR^{a}C(═O)OR^{a}. In certain embodiments, at least one instance of R¹⁹ is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, at least one instance of R¹⁹ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, at least one instance of R¹⁹ is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)).

In certain embodiments, R¹⁷ is hydrogen. In certain embodiments, R¹⁷ is halogen (e.g., F, Cl, or Br). In certain embodiments, R¹⁷ is substituted alkyl (e.g., alkyl substituted with one or more instances of halogen (e.g., F)). In certain embodiments, R¹⁷ is unsubstituted alkyl. In certain embodiments, R¹⁷ is unsubstituted, C₁₋₆ alkyl. In certain embodiments, R¹⁷ is Me. In certain embodiments, R¹⁷ is Et, Pr, or Bu. In certain embodiments, R¹⁷ is substituted C₁₋₆ alkyl. In certain embodiments, R¹⁷ is substituted methyl. In certain embodiments, R¹⁷ is substituted ethyl, substituted propyl, or substituted butyl.

In certain embodiments, B is C(R³)(R⁴). In certain embodiments, B is C(R³)(R⁴), wherein at least one of R³ and R⁴ is hydrogen, halogen, substituted or unsubstituted alkyl, or —OR^{a}. In certain embodiments, B is CH₂. In certain embodiments, B is CH(substituted or unsubstituted alkyl). In certain embodiments, B is CH(unsubstituted C₁₋₆ alkyl). In certain embodiments, B is CHMe. In certain embodiments, B is C(substituted or unsubstituted alkyl)₂. In certain embodiments, B is C(unsubstituted C₁₋₆ alkyl)₂. In certain embodiments, B is C(Me)₂. In certain embodiments, B is C(═O). In certain embodiments, B is NR¹⁸. In certain embodiments, B is NH. In certain embodiments, B is N(substituted or unsubstituted alkyl). In certain embodiments, B is N(unsubstituted C₁₋₆ alkyl) (e.g., NMe)). In certain embodiments, B is a single bond. In certain embodiments, in Formula (V), B is a single bond. In certain embodiments, in Formula (I), (II), (III), or (IV), B is not a single bond.

In certain embodiments, A and B are joined to form substituted or unsubstituted, monocyclic carbocyclyl (e.g., substituted or unsubstituted, monocyclic, 3- to 7-membered carbocyclyl comprising 0, 1, or 2 double bonds in the carbocyclic ring system, as valency permits). In certain embodiments, A and B are joined to form substituted or unsubstituted cyclopropyl. In certain embodiments, A and B are joined to form substituted or unsubstituted cyclobutyl, substituted or unsubstituted cyclopentyl, substituted or unsubstituted cyclohexyl, or substituted or unsubstituted cycloheptyl. In certain embodiments,

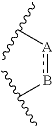

is

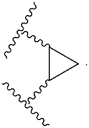

In certain embodiments,

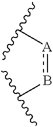

is

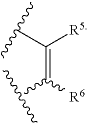

In certain embodiments,

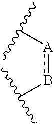

is

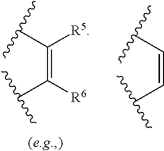

In certain embodiments,

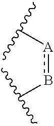

is

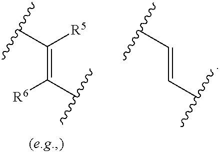

In certain embodiments,

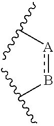

is

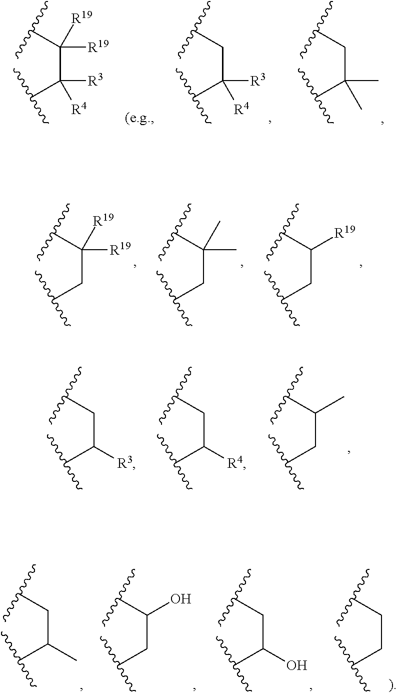

In certain embodiments,

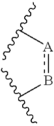

is

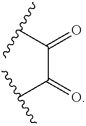 [SMILES: *C(=O)C(*)=O]

In certain embodiments,

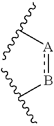

is

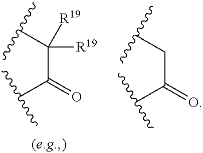

In certain embodiments,

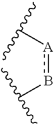

is

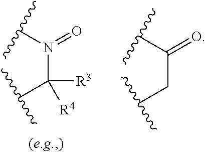

In certain embodiments,

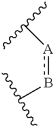

is

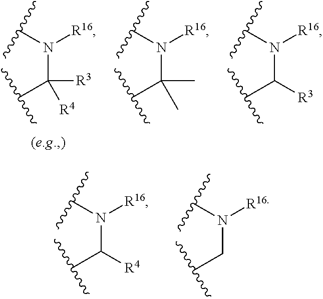

In certain embodiments,

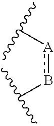

is

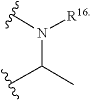

In certain embodiments,

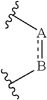

is

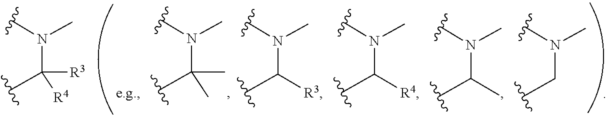

In certain embodiments,

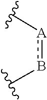

is

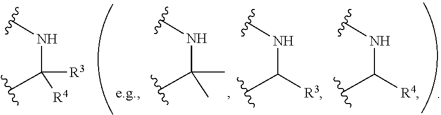

In certain embodiments

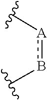

is

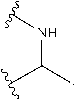

In certain embodiments,

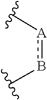

is

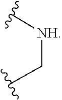 [SMILES: *CN*]

In certain embodiments,

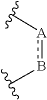

is

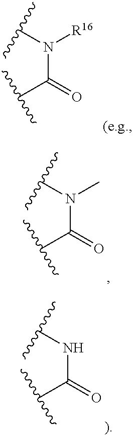

In certain embodiments,

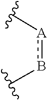

is

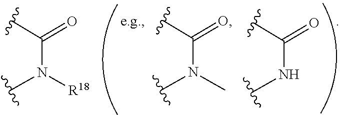

In certain embodiments,

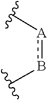

is

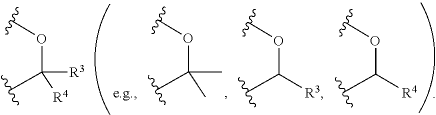

In certain embodiments,

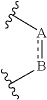

is

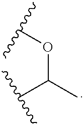 [SMILES: *OC(*)C]

In certain embodiments,

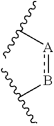

is

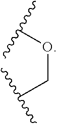 [SMILES: *CO*]

In certain embodiments,

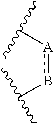

is

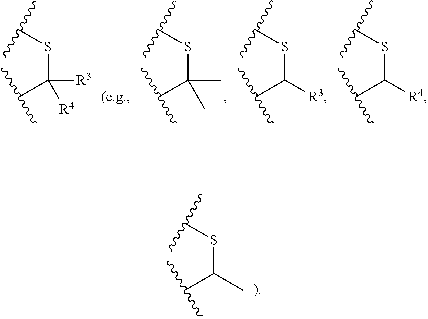

In certain embodiments,

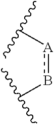

is

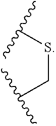 [SMILES: *CS*]

In certain embodiments,

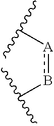

is

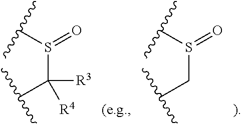

In certain embodiments,

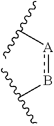

is —O—. In certain embodiments,

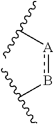

is —S—. In certain embodiments,

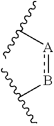

is —N(R¹⁶)— (e.g., —N(Me)-). In certain embodiments,

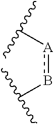

is —NH—. In certain embodiments,

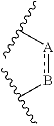

is —C(R¹⁹)₂— (e.g., —C(Me)₂—CH(R¹⁹)—, —CH(Me)-, —CH(OH)—, —CH₂—). In certain embodiments,

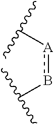

### is C(═O) or S(═O).

In certain embodiments, R³ is hydrogen. In certain embodiments, R³ is halogen (e.g., F, Cl, or Br). In certain embodiments, R³ is substituted alkyl (e.g., alkyl substituted with one or more instances of halogen (e.g., F)). In certain embodiments, R³ is —CF₃. In certain embodiments, R³ is unsubstituted alkyl. In certain embodiments, R³ is unsubstituted, C₁₋₆ alkyl. In certain embodiments, R³ is Me. In certain embodiments, R³ is Et, Pr, or Bu. In certain embodiments, R³ is substituted C₁₋₆ alkyl. In certain embodiments, R³ is substituted methyl. In certain embodiments, R³ is substituted ethyl, substituted propyl, or substituted butyl. In certain embodiments, R³ is substituted or unsubstituted alkenyl. In certain embodiments, R³ is substituted or unsubstituted, C₂₋₆ alkenyl (e.g., substituted or unsubstituted vinyl or substituted or unsubstituted allyl). In certain embodiments, R³ is substituted or unsubstituted alkynyl. In certain embodiments, R³ is substituted or unsubstituted, C₂₋₆ alkynyl (substituted or unsubstituted ethynyl). In certain embodiments, R³ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, R³ is —OMe. In certain embodiments, R³ is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, R³ is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, R³ is —CN or —SCN. In certain embodiments, R³ is —NO₂. In certain embodiments, R³ is —C(═NR^{a})R^{a}, —C(═NR^{a})OR^{a}, or —C(═NR^{a})N(R^{a})₂. In certain embodiments, R³ is —C(═O)R^{a} (e.g., —C(═O)(substituted or unsubstituted alkyl) (e.g., —C(═O)Me) or —C(═O)(substituted or unsubstituted phenyl)). In certain embodiments, R³ is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, R³ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R³ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, R³ is —NR^{a}C(═O)OR^{a}. In certain embodiments, R³ is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, R³ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, R³ is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)).

In certain embodiments, R³ is hydrogen, —OCH₃, —CH₃, F, Cl, —CN, —CF₃, —OCF₃, —OC₂H₅, —OC₃H₇, —NH₂, —NHCH₃, —N(CH₃)₂, —C(═O)CH₃, —C(═O)NH₂, —SCH₃, —S(═O)CH₃, —S(═O)₂CH₃, —C(═O)OH, —CH₂OCH₃, or —OCH₂CH₂OCH₃. In certain embodiments, R³ is hydrogen, —CH₃, —OCH₃, —OH, F, or C₁.

In certain embodiments, R⁴ is hydrogen. In certain embodiments, R⁴ is halogen (e.g., F, Cl, or Br). In certain embodiments, R⁴ is substituted alkyl (e.g., alkyl substituted with one or more instances of halogen (e.g., F)). In certain embodiments, R⁴ is —CF₃. In certain embodiments, R⁴ is unsubstituted alkyl. In certain embodiments, R⁴ is unsubstituted, C₁₋₆ alkyl. In certain embodiments, R⁴ is Me. In certain embodiments, R⁴ is Et, Pr, or Bu. In certain embodiments, R⁴ is substituted C₁₋₆ alkyl. In certain embodiments, R⁴ is substituted methyl. In certain embodiments, R⁴ is substituted ethyl, substituted propyl, or substituted butyl. In certain embodiments, R⁴ is substituted or unsubstituted alkenyl. In certain embodiments, R⁴ is substituted or unsubstituted, C₂₋₆ alkenyl (e.g., substituted or unsubstituted vinyl or substituted or unsubstituted allyl). In certain embodiments, R⁴ is substituted or unsubstituted alkynyl. In certain embodiments, R⁴ is substituted or unsubstituted, C₂₋₆ alkynyl (substituted or unsubstituted ethynyl). In certain embodiments, R⁴ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, R⁴ is —OMe. In certain embodiments, R⁴ is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, R⁴ is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, R⁴ is —CN or —SCN. In certain embodiments, R⁴ is —NO₂. In certain embodiments, R⁴ is —C(═NR^{a})R^{a}, —C(═NR^{a})OR^{a}, or —C(═NR^{a})N(R^{a})₂. In certain embodiments, R⁴ is —C(═O)R^{a} (e.g., —C(═O)(substituted or unsubstituted alkyl) (e.g., —C(═O)Me) or —C(═O)(substituted or unsubstituted phenyl)). In certain embodiments, R⁴ is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, R⁴ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R⁴ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, R⁴ is —NR^{a}C(═O)OR^{a}. In certain embodiments, R⁴ is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, R⁴ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, R⁴ is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)).

In certain embodiments, R⁴ is hydrogen, —OCH₃, —CH₃, F, Cl, —CN, —CF₃, —OCF₃, —OC₂H₅, —OC₃H₇, —NH₂, —NHCH₃, —N(CH₃)₂, —C(═O)CH₃, —C(═O)NH₂, —SCH₃, —S(═O)CH₃, —S(═O)₂CH₃, —C(═O)OH, —CH₂OCH₃, or —OCH₂CH₂OCH₃. In certain embodiments, R⁴ is hydrogen, —CH₃, —OCH₃, —OH, F, or C₁.

In certain embodiments, each of R³ and R⁴ is independently hydrogen, —CH₃, —OCH₃, —OH, F, or C₁. In certain embodiments, each of R³ and R⁴ is hydrogen.

In certain embodiments, R¹⁸ is hydrogen. In certain embodiments, R¹⁸ is substituted or unsubstituted acyl. In certain embodiments, R¹⁸ is —C(═O)R^{a} (e.g., —C(═O)(substituted or unsubstituted alkyl) (e.g., —C(═O)Me) or —C(═O)(substituted or unsubstituted phenyl)). In certain embodiments, R¹⁸ is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, R¹⁸ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R¹⁸ is unsubstituted alkyl. In certain embodiments, R¹⁸ is unsubstituted, C₁₋₆ alkyl. In certain embodiments, R¹⁸ is Me. In certain embodiments, R¹⁸ is Et, Pr, or Bu. In certain embodiments, R¹⁸ is substituted C₁₋₆ alkyl. In certain embodiments, R¹⁸ is substituted methyl. In certain embodiments, R¹⁸ is substituted ethyl, substituted propyl, or substituted butyl. In certain embodiments, R¹⁸ is a nitrogen protecting group (e.g., Bn, Boc, Cbz, Fmoc, trifluoroacetyl, triphenylmethyl, acetyl, or Ts).

In certain embodiments, R⁵ is hydrogen. In certain embodiments, R⁵ is halogen (e.g., F, Cl, or Br). In certain embodiments, R⁵ is substituted alkyl (e.g., alkyl substituted with one or more instances of halogen (e.g., F)). In certain embodiments, R⁵ is —CF₃. In certain embodiments, R⁵ is unsubstituted alkyl. In certain embodiments, R⁵ is unsubstituted, C₁₋₆ alkyl. In certain embodiments, R⁵ is Me. In certain embodiments, R⁵ is Et, Pr, or Bu. In certain embodiments, R⁵ is substituted C₁₋₆ alkyl. In certain embodiments, R⁵ is substituted methyl. In certain embodiments, R⁵ is substituted ethyl, substituted propyl, or substituted butyl. In certain embodiments, R⁵ is substituted or unsubstituted alkenyl. In certain embodiments, R⁵ is substituted or unsubstituted, C₂₋₆ alkenyl (e.g., substituted or unsubstituted vinyl or substituted or unsubstituted allyl). In certain embodiments, R⁵ is substituted or unsubstituted alkynyl. In certain embodiments, R⁵ is substituted or unsubstituted, C₂₋₆ alkynyl (substituted or unsubstituted ethynyl). In certain embodiments, R⁵ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, R⁵ is —OMe. In certain embodiments, R⁵ is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, R⁵ is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, R⁵ is —CN or —SCN. In certain embodiments, R⁵ is —NO₂. In certain embodiments, R⁵ is —C(═NR^{a})R^{a}, —C(═NR^{a})OR^{a}, or —C(═NR^{a})N(R^{a})₂. In certain embodiments, R⁵ is —C(═O)R^{a} (e.g., —C(═O)(substituted or unsubstituted alkyl) (e.g., —C(═O)Me) or —C(═O)(substituted or unsubstituted phenyl)). In certain embodiments, R⁵ is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, R⁵ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R⁵ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, R⁵ is —NR^{a}C(═O)OR^{a}. In certain embodiments, R⁵ is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, R⁵ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, R⁵ is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)).

In certain embodiments, R⁵ is hydrogen, —OCH₃, —CH₃, F, Cl, —CN, —CF₃, —OCF₃, —OC₂H₅, —OC₃H₇, —NH₂, —NHCH₃, —N(CH₃)₂, —C(═O)CH₃, —C(═O)NH₂, —SCH₃, —S(═O)CH₃, —S(═O)₂CH₃, —C(═O)OH, —CH₂OCH₃, or —OCH₂CH₂OCH₃. In certain embodiments, R⁵ is hydrogen, —CH₃, —OCH₃, —OH, F, or C₁.

In certain embodiments, R⁶ is hydrogen. In certain embodiments, R⁶ is halogen (e.g., F, Cl, or Br). In certain embodiments, R⁶ is substituted alkyl (e.g., alkyl substituted with one or more instances of halogen (e.g., F)). In certain embodiments, R⁶ is —CF₃. In certain embodiments, R⁶ is unsubstituted alkyl. In certain embodiments, R⁶ is unsubstituted, C₁₋₆ alkyl. In certain embodiments, R⁶ is Me. In certain embodiments, R⁶ is Et, Pr, or Bu. In certain embodiments, R⁶ is substituted C₁₋₆ alkyl. In certain embodiments, R⁶ is substituted methyl. In certain embodiments, R⁶ is substituted ethyl, substituted propyl, or substituted butyl. In certain embodiments, R⁶ is substituted or unsubstituted alkenyl. In certain embodiments, R⁶ is substituted or unsubstituted, C₂₋₆ alkenyl (e.g., substituted or unsubstituted vinyl or substituted or unsubstituted allyl). In certain embodiments, R⁶ is substituted or unsubstituted alkynyl. In certain embodiments, R⁶ is substituted or unsubstituted, C₂₋₆ alkynyl (substituted or unsubstituted ethynyl). In certain embodiments, R⁶ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, R⁶ is —OMe. In certain embodiments, R⁶ is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, R⁶ is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, R⁶ is —CN or —SCN. In certain embodiments, R⁶ is —NO₂. In certain embodiments, R⁶ is —C(═NR^{a})R^{a}, —C(═NR^{a})OR^{a}, or —C(═NR^{a})N(R^{a})₂. In certain embodiments, R⁶ is —C(═O)R^{a} (e.g., —C(═O)(substituted or unsubstituted alkyl) (e.g., —C(═O)Me) or —C(═O)(substituted or unsubstituted phenyl)). In certain embodiments, R⁶ is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, R⁶ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R⁶ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, R⁶ is —NR^{a}C(═O)OR^{a}. In certain embodiments, R⁶ is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, R⁶ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, R⁶ is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)).

In certain embodiments, R⁶ is hydrogen, —OCH₃, —CH₃, F, Cl, —CN, —CF₃, —OCF₃, —OC₂H₅, —OC₃H₇, —NH₂, —NHCH₃, —N(CH₃)₂, —C(═O)CH₃, —C(═O)NH₂, —SCH₃, —S(═O)CH₃, —S(═O)₂CH₃, —C(═O)OH, —CH₂OCH₃, or —OCH₂CH₂OCH₃. In certain embodiments, R⁶ is hydrogen, —CH₃, —OCH₃, —OH, F, or C₁.

In certain embodiments, each of R⁵ and R⁶ is hydrogen. In certain embodiments, R⁵ and R⁶ are cis to each other. In certain embodiments, R⁵ and R⁶ are trans to each other. In certain embodiments, R⁵ and R⁶ are E to each other. In certain embodiments, R⁵ and R⁶ are Z to each other.

In certain embodiments, Cy¹ is heteroaryl. In certain embodiments, Cy¹ is monocyclic heteroaryl. In certain embodiments, Cy¹ is 5- or 6-membered, monocyclic heteroaryl. In certain embodiments, Cy¹ is furanyl, thienyl, pyrrolyl, imidazolyl, oxazolyl, isoxazolyl, thiazolyl, or isothiazolyl. In certain embodiments, Cy¹ is pyridinyl, pyrazinyl, pyrimidinyl, or pyridazinyl. In certain embodiments, Cy¹ is 9- or 10-membered, bicyclic heteroaryl. In certain embodiments, Cy¹ is aryl. In certain embodiments, Cy¹ is phenyl.

In certain embodiments, Cy¹ is naphthyl. In certain embodiments, Cy¹ is carbocyclyl. In certain embodiments, Cy¹ is monocyclic carbocyclyl (e.g., monocyclic, 3- to 7-membered carbocyclyl comprising 0, 1, or 2 double bonds in the carbocyclic ring system, as valency permits). In certain embodiments, Cy¹ is cyclopropyl, cyclobutyl, cyclopentyl, cyclohexyl, or cycloheptyl. In certain embodiments, Cy¹ is cyclopropyl. In certain embodiments, Cy¹ is cyclohexyl. In certain embodiments, Cy¹ is bicyclic carbocyclyl (e.g., bicyclic, 5- to 13-membered carbocyclyl comprising 0, 1, or 2 double bonds in the carbocyclic ring system, as valency permits). In certain embodiments, Cy¹ is tricyclic carbocyclyl (e.g., tricyclic, 6- to 13-membered carbocyclyl comprising 0, 1, or 2 double bonds in the carbocyclic ring system, as valency permits). In certain embodiments, Cy¹ is adamantyl. In certain embodiments, Cy¹ is heterocyclyl. In certain embodiments, Cy¹ is monocyclic heterocyclyl (e.g., 3- to 7-membered, monocyclic heterocyclyl). In certain embodiments, Cy¹ is oxetanyl, tetrahydrofuranyl, tetrahydropyranyl, azetidinyl, pyrrolidinyl, piperidinyl, morpholinyl, or piperazinyl. In certain embodiments, Cy¹ is pyrrolidinyl (e.g., 1-pyrrolidinyl, 2-pyrrolidinyl, 3-pyrrolidinyl). In certain embodiments, Cy¹ is tetrahydrofuranyl (e.g., 2-tetrahydrofuranyl, 3-tetrahydrofuranyl). In certain embodiments, Cy¹ is tetrahydrothienyl (e.g., 2-tetrahydrothienyl, 3-tetrahydrothienyl). In certain embodiments, Cy¹ is piperidinyl (e.g., 1-piperidinyl, 2-piperidinyl, 3-piperidinyl, 4-piperidinyl). In certain embodiments, Cy¹ is 5- or 6-membered, monocyclic heteroaryl or phenyl. In certain embodiments, Cy¹ is 2-thienyl, 2-furanyl, or 3-furanyl. In certain embodiments, Cy¹ is 2-thienyl. In certain embodiments, Cy¹ is 3-thienyl. In certain embodiments, Cy¹ is 2-furanyl. In certain embodiments, Cy¹ is 3-furanyl. In certain embodiments, Cy¹ is pyrrolyl (e.g., 1-pyrrolyl, 2-pyrrolyl, 3-pyrrolyl). In certain embodiments, Cy¹ is pyrazolyl (e.g., 1-pyrazolyl, 3-pyrazolyl, 4-pyrazolyl, 5-pyrazolyl). In certain embodiments, Cy¹ is imidazolyl (e.g., 1-imidazolyl, 2-imidazolyl, 4-imidazolyl, 5-imidazolyl). In certain embodiments, Cy¹ is oxazolyl (e.g., 2-oxazolyl, 4-oxazolyl, 5-oxazolyl). In certain embodiments, Cy¹ is isoxazolyl (e.g., 3-isoxazolyl, 4-isoxazolyl, 5-isoxazolyl). In certain embodiments, Cy¹ is thiazolyl (e.g., 2-thiazolyl, 4-thiazolyl, 5-thiazolyl). In certain embodiments, Cy¹ is isothiazolyl (e.g., 3-isothiazolyl, 4-isothiazolyl, 5-isothiazolyl). In certain embodiments, Cy¹ is pyridinyl (e.g., 2-pyridinyl, 3-pyridinyl, 4-pyridinyl).

In certain embodiments, each instance of R⁹ is hydrogen. In certain embodiments, at least one R⁹ is hydrogen. In certain embodiments, at least one R⁹ is not hydrogen. In certain embodiments, at least one instance of R⁹ is halogen (e.g., F, Cl, or Br). In certain embodiments, at least one instance of R⁹ is substituted alkyl (e.g., alkyl substituted with one or more instances of halogen (e.g., F)). In certain embodiments, at least one instance of R⁹ is —CF₃. In certain embodiments, at least one instance of R⁹ is unsubstituted alkyl. In certain embodiments, at least one instance of R⁹ is unsubstituted, C₁₋₆ alkyl. In certain embodiments, at least one instance of R⁹ is Me. In certain embodiments, at least one instance of R⁹ is Et, Pr, or Bu. In certain embodiments, at least one instance of R⁹ is substituted C₁₋₆ alkyl. In certain embodiments, at least one instance of R⁹ is substituted methyl. In certain embodiments, at least one instance of R⁹ is substituted ethyl, substituted propyl, or substituted butyl. In certain embodiments, at least one instance of R⁹ is substituted or unsubstituted alkenyl. In certain embodiments, at least one instance of R⁹ is substituted or unsubstituted, C₂₋₆ alkenyl (e.g., substituted or unsubstituted vinyl or substituted or unsubstituted allyl). In certain embodiments, at least one instance of R⁹ is substituted or unsubstituted alkynyl. In certain embodiments, at least one instance of R⁹ is substituted or unsubstituted, C₂₋₆ alkynyl (substituted or unsubstituted ethynyl). In certain embodiments, at least one instance of R⁹ is substituted or unsubstituted, monocyclic carbocyclyl (e.g., substituted or unsubstituted, monocyclic, 3- to 7-membered carbocyclyl comprising 0, 1, or 2 double bonds in the carbocyclic ring system, as valency permits). In certain embodiments, at least one instance of R⁹ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R⁹ is substituted or unsubstituted, monocyclic heterocyclyl (e.g., substituted or unsubstituted, 3- to 7-membered, monocyclic heterocyclyl). In certain embodiments, at least one instance of R⁹ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R⁹ is substituted or unsubstituted phenyl. In certain embodiments, at least one instance of R⁹ is substituted or unsubstituted, monocyclic heteroaryl. In certain embodiments, at least one instance of R⁹ is substituted or unsubstituted, 5- to 6-membered, monocyclic heteroaryl. In certain embodiments, at least one instance of R⁹ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R⁹ is substituted or unsubstituted pyridinyl, substituted or unsubstituted pyrazinyl, substituted or unsubstituted pyrimidinyl, or substituted or unsubstituted pyridazinyl. In certain embodiments, at least one instance of R⁹ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, at least one instance of R⁹ is —OMe. In certain embodiments, at least one instance of R⁹ is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, at least one instance of R⁹ is —N(R^{a})₂ (e.g., —NH₂, —NH(substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, at least one instance of R⁹ is —CN or —SCN. In certain embodiments, at least one instance of R⁹ is —NO₂. In certain embodiments, at least one instance of R⁹ is —C(═NR^{a})R^{a}, —C(═NR^{a})OR^{a}, or —C(═NR^{a})N(R^{a})₂. In certain embodiments, at least one instance of R⁹ is —C(═O)R^{a} (e.g., —C(═O)(substituted or unsubstituted alkyl) (e.g., —C(═O)Me) or —C(═O)(substituted or unsubstituted phenyl)). In certain embodiments, at least one instance of R⁹ is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, at least one instance of R⁹ is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, at least one instance of R⁹ is —NR^{a}C(═O)R^{a}(e.g., —NHC(═O)(substituted or unsubstituted, C₁₋₆ alkyl) (e.g., —NHC(═O)Me) or —NHC(═O)(substituted or unsubstituted phenyl)). In certain embodiments, at least one instance of R⁹ is —NR^{a}C(═O)OR^{a}. In certain embodiments, at least one instance of R⁹ is —NR^{a}C(═O)N(R^{a})₂ (e.g., —NHC(═O)NH₂, —NHC(═O)NH(substituted or unsubstituted, C₁₋₆ alkyl) (e.g., —NHC(═O)NHMe)). In certain embodiments, at least one instance of R⁹ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)). In certain embodiments, at least one instance of R⁹ is —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂ (optionally wherein at least one R^{a} is substituted or unsubstituted alkyl (e.g., unsubstituted C₁₋₆ alkyl)). In certain embodiments, two instances of R⁹ on the same carbon atom taken together with the carbon atom form C(═O).

In certain embodiments, at least one instance of R⁹ is F, Cl, —OCH₃, —OC₂H₅, —OC₃H₇, —CH₃, —OH, —NH₂, —NHCH₃, —N(CH₃)₂, —CN, —CF₃, —OCF₃, —C(═O)CH₃, —C(═O)OH, —SCH₃, —S(═O)CH₃, or —S(═O)₂CH₃. In certain embodiments, at least one instance of R⁹ is F, Cl, —OCH₃, or —CH₃.

In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10. In certain embodiments, n is 11.

In certain embodiments,

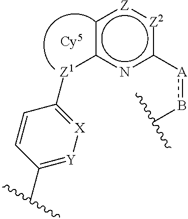

is not

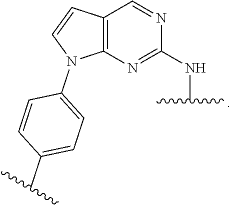 [SMILES: *Nc1ncc2ccn(-c3ccc(*)cc3)c2n1]

In certain embodiments,

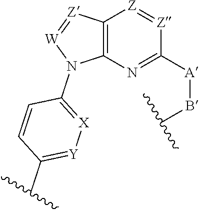

is not

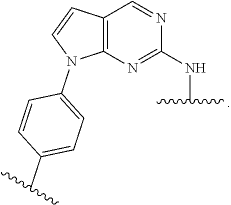 [SMILES: *Nc1ncc2ccn(-c3ccc(*)cc3)c2n1]

In certain embodiments, the molecular weight of the compound is not more than 450 g/mol. In certain embodiments, the molecular weight of the compound is not more than 400 g/mol. In certain embodiments, the molecular weight of the compound is not more than 350 g/mol. In certain embodiments, the molecular weight of the compound is not more than 300 g/mol. In certain embodiments, the molecular weight of the compound is between 250 and 300, between 300 and 350, between 350 and 400, between 400 and 450, or between 450 and 500, inclusive, g/mol. In certain embodiments, the molecular weight of the compound is between 300 and 400, inclusive, g/mol. In certain embodiments, the molecular weight of the compound is between 300 and 450, inclusive, g/mol. In certain embodiments, a compound described herein is able to pass the blood-brain-barrier (bbb).

In certain embodiments, the compound is of the formula:

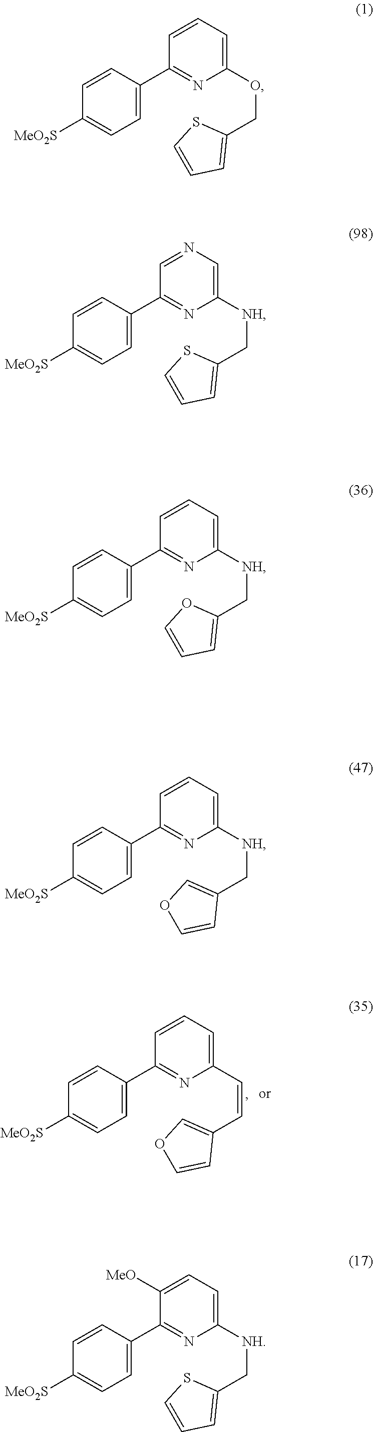 [SMILES: CS(=O)(=O)c1ccc(-c2cccc(OCc3cccs3)n2)cc1 ; CS(=O)(=O)c1ccc(-c2cncc(NCc3cccs3)n2)cc1 ; CS(=O)(=O)c1ccc(-c2cccc(NCc3ccco3)n2)cc1 ; CS(=O)(=O)c1ccc(-c2cccc(NCc3ccoc3)n2)cc1 ; CS(=O)(=O)c1ccc(-c2cccc(/C=C\c3ccoc3)n2)cc1 ; COc1ccc(NCc2cccs2)nc1-c1ccc(S(C)(=O)=O)cc1]

In certain embodiments, the compound is of Formula (36). In certain embodiments, the compound is of Formula (47). In certain embodiments, the compound is of Formula (98). In certain embodiments, the present disclosure provides a compound of Formula (36), or a pharmaceutically acceptable salt thereof. In certain embodiments, the present disclosure provides a compound of Formula (47), or a pharmaceutically acceptable salt thereof. In certain embodiments, the present disclosure provides a compound of Formula (98), or a pharmaceutically acceptable salt thereof.

In certain embodiments, the compound is of the formula:

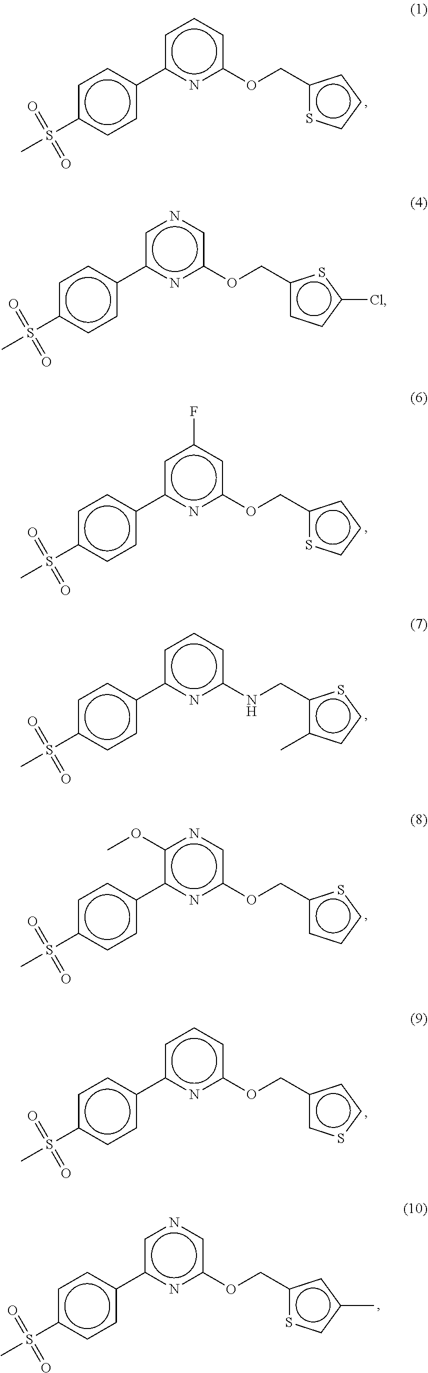

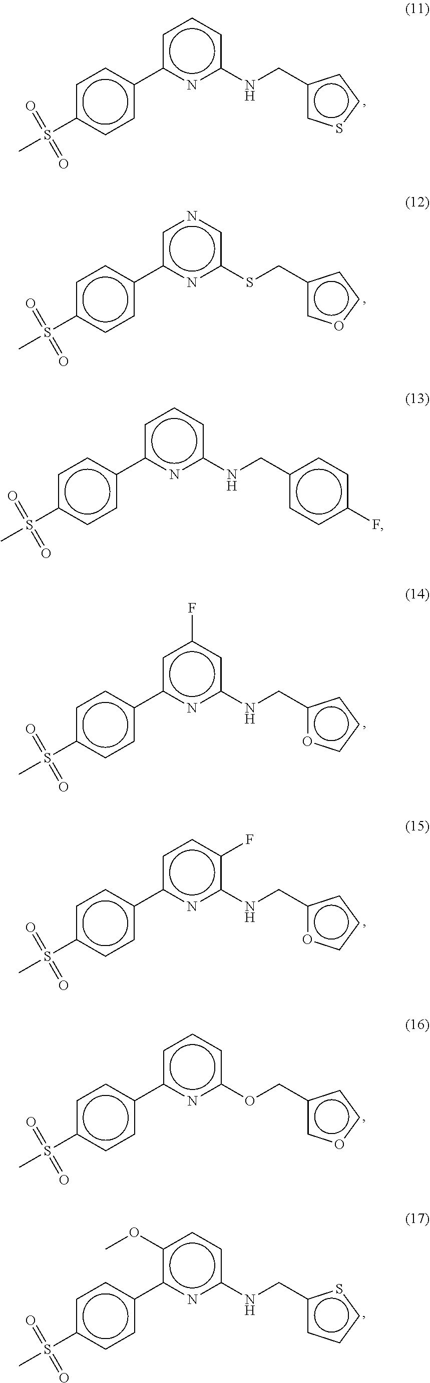

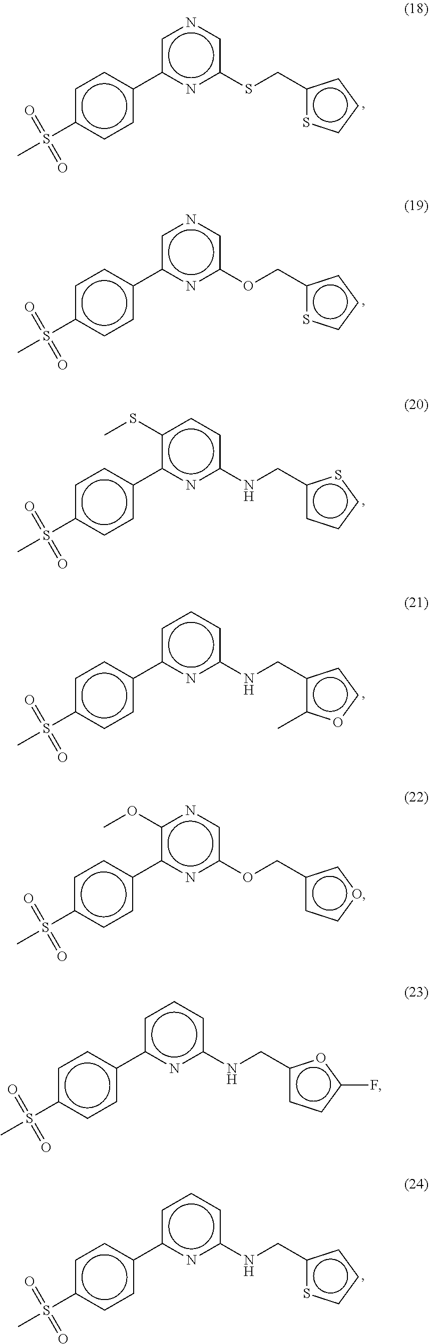

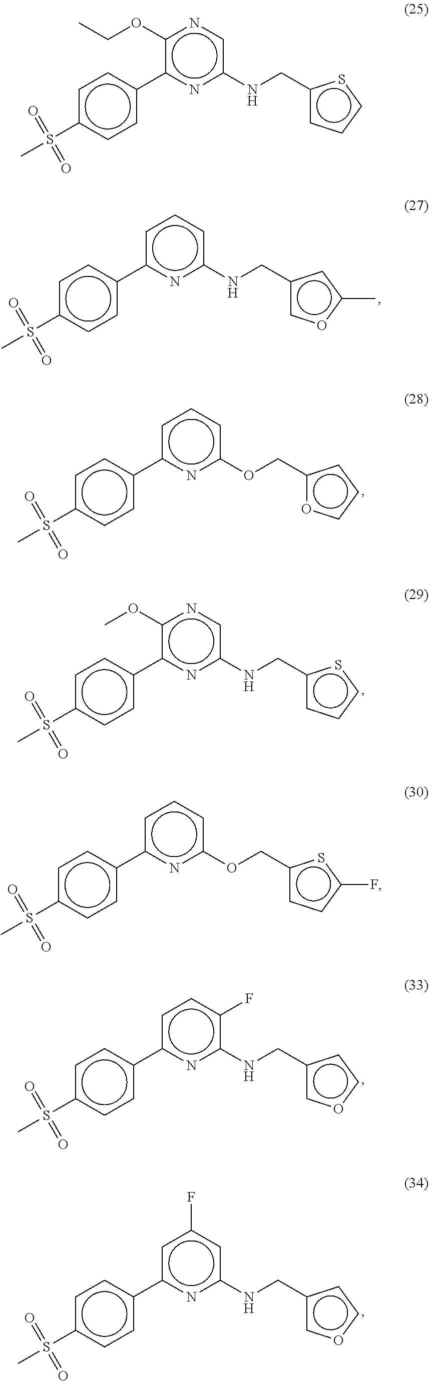

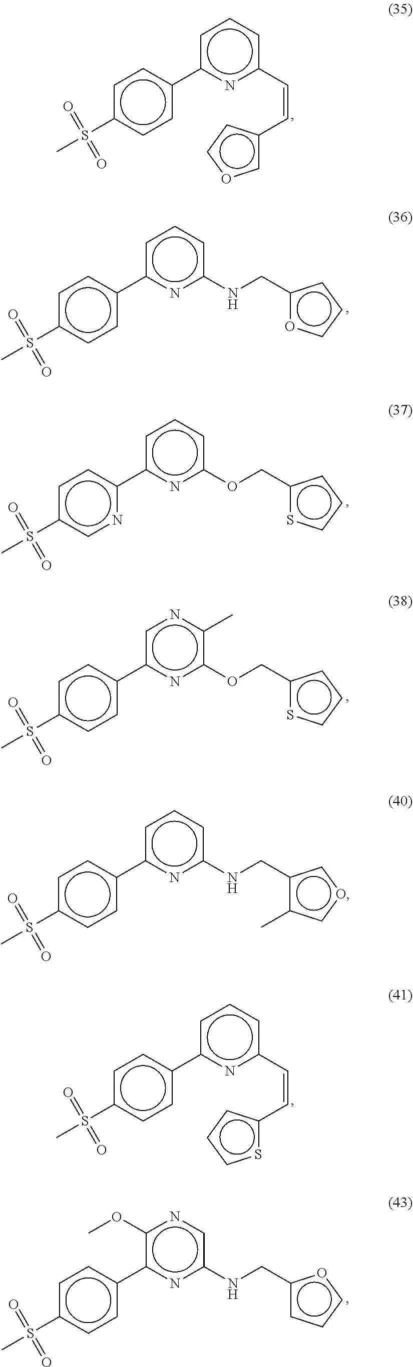

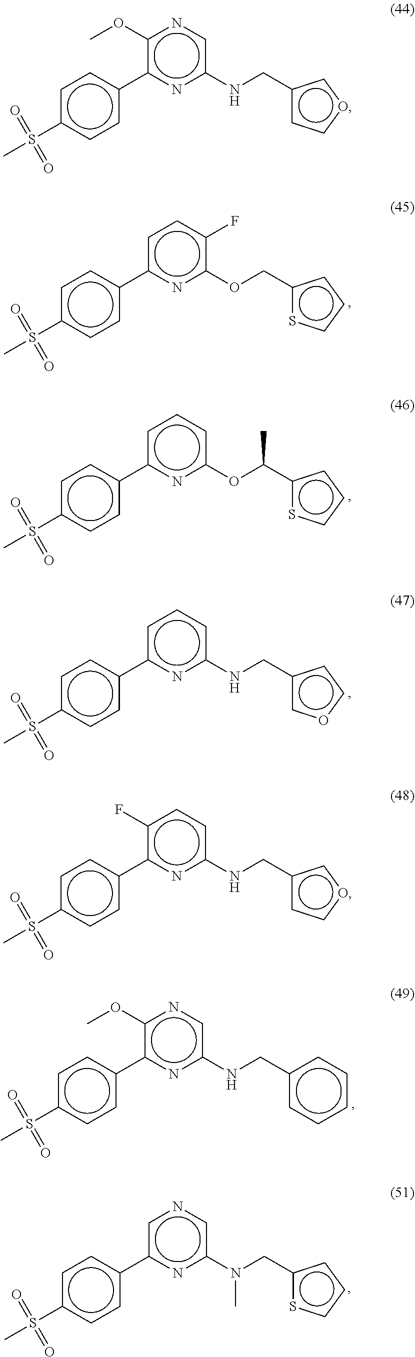

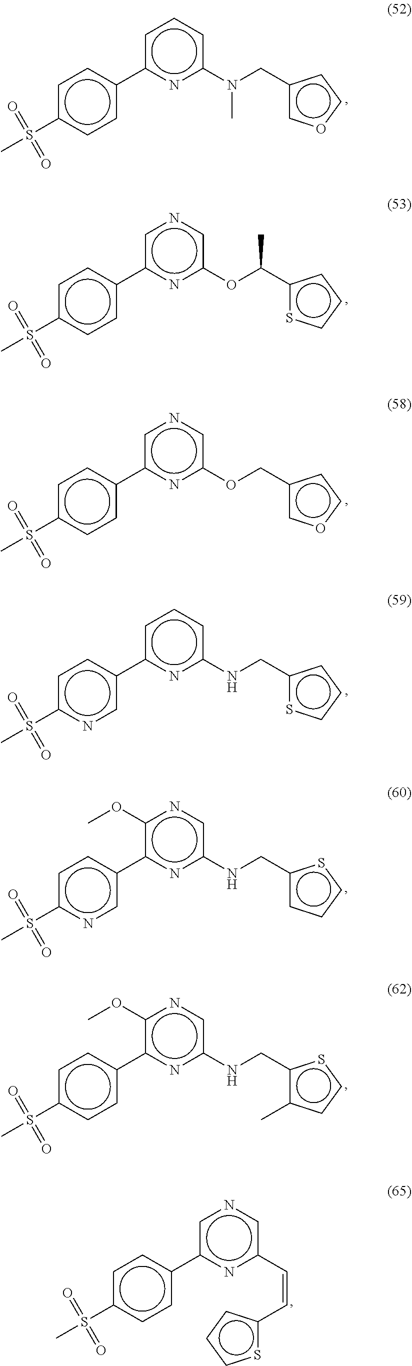

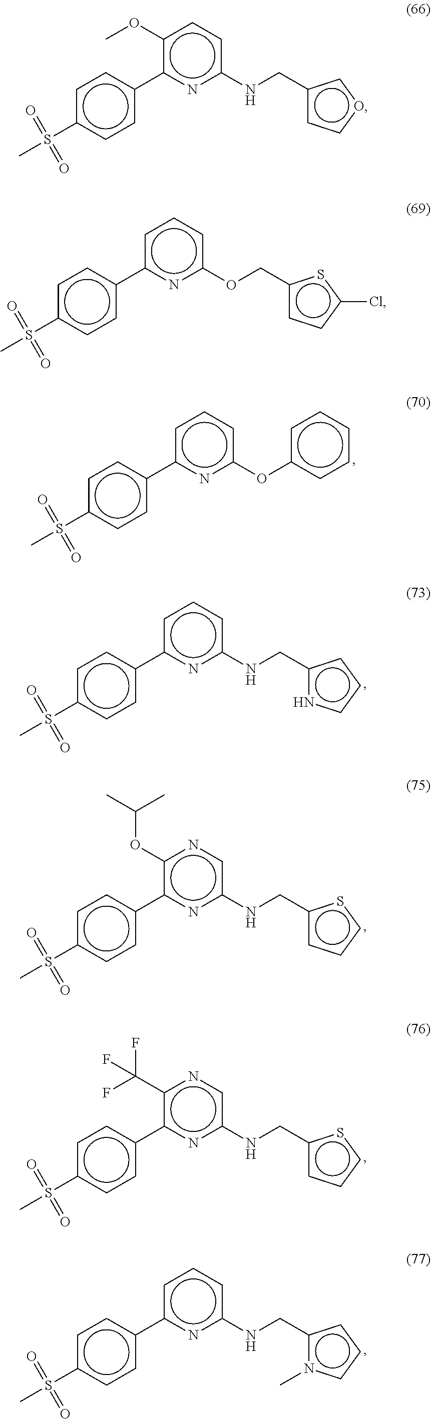

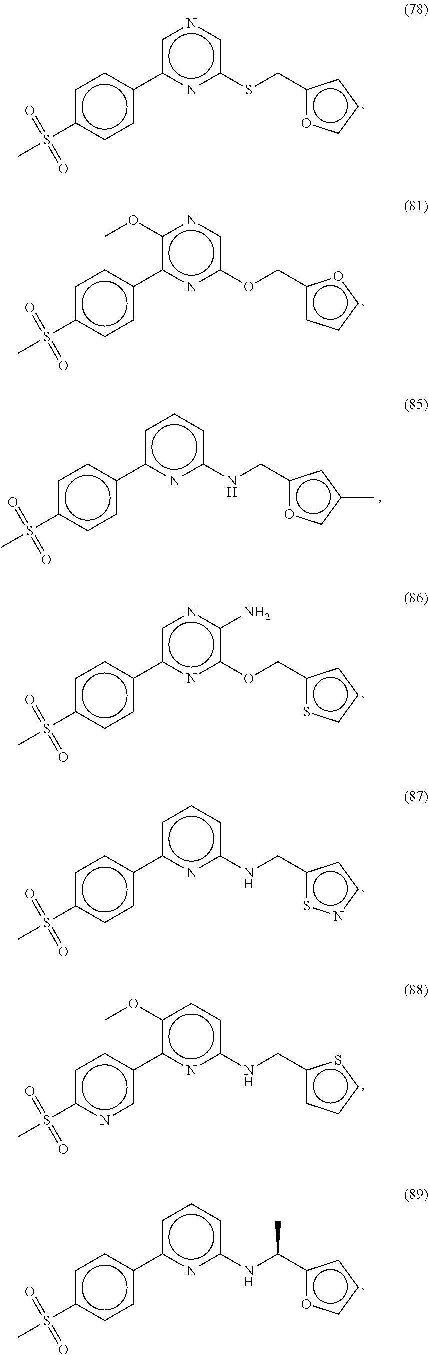

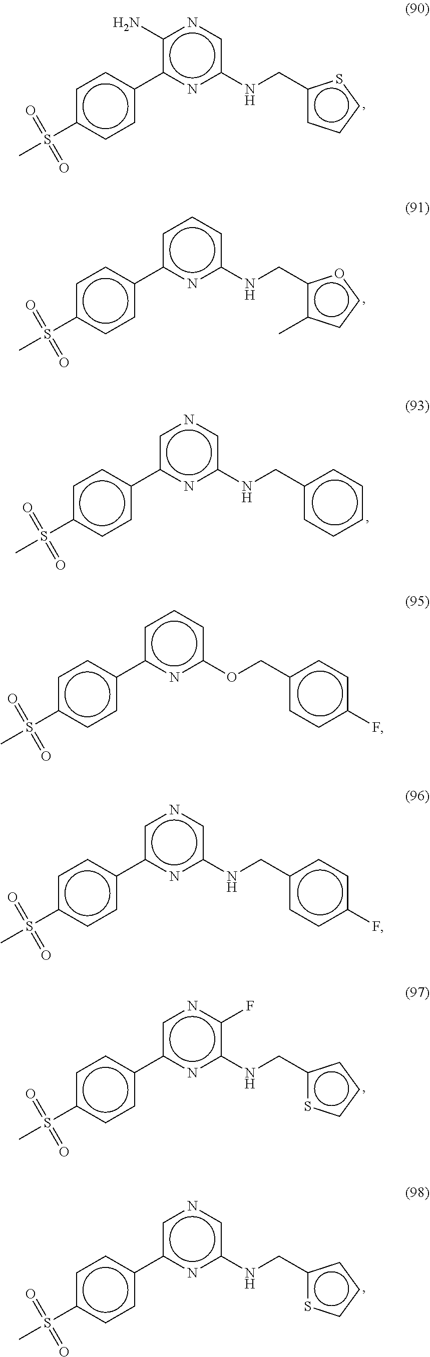

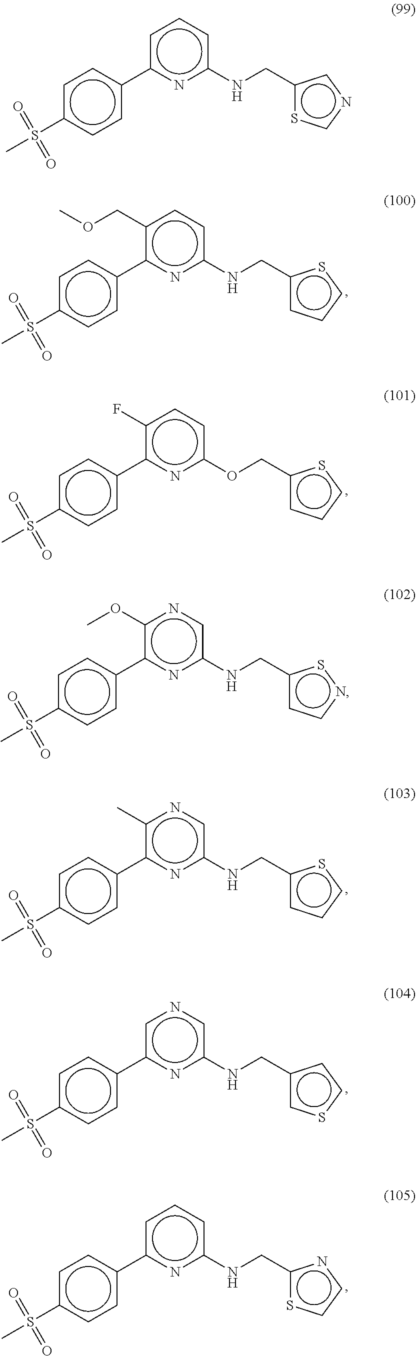

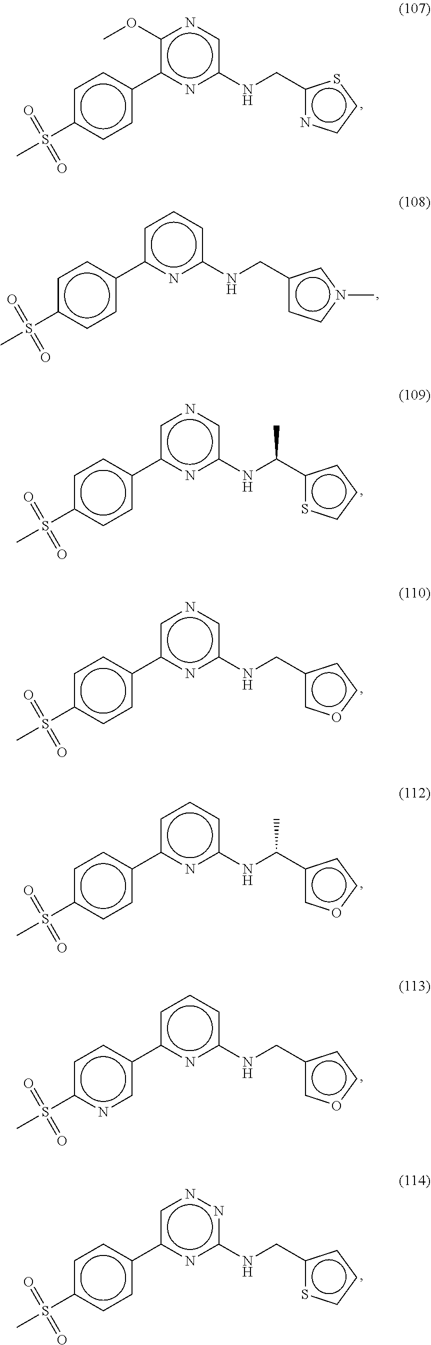

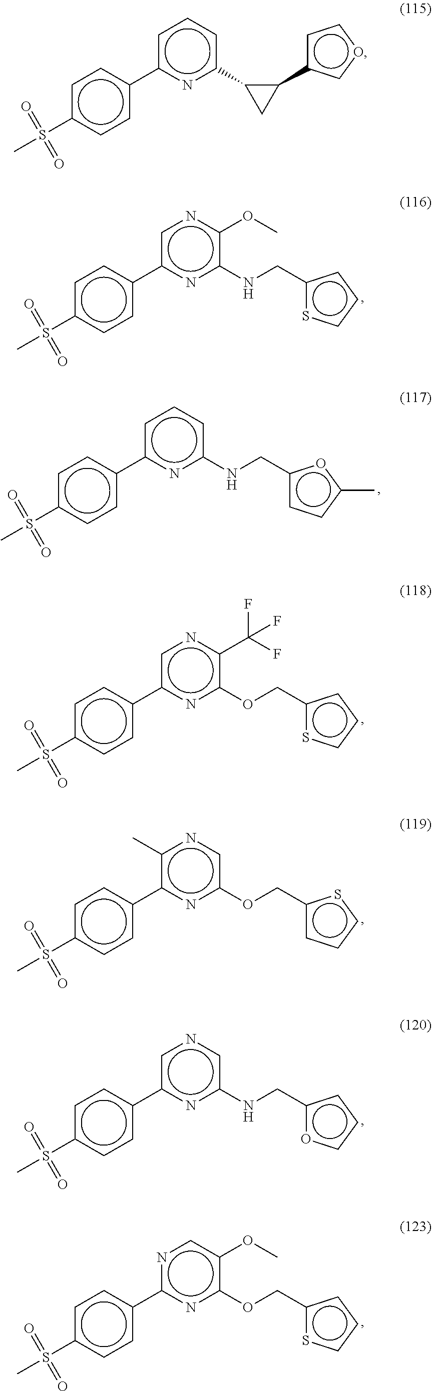

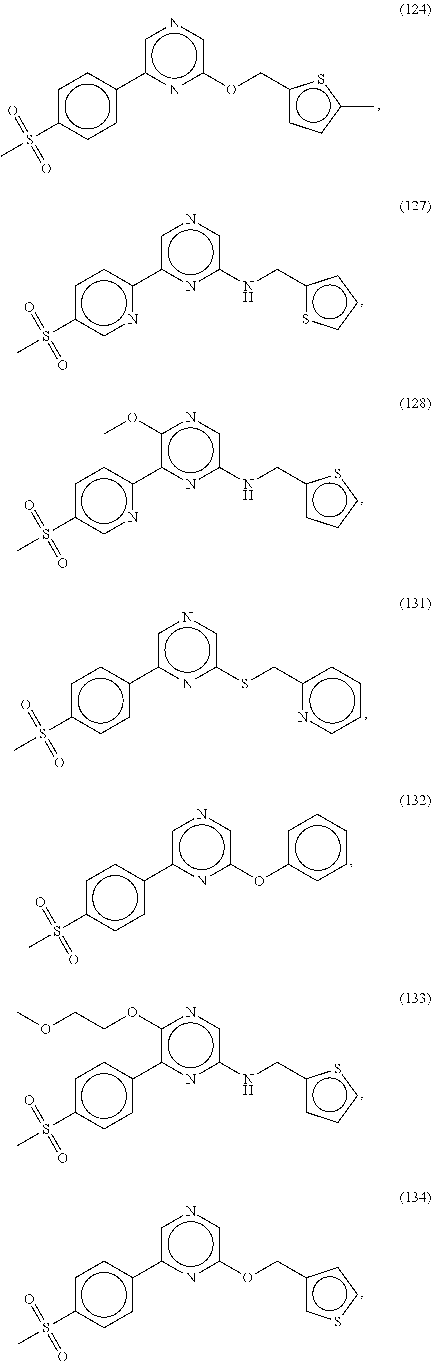

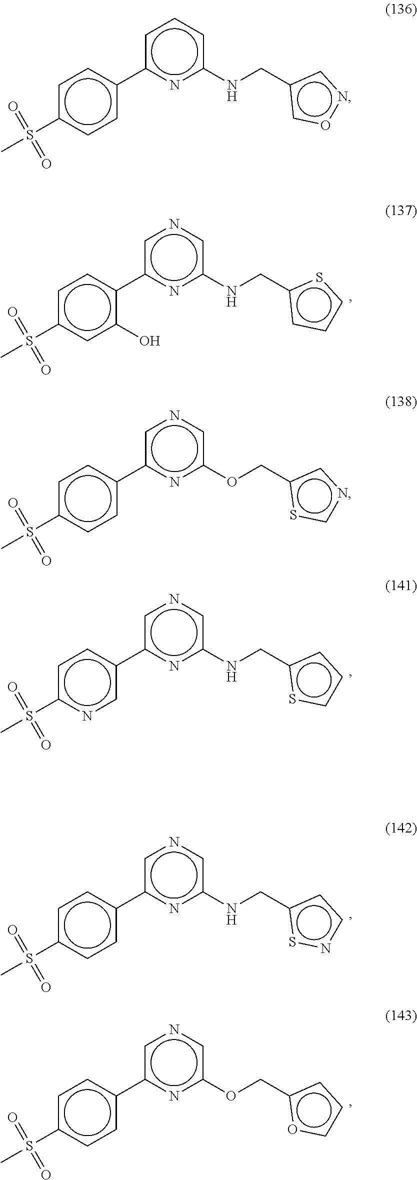

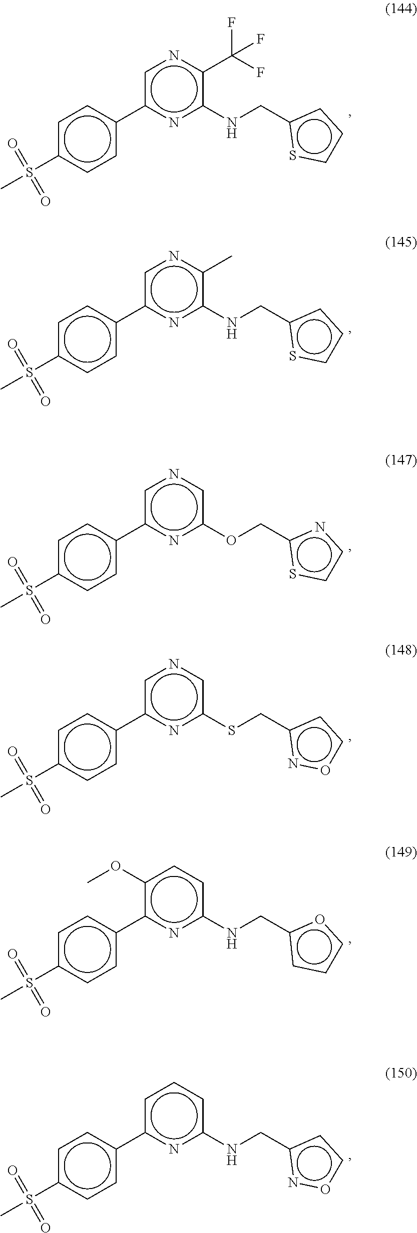

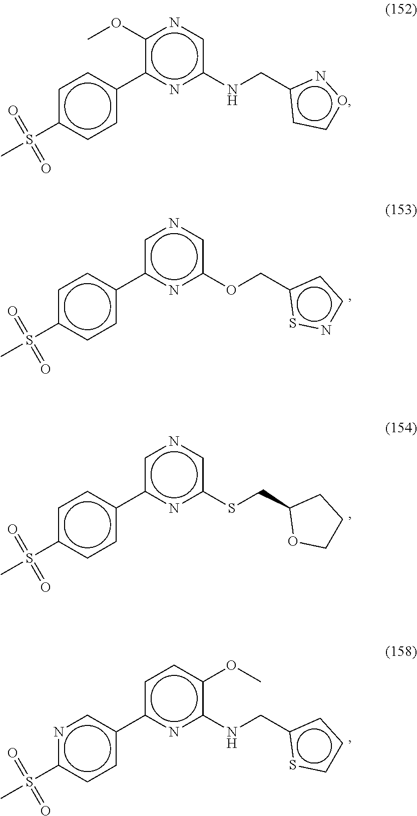

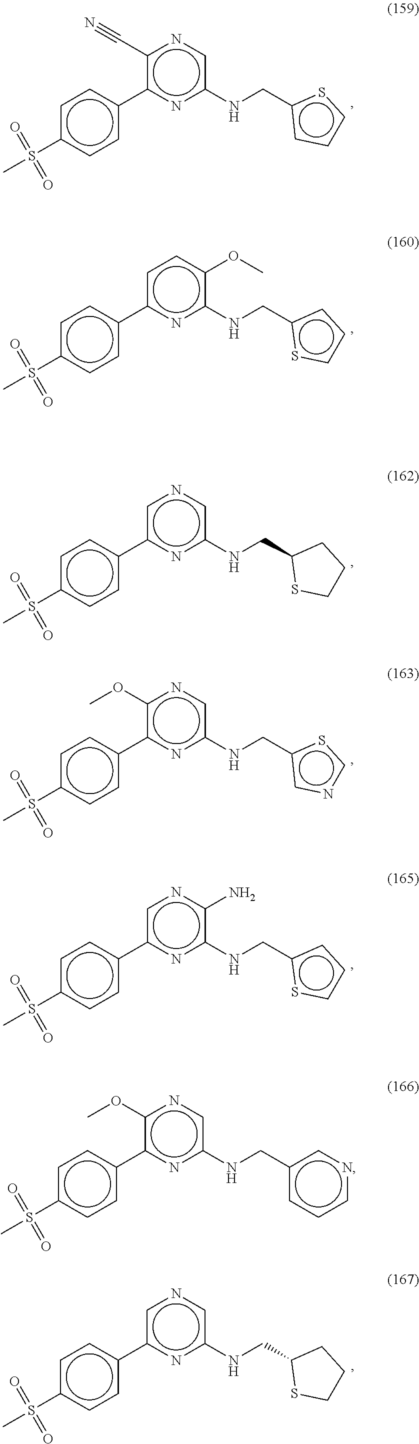

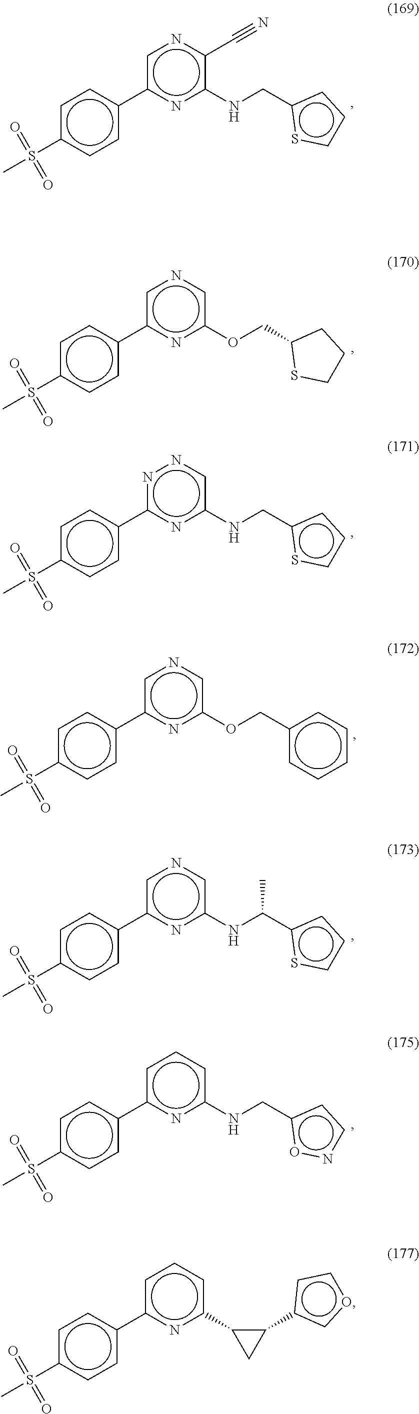

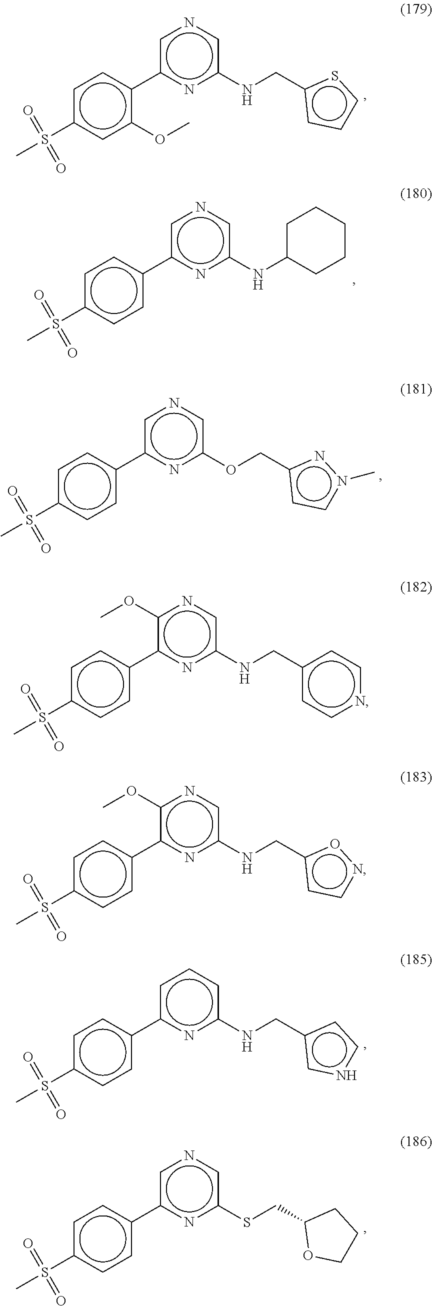

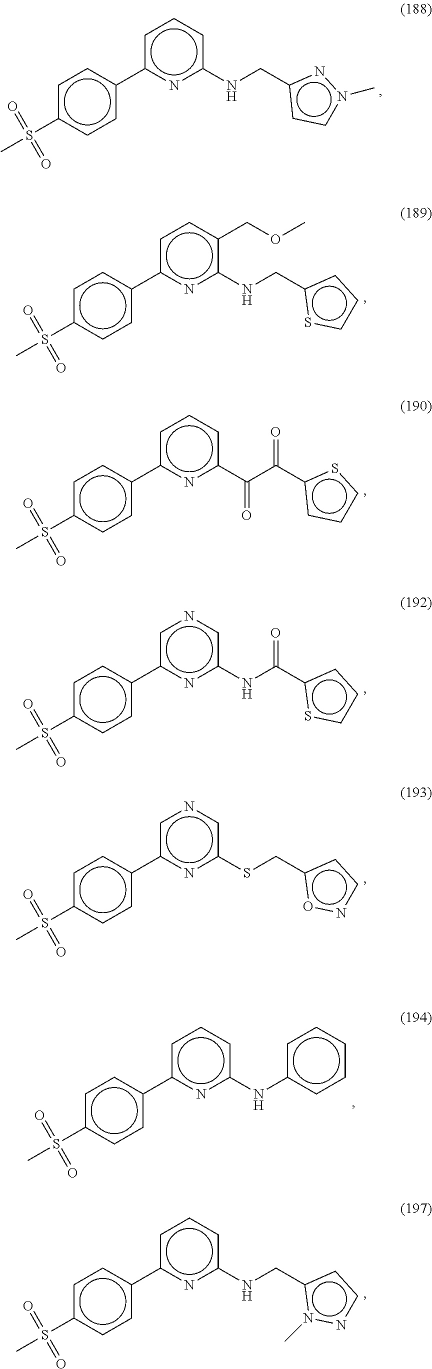

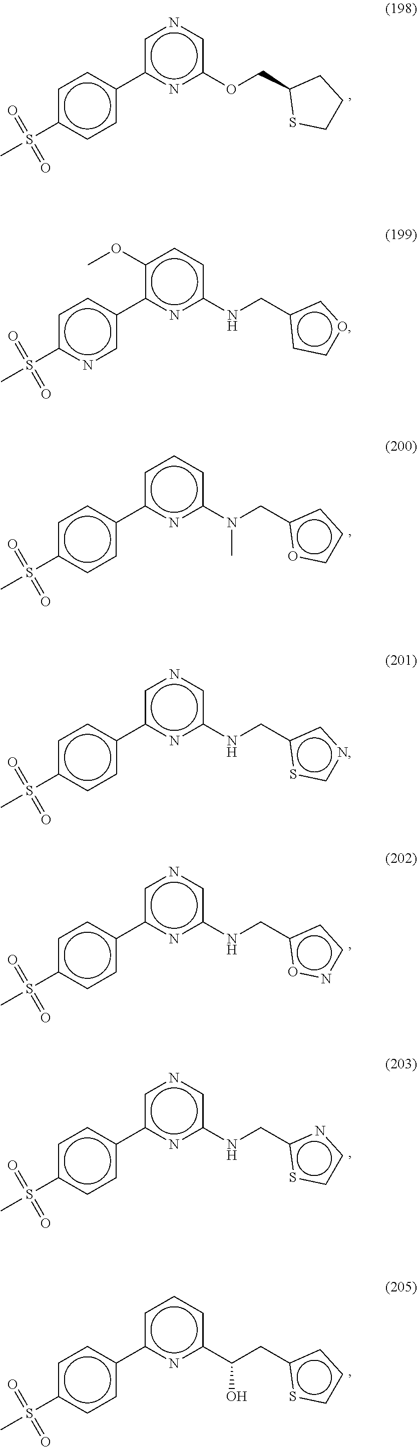

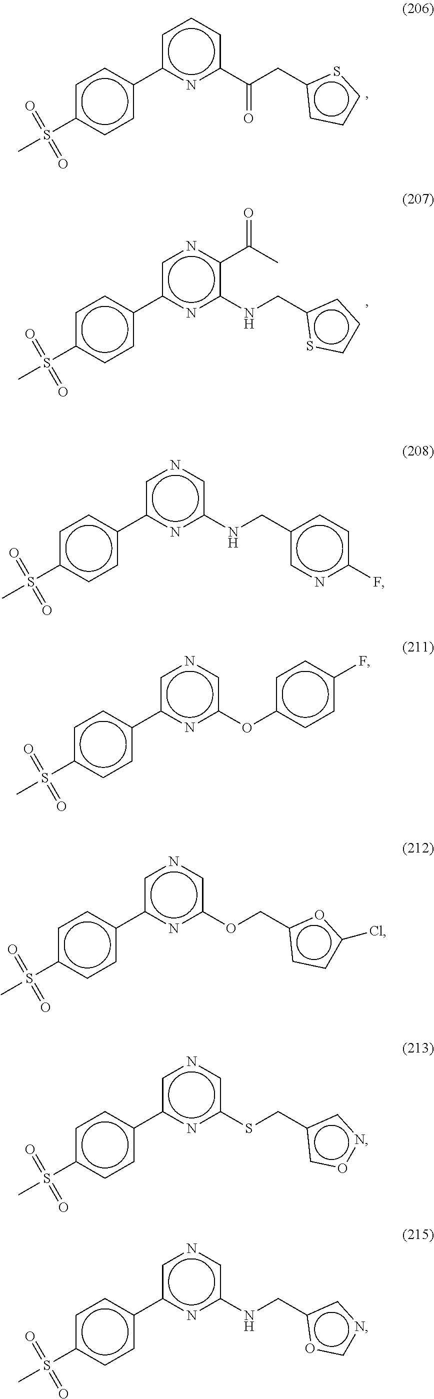

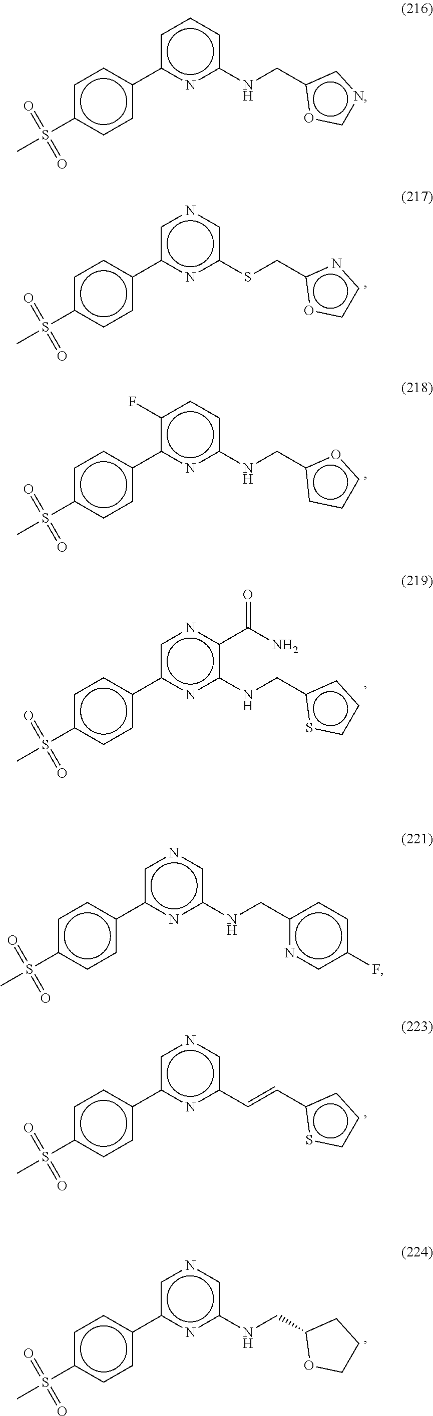

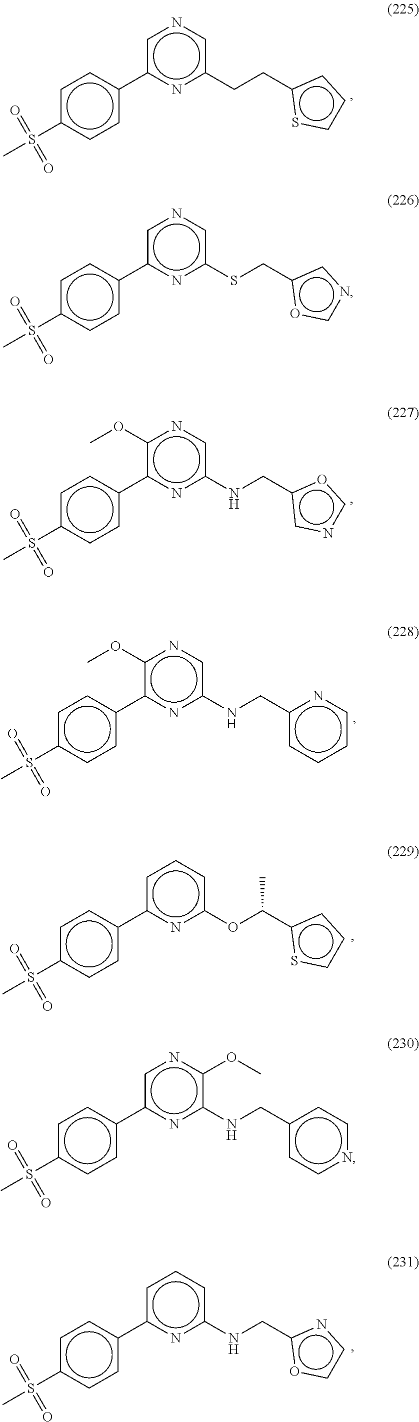

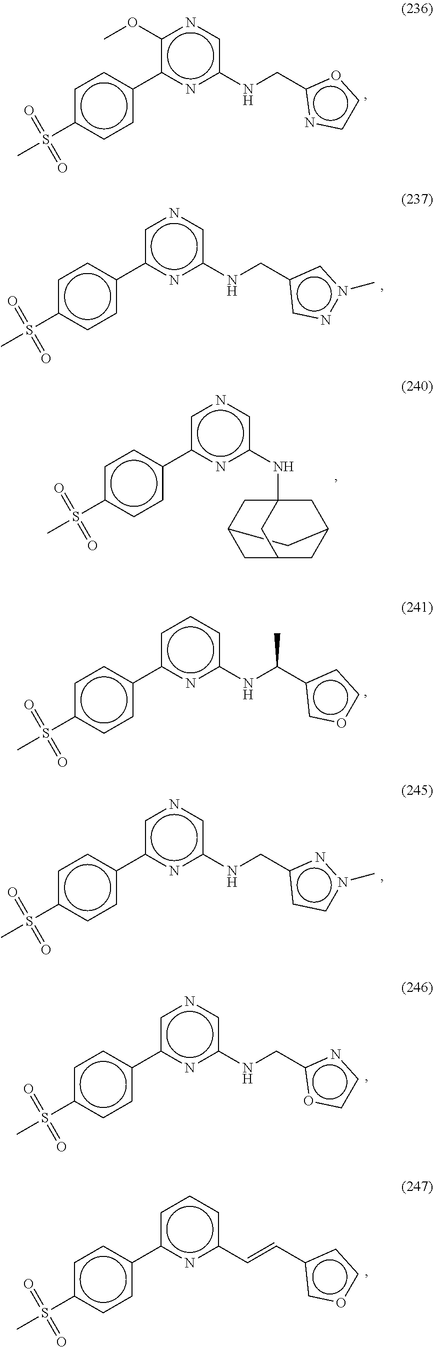

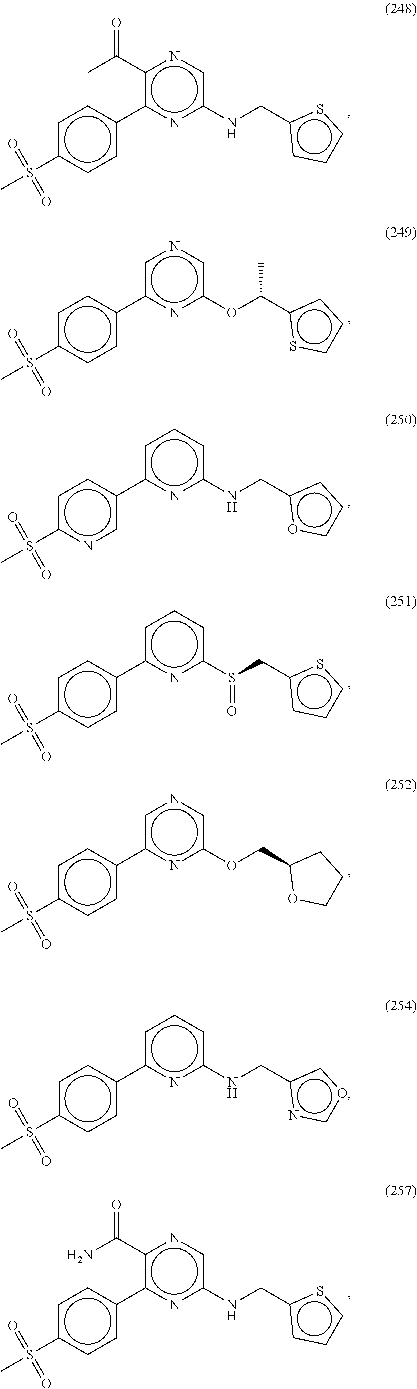

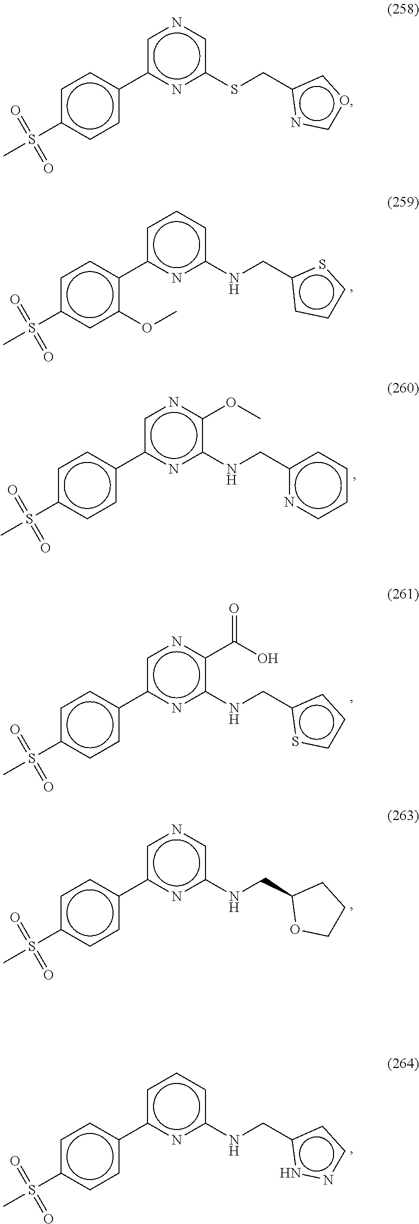

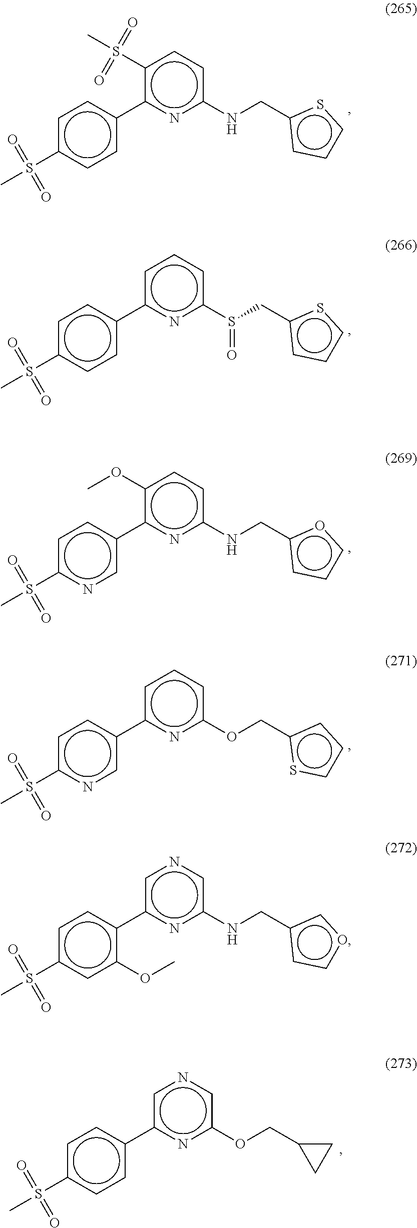

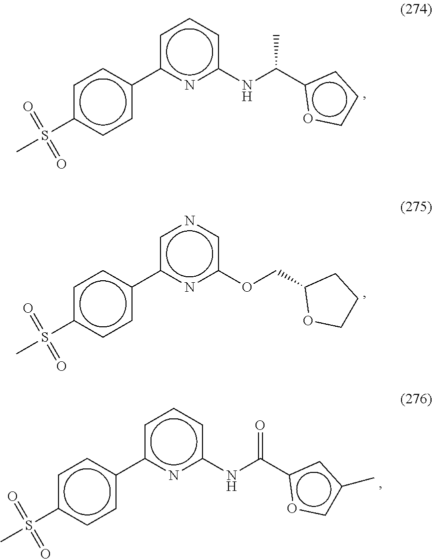

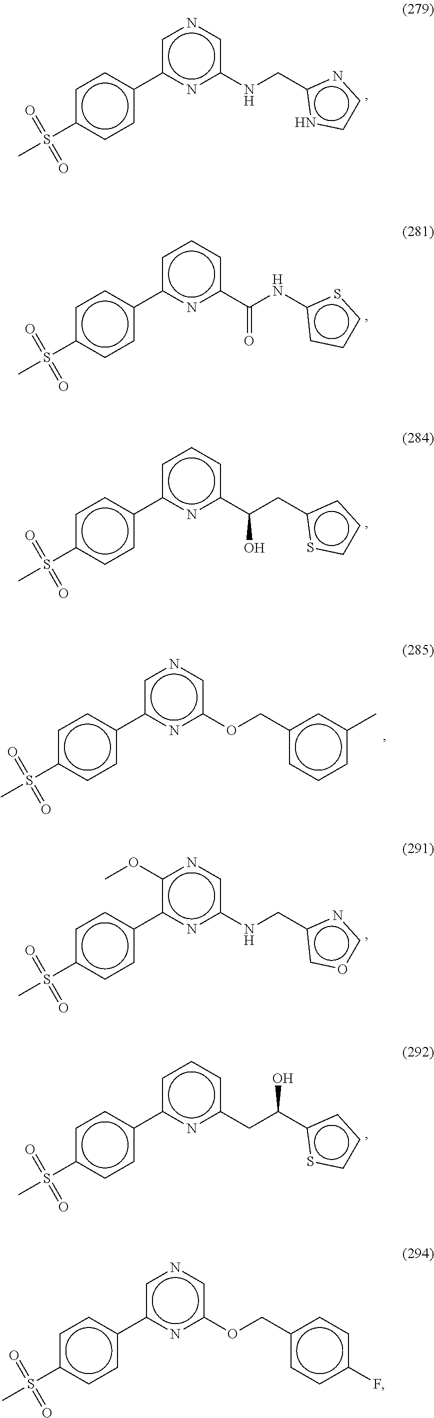

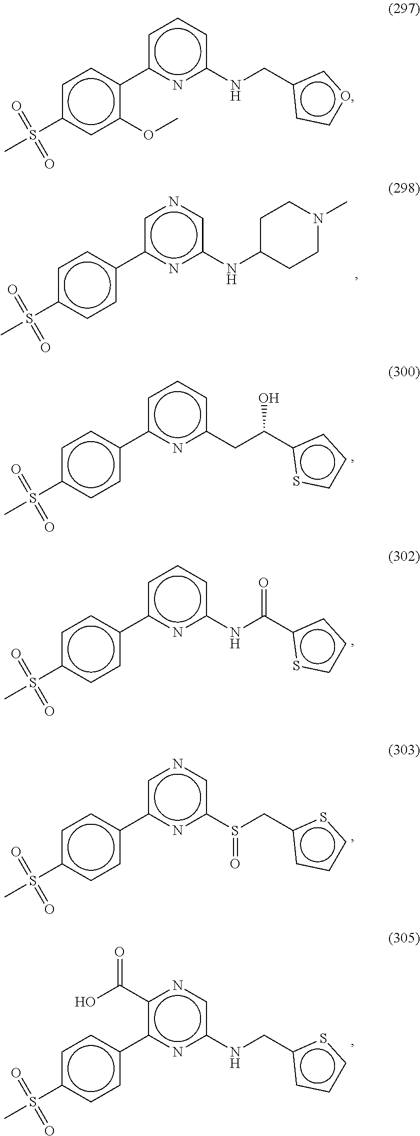

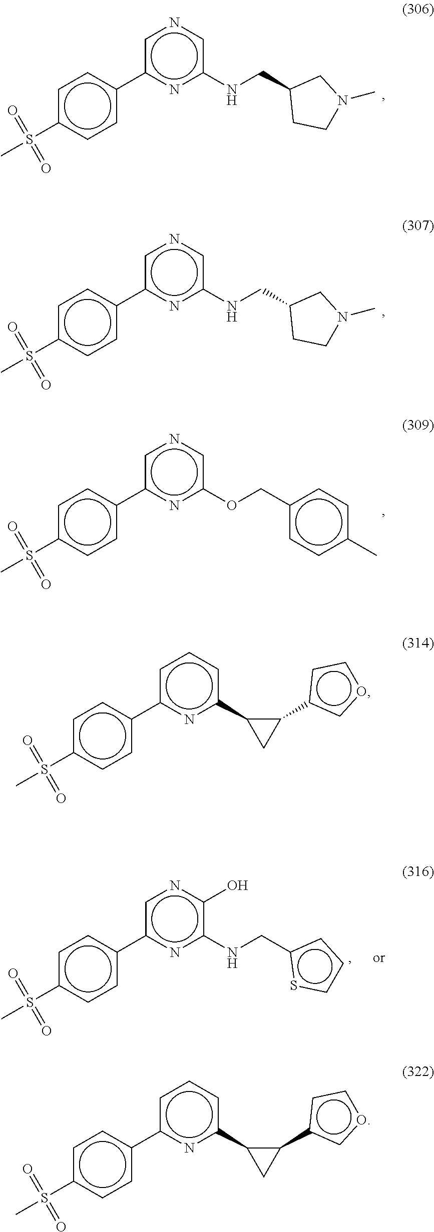

In certain embodiments, the compound is of the formula:

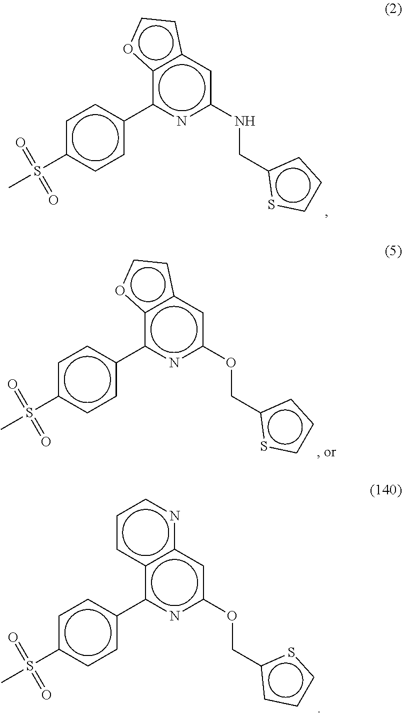

In certain embodiments, the compound is of the formula:

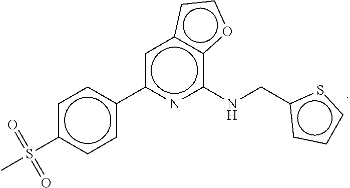

In certain embodiments, the compound is of the formula:

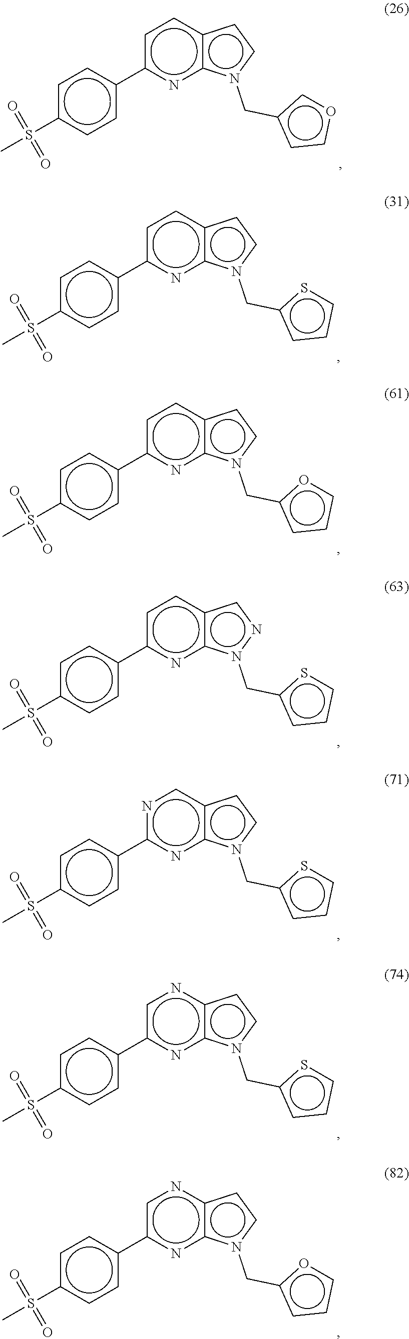

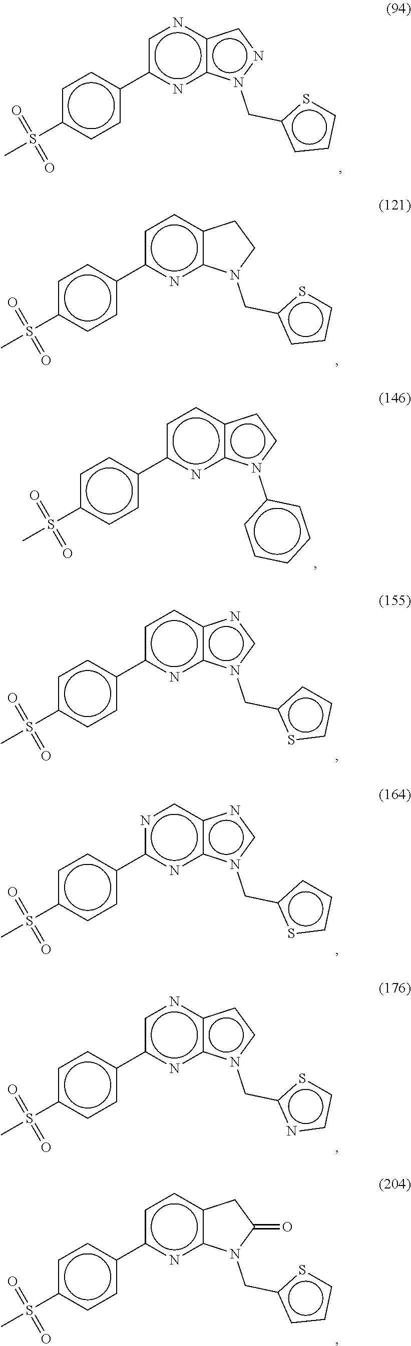

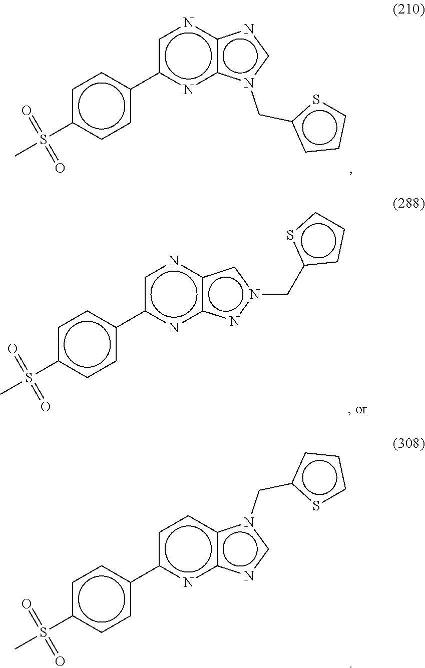

In certain embodiments, the compound is of the formula:

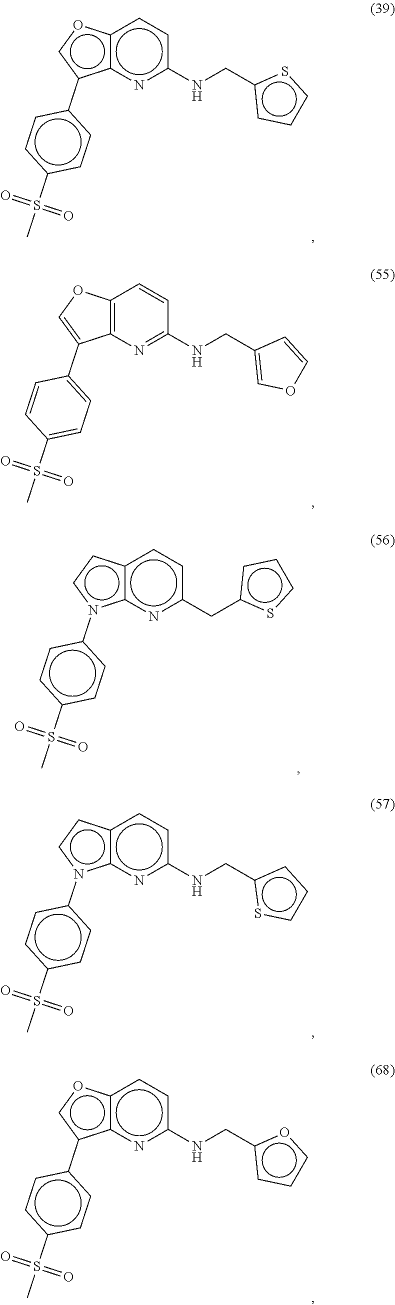

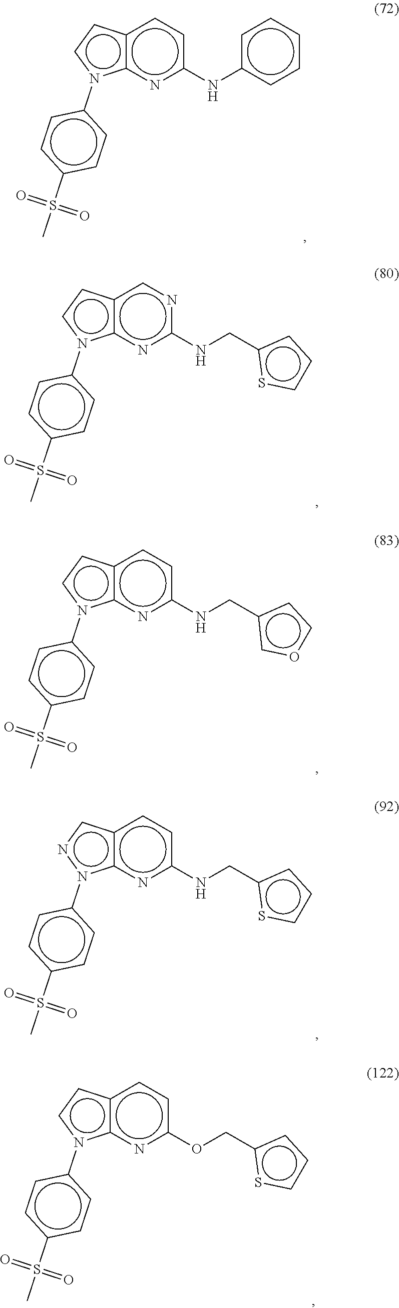

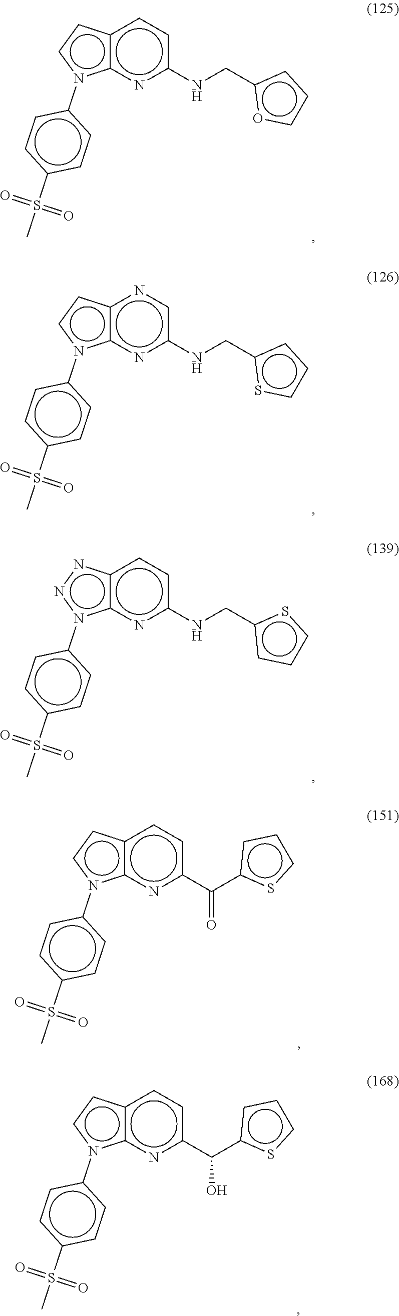

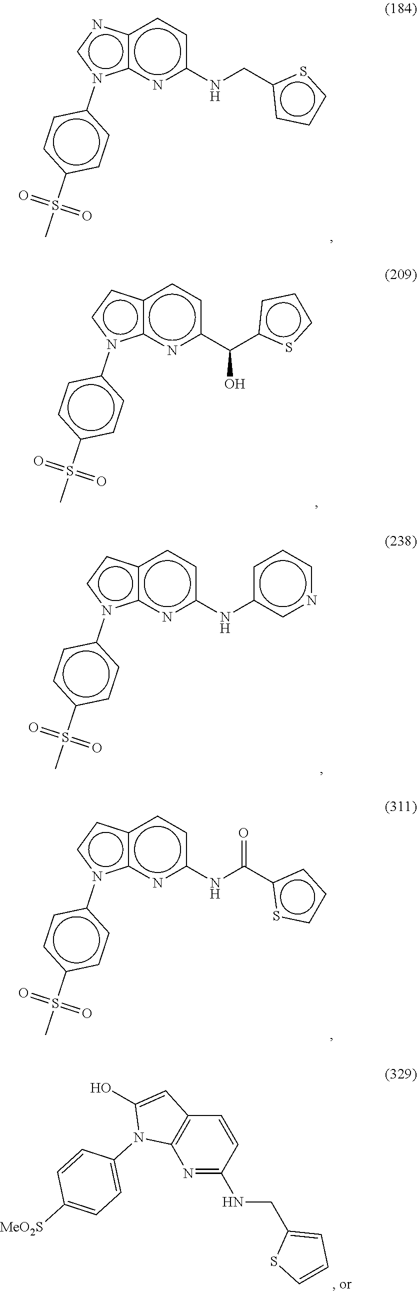

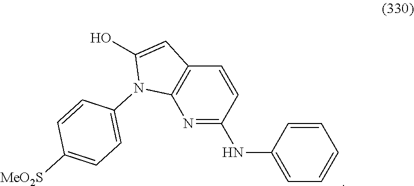 [SMILES: CS(=O)(=O)c1ccc(-n2c(O)cc3ccc(Nc4ccccc4)nc32)cc1 ; "."]

The present disclosure also describes a compound of the formula:

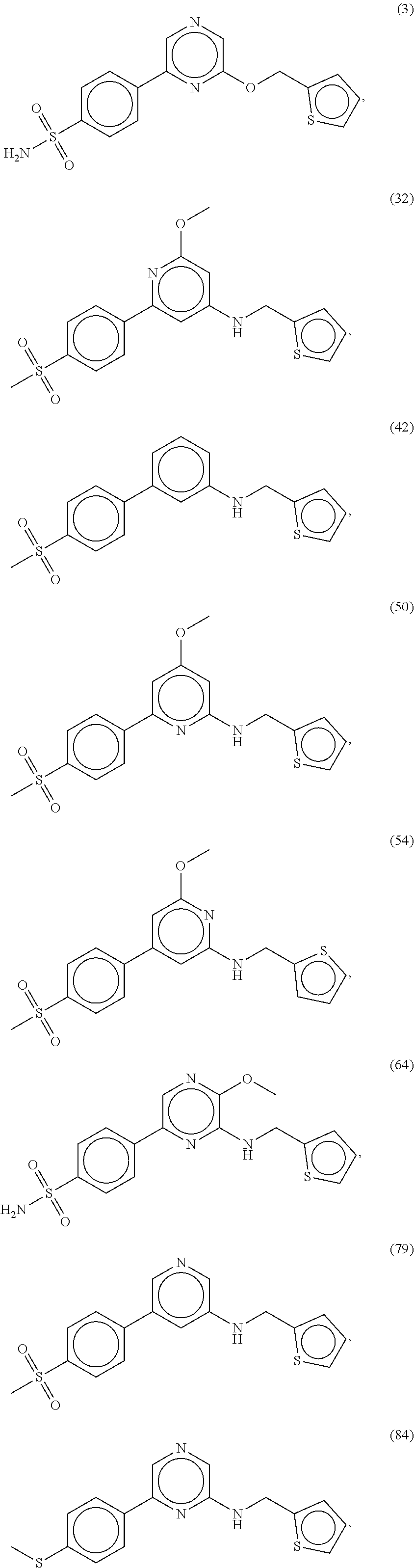

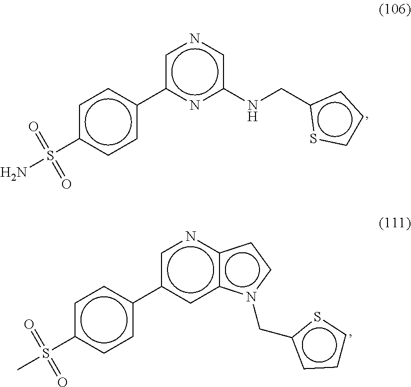

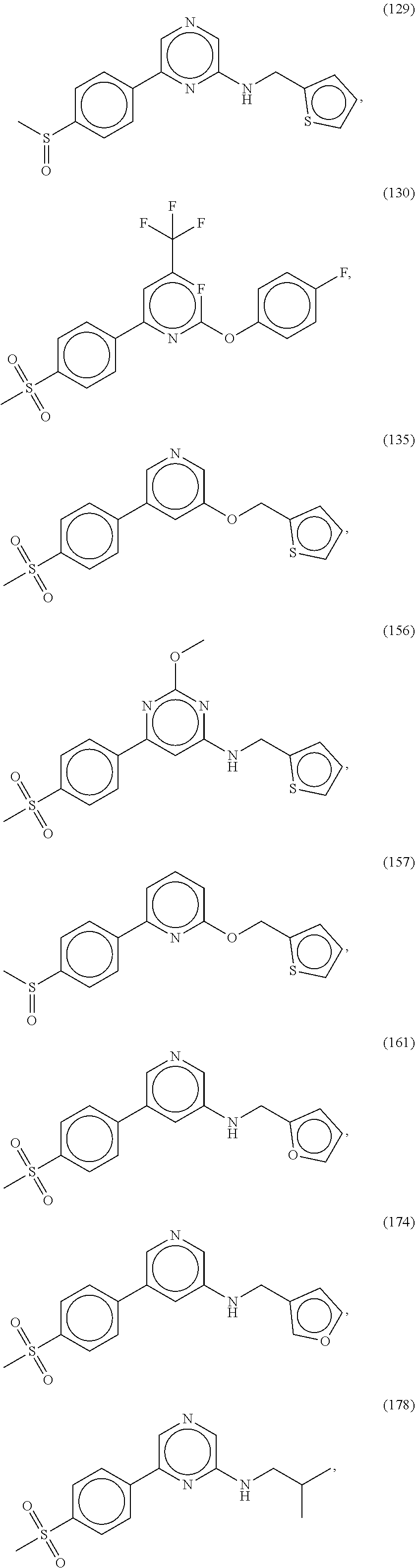

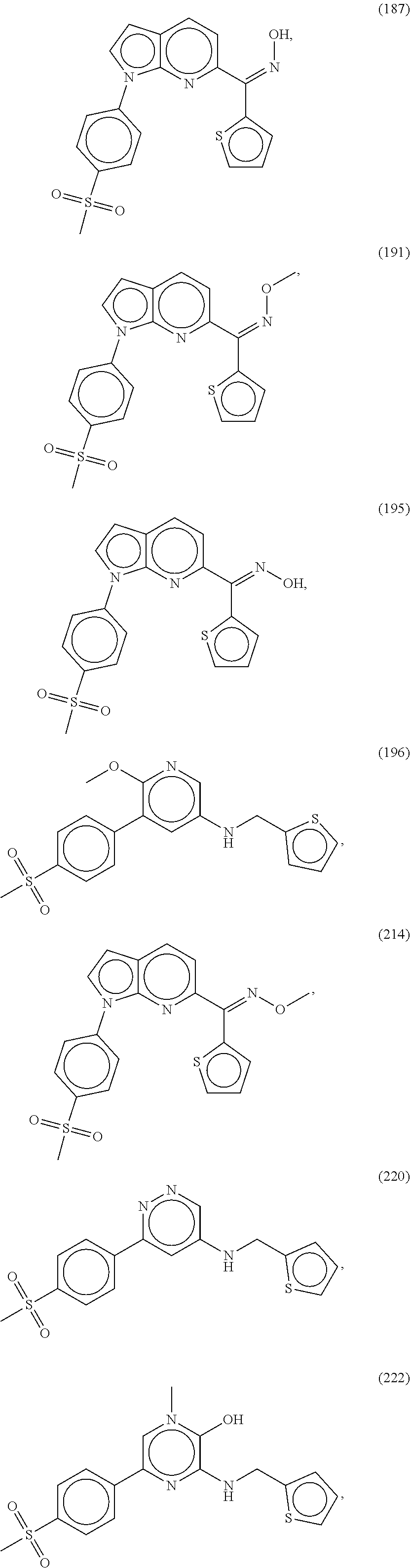

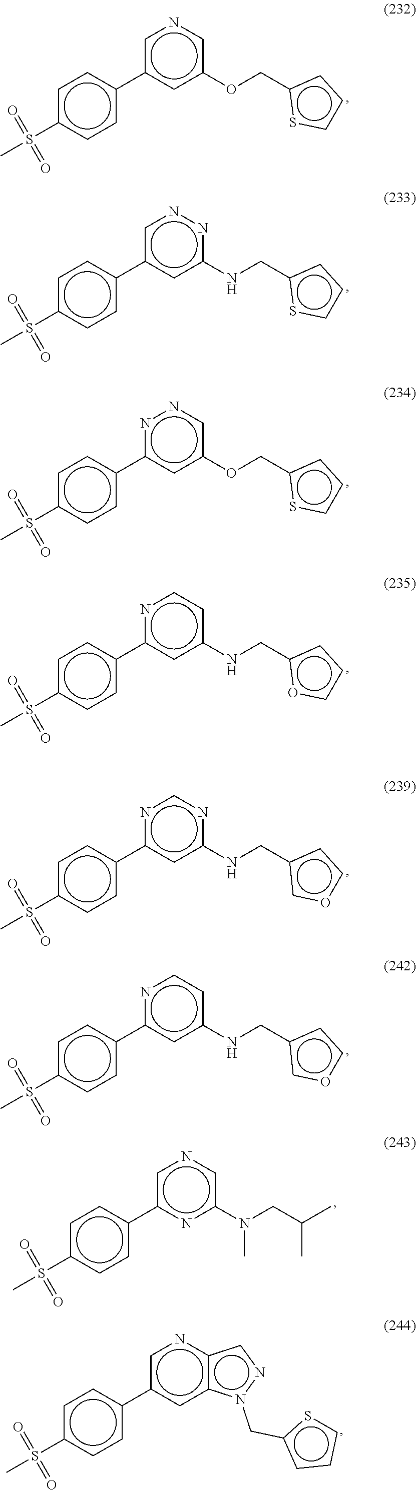

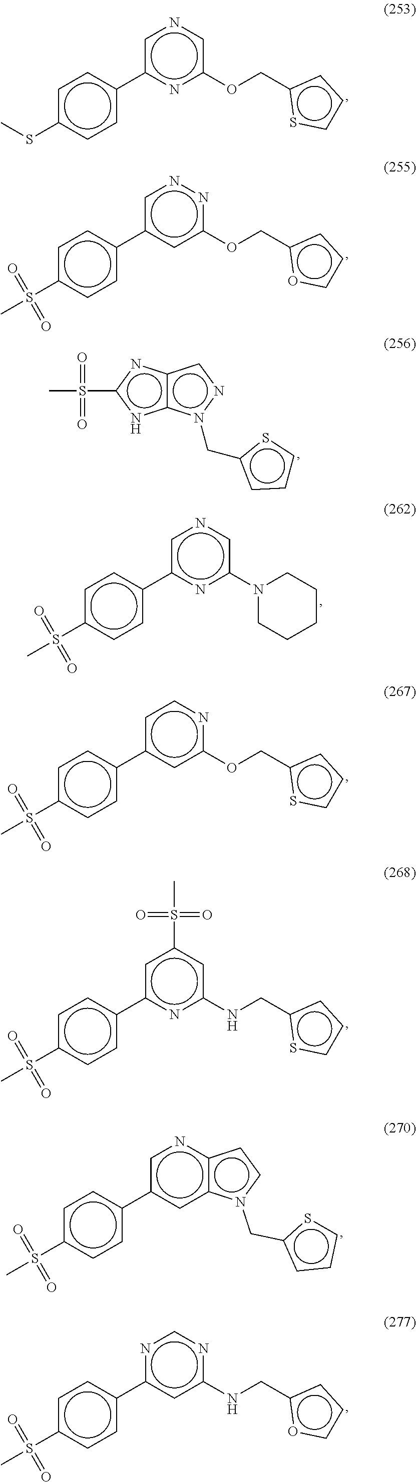

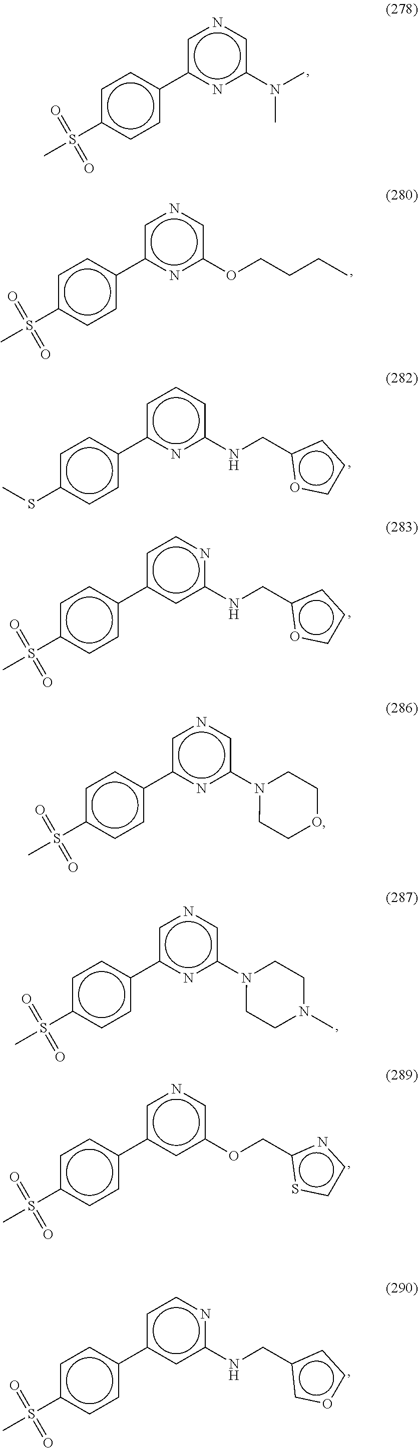

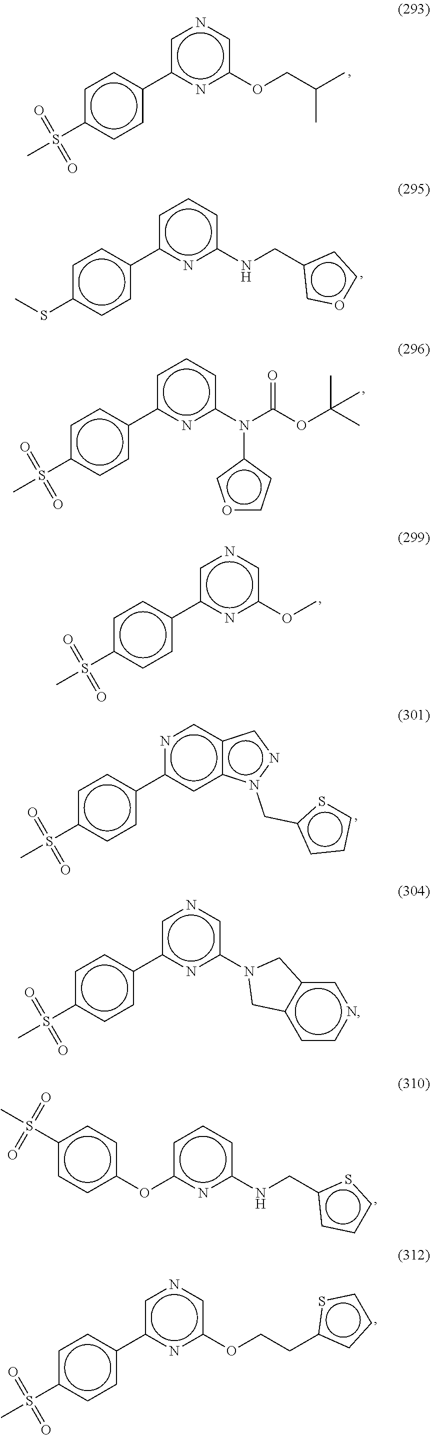

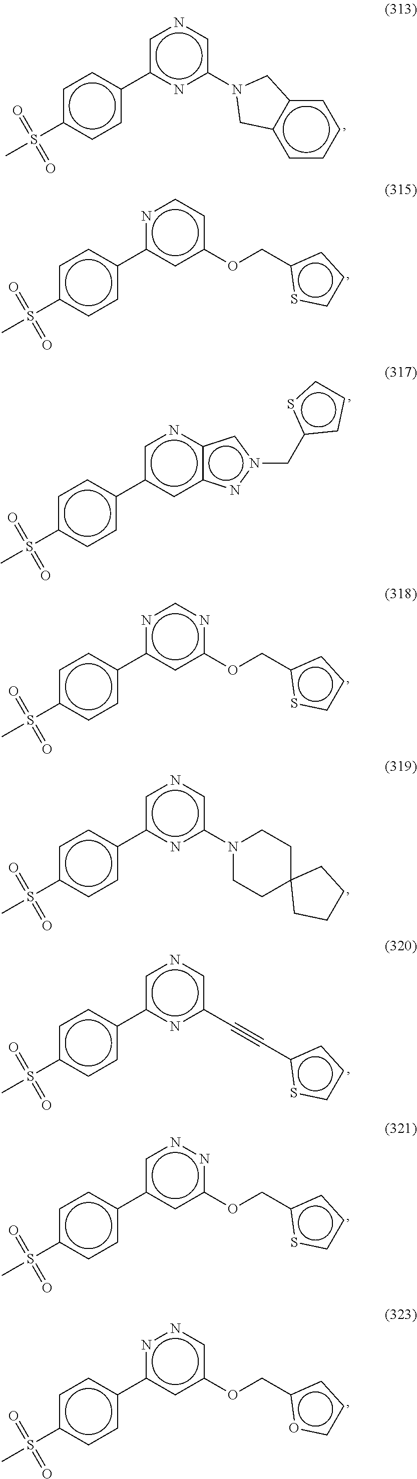

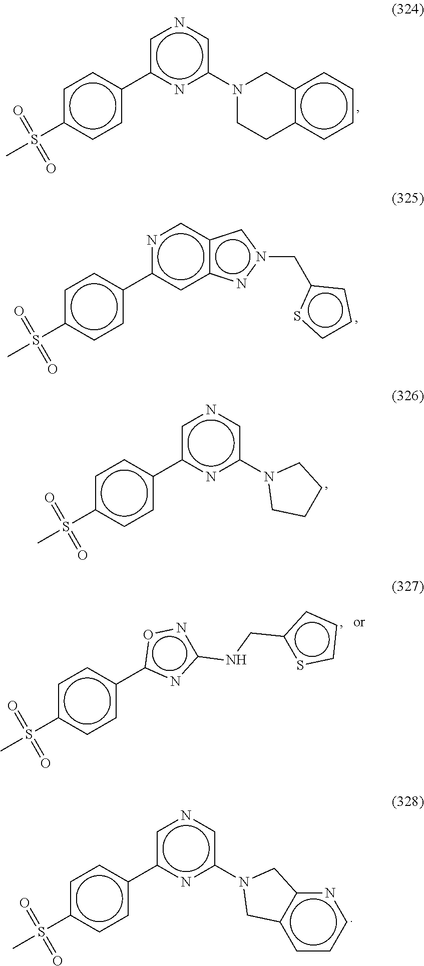

or a salt, solvate, hydrate, polymorph, co-crystal, tautomer, stereoisomer, isotopically labeled derivative, or prodrug thereof.

The present disclosure also describes a compound of the formula:

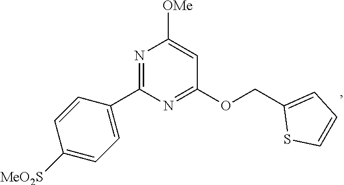 [SMILES: COc1cc(OCc2cccs2)nc(-c2ccc(S(C)(=O)=O)cc2)n1]

or a salt, solvate, hydrate, polymorph, co-crystal, tautomer, stereoisomer, isotopically labeled derivative, or prodrug thereof.

In certain embodiments, at least one atom of the compound is isotopically enriched (e.g., with a radioactive isotope) above the natural abundance. In certain embodiments, at least one carbon atom or at least one hydrogen atom of the compound is isotopically enriched with carbon-11 or hydrogen-3, respectively, above the natural abundance. In certain embodiments, at least one hydrogen atom (e.g., at least one of the hydrogen atoms marked with “b”) of the compound is isotopically enriched with ³H above the natural abundance. In certain embodiments, the compound comprises at least one fluorine atom isotopically enriched (e.g., with ¹⁸F) above the natural abundance. In certain embodiments, at least one carbon atom (e.g., the carbon atom marked with “a”) of the compound is isotopically enriched (e.g., with ¹¹C) above the natural abundance. In certain embodiments, the radioactive isotope is ¹³N, or ¹⁵O. In certain embodiments, the abundance of the radioactive isotope in the compound is between 50% and 70%, between 70% and 80%, between 80% and 90%, between 90% and 95%, or between 95% and 99%.

Compound number “[¹¹C]36” refers to compound number 36 where the carbon atom marked with “a” of the compound is isotopically enriched with ¹¹C above the natural abundance. Compound number “[3H]47” refers to compound number 47 where each of the hydrogen atoms marked with “b” of the compound is isotopically enriched with ³H above the natural abundance. The compounds described herein may follow these naming conventions.

In certain embodiments, a compound described herein is a compound of a formula described herein, or a salt, solvate, hydrate, polymorph, co-crystal, tautomer, stereoisomer, isotopically labeled derivative, or prodrug thereof. In certain embodiments, a compound described herein is a compound of a formula described herein, or a salt, solvate, hydrate, polymorph, co-crystal, tautomer, stereoisomer, or isotopically labeled derivative thereof. In certain embodiments, an isotopically labeled derivative is an isotopically labeled compound. In certain embodiments, a compound described herein is a compound of a formula described herein, or a salt (e.g., pharmaceutically acceptable salt) thereof.

In certain embodiments, a described herein inhibits a COX. In certain embodiments, a provided compound inhibits the activity (e.g., aberrant activity (e.g., higher-than-normal activity, increase activity)) of a COX. In certain embodiments, a provided compound inhibits the overexpression of a COX. In certain embodiments, the COX is a human COX. In certain embodiments, the COX is a non-human mammal COX. In certain embodiments, the COX is a wild type COX. In certain embodiments, the COX is a mutant COX. In certain embodiments, a provided compound inhibits a COX as measured in an assay described herein or known in the art. In certain embodiments, a provided compound inhibits the COX at an IC₅₀ less than or equal to 30 μM, less than or equal to 10 μM, less than or equal to 3 μM, less than or equal to 1 μM, less than or equal to 0.3 μM, or less than or equal to 0.1 μM. In certain embodiments, the COX is COX1. In certain embodiments, the COX is COX2. In certain embodiments, a provided compound is selective for inhibiting COX2 over a different enzyme (e.g., COX1). In certain embodiments, a provided compound is selective for inhibiting COX2 over the different enzyme (e.g., COX1) by at least 2-fold, at least 3-fold, at least 4-fold, at least 5-fold, at least 7-fold, at least 10-fold, at least 20-fold, at least 50-fold, at least 100-fold, at least 300-fold, or at least 1,000-fold (e.g., in an in vitro assay or an assay described herein). In certain embodiments, a provided compound reversibly binds to a COX. In certain embodiments, a provided compound irreversibly binds to a COX.

In certain embodiments, in the section “Compounds”, articles such as “a,” “an,” and “the” mean one or more than one. In certain embodiments, in the section “Compounds”, articles such as “a,” “an,” and “the” mean one.

### Pharmaceutical Compositions, Administration, and Kits

The present disclosure also provides pharmaceutical compositions comprising a compound described herein and optionally a pharmaceutically acceptable excipient. In certain embodiments, the pharmaceutical composition further comprises an additional pharmaceutical agent.

In certain embodiments, the compound described herein is provided in an effective amount in the pharmaceutical composition. In certain embodiments, the effective amount is a therapeutically effective amount. In certain embodiments, a therapeutically effective amount is an amount effective for inhibiting a COX (e.g., COX2). In certain embodiments, a therapeutically effective amount is an amount effective for treating a disease (e.g., a disease associated with aberrant activity of a COX (e.g., proliferative disease, neurological disease, psychiatric disease)). In certain embodiments, a therapeutically effective amount is an amount effective for inhibiting the activity or decreasing the level of a COX and treating a disease (e.g., a disease associated with aberrant activity of a COX (e.g., proliferative disease, neurological disease, psychiatric disease)). In certain embodiments, a therapeutically effective amount is an amount effective for inducing apoptosis in a cell (e.g., cancer cell, premalignant cell). In certain embodiments, the effective amount is a diagnostically effective amount (effective in diagnosing a disease). In certain embodiments, a therapeutically effective amount is an amount effective for diagnosing a disease.

In certain embodiments, the effective amount is an amount effective for inhibiting the activity or decreasing the level of a COX by at least 10%, at least 20%, at least 30%, at least 40%, at least 50%, at least 60%, at least 70%, at least 80%, at least 90%, at least 95%, or at least 98%. In certain embodiments, the effective amount is an amount effective for inhibiting the activity or decreasing the level of a COX by not more than 10%, not more than 20%, not more than 30%, not more than 40%, not more than 50%, not more than 60%, not more than 70%, not more than 80%, not more than 90%, not more than 95%, or not more than 98%.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a dog. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice, transgenic pigs). In certain embodiments, the subject is a fish or reptile.

In certain embodiments, the biological sample or cell (e.g., the biological sample or cell being contacted with a compound or pharmaceutical composition described herein) is in vitro. In certain embodiments, the biological sample or cell is in vivo or ex vivo. In certain embodiments, the biological sample or cell is a malignant cell or premalignant cell.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol*), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip©, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample or cell, any two doses of the multiple doses include different or substantially the same amounts of a compound described herein. In certain embodiments, when multiple doses are administered to a subject or applied to a biological sample or cell, the frequency of administering the multiple doses to the subject or applying the multiple doses to the biological sample or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample or cell is one dose per day. In certain embodiments, the frequency of administering the multiple doses to the subject or applying the multiple doses to the biological sample or cell is two doses per day. In certain embodiments, the frequency of administering the multiple doses to the subject or applying the multiple doses to the biological sample or cell is three doses per day. In certain embodiments, when multiple doses are administered to a subject or applied to a biological sample or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject or cell. In certain embodiments, the duration between the first dose and last dose of the multiple doses is three months, six months, or one year. In certain embodiments, the duration between the first dose and last dose of the multiple doses is the lifetime of the subject or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in inhibiting the activity or decreasing the level of a COX in a subject, biological sample, or cell), improve bioavailability, improve safety, reduce drug resistance, reduce and/or modify metabolism, inhibit excretion, and/or modify distribution in a subject, biological sample, or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, neurological disease, psychiatric disease, cancer, inflammatory disease, autoimmune disease, genetic disease, hematological disease, neurological disease, painful condition, psychiatric disorder, or metabolic disorder) or premalignant condition. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, cytotoxic chemotherapeutic agents, epigenetic modifiers, glucocorticoids, immunotherapeutic agents, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In certain embodiments, the additional pharmaceutical agent is an anti-proliferative agent (e.g., anti-cancer agent). In certain embodiments, the additional pharmaceutical agent is an anti-leukemia agent. In certain embodiments, the additional pharmaceutical agent is ABITREXATE (methotrexate), ADE, Adriamycin RDF (doxorubicin hydrochloride), Ambochlorin (chlorambucil), ARRANON (nelarabine), ARZERRA (ofatumumab), BOSULIF (bosutinib), BUSULFEX (busulfan), CAMPATH (alemtuzumab), CERUBIDINE (daunorubicin hydrochloride), CLAFEN (cyclophosphamide), CLOFAREX (clofarabine), CLOLAR (clofarabine), CVP, CYTOSAR-U (cytarabine), CYTOXAN (cyclophosphamide), ERWINAZE (Asparaginase Erwinia Chrysanthemi), FLUDARA (fludarabine phosphate), FOLEX (methotrexate), FOLEX PFS (methotrexate), GAZYVA (obinutuzumab), GLEEVEC (imatinib mesylate), Hyper-CVAD, ICLUSIG (ponatinib hydrochloride), IMBRUVICA (ibrutinib), LEUKERAN (chlorambucil), LINFOLIZIN (chlorambucil), MARQIBO (vincristine sulfate liposome), METHOTREXATE LPF (methorexate), MEXATE (methotrexate), MEXATE-AQ (methotrexate), mitoxantrone hydrochloride, MUSTARGEN (mechlorethamine hydrochloride), MYLERAN (busulfan), NEOSAR (cyclophosphamide), ONCASPAR (Pegaspargase), PURINETHOL (mercaptopurine), PURIXAN (mercaptopurine), Rubidomycin (daunorubicin hydrochloride), SPRYCEL (dasatinib), SYNRIBO (omacetaxine mepesuccinate), TARABINE PFS (cytarabine), TASIGNA (nilotinib), TREANDA (bendamustine hydrochloride), TRISENOX (arsenic trioxide), VINCASAR PFS (vincristine sulfate), ZYDELIG (idelalisib), or a combination thereof. In certain embodiments, the additional pharmaceutical agent is an anti-lymphoma agent. In certain embodiments, the additional pharmaceutical agent is ABITREXATE (methotrexate), ABVD, ABVE, ABVE-PC, ADCETRIS (brentuximab vedotin), ADRIAMYCIN PFS (doxorubicin hydrochloride), ADRIAMYCIN RDF (doxorubicin hydrochloride), AMBOCHLORIN (chlorambucil), AMBOCLORIN (chlorambucil), ARRANON (nelarabine), BEACOPP, BECENUM (carmustine), BELEODAQ (belinostat), BEXXAR (tositumomab and iodine I 131 tositumomab), BICNU (carmustine), BLENOXANE (bleomycin), CARMUBRIS (carmustine), CHOP, CLAFEN (cyclophosphamide), COPP, COPP-ABV, CVP, CYTOXAN (cyclophosphamide), DEPOCYT (liposomal cytarabine), DTIC-DOME (dacarbazine), EPOCH, FOLEX (methotrexate), FOLEX PFS (methotrexate), FOLOTYN (pralatrexate), HYPER-CVAD, ICE, IMBRUVICA (ibrutinib), INTRON A (recombinant interferon alfa-2b), ISTODAX (romidepsin), LEUKERAN (chlorambucil), LINFOLIZIN (chlorambucil), Lomustine, MATULANE (procarbazine hydrochloride), METHOTREXATE LPF (methotrexate), MEXATE (methotrexate), MEXATE-AQ (methotrexate), MOPP, MOZOBIL (plerixafor), MUSTARGEN (mechlorethamine hydrochloride), NEOSAR (cyclophosphamide), OEPA, ONTAK (denileukin diftitox), OPPA, R-CHOP, REVLIMID (lenalidomide), RITUXAN (rituximab), STANFORD V, TREANDA (bendamustine hydrochloride), VAMP, VELBAN (vinblastine sulfate), VELCADE (bortezomib), VELSAR (vinblastine sulfate), VINCASAR PFS (vincristine sulfate), ZEVALIN (ibritumomab tiuxetan), ZOLINZA (vorinostat), ZYDELIG (idelalisib), or a combination thereof. In certain embodiments, the additional pharmaceutical agent is REVLIMID (lenalidomide), DACOGEN (decitabine), VIDAZA (azacitidine), CYTOSAR-U (cytarabine), IDAMYCIN (idarubicin), CERUBIDINE (daunorubicin), LEUKERAN (chlorambucil), NEOSAR (cyclophosphamide), FLUDARA (fludarabine), LEUSTATIN (cladribine), or a combination thereof. In certain embodiments, the additional pharmaceutical agent is ABITREXATE (methotrexate), ABRAXANE (paclitaxel albumin-stabilized nanoparticle formulation), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), AVASTIN (bevacizumab), BECENUM (carmustine), BEP, BICNU (carmustine), BLENOXANE (bleomycin), CAF, CAMPTOSAR (irinotecan hydrochloride), CAPOX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CLAFEN (cyclophosphamide), CMF, COMETRIQ (cabozantinib-s-malate), COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), ERBITUX (cetuximab), ERIVEDGE (vismodegib), ETOPOPHOS (etoposide phosphate), EVACET (doxorubicin hydrochloride liposome), FARESTON (toremifene), FASLODEX (fulvestrant), FEC, FEMARA (letrozole), FLUOROPLEX (fluorouracil), FOLEX (methotrexate), FOLEX PFS (methotrexate), FOLFIRI, FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOVALDEX (tamoxifen citrate), OFF, PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), REVLIMID (lenalidomide), RUBIDOMYCIN (daunorubicin hydrochloride), SPRYCEL (dasatinib), STIVARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TRISENOX (arsenic trioxide), TYKERB (lapatinib ditosylate), VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZOLADEX (goserelin acetate), ZOMETA (zoledronic acid), ZYKADIA (ceritinib), ZYTIGA (abiraterone acetate), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055, BEZ²³⁵, BGT226, XL765, PF-4691502, GDC0980, SF1126, and OSI-027), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, or a combination thereof. In certain embodiments, the additional pharmaceutical agent is a cytotoxic chemotherapeutic agent (e.g., gemcitabine, cytarabine, daunorubicin, doxorubicin, vincristine, 1-asparaginase, cyclophosphamide, or etoposide). In certain embodiments, the additional pharmaceutical agent is an epigenetic modifier such as azacitidine or romidepsin. In certain embodiments, the additional pharmaceutical agent is ruxolitinib, BBT594, CHZ⁸⁶⁸, CYT387, or BMS911543. In certain embodiments, the additional pharmaceutical agent is an inhibitor of a tyrosine enzyme. In some embodiments, the additional pharmaceutical agent is a topoisomerase inhibitor, a MCL1 inhibitor, a BCL-2 inhibitor, a BCL-xL inhibitor, a BRD4 inhibitor, a BRCA1 inhibitor, BRCA2 inhibitor, HER1 inhibitor, HER2 inhibitor, a CDK9 inhibitor, a Jumonji histone demethylase inhibitor, or a DNA damage inducer. In some embodiments, the additional pharmaceutical agent is etoposide, obatoclax, navitoclax, JQ1, 4-(((5′-chloro-2′-(((1R,4R)-4-(((R)-1-methoxypropan-2-yl)amino)cyclohexyl)amino)-[2,4′-bipyridin]-6-yl)amino)methyl)tetrahydro-2H-pyran-4-carbonitrile, JIB04, or cisplatin. In certain embodiments, the additional pharmaceutical agent is a binder or inhibitor of a COX. In certain embodiments, the additional pharmaceutical agent is an antibody or a fragment thereof (e.g., monoclonal antibody). In certain embodiments, the additional pharmaceutical agent is a tyrosine enzyme inhibitor. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein enzyme inhibitors), modulators of protein stability (e.g., proteasome inhibitors), Hsp90 inhibitors, glucocorticoids, all-trans retinoic acids, and other agents that promote differentiation. In certain embodiments, the additional pharmaceutical agent is a glucocorticoid (e.g., cortisol, cortisone, prednisone, methylprednisolone, dexamethasone, betamethasone, triamcinolone, fludrocortisone acetate, or deoxycorticosterone acetate). In certain embodiments, the additional therapy is an immunotherapy (e.g., an immunotherapeutic monoclonal antibody). In certain embodiments, the additional pharmaceutical agent is an immunomodulator. In certain embodiments, the additional pharmaceutical agent is an immune checkpoint inhibitor. In certain embodiments, the additional pharmaceutical agent is a programmed cell death 1 protein (PD-1) inhibitor. In certain embodiments, the additional pharmaceutical agent is a programmed cell death 1 protein ligand 1 (PD-L1) inhibitor. In certain embodiments, the additional pharmaceutical agent is a cytotoxic T-lymphocyte-associated protein 4 (CTLA-4) inhibitor. In certain embodiments, the additional pharmaceutical agent is a T-cell immunoglobulin domain and mucin domain 3 (TIM3) inhibitor, lymphocyte activation gene-3 (LAG3) inhibitor, V-set domain-containing T-cell activation inhibitor 1 (VTCN1 or B7-H4) inhibitor, cluster of differentiation 276 (CD276 or B7-H3) inhibitor, B and T lymphocyte attenuator (BTLA) inhibitor, galectin-9 (GAL9) inhibitor, checkpoint enzyme 1 (Chk1) inhibitor, adenosine A2A receptor (A2AR) inhibitor, indoleamine 2,3-dioxygenase (IDO) inhibitor, killer-cell immunoglobulin-like receptor (KIR) inhibitor, or V-domain Ig suppressor of T cell activation (VISTA) inhibitor. In certain embodiments, the PD-1 inhibitor is nivolumab, pidilizumab, pembrolizumab, MEDI-0680, REGN2810, or AMP-224. In certain embodiments, the PD-L1 inhibitor is atezolizumab, durvalumab, BMS-936559, avelumab, or CA-170. In certain embodiments, the CTLA-4 inhibitor is ipilimumab or tremelimumab. In certain embodiments, the compounds described herein or pharmaceutical compositions can be administered in combination with an anti-cancer therapy including, but not limited to, surgery, radiation therapy, and transplantation (e.g., stem cell transplantation, bone marrow transplantation).

Also encompassed by the present disclosure are kits (e.g., pharmaceutical packs). In certain embodiments, the kit comprises a compound or pharmaceutical composition described herein, and instructions for using the compound or pharmaceutical composition. In certain embodiments, the kit comprises a first container, wherein the first container includes the compound or pharmaceutical composition. In some embodiments, the kit further comprises a second container. In certain embodiments, the second container includes an excipient (e.g., an excipient for dilution or suspension of the compound or pharmaceutical composition). In certain embodiments, the second container includes an additional pharmaceutical agent. In some embodiments, the kit further comprises a third container. In certain embodiments, the third container includes an additional pharmaceutical agent. In some embodiments, the compound or pharmaceutical composition included in the first container and the excipient or additional pharmaceutical agent included in the second container are combined to form one unit dosage form. In some embodiments, the compound or pharmaceutical composition included in the first container, the excipient included in the second container, and the additional pharmaceutical agent included in the third container are combined to form one unit dosage form. In certain embodiments, each of the first, second, and third containers is independently a vial, ampule, bottle, syringe, dispenser package, tube, or inhaler.

In certain embodiments, the instructions are for administering the compound or pharmaceutical composition to a subject (e.g., a subject in need of treatment or prevention of a disease described herein). In certain embodiments, the instructions are for contacting a biological sample or cell with the compound or pharmaceutical composition. In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

In certain embodiments, in the section “Pharmaceutical Compositions, Administration, and Kits”, articles such as “a,” “an,” and “the” mean one or more than one.

### Methods of Use and Uses

The present disclosure provides methods of inhibiting the activity (e.g., aberrant activity, such as increased activity) of a COX (e.g., COX2)). The present disclosure provides methods of inhibiting the activity (e.g., aberrant activity) or decreasing the level of a COX in a subject, biological sample, or cell. The present disclosure also provides methods for the treatment of a range of diseases and conditions, such as diseases and conditions associated with undesired or aberrant activity (e.g., increased activity) or overexpression of a COX. In certain embodiments, the diseases include proliferative diseases, musculoskeletal diseases, genetic diseases, hematological diseases, neurological diseases, painful conditions, psychiatric disorders, metabolic disorders, benign neoplasms, diseases associated with angiogenesis, inflammatory diseases, autoinflammatory diseases, autoimmune diseases, and premalignant conditions.

In another aspect, the present disclosure provides methods of treating a disease in a subject in need thereof, the method comprising administering to the subject in need thereof an effective amount (e.g., therapeutically effective amount) of a compound described herein or a pharmaceutical composition described herein.

In another aspect, the present disclosure provides methods of preventing a disease in a subject in need thereof, the method comprising administering to the subject in need thereof an effective amount (e.g., prophylactically effective amount) of a compound described herein or a pharmaceutical composition described herein.

In another aspect, the present disclosure provides methods of diagnosing a disease in a subject in need thereof, the method comprising administering to the subject in need thereof an effective amount (e.g., diagnostically effective amount) of a compound described herein or a pharmaceutical composition described herein.

In certain embodiments, the administration is oral administration. In certain embodiments, the administration is by injection (e.g., intravenous administration). In certain embodiments, the injection is an injection into an arm (e.g., an artery or vein in an arm) of the subject in need thereof. In certain embodiments, the administration is over a time period of between 1 and 5 seconds, between 5 and 10 seconds, between 10 and 30 seconds, between 30 and 60 seconds, between 1 and 2 minutes, between 2 and 5 minutes, or between 5 and 10 minutes, inclusive. In certain embodiments, the administration is over a time period of between 10 seconds and 2 minutes, inclusive. In certain embodiments, the methods described herein further comprise acquiring a positron emission tomography (PET) scan image of the subject in need thereof or a region thereof (the step of acquiring a PET scan image). In certain embodiments, the step of acquiring a PET scan image begins at between 1 and 5 minutes, between 5 and 10 minutes, between 10 and 30 minutes, between 30 and 60 minutes, between 1 and 2 hours, between 2 and 4 hours, between 4 and 6 hours, between 6 and 12 hours, between 12 and 24 hours, or between 1 and 2 days, inclusive, after the administration. In certain embodiments, the step of acquiring a PET scan image begins at between 10 and 60 minutes, inclusive, after the administration. In certain embodiments, the step of acquiring a PET scan image lasts between 1 and 5 minutes, between 5 and 10 minutes, between 10 and 20 minutes, between 20 and 40 minutes, between 40 and 60 minutes, or between 1 and 2 hours, inclusive. In certain embodiments, the step of acquiring a PET scan image lasts between 5 and 40 minutes, inclusive. In certain embodiments, the subject has substantially fasted from food and/or drink (e.g., water) during the time period between 4 and 24 hours, inclusive, before the administration. In certain embodiments, the subject has substantially fasted from food and/or drink (e.g., water) during the time period between 1 and 2 hours, between 2 and 4 hours, between 4 and 6 hours, between 6 and 8 hours, between 8 and 12 hours, or between 12 and 24 hours, inclusive, before the administration. In certain embodiments, the stomach of the subject in need thereof is substantially empty substantially during the step of acquiring a PET scan image. In certain embodiments, the bladder of the subject in need thereof is substantially empty substantially during the step of acquiring a PET scan image. In certain embodiments, the subject in need thereof is lying down in a substantially horizontal position substantially during the step of acquiring a PET scan image. In certain embodiments, both arms of the subject in need thereof are in a substantially overhead position substantially during the step of acquiring a PET scan image. In certain embodiments, both arms of the subject in need thereof are positioned adjacent to the torso of the subject in need thereof substantially during the step of acquiring a PET scan image.

In certain embodiments, the uptake of the compound in the region (e.g., an organ or tissue in the region) is more than the uptake of the compound in at least one of the other regions (e.g., an organ or tissue in at least one of the other regions). In certain embodiments, the uptake of the compound in the region (e.g., an organ or tissue in the region) represents specific binding to a COX. In certain embodiments, the region is the head. In certain embodiments, the region is the brain. In certain embodiments, the region is the neck. In certain embodiments, the region is the chest. In certain embodiments, the region is the abdomen. In certain embodiments, the region is the pelvic region.

In certain embodiments, the methods described herein further comprise acquiring a magnetic resonance imaging (MRI) scan image of the subject in need thereof or a region thereof (the step of acquiring an MRI scan image). In certain embodiments, the methods described herein further comprise acquiring an X-ray computed tomography (CT) scan image of the subject in need thereof or a region thereof (the step of acquiring a CT scan image). In certain embodiments, the step of acquiring a PET scan image is performed prior or subsequent to the step of acquiring an MRI scan image. In certain embodiments, the step of acquiring a PET scan image is performed prior or subsequent to the step of acquiring an CT scan image. In certain embodiments, the step of acquiring a PET scan image is performed substantially concurrently with the step of acquiring a CT scan image.

In certain embodiments, the methods described herein further comprise interpreting the scan image(s) (e.g., PET scan image, MRI scan image, CT scan image). In certain embodiments, the step of interpreting the scan image(s) comprises quantifying a COX.

In another aspect, the present disclosure provides methods of inhibiting the activity or decreasing the level of a COX in a subject in need thereof, the method comprising administering to the subject in need thereof an effective amount of a compound described herein or a pharmaceutical composition described herein.

In another aspect, the present disclosure provides methods of inhibiting the activity or decreasing the level of a COX in a biological sample (e.g., an in vitro biological sample), the method comprising contacting the biological sample with an effective amount of a compound described herein or a pharmaceutical composition described herein.

In another aspect, the present disclosure provides methods of inhibiting the activity or decreasing the level of a COX in a cell (e.g., an in vitro cell), the method comprising contacting the cell with an effective amount of a compound described herein or a pharmaceutical composition described herein.

Without wishing to be bound by any particular theory, in certain embodiments the compounds described herein are able to bind a COX. In certain embodiments, the disease is a disease associated with aberrant activity or elevated level of a COX (e.g., COX2). In certain embodiments, the effective amount is further effective for inhibiting the activity or decreasing the level of COX2. In certain embodiments, the effective amount is more effective for inhibiting the activity or decreasing the level of COX2 than COX1.

In certain embodiments, provided are methods of decreasing the activity of a COX in a subject, biological sample, or cell by at least about 1%, at least about 3%, at least about 10%, at least about 20%, at least about 30%, at least about 40%, at least about 50%, at least about 60%, at least about 70%, at least about 80%, or at least about 90%. In certain embodiments, the activity of a COX in a subject, biological sample, or cell is decreased by at least about 1%, at least about 3%, at least about 10%, at least about 20%, at least about 30%, at least about 40%, at least about 50%, at least about 60%, at least about 70%, at least about 80%, or at least about 90%. In some embodiments, the activity of a COX in a subject, biological sample, or cell is selectively inhibited by the method. In some embodiments, the activity of a COX in a subject, biological sample, or cell is selectively decreased by a compound or pharmaceutical composition described herein.

In certain embodiments, the disease (e.g., the disease to be treated or prevented by a method described herein) is associated with the increased level of a COX. In certain embodiments, the disease (e.g., the disease to be treated or prevented by a method described herein) is associated with the increased activity of a COX. In certain embodiments, the disease is associated with overexpression of a COX.

In certain embodiments, the disease is a proliferative disease.

In certain embodiments, the cancer is a hematological malignancy. In certain embodiments, the cancer is a solid tumor.

In some embodiments, the disease is a benign neoplasm.

In certain embodiments, the disease is an inflammatory disease.

In some embodiments, the disease is an autoinflammatory disease.

In certain embodiments, the disease is a premalignant condition.

In certain embodiments, the disease is a central nervous system (CNS) disease. In certain embodiments, the disease is a brain disease. In certain embodiments, the disease (e.g., brain disease) is Aicardi-Goutieres syndrome, Alpers-Huttenlocher syndrome, anencephaly, aphasia, Balo concentric sclerosis, basal ganglia disease, blood-brain barrier injury, bovine spongiform encephalopathy, brain abscess, brain atrophy, brain death, brain edema, brain hippocampus disease, brain infarction, brain infection, brain injury, brain insufficiency, brain ischemia, brain lesion, brain neoplasm, brain stem disease, central pontine myelinolysis, cerebellar disease, cerebral amyloidosis, cerebral creatine deficiency syndrome-1, cerebral involution, cerebrocortical disease, cerebrovascular disease, coma, concussion, dementia, diffuse cerebral sclerosis of Schilder, encephalitis, encephalocele, encephalomalacia, epilepsy, exencephaly, extrapontine myelinolysis, Gerstmann-Straussler syndrome, Gilles de la Tourette syndrome, Harada syndrome, headache, hemimegalencephaly, hepatic encephalopathy, holoprosencephaly, hydrocephalus, hypertensive encephalopathy, hypothalamic disease, kernicterus, kuru, Leigh disease, leukodystrophy, leukoencephalopathy, limbic system disease, lissencephaly, memory disorders, metabolic encephalopathy, micrencephaly, Miller-Dieker syndrome, mitochondrial encephalomyopathy, neuritic plaque, neurodegenerative brain disease, neurofibrillary tangle, pellagra-like encephalopathy, periventricular leukomalacia, Rett syndrome, Reye syndrome, scrapie, spongiform encephalopathy, subarachnoid hemorrhage, syncope, vegetative state, Walker-Warburg syndrome, Wilson disease, X-linked agenesis of corpus callosum, or X-linked hydrocephalus.

In certain embodiments, the disease (e.g., brain disease) is dementia. In certain embodiments, the disease (e.g., dementia) is frontal lobe dementia, Creutzfeldt-Jakob disease, familial British dementia, familial Danish dementia, frontotemporal lobe dementia, Pick disease, primary progressive aphasia, progressive supranuclear palsy, Gerstmann-Straussler syndrome, Guamanian parkinsonism-dementia, Huntington disease, Nasu-Hakola disease, Pick disease, Alzheimer disease, Lewy body dementia, CADASIL, AIDS dementia complex, subcortical dementia, Binswanger disease, or multi-infarct dementia. In certain embodiments, the disease is Huntington's disease.

In certain embodiments, the disease (e.g., CNS disease) is Edwards syndrome, gliosis, hyperekplexia, Meckel syndrome, myoclonic epilepsy myopathy sensory ataxia, narcolepsy, prion diseases, serotonin syndrome, or spinal cord disease.

In certain embodiments, the disease is a psychiatric disorder. In certain embodiments, the disease is schizophrenia.

In certain embodiments, the disease is an inflammatory disease. In certain embodiments, the disease is neuroinflammation.

In certain embodiments, the method described herein superior (e.g., showing improved safety and/or therapeutic effects) or comparable to existing therapy (e.g., chemotherapy).

In certain embodiments, the subject (e.g., the subject in need of treatment or prevention) is a mammal. In certain embodiments, the subject is a human (e.g., an adult, juvenile, or child). In certain embodiments, the subject is a non-human mammal. In certain embodiments, the subject is a dog.

In certain embodiments, the biological sample or cell (e.g., the biological sample or cell being contacted with a compound or pharmaceutical composition described herein) is in vitro. In certain embodiments, the biological sample or cell is in vivo. In certain embodiments, the biological sample or cell is ex vivo.

In certain embodiments, the cell is a malignant cell (e.g., cancer cell). In certain embodiments, the cell is a malignant blood cell. In certain embodiments, the cell is a malignant bone marrow cell. In certain embodiments, the cell is an adenocarcinoma cell, blastoma cell, carcinoma cell, or sarcoma cell. In certain embodiments, the cell is a pre-malignant cell (e.g., pre-cancerous cell).

In certain embodiments, the method described herein further comprises administering to the subject in need thereof an additional therapy. In certain embodiments, the additional therapy is an additional pharmaceutical agent described herein. In certain embodiments, the additional therapy is a cytotoxic chemotherapy (e.g., gemcitabine, cytarabine, daunorubicin, doxorubicin, vincristine, 1-asparaginase, cyclophosphamide, or etoposide). In certain embodiments, the additional therapy is an epigenetic modifier (e.g., azacitidine or romidepsin). In certain embodiments, the additional therapy is a glucocorticoid. In certain embodiments, the additional therapy is an immunotherapy (e.g., an immunotherapeutic monoclonal antibody). In some embodiments, the additional pharmaceutical agent is etoposide, obatoclax, or navitoclax, and optionally the disease is breast cancer, e.g., triple-negative breast cancer, HER2 positive breast cancer, HER2 negative breast cancer, ER-positive breast cancer, ER-negative breast cancer, or ER/PR-positive breast cancer. In some embodiments, the additional pharmaceutical agent is etoposide, JIB04, or cisplatin, and optionally the disease is Ewing's sarcoma. In some embodiments, the additional pharmaceutical agent is JQ1 or NVP2, and optionally the disease is leukemia, e.g., acute myelogenous leukemia, myeloblastic leukemia, promyelocytic leukemia, myelomonocytic leukemia, monocytic leukemia, monoblastic leukemia, or megakaryoblastic leukemia.

In yet another aspect, the present invention provides compounds and pharmaceutical compositions described herein for use in the treatment of a disease (e.g., a proliferative disease, such as cancer) in a subject in need thereof.

In yet another aspect, the present invention provides compounds and pharmaceutical compositions described herein for use in the prevention of a disease (e.g., a proliferative disease, such as cancer) in a subject in need thereof.

In another aspect, the present disclosure provides compounds and pharmaceutical compositions described herein for use in inhibiting the activity or decreasing the level of a COX in a subject in need thereof.

In another aspect, the present disclosure provides compounds and pharmaceutical compositions described herein for use in inhibiting the activity or decreasing the level of a COX in a biological sample (e.g., an in vivo or ex vivo biological sample).

In another aspect, the present disclosure provides compounds and pharmaceutical compositions described herein for use in inhibiting the activity or decreasing the level of a COX in a cell (e.g., an in vivo or ex vivo cell).

In another aspect, the present disclosure provides uses of compounds and pharmaceutical compositions described herein in the manufacture of a medicament for treating a disease in a subject in need thereof.

In another aspect, the present disclosure provides uses of compounds and pharmaceutical compositions described herein in the manufacture of a medicament for preventing a disease in a subject in need thereof.

The compounds, pharmaceutical compositions, and kits described herein may synergistically augment inhibition of a COX induced by the additional pharmaceutical agent(s) in the biological sample or subject. Thus, the combination of the compounds, pharmaceutical compositions, or kits with additional pharmaceutical agent(s) may be useful in treating diseases resistant to a treatment using the additional pharmaceutical agent(s) without the compounds, pharmaceutical compositions, or kits described herein.

In certain embodiments, in the section “Methods of Use and Uses”, articles such as “a,” “an,” and “the” mean one or more than one.

### EXAMPLES

In order that the invention described herein may be more fully understood, the following examples are set forth. It should be understood that these examples are for illustrative purposes only and are not to be construed as limiting this invention in any manner.

### Example 1

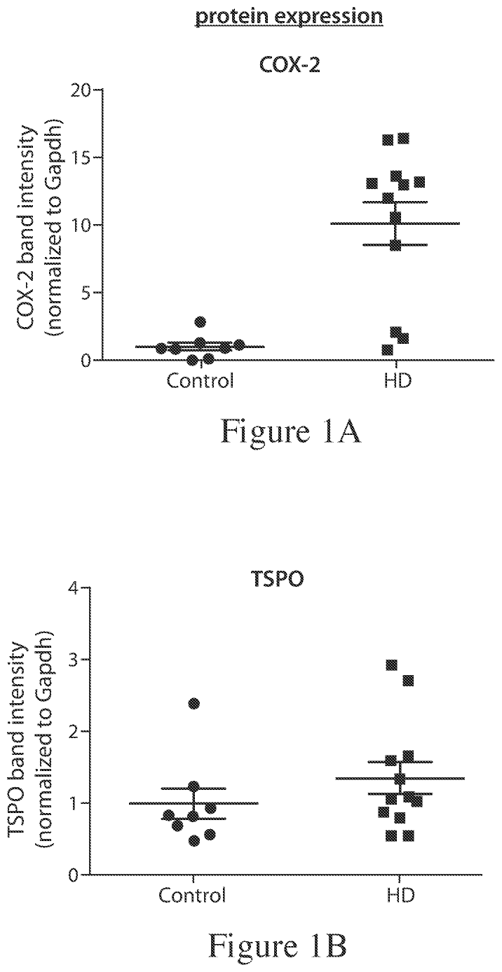

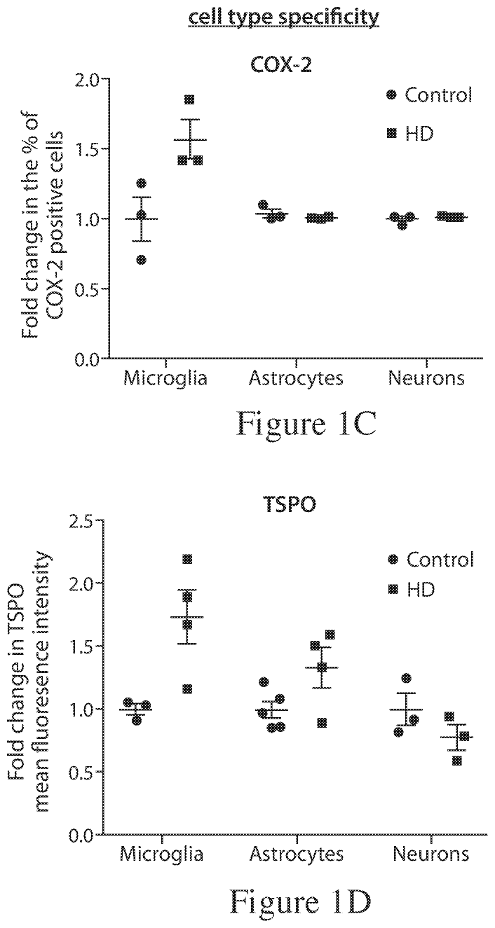

Using human post-mortem globus pallidus tissue from healthy controls and HD patients, we have demonstrated that COX2 expression is increased approximately 10-fold with disease (FIG. 1A) while TSPO levels only increased 1.3-fold (FIG. 1B). This supports our hypothesis that COX2 targeting will provide larger, more robust imaging signals when compared to TSPO. Using immunohistochemical staining, we further demonstrated that increases in COX2 localize to microglia, whereas TSPO expression changes increased in microglia and astrocytes, and slightly decreased in neurons (FIGS. 1C and 1D).

We also performed quantitative RT-PCR for COX2 using RNA extracted from the same post-mortem tissue and again found a robust increase in expression in the caudate nucleus of HD samples (FIG. 1E). This COX2 increase was also observed in two rare samples of post-mortem tissue from pre-symptomatic HD patients, in which we found protein levels of synaptic markers were also reduced, confirming early disease pathology (FIG. 1F). Significant transcript increases were not observed for other highly expressed microglia specific genes, suggesting that COX2 has the unique potential to be an early biomarker of a microglial phenotype associated with development of synaptic pathology (as seen in the HD mouse models).

Similar microglia-specific COX2 upregulation was observed in two HD mouse models (zQ175 and DN17) in disease-affected regions such as the striatum, but not in less affected regions like the cerebellum (data not shown). Together these data shape the scientific premise that COX2 may be a mechanistically relevant biomarker of neuroinflammation in HD, and potentially in other CNS disorders.

Development of COX2 PET ligand. Although highly potent and selective COX2 ligands (coxibs) were developed by the pharmaceutical industry, none of these have been successfully translated to clinical application as PET imaging probes. A majority of these compounds were not designed with fast binding kinetics and brain penetrance as primary optimization parameters [Tietz, 2013]. We have developed a unique design strategy for the optimization of potential COX2 PET radioligands starting from known coxibs. Our strategy prioritizes binding affinity (Kd<1 nM), isoform selectivity (>1000-fold against COX-1), fast binding kinetics (on-rate kon), low non-specific binding (NSB<95%), high brain penetration (CNS-PET-MPO score>4), and other key radiopharmacological criteria necessary for a successful PET tracer. The design of novel analogs was also strongly guided by computational modeling using the Celecoxib-mCOX2 co-crystal (PDB: 3LN1) and the rofecoxib-hCOX2 co-crystal (PDB: 5KIR) [Orlando, 2016].

We have adapted a previously reported cyclooxygenase enzymatic assay utilizing LCMS quantification of PGE2 as the product of COX-1 and COX2 catalyzed conversion of arachidonic acid to evaluate the binding properties of newly synthesized ligands [Cao, 2011]. These optimized and miniaturized (384-well plates) LCMS-based COX-1 and COX2 enzymatic assays enabled us to quickly evaluate newly synthesized ligands for COX2 binding affinity and selectivity vs COX-1. Using a shorter ligand-enzyme incubation time in the LCMS assay (2 minutes vs 10 minutes) also permitted us to prioritize ligands with faster binding kinetics. Considering the relatively slow on-rate kinetics of known coxibs, due to their 3-step binding mechanism [Marnett, 2009], we believe that optimizing this critical parameter will be key to the successful development of a COX2 PET tracer. Using these enzymatic assays, we have evaluated>200 newly synthesized ligands against COX2 and >20 ligands against COX-1. Another critical property necessary for a good PET radiotracer is to have low non-specific binding (NSB) to increase the free fraction and maximize the signal to noise ratio. This property can be evaluated using in-vitro protein binding assays, and we have now evaluated>50 newly synthesized ligands in mouse, rat and human plasma protein binding (PPB) assays as well as >15 ligands in a mouse brain protein binding (BPB) assay.

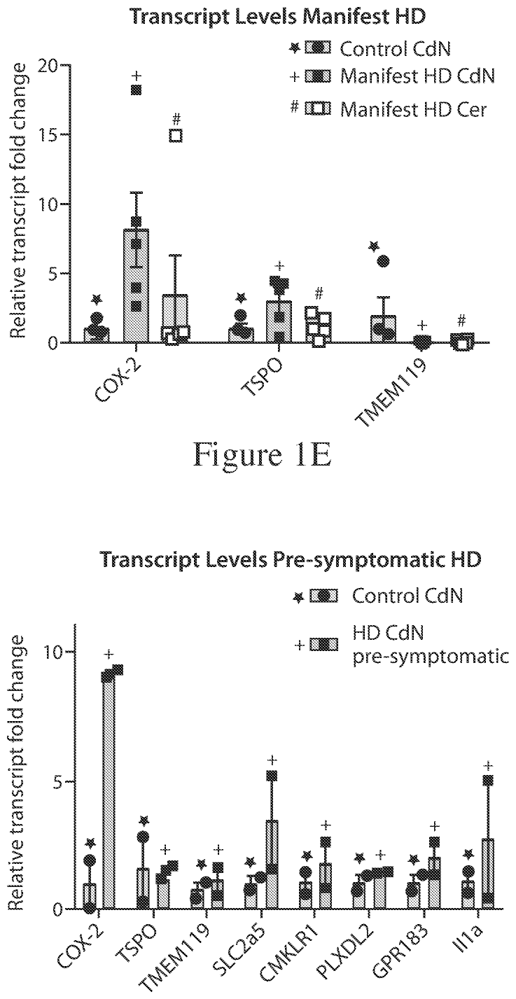

Three chemical series were investigated; Rofecoxib, Lumiracoxib and MC1 (FIG. 2). Based on a compromise of potency, selectivity, binding kinetics and non-specific binding, the MC1 chemical series was prioritized. Extensive structure-activity-relationship (SAR) study led to a 10-fold improvement in potency (331) or 3-fold improvement in free fraction (98, FIG. 2).

Three ligands have been evaluated with in-vitro autoradiography in rat brain slices. Radiolabeling was accomplished via methylation of either a sulfinate precursor ([¹¹C]Rofecoxib), hydroxyl precursor ([¹¹C]MC1) or thiol precursor ([¹¹C]98) using [¹¹C]-methyl iodide and offers chemical flexibility for radiolabeling of future candidates. The pyrazine core of 98 provides additional free fraction compared to MC1 (˜3-fold improvement) while maintaining potency. This translated into a ˜2-fold increase in COX2 specific binding in a rat brain slice in-vitro autoradiography. 116, which maintains MC1 free fraction (2.5%) but with a ˜6-fold improvement in potency was radiolabeled and evaluated in the autoradiography experiment (e.g., as discussed in Example 2). These properties enable us to better understand the contribution of potency and free fraction to the ligand's specific binding.

### Example 2

Dysregulated synaptic pruning is observed in a wide range of psychiatric disorders, and emerging evidence suggests a potential role of this process in the etiology of schizophrenia. Microglia, the resident immune cells of the CNS, are key regulators of synaptic refinement mediated via the complement system. Biomarkers are necessary to study microglia activation in humans and to understand how and if it relates to disease manifestation and progression and therapeutic interventions. Current efforts in the Stevens lab are focused on the potential to identify microglia activation biomarkers in the CSF of patients suffering from schizophrenia. As a complementary approach, PET imaging enables in vivo longitudinal measurements of molecular changes in healthy and diseased brains, facilitating the study of disease progression and the identification of molecular markers associated with diseases where activated microglia have been implicated.

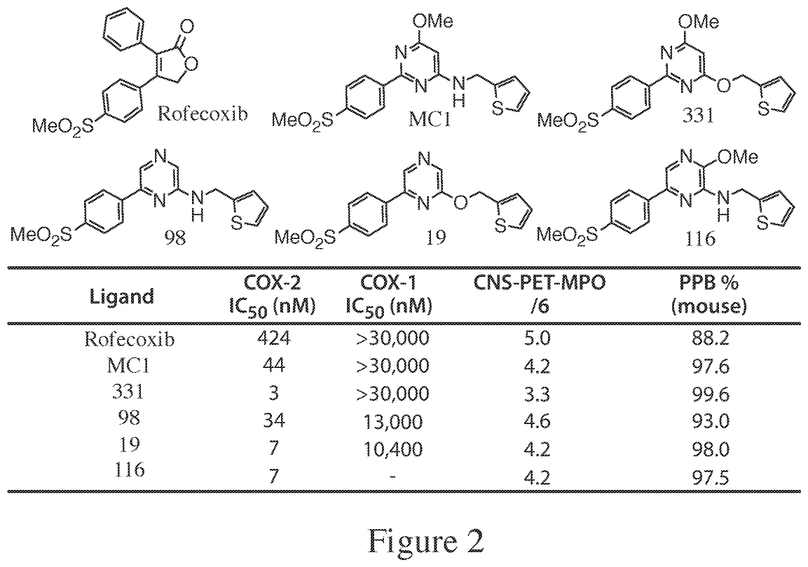

In Example 1, we showed that COX2 protein levels were specifically increased in microglia cells of globus pallidus from post-mortem huntington's disease (HD) brain slices, and COX2 transcripts were significantly upregulated in the caudate nucleus of manifest and pre-symptomatic HD brain slices, suggesting that COX2 as the potential to be an early biomarker of microglia activation in HD. As a result, we initiated a medicinal chemistry effort to develop a brain penetrant COX2 radiotracer. Critically, developing a PET tracer is not as straightforward as labelling a known inhibitor (e.g., rofecoxib or MC-1). Several properties are crucial for the development of a successful PET tracer, amongst which high binding affinity (which rofecoxib lacks) and selectivity for the target (to ensure maximal binding potential BP=Bmax/Kd), as well as high fraction unbound (to ensure low non-specific binding and fast clearance in vivo), are paramount. Concentrating our chemistry efforts on these properties, we developed 98, with equivalent binding affinity and improved fraction unbound (3-fold) vs MC-1 (FIG. 3) and demonstrated that these improvements lead to an increase in specific binding using in vitro rat brain slices autoradiography.

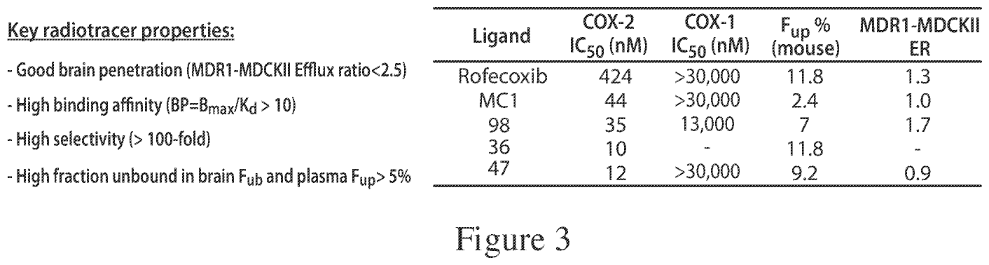

Medicinal chemistry efforts built upon previous observations that increasing binding affinity for COX2 and fraction unbound lead to higher specific binding in rat brain slices. Structure based drug design (SBDD) and structure activity relationship (SAR) studies led to the identification of 36 and 47 as our current lead COX2 ligands, with improved potency (˜4-fold) and fraction unbound (˜4-fold, FIGS. 3 and 4). Both radiotracers were synthesized using a thioester precursor that can rapidly be converted to the desired sulfone in 3 steps, resulting in the incorporation of radioactivity through the use of 11C-methyl iodide (FIG. 4).

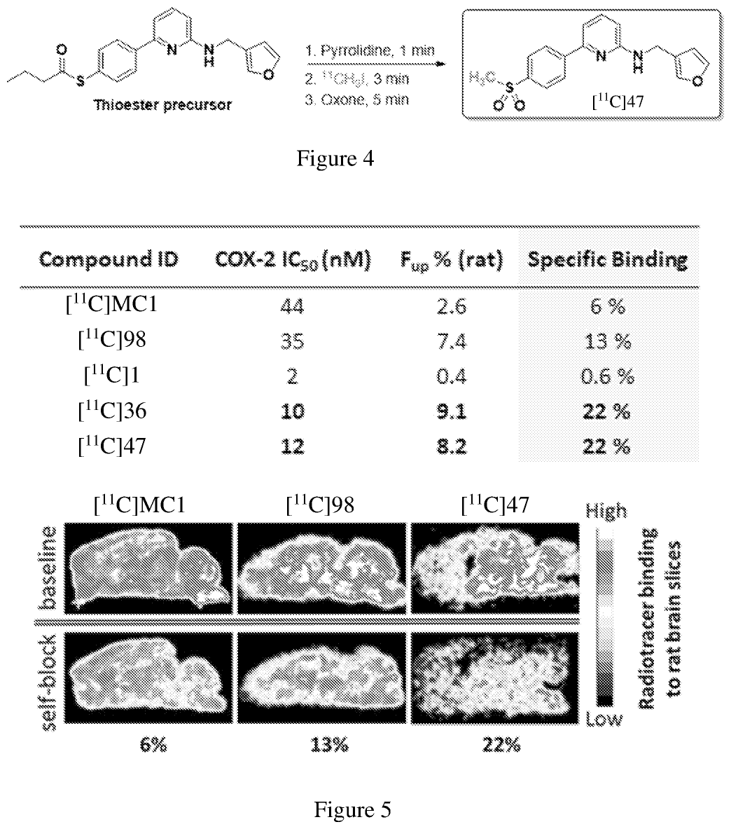

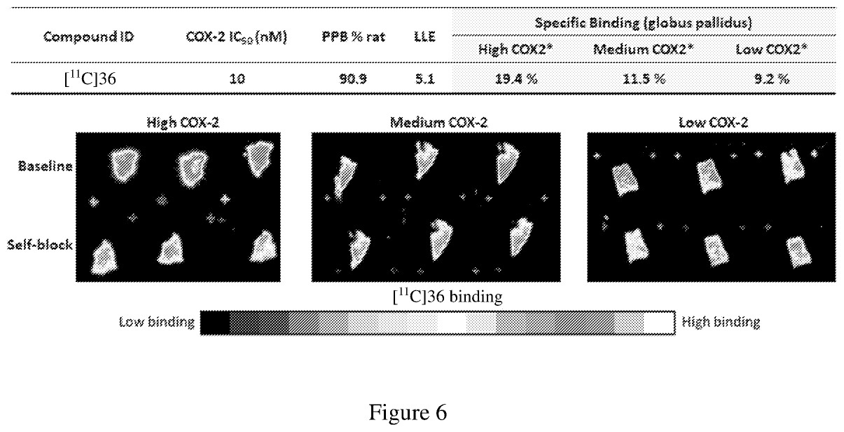

Three newly synthesized COX2 ligands were successfully radiolabeled using this strategy and first evaluated by in-vitro autoradiography in rat brain slices using self-block experiments (FIG. 5). [¹¹C]36 and [¹¹C]47 both showed significantly increased specific binding compared to [¹¹C]MC1 and last year's lead tracer [¹¹C]98, further validating our strategy to optimize binding affinity and fraction unbound. Brain pharmacokinetic of [¹¹C]47 was evaluated using rat in-vivo PET imaging and demonstrated high and rapid brain uptake followed by fast clearance, a desired profile for a PET radiotracer, in order to quickly saturate specific binding and maximize elimination of excess radiotracer to achieve optimal imaging contrast and limit scanning duration. Using a self-block experiment, a modest but consistent specific binding was also observed between 30-60 minutes. [¹¹C]36 was also evaluated for specific binding using self-block experiments in human post-mortem brain tissues (globus pallidus) from three Huntington's disease (HD) patients (FIG. 6). COX2 protein levels were quantified using immunostaining and defined as high, medium and low, respectively. Interestingly, COX2 protein levels quantified using [¹¹C]36 were in agreement with immunostaining observations, further confirming that specific binding of one of our lead tracers is COX2 dependent in a human brain tissue region known to be affected in disease.

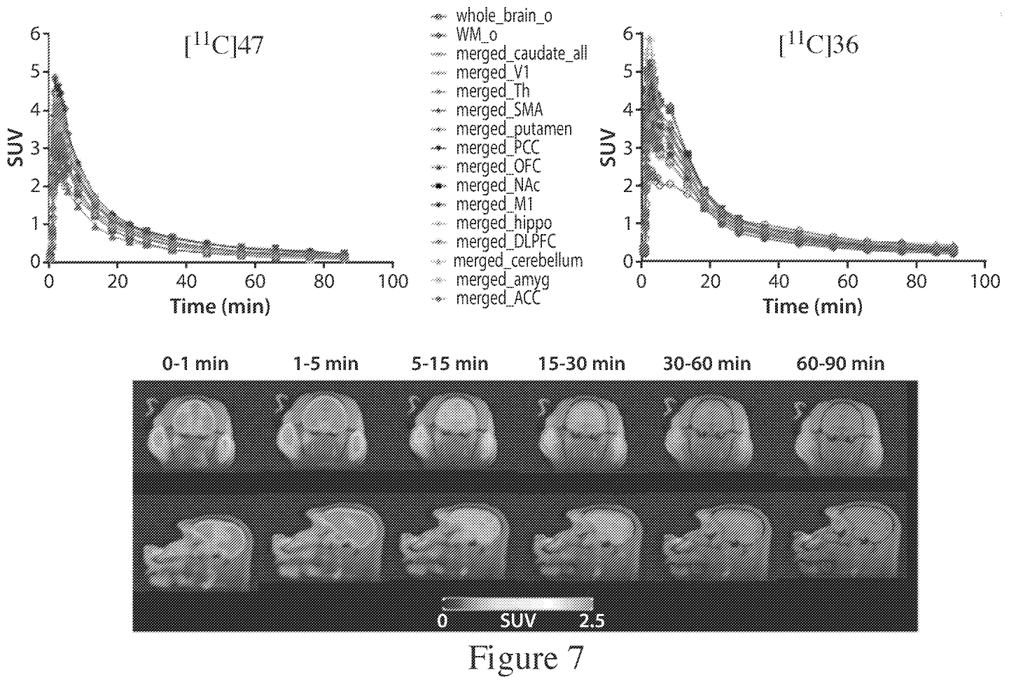

Additionally, we demonstrated high, fast and homogeneous brain uptake along with fast clearance in the non-human primate (baboon) for both of our lead tracers [¹¹C]36 and [¹¹C]47 (Standard Uptake Volume (SUV) ranging from 2-6 across brain regions, FIG. 7). This ideal pharmacokinetic profile in non-human primate positions these two optimized lead radiotracers for further evaluation using kinetic modeling with an arterial plasma input function and correction for plasma fraction unbound to obtain total volume of distribution (VT), the gold standard measure for PET quantification.

### Example 3. Synthesis of Compounds

If the absolute stereochemistry of a compound is arbitrarily assigned, then the absolute stereochemistry of the compound has not been determined, and the arbitrarily assigned absolute stereochemistry may be correct or incorrect. In case the arbitrarily assigned absolute stereochemistry is incorrect, the absolute stereochemistry is identified by the order of elution (from the first to the last) of the compound from the chiral chromatography that was used for purifying the compound. For example, Compound 115 is one of the two stereoisomers of

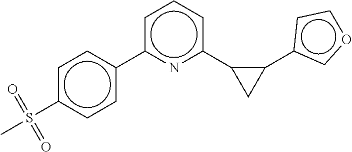

that includes a trans cyclopropylene moiety (trans

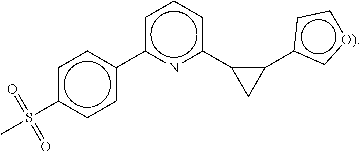

However, Compound 115's absolute stereochemistry has not been determined. That is, it has not been determined whether Compound 115 is of the formula:

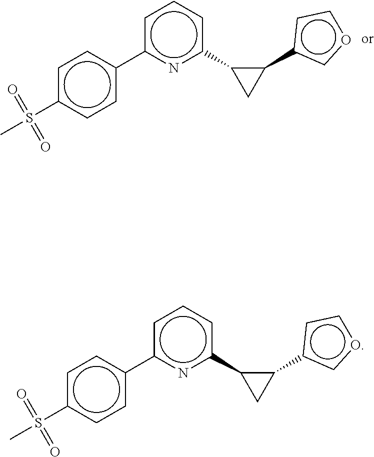

In case the arbitrarily assigned absolute stereochemistry of Compound 115 is incorrect, the absolute stereochemistry of Compound 115 is identified by that Compound 115 was the first eluting stereoisomer of trans

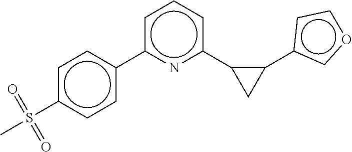

when purified by chiral SFC purification by CO2 and 0.1% diethyl amine in methanol as a co-solvent using Chiralcel OJ-H (250 mm*4.6 mm*5 m).

### 2-chloro-6-[4-(methylsulfanyl)phenyl]pyridine

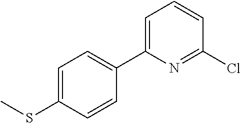

In a flask under nitrogen were dissolved 2,6-dichloropyridine (2.0 g, 13.5 mmol, 1.0 eq), [4-(methylsulfanyl)phenyl]boronic acid (2.26 g, 13.5 mmol, 1.0 eq), potassium carbonate (3.73 g, 27.0 mmol, 2.0 eq) and Pd(dppf)Cl₂ (275 mg, 0.338 mmol, 2.5 mol %) in degassed 1,4-dioxane/water (1:1, 50 mL). The mixture was stirred overnight at 65° C., then brought back to room temperature. Water (65 mL) was added and the aqueous layer was extracted with DCM (3×100 mL). The combined organic layers were dried over MgSO₄, filtered and evaporated under reduced pressure. The crude material was purified by normal phase chromatography (hexanes/ethyl acetate, 0-15%) to afford 2-chloro-6-[4-(methylsulfanyl)phenyl]pyridine (1.8328 g, 87% purity, 50.0% yield) as a white solid.

MS: [M+H]+ 236.10

¹H NMR (400 MHz, Chloroform-d) δ 7.93 (d, J=8.5 Hz, 2H), 7.68 (t, J=7.7 Hz, 1H), 7.61 (dd, J=7.8, 0.9 Hz, 1H), 7.32 (d, J=8.5 Hz, 2H), 7.23 (dd, J=7.7, 0.9 Hz, 1H), 2.53 (s, 3H).

### 2-bromo-6-[4-(methylsulfanyl)phenyl]pyridine

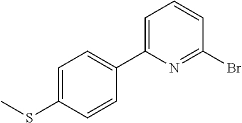

In a flask under nitrogen were dissolved 2,6-dibromopyridine (2.0 g, 8.44 mmol, 1.0 eq), [4-(methylsulfanyl)phenyl]boronic acid (1.41 g, 8.44 mmol, 1.0 eq), potassium carbonate (2.32 g, 16.8 mmol, 2.0 eq) and Pd(dppf)Cl₂ (308 mg, 0.422 mmol, 5 mol %) in degassed 1,4-dioxane/water (1:1, 50 mL). The mixture was stirred overnight at 70° C., then brought back to room temperature. Water (80 mL) was added and the aqueous layer was extracted with DCM (3×100 mL). The combined organic layers were dried over MgSO₄, filtered and evaporated under reduced pressure. The crude material was purified by normal phase chromatography (hexanes/ethyl acetate, 20-40%) to afford 2-bromo-6-[4-(methylsulfanyl)phenyl]pyridine (1.463 g, 91% purity, 56.3% yield) as a white solid.

MS: [M+H]+ 282.10

¹H NMR (400 MHz, Chloroform-d) δ 7.93 (d, J=8.5 Hz, 2H), 7.65 (dd, J=7.7, 0.9 Hz, 1H), 7.57 (t, J=7.8 Hz, 1H), 7.38 (dd, J=7.7, 0.9 Hz, 1H), 7.32 (d, J=8.5 Hz, 2H), 2.53 (s, 3H).

### 2-chloro-6-(4-methanesulfonylphenyl)pyridine

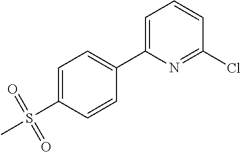 [SMILES: CS(=O)(=O)c1ccc(-c2cccc(Cl)n2)cc1]

In a flask under nitrogen were dissolved 2,6-dichloropyridine (2.0 g, 13.5 mmol, 1.0 eq), (4-methanesulfonylphenyl)boronic acid (2.70 g, 13.5 mmol, 1.0 eq), potassium carbonate (3.73 g, 27.0 mmol, 2.0 eq) and Pd(dppf)Cl₂ (98.7 mg, 0.135 mmol, 1 mol %) in degassed 1,4-dioxane/water (1:1, 20 mL). The mixture was stirred overnight at 45° C. Water was added and extracted with DCM. The combined organic layers were dried over MgSO₄, filtered and evaporated. The crude material was purified by normal phase chromatography (hexanes/ethyl acetate, 20-65%) to afford 2-chloro-6-(4-methanesulfonylphenyl)pyridine (1.8075 g, 100% purity, 49.8% yield) as a white solid.

MS: [M+H]+ 367.78

¹H NMR (400 MHz, Chloroform-d) δ 8.21 (d, J=8.5 Hz, 2H), 8.05 (d, J=8.5 Hz, 2H), 7.79 (t, J=7.7 Hz, 1H), 7.73 (dd, J=7.7, 0.9 Hz, 1H), 7.37 (dd, J=7.7, 0.9 Hz, 1H), 3.09 (s, 3H).

### 2-bromo-6-(4-methanesulfonylphenyl)pyridine

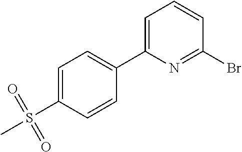 [SMILES: CS(=O)(=O)c1ccc(-c2cccc(Br)n2)cc1]

In a flask under nitrogen were dissolved 2,6-dibromopyridine (2.0 g, 8.44 mmol, 1.0 eq), (4-methanesulfonylphenyl)boronic acid (1.68 g, 8.44 mmol, 1.0 eq), potassium carbonate (2.32 g, 16.8 mmol, 2.0 eq) and Pd(dppf)Cl₂ (61.7 mg, 0.08439 mmol, 1 mol %) in degassed 1,4-dioxane/water (1:1, 20 mL). The mixture was stirred at 45° C. for 2 h. Water was added and extracted with DCM. The combined organic layers were dried over MgSO₄, filtered and evaporated. The crude material was purified by normal phase chromatography (hexanes/ethyl acetate, 15-60%) to afford 2-bromo-6-(4-methanesulfonylphenyl)pyridine (1.2211 g, 95.3% purity, 44.1% yield) as a white solid.

MS:[M+H]+ 312.07

¹H NMR (400 MHz, Chloroform-d) δ 8.20 (d, J=8.5 Hz, 2H), 8.04 (d, J=8.5 Hz, 2H), 7.76 (d, J=7.6 Hz, 1H), 7.67 (t, J=7.7 Hz, 1H), 7.52 (d, J=7.7 Hz, 1H), 3.09 (s, 3H).

### N-(furan-3-ylmethyl)-6-(4-(methylsulfonyl)phenyl)pyridin-2-amine (47)

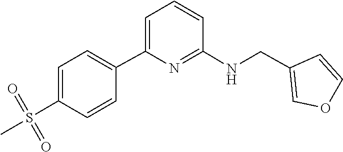 [SMILES: CS(=O)(=O)c1ccc(-c2cccc(NCc3ccoc3)n2)cc1]

In a flask under nitrogen were dissolved 2-bromo-6-(4-methanesulfonylphenyl)pyridine (300 mg, 0.9609 mmol, 1 eq), (tert-butoxy)sodium (138 mg, 1.44 mmol, 1.5 eq), tBuBrettPhos Pd G3 (123 mg, 0.1441 mmol, 15 mol %), tBuBrettPhos (69.8 mg, 0.1441 mmol, 15 mol %) and 1-(furan-3-yl)methanamine (132 μL, 1.44 mmol, 1.5 eq) in degassed 1,4-dioxane (5 mL). The mixture was stirred at 60° C. for 1 h. The solvent was evaporated and the crude material was purified by reverse phase chromatography (water/acetonitrile+0.1% formic acid, 30-80%) to afford the desired product. It was then washed a few times with little amounts of diethyl ether to remove remaining traces of ligand and afford N-[(furan-3-yl)methyl]-6-(4-methanesulfonylphenyl)pyridin-2-amine (193.3 mg, 100% purity, 61.2% yield) as a white solid.

MS: [M+H]+ 328.76

¹H NMR (400 MHz, Chloroform-d) δ 8.19 (d, J=8.7 Hz, 2H), 8.00 (d, J=8.7 Hz, 2H), 7.54 (t, J=7.9 Hz, 1H), 7.44 (s, 1H), 7.40 (t, J=1.6 Hz, 1H), 7.13 (d, J=7.5 Hz, 1H), 6.46 (d, J=8.2 Hz, 1H), 6.44 (s, 1H), 4.78 (s, 1H), 4.47 (d, J=4.5 Hz, 2H), 3.08 (s, 2H).

### 2-(4-methanesulfonylphenyl)-6-[(thiophen-2-yl)methoxy]pyridine (1)

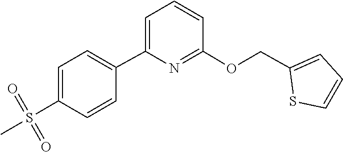 [SMILES: CS(=O)(=O)c1ccc(-c2cccc(OCc3cccs3)n2)cc1]

In a sealed tube under nitrogen was suspended sodium hydride (21.5 mg, 60% in mineral oil, 0.5378 mmol, 1.2 eq) in anhydrous THF (3 mL) and (thiophen-2-yl)methanol (50.8 μL, 0.5378 mmol, 1.2 eq) was added. The mixture was stirred at room temperature for 5 min, then a solution of 2-chloro-6-(4-methanesulfonylphenyl)pyridine (120 mg, 0.4482 mmol, 1 eq) in anhydrous THF (1 mL) was added and the mixture was stirred at 120° C. (micro-wave) for 2 h. The solvent was evaporated and the crude material was purified by normal phase chromatography (hexane/ethyl acetate, 0-90%) to afford 2-(4-methanesulfonylphenyl)-6-[(thiophen-2-yl)methoxy]pyridine (121.1 mg, 100% purity, 78.5% yield) as a white solid.

MS: [M+H]+ 345.98

¹H NMR (400 MHz, Chloroform-d) δ 8.27 (d, J=8.5 Hz, 2H), 8.04 (d, J=8.5 Hz, 2H), 7.71 (dd, J=8.2, 7.4 Hz, 1H), 7.45 (d, J=7.4 Hz, 1H), 7.30 (dd, J=5.1, 1.2 Hz, 1H), 7.19 (d, J=3.1 Hz, 1H), 7.01 (dd, J=5.1, 3.5 Hz, 1H), 6.82 (d, J=8.2 Hz, 1H), 5.69 (s, 2H), 3.10 (s, 3H).

General scheme:

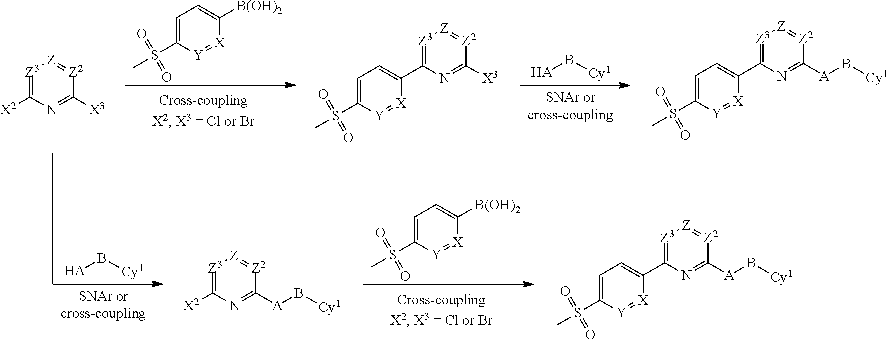

### 2-chloro-6-[4-(methylsulfanyl)phenyl]pyridine: Intermediate-1

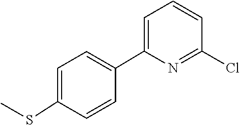

### (Method A)

In a flask under nitrogen were dissolved 2,6-dichloropyridine (2.0 g, 13.5 mmol, 1.0 eq), [4-(methylsulfanyl)phenyl]boronic acid (2.26 g, 13.5 mmol, 1.0 eq), potassium carbonate (3.73 g, 27.0 mmol, 2.0 eq) and Pd(dppf)Cl₂ (275 mg, 0.338 mmol, 2.5 mol %) in degassed 1,4-dioxane/water (1:1, 50 mL). The mixture was stirred overnight at 65° C., then brought back to room temperature. Water (65 mL) was added and the aqueous layer was extracted with DCM (3×100 mL). The combined organic layers were dried over MgSO₄, filtered and evaporated under reduced pressure. The crude material was purified by normal phase chromatography (hexanes/ethyl acetate, 0-15%) to afford 2-chloro-6-[4-(methylsulfanyl)phenyl]pyridine (1.83 g, 50% yield) as a white solid. MS: [M+H]+ 236.10.

¹H NMR (400 MHz, Chloroform-d) δ 7.93 (d, J=8.5 Hz, 2H), 7.68 (t, J=7.7 Hz, 1H), 7.61 (dd, J=7.8, 0.9 Hz, 1H), 7.32 (d, J=8.5 Hz, 2H), 7.23 (dd, J=7.7, 0.9 Hz, 1H), 2.53 (s, 3H).

### 2-bromo-6-[4-(methylsulfanyl)phenyl]pyridine: Intermediate-2

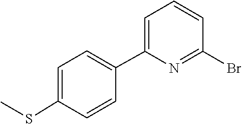

Obtained from 2,6-dibromopyridine (2.0 g, 8.44 mmol, 1.0 eq) and [4-(methylsulfanyl)phenyl]boronic acid (1.41 g, 8.44 mmol, 1.0 eq), using method A to afford 2-bromo-6-[4-(methylsulfanyl)phenyl]pyridine (1.46 g, 56% yield) as a white solid. MS: [M+H]+ 282.10. ¹H NMR (400 MHz, Chloroform-d) δ 7.93 (d, J=8.5 Hz, 2H), 7.65 (dd, J=7.7, 0.9 Hz, 1H), 7.57 (t, J=7.8 Hz, 1H), 7.38 (dd, J=7.7, 0.9 Hz, 1H), 7.32 (d, J=8.5 Hz, 2H), 2.53 (s, 3H).

### 2-chloro-6-(4-methanesulfonylphenyl)pyridine: Intermediate-3

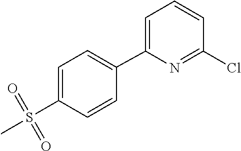 [SMILES: CS(=O)(=O)c1ccc(-c2cccc(Cl)n2)cc1]

Obtained from 2,6-dichloropyridine (2.0 g, 13.5 mmol, 1.0 eq), (4-methanesulfonylphenyl)boronic acid (2.70 g, 13.5 mmol, 1.0 eq), using method A to afford 2-chloro-6-(4-methanesulfonylphenyl)pyridine (1.81 g, 50% yield) as a white solid. MS: [M+H]+ 367.78. ¹H NMR (400 MHz, Chloroform-d) δ 8.21 (d, J=8.5 Hz, 2H), 8.05 (d, J=8.5 Hz, 2H), 7.79 (t, J=7.7 Hz, 1H), 7.73 (dd, J=7.7, 0.9 Hz, 1H), 7.37 (dd, J=7.7, 0.9 Hz, 1H), 3.09 (s, 3H).

### 2-bromo-6-(4-methanesulfonylphenyl)pyridine: Intermediate-4

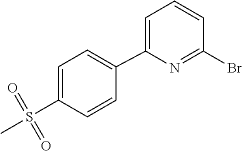 [SMILES: CS(=O)(=O)c1ccc(-c2cccc(Br)n2)cc1]

Obtained from 2,6-dibromopyridine (2.0 g, 8.44 mmol, 1.0 eq), (4-methanesulfonylphenyl) boronic acid (1.68 g, 8.44 mmol, 1.0 eq), using method A to afford 2-bromo-6-(4-methanesulfonylphenyl)pyridine (1.22 g, 44% yield) as a white solid. MS:[M+H]+ 312.07. ¹H NMR (400 MHz, Chloroform-d) δ 8.20 (d, J=8.5 Hz, 2H), 8.04 (d, J=8.5 Hz, 2H), 7.76 (d, J=7.6 Hz, 1H), 7.67 (t, J=7.7 Hz, 1H), 7.52 (d, J=7.7 Hz, 1H), 3.09 (s, 3H).

### 2-chloro-6-(4-(methylthio)phenyl)pyrazine: Intermediate-5

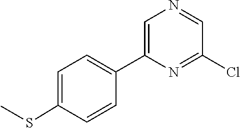

Obtained from 2,6-dichloropyrazine (50 mg, 0.3356 mmol, 2 eq) and (4-methanesulfonylphenyl)boronic acid (1 eq), using method A to afford 2-chloro-6-(4-(methylthio)phenyl)pyrazine (50 mg, 63% yield) as a white solid. MS: 237.63 [M+H]+. ¹H NMR (300 MHz, Chloroform-d) δ 8.89 (s, 1H), 8.48 (s, 1H), 7.96 (dt, J=13.1, 2.0 Hz, 2H), 7.35 (dt, J=13.2, 1.9 Hz, 2H), 2.54 (s, 3H).

### 2-chloro-6-(4-(methylsulfonyl)phenyl)pyrazine: Intermediate-7

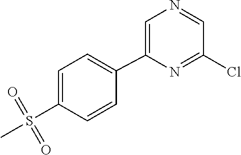 [SMILES: CS(=O)(=O)c1ccc(-c2cncc(Cl)n2)cc1]

Obtained from 2,6-dichloropyrazine (2 g, 13.4 mmol, 2 eq), (4-methanesulfonylphenyl)boronic acid (1.34 g, 6.70 mmol, 1.0 eq), using method A, to afford 2-chloro-6-(4-methanesulfonylphenyl)pyrazine (912 mg, 51% yield) as a white solid. MS: [M+H]+ 269.08. ¹H NMR (300 MHz, Chloroform-d) δ 9.00 (s, 1H), 8.63 (s, 1H), 8.27-8.22 (m, 2H), 8.12-8.07 (m, 2H), 3.11 (s, 3H).

### 2-bromo-4-fluoro-6-(4-(methylsulfonyl)phenyl)pyridine: Intermediate-9

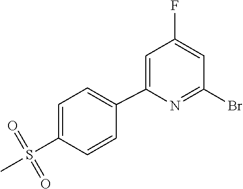 [SMILES: CS(=O)(=O)c1ccc(-c2cc(F)cc(Br)n2)cc1]

2,6-dibromopyridin-4-amine (1.0 g, 3.96 mmol, 1 eq) and (4-methanesulfonylphenyl)boronic acid (792 mg, 3.96 mmol, 1.0 eq) were reacted using method A. The crude material was purified by normal phase chromatography (hexane/EtOAc, 10-60%) to afford 2-bromo-6-(4-methanesulfonylphenyl)pyridin-4-amine (452 mg, 34.2% yield) as a white solid. MS: [M+H]+ 326.99. ¹H NMR (400 MHz, DMSO-d6) δ 8.10 (d, J=8.3 Hz, 2H), 8.01 (d, J=8.6 Hz, 2H), 7.12 (d, J=1.7 Hz, 1H), 6.70 (d, J=1.6 Hz, 1H), 6.55 (s, 2H), 3.25 (s, 3H).

To a suspension of 2-bromo-6-(4-methanesulfonylphenyl)pyridin-4-amine (50 mg, 0.1528 mmol, 1 eq) in fluoboric acid (48 wt. % in water, 0.5 mL) at 0° C. was added dropwise a solution of sodium nitrite (15.8 mg, 0.2292 mmol, 1.5 eq) in water (0.3 mL). The mixture was let to warm to room temperature and stirred overnight. The temperature was raised to 60° C. and stirring was pursued for 8 h during which the yellow-greenish solid slowly dissolved and bubbling was observed. The mixture was cooled to 0° C. and quenched by addition of small portion of solid NaHCO₃ until bubbling stopped. The mixture was diluted with water and extracted with DCM. The combined organic layers were dried over MgSO₄, filtered and evaporated. The crude material was purified by normal phase chromatography (hexane/EtOAc, 10-80%) to afford 2-bromo-4-fluoro-6-(4-methanesulfonylphenyl)pyridine (11 mg, 21.0% yield) as a white solid. MS: [M+H]+ 329.98. ¹H NMR (400 MHz, Chloroform-d) δ 8.18 (d, J=8.5 Hz, 2H), 8.06 (d, J=8.5 Hz, 2H), 7.49 (dd, J=9.1, 2.0 Hz, 1H), 7.29 (dd, J=7.4, 2.0 Hz, 1H), 3.09 (s, 3H).

19F NMR (376 MHz, Chloroform-d) 6-101.98 (t, J=8.2 Hz).

### 5-chloro-2-methoxy-3-(4-(methylsulfonyl)phenyl)pyrazine: Intermediate-10

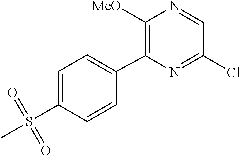 [SMILES: COc1ncc(Cl)nc1-c1ccc(S(C)(=O)=O)cc1]

Obtained from 3,5-dichloro-2-methoxypyrazine (1.4 g, 7.82 mmol, 1 eq), (4-methanesulfonylphenyl)boronic acid (1.56 g, 7.82 mmol, 1 eq) using method A, to provide 5-chloro-3-(4-methanesulfonylphenyl)-2-methoxypyrazine (0.7 g, 30% yield) as a white solid.

¹H NMR (400 MHz, DMSO-d6) δ 8.46 (s, 1H), 8.24 (d, J=8 Hz, 2H), 8.06 (d, J=16 Hz, 2H), 4.02 (s, 3H), 3.28 (s, 3H).

### 6-(4-(methylsulfonyl)phenyl)pyrazine-2-thiol: Intermediate-11

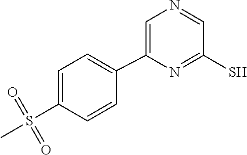 [SMILES: CS(=O)(=O)c1ccc(-c2cncc(S)n2)cc1]

A solution of intermediate-7 (1 g, 3.72 mmol, 1 eq), N,N-Dimethylformamide (6 mL) and sodium hydrate sulfane (1.11 g, 14.8 mmol, 4.0 eq) was stirred at room temperature for 16 hours. After completion of reaction, the reaction mixture was quenched in water (100 mL) and extracted with ethyl acetate (3×100 mL), combine organic layer was dried over Na2SO4 and evaporated. The residue was purified via Biotage (2:1 Hex/EtOAc; 12S column) provide sodium hydrate sulfane (0.200 g, purity, 20.2% yield) as a light yellow solid. This intermediate is used in the next step right away.

### 2-bromo-3-fluoro-6-(4-(methylsulfonyl)phenyl)pyridine and 6-bromo-3-fluoro-2-(4-(methylsulfonyl)phenyl)pyridine: Intermediate-12 and -13

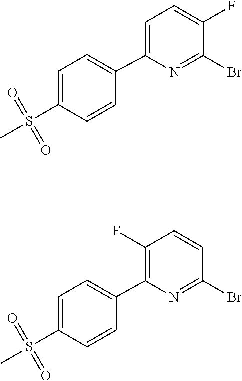 [SMILES: CS(=O)(=O)c1ccc(-c2ccc(F)c(Br)n2)cc1 ; CS(=O)(=O)c1ccc(-c2nc(Br)ccc2F)cc1]

Obtained from 2,6-dibromo-3-fluoropyridine (1 g, 3.92 mmol, 1 eq), (4-methanesulfonylphenyl)boronic acid (1.17 g, 5.88 mmol, 1.5 eq) using method A, to provide 2-bromo-3-fluoro-6-(4-methanesulfonylphenyl)pyridine (150 mg, 12% yield) and 6-bromo-3-fluoro-2-(4-methanesulfonylphenyl)pyridine (450 mg, 35% yield) as off white solids. Intermediate-12: MS: [M−H]⁻329.9. Intermediate-13: MS: [M−H]⁻329.9.

### 6-bromo-5-methoxy-N-(thiophen-2-ylmethyl)pyridin-2-amine: Intermediate-14

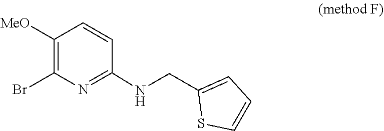 [SMILES: COc1ccc(NCc2cccs2)nc1Br]

A stirred mixture of 6-bromo-5-methoxypyridin-2-amine (0.2 g, 0.9850 mmol, 1 eq) and 2-(bromomethyl)thiophene (174 mg, 0.985 mmol, 1.0 eq) in dimethylformamide (3 mL) was added, portion wise sodium hydride (35.2 mg, 1.47 mmol, 1.5 eq) at 0° C. and stirred for 15 minutes. After 15 minutes, the reaction mixture was stirred at room temperature for 2 h. The reaction mixture was diluted with water (60 ml) and extracted with EtOAc (2×30 ml). The organics were dried over Na2SO₄ and evaporated. The residue was purified via Biotage (5:1 Hex/EtOAc; 12S column) to provide 6-bromo-5-methoxy-N-[(thiophen-2-yl)methyl]pyridin-2-amine (0.12 g, 38.4% yield) as a light yellow solid. MS(ESI): 301.0[M+H]+.

### 6-(4-(methylsulfonyl)phenyl)-5-(methylthio)pyridin-2-amine: Intermediate-15

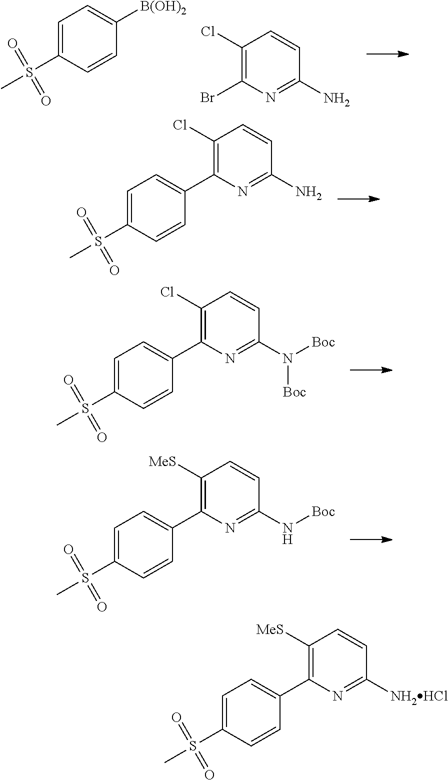

To a stirred solution of 6-bromo-5-chloropyridin-2-amine (1 g, 4.82 mmol) and (4-methanesulfonylphenyl)boronic acid (964 mg, 4.82 mmol) in 1,4-Dioxane (20 mL) was added solution of sodium carbonate (1.52 g, 14.4 mmol) in Water (10 mL) and degassed with nitrogen for 15 min. After 15 min, Pd(dppf)Cl₂·DCM (78.7 mg, 0.09640 mmol) was added to it and heated it at 100° C. for 3 h. After 3 h, the reaction mixture was filtered through celite and filtrate was evaporated to provide 5-chloro-6-(4-methanesulfonylphenyl)pyridin-2-amine (1 g, 60.2%) as a brown solid. MS(ESI): 283.0 [M+H]+.

To a stirred solution of 5-chloro-6-(4-methanesulfonylphenyl)pyridin-2-amine (1 g, 2.89 mmol) in DCM (20 mL) was added 4-dimethylaminopyridine (35.3 mg, 0.2890 mmol) and N,N-diisopropylethylamine (1.12 g, 8.67 mmol) followed by addition of di-tert-butyl dicarbonate (1.26 g, 5.78 mmol). The reaction mixture was allowed to stir at RT for 16 h. After 16 h, The reaction mixture was poured into water (100 mL) and extracted with DCM (3×30 mL). The combined organics were dried over Na2SO₄ and evaporated. The residue was purified via Biotage (10:1 Hex/EtOAc; 40+S column) to provide tert-butyl N-[(tert-butoxy)carbonyl]-N-[5-chloro-6-(4-methanesulfonylphenyl)pyridin-2-yl]carbamate (0.7 g, 38.0%) as a yellow solid. MS(ESI): 482.3 [M+H]+.

To a stirred solution tert-butyl N-[(tert-butoxy)carbonyl]-N-[5-chloro-6-(4-methanesulfonylphenyl)pyridin-2-yl]carbamate (0.5 g, 1.03 mmol) in DMF (10 mL) was added caesium carbonate (1 g, 3.09 mmol), sodium methylsulfanide (144 mg, 2.06 mmol) and heated it at 110° C. in microwave for 2 h. After completion of the reaction, the reaction mixture was poured into water (50 mL) and extracted with ethyl acetate (3×25 mL). The combined organics were dried over Na2SO4 and evaporated. The residue was purified via Biotage (2:1 Hex/EtOAc; 12M column) to provide tert-butyl N-[6-(4-methanesulfonylphenyl)-5-(methylsulfanyl)pyridin-2-yl]carbamate (0.3 g, 54.4%) as a yellow solid. MS(ESI): 395.0 [M+H]+.

To a stirred solution of tert-butyl N-[6-(4-methanesulfonylphenyl)-5-(methylsulfanyl)pyridin-2-yl]carbamate (0.3 g, 0.7604 mmol) in DCM (10 mL) was added 4M HCl in 1,4-Dioxane (6.07 mL, 24.3 mmol, 32 eq) and allowed it to stir at RT for 16 h. After completion of the reaction, the reaction mixture was filtered through Buchner funnel and washed with DCM (3×10 mL) to provide 6-(4-methanesulfonylphenyl)-5-(methylsulfanyl)pyridin-2-amine hydrochloride (0.2 g, 73.3%) as a yellow solid. MS(ESI): 295.0 [M−HCl]⁺.

### 6-(4-(methylsulfonyl)phenyl)pyridin-2-amine: Intermediate-16

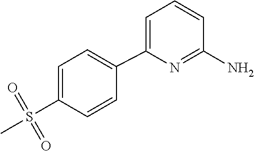 [SMILES: CS(=O)(=O)c1ccc(-c2cccc(N)n2)cc1]

Obtained from 6-bromopyridin-2-amine (1.5 g, 8.66 mmol) and (4-methanesulfonylphenyl)boronic acid (1.73 g, 8.66 mmol) using method A, to provide 6-(4-methanesulfonylphenyl)pyridin-2-amine (1.5 g, 61.3%) as a white solid. MS(ESI): 249.0 [M+H]⁺.

### 5-bromo-3-(4-(methylsulfonyl)phenyl)pyrazin-2-amine: Intermediate-17

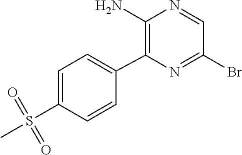 [SMILES: CS(=O)(=O)c1ccc(-c2nc(Br)cnc2N)cc1]

Obtained from 3,5-dibromopyrazin-2-amine (2 g, 7.90 mmol, 1 eq), (4-methanesulfonylphenyl)boronic acid (1.58 g, 7.90 mmol, 1 eq), using method A, to give 5-bromo-3-(4-methanesulfonylphenyl)pyrazin-2-amine (910 mg, 35.1% yield) as a yellow solid. MS: [M+H]+ 330.05. ¹H NMR (400 MHz, Chloroform-d) δ 8.15 (s, 1H), 8.09-8.07 (m, 2H), 7.99-7.96 (m, 2H), 4.79 (s, 2H), 3.09 (s, 3H).

### 5-bromo-2-ethoxy-3-(4-(methylsulfonyl)phenyl)pyrazine: Intermediate-18

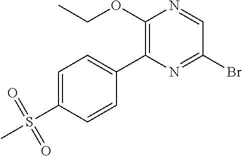 [SMILES: CCOc1ncc(Br)nc1-c1ccc(S(C)(=O)=O)cc1]

To a solution of 5-bromo-3-(4-methanesulfonylphenyl)pyrazin-2-amine (150 mg, 0.4570 mmol, 1 eq) and trifluoroacetic acid (138 μL, 1.82 mmol, 4 eq) in ethanol (2.4 mL) at 0° C. was slowly added isoamyl nitrite (244 μL, 1.82 mmol, 4 eq). The ice bath was removed and the mixture was allowed to warm up to room temperature. The mixture was stirred for 30 min and trifluoroacetic acid (138 μL, 1.82 mmol, 4 eq) and isoamyl nitrite (244 μL, 1.82 mmol, 4 eq) were added again. The mixture was stirred for 27 h. The solvent was evaporated. A saturated solution of sodium bicarbonate was added to the mixture. DCM was added and the layers were separated. The aqueous layer was extracted with DCM. The combined organics were dried over MgSO4 and the solvent was evaporated. The crude was purified by flash chromatography (Hexanes/EtOAc, 20%-100%) to give 5-bromo-2-ethoxy-3-(4-methanesulfonylphenyl)pyrazine (26 mg, 16% yield) as a colorless oil. MS: [M+H]⁺ 358.98. ¹H NMR (400 MHz, Chloroform-d) δ 8.35-8.32 (m, 2H), 8.21 (s, 1H), 8.04-8.00 (m, 2H), 4.51 (q, J=7.1 Hz, 2H), 3.09 (s, 3H), 1.47 (t, J=7.1 Hz, 3H).

### 6-(4-(methylsulfonyl)phenyl)-1H-pyrrolo[2,3-b]pyridine: Intermediate-19

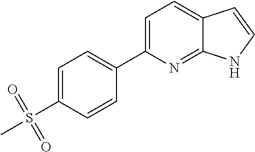 [SMILES: CS(=O)(=O)c1ccc(-c2ccc3cc[nH]c3n2)cc1]

Obtained from 6-chloro-1H-pyrrolo[2,3-b]pyridine (0.5 g, 3.27 mmol) and (4-methanesulfonylphenyl)boronic acid (654 mg, 3.27 mmol) using method A, to provide 6-(4-methanesulfonylphenyl)-1H-pyrrolo[2,3-b]pyridine (0.4 g, 43.0% yield) as a light yellow solid. MS(ESI): 273.0 [M+H]+.

### 6-chloro-1-(thiophen-2-ylmethy)-1H-pyrrolo[2,3-b]pyridine: Intermediate-20

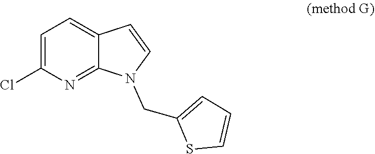 [SMILES: Clc1ccc2ccn(Cc3cccs3)c2n1]

A stirred solution of 6-chloro-1H-pyrrolo[2,3-b]pyridine (0.3 g, 1.96 mmol, 1 eq) in THF (5 mL) was added, portion wise sodium hydride (70.5 mg, 2.94 mmol, 1.5 eq) at 0° C. and stirred for 1 h. After 1 h, added (thiophen-2-yl)methyl methanesulfonate (451 mg, 2.35 mmol, 1.2 eq) at room temperature and stirred for 12 h. After 12 h, the reaction mixture was poured in to water (100 ml) and extracted with EtOAc (2×30 ml). The organic layers was dried over Na2SO4 and evaporated. The residue was purified via Biotage (5:1 Hex/EtOAc; 12S column) to provide 6-chloro-1-[(thiophen-2-yl)methyl]-1H-pyrrolo[2,3-b]pyridine (0.4 g, 69.1% yield) as a off white solid. MS(ESI):249.0[M+H]⁺.

### 6′-chloro-5-(methylthio)-2,2′-bipyridine: Intermediate-21

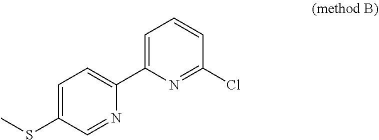

A stirred suspension of 2,6-dichloropyridine (1 g, 6.75 mmol, 1 eq) and 5-(methylthio)-2-(tributylstannyl)pyridine (2.79 g, 6.75 mmol, 1 eq) in dioxane (50 mL) was degassed with nitrogen gas for 15 min. After 15 min, palladium(II) acetate (151 mg, 0.675 mmol, 0.1 eq) and xphos (321 mg, 0.675 mmol, 0.1 eq) was added to it and heated at 100° C. for 3 h. After 3 h, the reaction mixture was filtered through celite and wash with EtOAc (2×10 mL), the filtrate was evaporated. The residue was purified via Biotage (2:1 Hex/EtOAc; 12M column) to 6′-chloro-5-(methylthio)-2,2′-bipyridine (120 mg, 8% yield) as a brown semi-solid. MS(ESI):237.0[M+H]+.

### 6-bromo-1-(4-(methylsulfonyl)phenyl)-1H-pyrrolo[2,3-b]pyridine: Intermediate-22

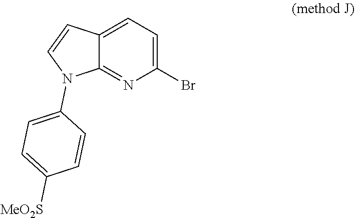 [SMILES: CS(=O)(=O)c1ccc(-n2ccc3ccc(Br)nc32)cc1]

To a stirred solution of 6-bromo-1H-pyrrolo[2,3-b]pyridine (0.5 g, 2.53 mmol, 1 eq) were added (4-methanesulfonylphenyl)boronic acid (1.01 g, 5.06 mmol, 2 eq) and sodium carbonate (0.536 g, 5.05 mmol, 1.996 eq) in Ethylene dichloride (5 mL) at room temperature and reaction mixture was degassed with argon for 20 min followed by copper(II) acetate (0.919 g, 5.05 mmol, 1.996 eq) and pyridine (0.599 g, 7.57 mmol, 2.992 eq) were added at room temperature and reaction mixture was heated at 100° C. for 16 h. After completion of reaction, the reaction mixture was poured in to water (25 mL) and extracted with EtOAc (3×25 mL). The organic layer was washed with brine solution (2×10 mL), dried over Na2SO4 and evaporated. The product was purified by column chromatography, product was eluted at 0-30% ethyl acetate in hexane provided 6-bromo-1-(4-methanesulfonylphenyl)-1H-pyrrolo[2,3-b]pyridine (0.066 g, purity, 7.43% yield) as a White solid. MS (ESI): 352[M+H]⁺.

### 6-bromo-6′-((methylsulfonyl)-2,3′_-bipyridine: Intermediate-23

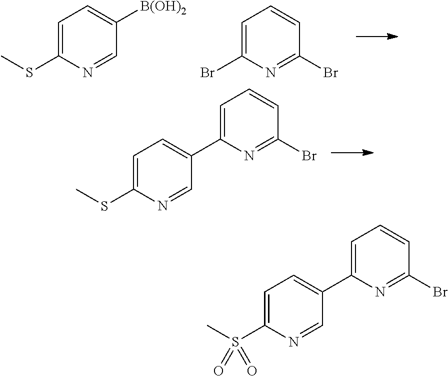

Obtained from 2,6-dibromopyridine (1 g, 4.22 mmol) and [6-(methylsulfanyl)pyridin-3-yl]boronic acid (713 mg, 4.22 mmol) using method A, to provide 6-bromo-6′-(methylsulfanyl)-2,3′-bipyridine (0.5 g, 41.1%) as a off white solid. MS(ESI): 282.9 [M+H]⁺. To a stirred solution of 6-bromo-6′-(methylsulfanyl)-2,3′-bipyridine (0.5 g, 1.77 mmol) in THF (10 mL) was added solution of Oxone (445 mg, 2.65 mmol) in Water (10 mL) and allowed to stir at RT for 16 h. After completion of the reaction; the reaction mixture was poured into water (20 mL) and extracted with ethyl acetate (3×20 mL). The combined organics were dried over Na2SO4 and evaporated to provide 6-bromo-6′-methanesulfonyl-2,3′-bipyridine (0.5 g, 85.1%) as an of white solid. MS(ESI): 312.9 [M+H]⁺.

### 5-chloro-2-methoxy-3-(6-(methylsulfonyl)pyridin-3-yl)pyrazine: Intermediate-24

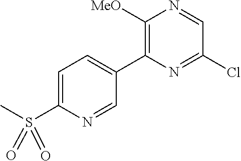 [SMILES: COc1ncc(Cl)nc1-c1ccc(S(C)(=O)=O)nc1]

Obtained from 3,5-dichloro-2-methoxypyrazine (0.2 g, 1.11 mmol, 1 eq), (6-methanesulfonylpyridin-3-yl)boronic acid (223 mg, 1.11 mmol, 1 eq) using method A, to provide 5-chloro-3-(6-methanesulfonylpyridin-3-yl)-2-methoxypyrazine (0.08, 24.0% yield) as a yellow solid.

### 5-bromo-2-methoxy-3-(4-methylsulfonyl)phenyl)pyrazine: Intermediate-25

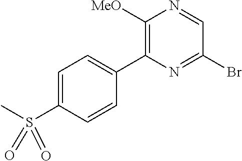 [SMILES: COc1ncc(Br)nc1-c1ccc(S(C)(=O)=O)cc1]

Obtained from 3,5-dibromo-2-methoxypyrazine (500 mg, 1.86 mmol, 1 eq) and (4-methanesulfonylphenyl)boronic acid (186 mg, 0.93 mmol, 0.5 eq), using method A, to provide 5-bromo-2-methoxy-3-(4-(methylsulfonyl)phenyl)pyrazine (125 mg, 18.1% yield). MS: 345.08 [M+H]⁺. ¹H NMR (400 MHz, Chloroform-d) δ 8.30 (dt, J=12.8, 2.1 Hz, 2H), 8.21 (s, 1H), 8.00 (dt, J=14.4, 2.2 Hz, 2H), 4.06 (s, 3H), 3.07 (s, 3H).

### 6-chloro-1-(thiophen-2-ylmethyl)-1H-pyrazolo[3,4-b]pyridine: Intermediate-26

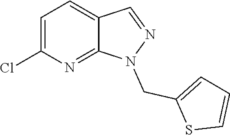 [SMILES: Clc1ccc2cnn(Cc3cccs3)c2n1]

Obtained from 6-chloro-1H-pyrazolo (100 mg, 0.6511 mmol, 1 eq) and 2-(chloromethyl)thiophene (172 mg, 1.30 mmol, 2 eq) using method F, to provide 6-chloro-1-[(thiophen-2-yl)methyl]-1H-pyrazolo[3,4-b]pyridine (50 mg, 29.9% yield). MS: 250.06 [M+H]⁺. ¹H NMR (400 MHz, Chloroform-d) δ 8.02 (s, 1H), 7.97 (d, J=8.3 Hz, 1H), 7.21 (dd, J=5.1, 1.0 Hz, 1H), 7.17-7.12 (m, 2H), 6.94 (dd, J=5.1, 3.5 Hz, 1H), 5.83 (s, 2H).

### 6-bromo-N-(furan-3-ylmethyl)-5-methoxypyridin-2-amine: Intermediate-27

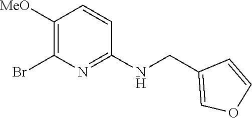 [SMILES: COc1ccc(NCc2ccoc2)nc1Br]

To stirred a mixture of 6-bromo-5-methoxypyridin-2-amine (0.2 g, 0.9850 mmol, 1 eq) and 3-hydroxymethylfuran (144 mg, 1.47 mmol, 1.5 eq) in toluene (2 mL), was added bis(lambda2-ruthenium(2+)) bis(cymene) tetrachloride (18.0 mg, 0.02955 mmol, 0.03 eq) at room temperature. The reaction mixture was stirred at 150° C. for 2 h in microwave irradiation. After completion of the reaction, the reaction mixture was put in water (50 mL) and it was extracted with ethyl acetate (3×20 mL). The organics were dried over Na2SO₄ and evaporated. The residue was purified via Biotage (2:1 Hex/EtOAc; 12S column) to provide 6-bromo-N-[(furan-3-yl)methyl]-5-methoxypyridin-2-amine (0.055 g, 19.7% yield) as a light yellow liquid. MS(ESI):384.9 [M+H]⁺.

### 7-bromo-5-(4-(methylsulfonyl)phenyl)furo[2,3-c]pyridine and 5-bromo-7-(4-(methylsulfonyl)phenyl)furo[2,3-c]pyridine: Intermediate-28 and -29

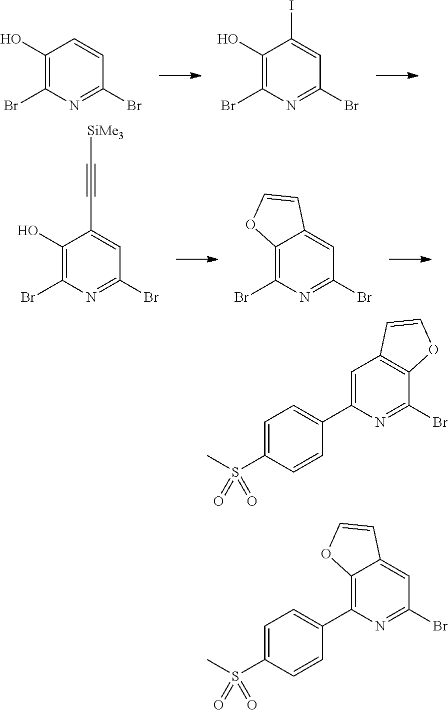

To a stirred solution of 2,6-dibromo-3-hydroxypyridine (5 g, 19.7 mmol, 1 eq), were added sodium bicarbonate (5.78 g, 68.9 mmol, 3.5 eq) and iodine (5.98 g, 23.6 mmol, 1.2 eq) in water (50 mL) and reaction mixture was stirred at room temperature for 12 h. After completion of reaction, the reaction mixture was poured in to Sodium thiosulfate (30 mL) and the pH was adjusted to 2 by addition of HCl. The solid was filtered to provide 2,6-dibromo-4-iodopyridin-3-ol (6 g, 80.4% yield) as a white solid. MS(ESI): 378 [M+H]⁺.

To a stirred solution of 2,6-dibromo-4-iodopyridin-3-ol (3 g, 7.91 mmol, 1 eq), were added lambda1-copper(1+) iodide (22.5 mg, 0.1186 mmol, 0.015 eq), trimethylsilylacetyl (1.15 g, 11.8 mmol, 1.5 eq), pdcl2(dppf).DCM (193 mg, 0.2373 mmol, 0.03 eq) and triethylamine (2.39 g, 23.7 mmol, 3 eq) in Chloroform (18 mL) and THF (12 mL). The reaction mixture was stirred at room temperature for 3 h. After completion of reaction, the reaction mixture was poured in to Water (30 mL) and extracted with EtOAc (3×40 mL). The organic layer was washed with brine solution (2×10 mL), dried over Na2SO4 and evaporated. The crude product was purify by combiflash using [0-30% EtOAc/Hexanes] to provide 2,6-dibromo-4-[2-(trimethylsilyl)ethynyl]pyridin-3-ol (0.812 g, 29.4% yield) as a yellow solid. MS(ESI): 349 [M+H]⁺.

To a stirred solution of 2,6-dibromo-4-[2-(trimethylsilyl)ethynyl]pyridin-3-ol (0.812 g, 2.32 mmol, 1 eq) (0.812 g, 2.32 mmol, 1 eq), were added lambda1-copper(1+) iodide (17.6 mg, 92.8 μmol, 0.04 eq) in Triethylamine (5 mL) and Ethanol (5 mL) reaction mixture was heated at 70° C. for 3.5 h. After completion of reaction, the reaction mixture was poured in to Water (30 mL) and extracted with EtOAc (3×40 mL). The organic layer was washed with brine solution (2×10 mL), dried over Na2SO4 and evaporated. The crude product was purify by combiflash using [0-10% EtOAc/Hexanes] to provide 5,7-dibromofuro[2,3-c]pyridine (0.3 g, purity, 46.7% yield) as a yellow solid. MS(ESI): 276 [M+H]⁺.

The final product was obtained from 5,7-dibromofuro[2,3-c]pyridine (0.3 g, 1.08 mmol, 1 eq), 4-methanesulfonylphenylboronic acid (216 mg, 1.08 mmol, 1 eq), using method A, to provide 5-bromo-7-(4-methanesulfonylphenyl)furo[2,3-c]pyridine (0.07 g, 18.4% yield) as a yellow solid and 7-bromo-5-(4-methanesulfonylphenyl)furo[2,3-c]pyridine (0.102 g, 26.5% yield) as yellow solid. MS(ESI): 352 [M+H]⁺.

### 2-chloro-7-(thiophen-2-ylmethyl)-7H-pyrrolo[2,3-d]pyrimidine: Intermediate-30

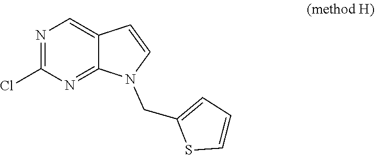 [SMILES: Clc1ncc2ccn(Cc3cccs3)c2n1]

To a stirred solution of 2-chloro-7H-pyrrolo[2,3-d]pyrimidine (0.2 g, 1.30 mmol, 1 eq) in THF (5 mL) was added, portion wise potassium tert-butoxide (175 mg, 1.56 mmol, 1.2 eq) at 0° C. and stirred for 1 h. After 1 h, added solution of 2-(bromomethyl)thiophene (345 mg, 1.95 mmol, 1.5 eq) at room temperature and stirred for 12 h. After 12 h, the reaction mixture was diluted with ethyl acetate (50 mL) and washed with water (2×40 mL). The organic layer was dried over Na2SO4 and evaporated. The residue was purified via Biotage (2:1 Hex/EtOAc; 12S column) to provide 2-chloro-7-[(thiophen-2-yl)methyl]-7H-pyrrolo[2,3-d]pyrimidine (0.3 g, 55.8% yield) as a white solid. MS(ESI): 250.0[M+H]⁺.

### 3-chloro-5-(thiophen-2-ylmethyl)-5H-pyrrolo[2,3-b]pyrazine: Intermediate-31

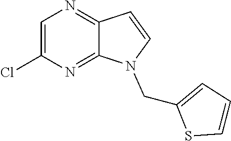 [SMILES: Clc1cnc2ccn(Cc3cccs3)c2n1]

Obtained from 3-chloro-5H-pyrrolo[2,3-b]pyrazine (0.4 g, 2.60 mmol, 1 eq) and (thiophen-2-yl)methyl methanesulfonate (749 mg, 3.90 mmol, 1.5 eq) using method G, to provide 3-chloro-5-[(thiophen-2-yl)methyl]-5H-pyrrolo[2,3-b]pyrazine (0.1 g, 6.0% yield) as a off white solid. MS(ESI): 350.0[M+H]⁺.

### 5-bromo-2-isopropoxy-3-(4-(methylsulfonyl)phenyl)pyrazine: Intermediate-32

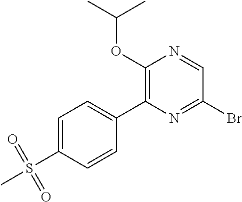 [SMILES: CC(C)Oc1ncc(Br)nc1-c1ccc(S(C)(=O)=O)cc1]

To a suspension of 5-bromo-3-(4-methanesulfonylphenyl)pyrazin-2-amine (150 mg, 0.4570 mmol, 1 eq) and trifluoroacetic acid (138 μL, 1.82 mmol, 4 eq) in isopropanol (2.4 mL) at 0° C. was slowly added isoamyl nitrite (244 μL, 1.82 mmol, 4 eq). The ice bath was removed and the mixture was allowed to warm up to room temperature. The mixture was stirred for 2 h and trifluoroacetic acid (138 μL, 1.82 mmol, 4 eq) and isoamyl nitrite (244 μL, 1.82 mmol, 4 eq) were added to the solution. The mixture was stirred at room temperature overnight. The solvent was evaporated. A solution of saturated sodium bicarbonate was added and DCM was added. The layers were separated. The aqueous layer was extracted with DCM. The combined organics were dried over MgSO₄ and the solvent was evaporated. The crude was purified by normal phase flash chromatography (Hexanes/EtOAc, 30%-100%) to give 5-bromo-3-(4-methanesulfonylphenyl)-2-(propan-2-yloxy)pyrazine (36 mg, 21.3% yield) as a white solid. MS: [M+H]⁺ 373.01. ¹H NMR (400 MHz, Chloroform-d) δ 8.32 (dt, J=8.7, 1.7 Hz, 2H), 8.20 (s, 1H), 8.03-8.00 (m, 2H), 5.40 (hept, J=6.2 Hz, 1H), 3.09 (s, 3H), 1.42 (d, J=6.2 Hz, 6H).

### 6-chloro-N-(thiophen-2-ylmethyl)-5-(trifluoromethyl)pyrazin-2-amine: Intermediate-33

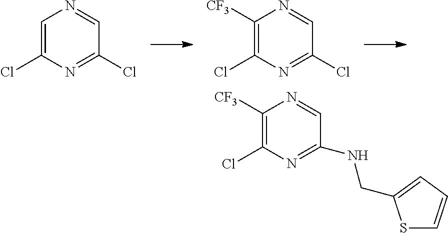

A mixture of dipotassium phosphate (1.74 g, 10.0 mmol) and tris(2-(4,6-difluorocyclohexa-1,2,4-trien-1-yl)pyridine) iridium (51.1 mg, 0.067 mmol) was back filled with nitrogen 3 times and then solution of 2,6-dichloropyrazine (500 mg, 3.35 mmol) in ACN (10 mL) was added to it. The reaction mixture was degassed with nitrogen u/v for 15 min at RT and 5 min at −78° C. and then allowed to warm up to RT. trifluoromethanesulfonyl chloride (2.25 g, 13.4 mmol) was drop wise added to it at RT and then heated it with 26 W bulb for 48 h. After 48 h, the reaction mixture was poured into water (25 mL) and extracted with petroleum ether (3×20 mL). The combine organics were dried over Na2SO4 and evaporated. The residue was purified via Biotage (50:1 Hex/EtOAc; 12M column) to provide 3,5-dichloro-2-(trifluoromethyl)pyrazine (70 mg, 10%) as a yellow liquid. ¹H NMR (400 MHz, CDCl3) δ 8.59 (s, 1H). 19FNMR (376 MHz, CDCl3) δ-66.36.

Then 3,5-dichloro-2-(trifluoromethyl)pyrazine (70 mg, 0.3226 mmol) and 1-(thiophen-2-yl)methanamine (36.5 mg, 0.3226 mmol) using method D, to provide 6-chloro-N-[(thiophen-2-yl)methyl]-5-(trifluoromethyl)pyrazin-2-amine (70 mg, 65.3% yield) as a white semi-solid. MS(ESI): 294.0 [M+H]⁺.

### 2-chloro-7-(4-(methylsulfonyl)phenyl)-7H-pyrrolo[2,3-d]pyrimidine: Intermediate-34

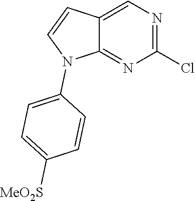 [SMILES: CS(=O)(=O)c1ccc(-n2ccc3cnc(Cl)nc32)cc1]

Obtained from 2-chloro-7H-pyrrolo[2,3-d]pyrimidine (0.05 g, 0.3255 mmol, 1 eq), (4-methanesulfonylphenyl)boronic acid (130 mg, 0.651 mmol, 2 eq) using method J, to provide 2-chloro-7-(4-methanesulfonylphenyl)-7H-pyrrolo[2,3-d]pyrimidine (0.055 g, 56.0% yield) as an off white solid. MS: [M+H]⁺ 308.0

### 6-bromo-N₂-(thiophen-2-ylmethyl)pyrazine-2,3-diamine: Intermediate-35

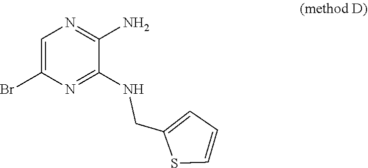 [SMILES: Nc1ncc(Br)nc1NCc1cccs1]

To stirred a mixture of 3,5-dibromopyrazin-2-amine (1 g, 3.95 mmol, 1 eq), 1-(thiophen-2-yl)methanamine (447 mg, 3.95 mmol, 1 eq) in dimethyl sulfoxide (5 mL) was added ceasium fluoride (1.20 g, 7.90 mmol, 2 eq) and stirred at 100° C. for 6 hours. After completion of reaction, the reaction mixture was quenched in water (100 mL) and extracted with ethyl acetate (3×40 mL), the organics was washed by brine (2×20 mL). The organics were dried over Na2SO₄ and evaporated. The residue was purified via Biotage (5:1 Hex/EtOAc; 12M column) to provide 6-bromo-N₂-[(thiophen-2-yl)methyl]pyrazine-2,3-diamine (0.5 g, 44.6% yield) as a yellow semi-solid. ¹H NMR (400 MHz, DMSO-d6) δ 7.39 (dd, J=5.1, 1.3 Hz, 1H), 7.23 (s, 1H), 7.13 (t, J=5.5 Hz, 1H), 7.06 (dd, J=3.4, 1.2 Hz, 1H), 6.97 (dd, J=5.1, 3.4 Hz, 1H), 6.21 (s, 2H), 4.66 (d, J=5.4 Hz, 2H).

### 6-chloro-N-(thiophen-2-ylmethyl)pyrazin-2-amine: Intermediate-36

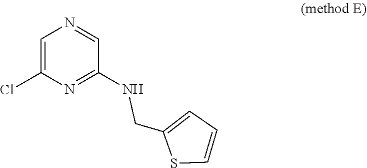 [SMILES: Clc1cncc(NCc2cccs2)n1]

To a mixture of 2,6-dichloropyrazine (250 mg, 1.67 mmol, 1 eq) in THF (5 mL) was added iPr2NEt (1.44 mL, 8.35 mmol, 5 eq) and thiophen-2-ylmethanamine (204 μL, 2.00 mmol, 1.2 eq). The yellow mixture was stirred at 65° C. for 2 days. The mixture was cooled at r.t., filtered through Celite and evaporated. 6-chloro-N-(thiophen-2-ylmethyl)pyrazin-2-amine (247 mg, 65.6% yield) was purified using flash column chromatography (SiO2, 0-100% EtOAc/Hexanes, eluted 25-30%). MS: 224.20 [M−H]⁻. ¹H NMR (300 MHz, Chloroform-d) δ 7.85 (s, 1H), 7.79 (s, 1H), 7.24 (d, J=1.3 Hz, 1H), 7.04 (dq, J=3.1, 1.0 Hz, 1H), 6.98 (dd, J=5.1, 3.5 Hz, 1H), 5.02 (s, 1H), 4.74 (dd, J=5.7, 0.6 Hz, 2H).

### 3-(methylsulfonyl)-5-(4-(methylsulfonyl)phenyl)-1,2,4-triazine: Intermediate-37

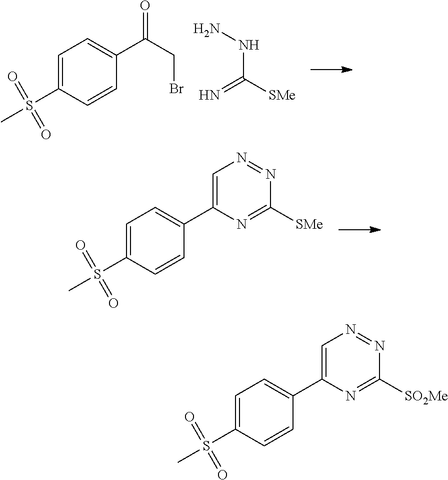

A solution of 2-bromo-1-(4-methanesulfonylphenyl)ethan-1-one (1 g, 3.60 mmol) in DMSO (5 mL) was heated at 50° C. for 30 min. (Solution-A). To a solution of {[(methylsulfanyl)methanimidoyl]amino}amine hydroiodide (839 mg, 3.60 mmol) in Ethanol (15 mL) and Water (7 mL) was added sodium bicarbonate (907 mg, 10.8 mmol) and allowed to stir at RT for 30 min. After 30 min, solution-A was added to it and allowed to stir at RT for 1 h. After 1 h, the reaction mixture was filtered through Buchner funnel to give 5-(4-methanesulfonylphenyl)-3-(methylsulfanyl)-1,2,4-triazine (0.5 g, 49.5%) as a light yellow solid. MS(ESI): 282 [M+H]⁺.

A mixture of mCPBA (1.52 g, 8.85 mmol) and DCM (10 mL) was stirred at RT for 15 min and filtered through Buchner funnel. The filtrate was drop wise added to a solution of 5-(4-methanesulfonylphenyl)-3-(methylsulfanyl)-1,2,4-triazine (0.5 g, 1.77 mmol) in DCM (5 mL) at −15° C. and allowed to stir at RT for 16 h. After 16 h, the reaction mixture was poured into saturated NaHCO₃ solution (50 mL) and extracted with the ethyl acetate (3×30 mL). The combined organics were dried over Na2SO4 and evaporated to give 3-methanesulfonyl-5-(4-methanesulfonylphenyl)-1,2,4-triazine (0.7 g, 41.3%) as a light yellow solid. MS(ESI): 314.0 [M+H]⁺.

### 2-(4-(methylsulfonyl)phenyl)-6-vinylpyridine: Intermediate-38

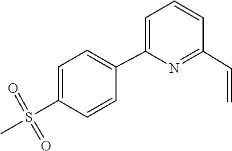 [SMILES: C=Cc1cccc(-c2ccc(S(C)(=O)=O)cc2)n1]

To a stirred solution of 2-bromo-6-(4-methanesulfonylphenyl)pyridine (500 mg, 1.60 mmol) and 2-ethenyl-4,4,5,5-tetramethyl-1,3,2-dioxaborolane (246 mg, 1.60 mmol) in 1,4-Dioxane (10 mL) was added solution of sodium carbonate (508 mg, 4.80 mmol) in Water (6 mL) and degassed with nitrogen for 15 min. After 15 min, Pd(dppf)Cl₂-DCM (1.30 g, 1.60 mmol) was added to it and heated at 100° C. for 3 h. After completion, the reaction mixture was diluted with ethyl acetate (50 mL) and washed with brine solution (50 mL). The organic layer was dried over Na2SO₄ and evaporated. The residue was purified via Biotage (2:1 Hex/EtOAc; 12M column) to provide 2-(4-(methylsulfonyl)phenyl)-6-vinylpyridine (350 mg, 81.8%) as an off white solid. MS(ESI): 260.0 [M+H]⁺.

### N′-(furan-3-ylmethylene)-4-methylbenzenesulfonohydrazide: Intermediate-39

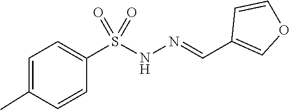 [SMILES: Cc1ccc(S(=O)(=O)N/N=C/c2ccoc2)cc1]

A solution of 4-methylbenzene-1-sulfonohydrazide (1 g, 5.36 mmol) and furan-3-carbaldehyde (515 mg, 5.36 mmol) in Methanol (10 mL) was heated at reflux temperature for 12 h. After completion, the reaction mixture was poured into water (20 mL) and resultant solid was filtered through Buchner funnel to provide N′-(furan-3-ylmethylene)-4-methylbenzenesulfonohydrazide (1 g, 70.9%) as an off white solid. MS(ESI): 265.0 [M+H]⁺. ¹H NMR (400 MHz, DMSO-d6) δ 11.25 (s, 1H), 8.02 (s, 1H), 7.85 (s, 1H), 7.73 (d, J=7.9 Hz, 2H), 7.68 (s, 1H), 7.40 (d, J=7.9 Hz, 2H), 6.62 (s, 1H), 2.36 (s, 3H).

### 6-chloro-3-methoxy-N-(thiophen-2-ylmethyl)pyrazin-2-amine: Intermediate-40

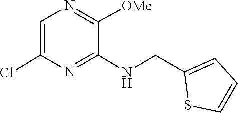 [SMILES: COc1ncc(Cl)nc1NCc1cccs1]

Obtained from 3,5-dichloro-2-methoxypyrazine (100 mg, 0.5586 mmol, 1 eq), 1-(thiophen-2-yl)methanamine (57.2 μL, 0.5586 mmol, 1 eq) using method D, to provide 6-chloro-3-methoxy-N-[(thiophen-2-yl)methyl]pyrazin-2-amine (111.7 mg, 95.6% purity, 74.6% yield) as a yellow oil. MS: [M+H]⁺ 257.98. ¹H NMR (300 MHz, Chloroform-d) δ 7.31 (s, 1H), 7.24 (dd, J=5.1, 1.2 Hz, 1H), 7.06 (dd, J=3.4, 1.1 Hz, 1H), 6.97 (dd, J=5.1, 3.5 Hz, 1H), 5.40 (s, 1H), 4.83-4.77 (m, 2H), 3.94 (s, 3H).

### 3-chloro-5-(4-(methylsulfonyl)phenyl)-2-(trifluoromethyl)pyrazine: Intermediate-41

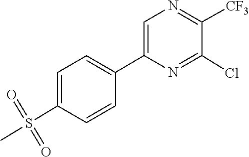 [SMILES: CS(=O)(=O)c1ccc(-c2cnc(C(F)(F)F)c(Cl)n2)cc1]

Obtained from 3,5-dichloro-2-(trifluoromethyl)pyrazine (150 mg, 0.6913 mmol) and (4-methanesulfonylphenyl)boronic acid (138 mg, 0.6913 mmol) using method A, to provide 3-chloro-5-(4-methanesulfonylphenyl)-2-(trifluoromethyl)pyrazine (125 mg, 53.8% yield) as a off white solid. MS(ESI): 354.0 [M+OH]⁺.

### 4-chloro-5-methoxy-2-(4-(methylsulfonyl)phenyl)pyrimidine: Intermediate-42

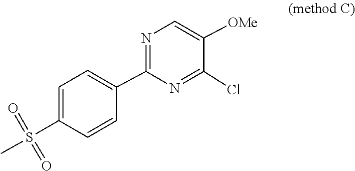 [SMILES: COc1cnc(-c2ccc(S(C)(=O)=O)cc2)nc1Cl]

In a flask under nitrogen were added 2,4-dichloro-5-methoxypyrimidine (500 mg, 2.79 mmol, 1 eq), (4-methanesulfonylphenyl)boronic acid (278 mg, 1.39 mmol, 0.5 eq), sodium carbonate (301 mg, 2.79 mmol, 1.0 eq) and Pd(PPh3) (51.4 mg, 0.1395 mmol, 0.05 eq) in degased dioxane/water (10 mL). The mixture was stirred at 65° C. for 92 h. The solvent was evaporated and the crude was purified by normal flash chromatography (Hexanes/EtOAc, 0-100%) to give 4-chloro-5-methoxy-2-(4-(methylsulfonyl)phenyl)pyrimidine (109 mg, 11.8% yield) as a yellow oil. MS: [M+H]⁺ 299.43. ¹H NMR (300 MHz, Chloroform-d) δ 8.41 (s, 1H), 8.33-8.28 (m, 2H), 8.05-8.01 (m, 2H), 4.04 (s, 3H), 3.09 (s, 3H).

### 3-chloro-5-(4-(methylsulfonyl)phenyl)-5H-pyrrolo[2,3-b]pyrazine: Intermediate-43

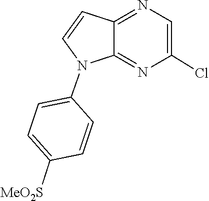 [SMILES: CS(=O)(=O)c1ccc(-n2ccc3ncc(Cl)nc32)cc1]

Obtained from 3-chloro-5H-pyrrolo[2,3-b]pyrazine (0.05 g, 0.3255 mmol, 1 eq), (4-methanesulfonylphenyl)boronic acid (130 mg, 0.651 mmol, 2 eq), using method J, to provide 3-chloro-5-(4-methanesulfonylphenyl)-5H-pyrrolo[2,3-b]pyrazine (0.055 g, 54.9% yield) as an off white solid. MS: [M+H]⁺ 308.0.

### 5-bromo-2-(2-methoxyethoxy)-3-(4-(methylsulfonyl)phenyl)pyrazine: Intermediate-44

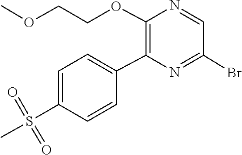 [SMILES: COCCOc1ncc(Br)nc1-c1ccc(S(C)(=O)=O)cc1]

To a solution at 0° C. of 5-bromo-3-(4-methanesulfonylphenyl)pyrazin-2-amine (200 mg, 0.6094 mmol, 1 eq) and trifluoroacetic acid (185 μL, 2.43 mmol, 4 eq) in 2-methoxyethanol (6.09 mL) was slowly added isoamyl nitrite (325 μL, 2.43 mmol, 4 eq). The ice bath was removed and the mixture was allowed to warm up to room temperature. The mixture was stirred for 19 h. The solvent was evaporated. The crude was purified by normal phase flash chromatography (Hexanes/EtOAc, 30-100%) to give 5-bromo-2-(2-methoxyethoxy)-3-(4-(methylsulfonyl)phenyl)pyrazine (135.5 mg, 57.4% yield) as a colorless oil. MS: [M+H]⁺ 389.07. ¹H NMR (400 MHz, Chloroform-d) δ 8.36 (d, J=8.6 Hz, 2H), 8.19 (s, 1H), 8.01 (d, J=8.6 Hz, 2H), 4.60-4.56 (m, 2H), 3.80-3.76 (m, 2H), 3.43 (s, 3H), 3.08 (s, 3H).

### 2-(6-chloropyrazin-2-yl)-5-(methylsulfonyl)phenol: Intermediate-45

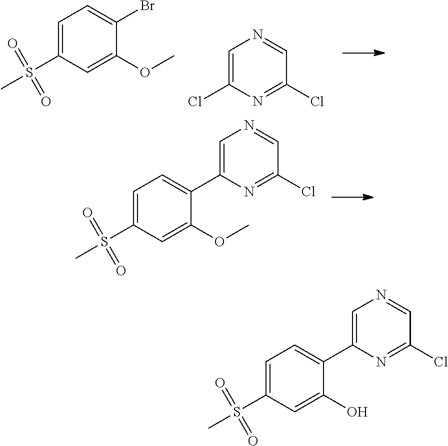

To a solution of 1-bromo-4-methanesulfonyl-2-methoxybenzene (1.3 g, 4.90 mmol) in 1,4-Dioxane (25 mL) was added potassium acetate (961 mg, 9.80 mmol), bis(pinacolato)diboron (3.73 g, 14.7 mmol) and degassed with nitrogen for 15 min. After 15 min, PdCl2(dppf).CH2Cl2 (80.0 mg, 0.098 mmol) and 2,6-dichloropyrazine (729 mg, 4.90 mmol) was added to it and heated at 100° C. for 2 h. After 2 h, the reaction mixture was filtered through celite and filtrate was evaporated. The residue was purified via Biotage (2:1 Hex/EtOAc; 40+S column) to provide 2-chloro-6-(4-methanesulfonyl-2-methoxyphenyl)pyrazine (600 mg, 29.7%) as a yellow oil. MS(ESI): 299.0 [M+H]⁺.

To a 1M boron tribromide solution in DCM (5.08 mL, 5.08 mmol, 6.0 eq) was drop wise added solution of 2-chloro-6-(4-methanesulfonyl-2-methoxyphenyl)pyrazine (350 mg, 0.8468 mmol) in DCM (2 mL) and allowed to stir at RT for 5 h. After completion, the reaction mixture was poured in to water (50 mL) and extracted with DCM (3×25 mL). The combined organic layer was dried over Na2SO4 and evaporated. The residue was purified via Biotage (1:1 Hex/EtOAc; 12M column) to provide 2-(6-chloropyrazin-2-yl)-5-methanesulfonylphenol (80 mg, 18.1%) as a brown semi-solid. MS(ESI): 284.9 [M+H]⁺, 283.0 [M−H]⁻.

### 7-chloro-5-(4-(methylsulfonyl)phenyl)-1,6-naphthyridine: Intermediate-46

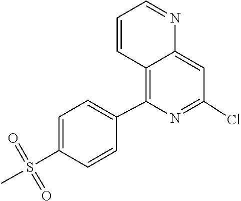 [SMILES: CS(=O)(=O)c1ccc(-c2nc(Cl)cc3ncccc23)cc1]

Obtained from 5,7-dichloro-1,6-naphthyridine (150 mg, 0.7536 mmol, 1 eq), (4-methanesulfonylphenyl)boronic acid (165 mg, 0.8289 mmol, 1.1 eq), using method A, to afford 7-chloro-5-(4-methanesulfonylphenyl)-1,6-naphthyridine (172.1 mg, 71.6% yield). MS: [M+H]⁺ 319.2.

### 5-chloro-3-(thiophen-2-ylmethyl)-3H-imidazo[4,5-b]pyridine: Intermediate-47

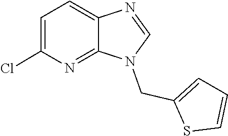 [SMILES: Clc1ccc2ncn(Cc3cccs3)c2n1]

Obtained from 5-chloro-3H-imidazo[4,5-b]pyridine (300 mg, 1.95 mmol, 1 eq) and 2-(chloromethyl)thiophene (517 mg, 3.90 mmol, 2 eq) using method F, to afford 5-chloro-3-[(thiophen-2-yl)methyl]-3H-imidazo[4,5-b]pyridine (130 mg, 26.7% yield). MS (N1): 250.06 [M+H]⁺. ¹H NMR (400 MHz, Chloroform-d) δ 8.07-7.98 (m, 2H), 7.31-7.27 (m, 2H), 7.14 (dd, J=3.5, 1.1 Hz, 1H), 6.99 (dd, J=5.1, 3.5 Hz, 1H), 5.61 (s, 2H).

### 6-chloro-3-methoxy-N-(thiophen-2-ylmethyl)pyrazin-2-amine: Intermediate-48

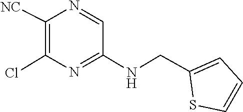 [SMILES: N#Cc1ncc(NCc2cccs2)nc1Cl]

Obtained from 3,5-dichloropyrazine-2-carbonitrile (0.3 g, 1.72 mmol, 1 eq) and 1-(thiophen-2-yl)methanamine (213 mg, 1.89 mmol, 1.1 eq) using method D, to provide 6-chloro-3-methoxy-N-(thiophen-2-ylmethyl)pyrazin-2-amine (0.3 g, 69.6% yield) as a light yellow solid. MS(ESI): 251.0[M+H]⁺ and 249.0[M−H]⁻.

### 3-methoxy-6-(4-(methylsulfonyl)phenyl)pyridin-2-amine: Intermediate-49

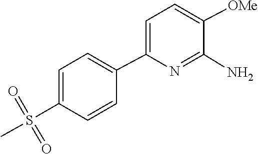 [SMILES: COc1ccc(-c2ccc(S(C)(=O)=O)cc2)nc1N]

Obtained from 6-bromo-3-methoxypyridin-2-amine (0.5 g, 2.46 mmol, 1 eq), (4-methanesulfonylphenyl)boronic acid (590 mg, 2.95 mmol, 1.2 eq) using method A, to provide 3-methoxy-6-(4-(methylsulfonyl)phenyl)pyridin-2-amine (0.6 g, 60.5% yield) as an off white solid. MS(ESI): 279.0 [M+H]⁺.

### 3-chloro-5-(4-(methylsulfonyl)phenyl)pyrazine-2-carbonitrile: Intermediate-50

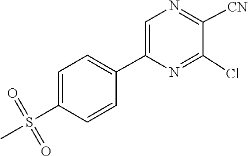 [SMILES: CS(=O)(=O)c1ccc(-c2cnc(C#N)c(Cl)n2)cc1]

Obtained from 3,5-dichloropyrazine-2-carbonitrile (0.2 g, 1.14 mmol, 1 eq), (4-(methylsulfonyl)phenyl)boronic acid (182 mg, 0.912 mmol, 0.8 eq) using method A, to provide 3-chloro-5-(4-(methylsulfonyl)phenyl)pyrazine-2-carbonitrile (0.12 g, 35.9% yield) as a off white solid. ¹H NMR (400 MHz, DMSO-d6) δ 9.61 (s, 1H), 8.48 (m, 2H), 8.15 (m, 2H), 3.55 (s, 3H).

### 3-chloro-N-(thiophen-2-ylmethyl)-1,2,4-triazin-5-amine: Intermediate-51

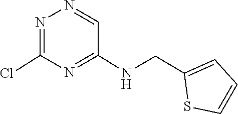 [SMILES: Clc1nncc(NCc2cccs2)n1]

To a solution of 2,3,4,5-tetrahydro-1,2,4-triazine-3,5-dione (200 mg, 1.76 mmol) in 1,4-Dioxane (2 mL) was added phosphorus oxychloride (1.96 mL, 21.1 mmol, 12 eq) and triethylamine (293 μL, 2.11 mmol, 1.2 eq) and heated at 100° C. for 1 h. After 1 h, color of reaction mixture converts into black which indicate the completion of the reaction. The reaction mixture was evaporated under reduced pressure and extracted with pet. ether (3×15 mL). The combined extracts were evaporated and residue was dissolved Acetonitrile (4 mL) at 0° C. and solution of 1-(thiophen-2-yl)methanamine (199 mg, 1.76 mmol) in Acetonitrile (2 mL) was added to it and allowed to stir at RT for 16 h. After completion, the reaction mixture was poured into water (30 mL) and extracted with ethyl acetate (3×15 mL). The combined organic layer was dried over Na2SO₄ and evaporated to provide 3-chloro-N-(thiophen-2-ylmethyl)-1,2,4-triazin-5-amine (60 mg, 15.0% yield) as a white solid. MS(ESI): 225.0 [M−H]⁻, 226.9 [M+H]⁺. ¹H NMR (400 MHz, DMSO-d6) 1H NMR (400 MHz, DMSO-d6) δ 9.29 (s, 1H), 8.53 (s, 1H), 7.46 (d, J=5.1 Hz, 1H), 7.10 (s, 1H), 7.05-6.97 (m, 1H), 4.70 (d, J=4.4 Hz, 2H).

### 5-chloro-3-(4-(methylsulfonyl)phenyl)furo[3,2-b]pyridine: Intermediate-52

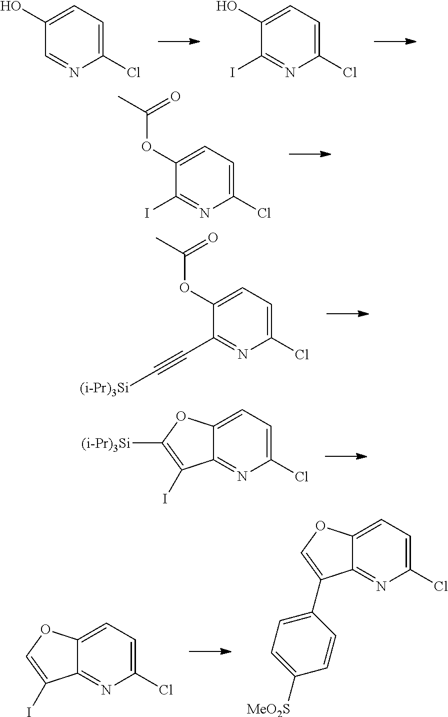

To a solution of 6-chloropyridin-3-ol (1.00 g, 7.72 mmol, 1.00 eq) in water (10 mL), Sodium carbonate (1.64 g, 15.4 mmol, 2.00 eq) and Iodine (1.96 g, 7.72 mmol, 1.00 eq) was added at 25° C. and the mixture was stirred for 16 hour at room temperature. After 16 hour the mixture was quenched with aqueous 1N hydrochloric acid (50 mL) and extracted with EtOAc (2×100 mL). The organics were dried with Na2SO4 and evaporated. The residue was purified via Biotage (20:1 Hex/EtOAc; 40Scolumn) to provide 6-Chloro-2-iodo-pyridin-3-ol (1.4 g, 68% yield) as a brown solid. MS(ESI): 255.8 [M+H]⁺.

A solution of 6-chloro-2-iodo-pyridin-3-ol (1.40 g, 5.27 mmol, 1.00 eq) and acetic anhydride (1.49 mL, 15.8 mmol, 3.00 eq) was stirred at 125° C. for 4 hours. After completion, the reaction mixture was quenched in water (50 mL) and extracted with ethyl acetate (3×150 mL) and washed with sat. NaHCO₃ solution (100 mL). organics were dried over Na2SO₄ and evaporated. The residue was purified via Biotage (9:1 Hex/EtOAc; 24S column) to provide (6-chloro-2-iodo-3-pyridyl) acetate (1.15 g, 72% yield) as an off white solid. 1H NMR (400 MHz, DMSO-d6) δ 7.73 (d, J=8.4 Hz, 1H), 7.63 (d, J=8.4 Hz, 1H), 2.36 (s, 3H), 2.22 (s, 2H).

To a degassed solution of (6-chloro-2-iodo-3-pyridyl) acetate (1.15 g, 3.79 mmol, 1.00 eq) in THF (15 mL) triethylamine (192 mg, 1.89 mmol, 0.500 eq) were added ethynyltriisopropylsilane (1.02 mL, 4.55 mmol, 1.20 eq), Copper(I) iodide (36 mg, 0.189 mmol, 0.0500 eq) and Bis(triphenylphosphine)palladium(II) dichloride (53 mg, 0.0758 mmol, 0.0200 eq) and the resulting mixture was stirred at 25° C. for 3 h. The solvent was evaporated in vacuo and the residue was purified by column chromatography (ethyl acetate/hexane, 1:9). The product was obtained (1.00 g, 72% yield) as a pale yellow solid. MS(ESI): 352.3 [M+H]⁺.

To a solution of [6-chloro-2-(2-triisopropylsilylethynyl)-3-pyridyl] acetate (1.00 g, 2.73 mmol, 1.00 eq) in methanol (10 mL) were added solution of Iodine (2.08 g, 8.19 mmol, 3.00 eq) in MeOH (10 mL) and Ceasium hydrogencarbonate (1.59 g, 8.19 mmol, 3.00 eq), and the resulting mixture was stirred at 40° C. for 120 minutes in a flask wrapped with aluminum foil. A solution of Na2S2O3 (5 g) in H2O (15 mL) was added and the mixture was concentrated. The residue was purified by column chromatography (ethyl acetate/hexane, 1:20) to afford the product (0.85 g, 66% yield) as a colorless oil.

To a solution of (5-chloro-3-iodo-furo[3,2-b]pyridin-2-yl)-triisopropyl-silane (0.85 g, 1.81 mmol, 1.00 eq) in THF (10 mL) was added Tetrabutylammonium fluoride solution (1 M in THF, 3.63 mL, 3.63 mmol, 2.00 eq) and the resulting mixture was stirred at 25° C. for 1 h. A saturated aqueous solution of NH4Cl (10 mL) was added, the mixture was concentrated, and the residue was purified by column chromatography (ethyl acetate/hexane, 1:20). The product (0.45 g, 84% yield) was obtained as a white solid. MS(ESI): 279.9 [M+H]⁺.

A stirred suspension of 5-chloro-3-iodo-furo[3,2-b]pyridine (0.45 g, 1.53 mmol, 1.00 eq) and 4-(Methylsulfinyl)benzeneboronic acid (0.25 g, 1.38 mmol, 0.900 eq) and Sodium carbonate (0.49 g, 4.59 mmol, 3.00 eq) in 1,4-Dioxane (4 mL) was degassed with nitrogen gas for 15 min. [1,1′-Bis(diphenylphosphino)ferrocene]dichloropalladium(II) complex with dichloromethane (0.037 g, 0.0459 mmol, 0.0300 eq) and Water (1 mL) was added to it and heated at 100° C. for 6 h. The reaction mixture was filtered through celite and wash with EtOAc (2×10 mL), the filtrate was evaporated. The residue was purified via Biotage (2:1 Hex/EtOAc; 12M column) to provide 5-chloro-3-(4-methylsulfonylphenyl)furo[3,2-b]pyridine (0.30 g, 60% yield) as a light brown semi-solid. MS(ESI): 308.2 [M+H]⁺.

### Compound 1: 2-(4-methanesulfonylphenyl)-6-[(thiophen-2-yl)methoxy]pyridine

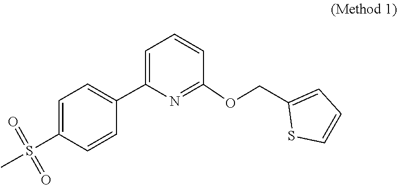 [SMILES: CS(=O)(=O)c1ccc(-c2cccc(OCc3cccs3)n2)cc1]

In a sealed tube under nitrogen was suspended sodium hydride (21.5 mg, 60% in mineral oil, 0.5378 mmol, 1.2 eq) in anhydrous THF (3 mL) and (thiophen-2-yl)methanol (50.8 μL, 0.5378 mmol, 1.2 eq) was added. The mixture was stirred at room temperature for 5 min, then a solution of intermediate-3 (120 mg, 0.4482 mmol, 1 eq) in anhydrous THF (1 mL) was added and the mixture was stirred at 120° C. (micro-wave) for 2 h. The solvent was evaporated and the crude material was purified by normal phase chromatography (hexane/ethyl acetate, 0-90%) to afford compound 1 (121.1 mg, 78.5% yield) as a white solid. MS: [M+H]⁺ 345.98. ¹H NMR (400 MHz, Chloroform-d) δ 8.27 (d, J=8.5 Hz, 2H), 8.04 (d, J=8.5 Hz, 2H), 7.71 (dd, J=8.2, 7.4 Hz, 1H), 7.45 (d, J=7.4 Hz, 1H), 7.30 (dd, J=5.1, 1.2 Hz, 1H), 7.19 (d, J=3.1 Hz, 1H), 7.01 (dd, J=5.1, 3.5 Hz, 1H), 6.82 (d, J=8.2 Hz, 1H), 5.69 (s, 2H), 3.10 (s, 3H).

### Compound 2

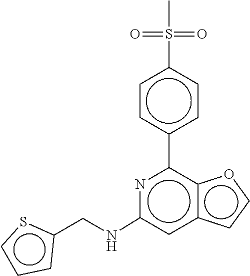

To a stirred solution of 5-bromo-7-(4-methanesulfonylphenyl)furo[2,3-c]pyridine (0.056 g, 0.1590 mmol, 1 eq), were added 1-(thiophen-2-yl)methanamine (21.5 mg, 0.1908 mmol, 1.2 eq) and caesium carbonate (103 mg, 0.318 mmol, 2 eq) in Dioxane (4 mL) reaction mixture was digassed with argon for 20 min and BrettPhos Pd G3 (14.4 mg, 0.01590 mmol, 0.1 eq) was added and reaction mixture was stirred at 100° C. for 12 h. After completion, the reaction mixture was poured in to water (20 mL) and extracted with EtOAc (3×20 mL). The organic layer was washed with brine solution (2×10 mL), dried over Na2SO4 and evaporated. The crude product was purify by combiflash using [0-30% EtOAc/Hexanes] to provide compound 2 (0.011 g, 18.1% yield) as a white solid. ¹H NMR (400 MHz, DMSO-d6) δ 8.62-8.52 (m, 2H), 8.13 (d, J=2.3 Hz, 1H), 8.11-8.02 (m, 2H), 7.12-7.02 (m, 2H) 6.99-6.87 (m, 2H) 6.79 (s, 1H) 4.76 (d, J=6.0 Hz, 2H), 3.27 (s, 3H).

### Compound 4

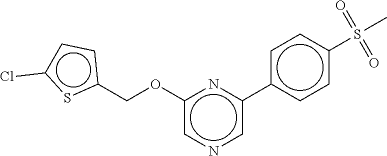

Was prepared using 5-chlorothiophene-2-methanol (44.2 mg, 0.2976 mmol, 2 eq) and intermediate-7 (40 mg, 0.1488 mmol, 1 eq) using method 1, to afford compound 4 (0.018 g, 32% yield) as a white solid. MS: 383.19 [M+H]⁺. ¹H NMR (300 MHz, Chloroform-d) δ 8.71 (s, 1H), 8.26 (dt, J=12.3, 2.0 Hz, 3H), 8.09 (dt, J=12.2, 1.8 Hz, 2H), 6.98 (d, J=3.8 Hz, 1H), 6.82 (d, J=3.8 Hz, 1H), 5.58 (s, 2H), 3.12 (s, 3H).

### Compound 5

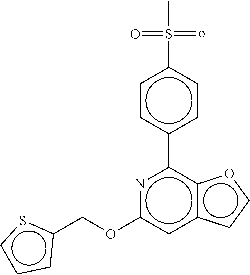

Obtained from (thiophen-2-yl)methanol (9.0 mg, 0.079 mmol, 1.1 eq) and 5-bromo-7-(4-methanesulfonylphenyl)furo[2,3-c]pyridine (25 mg, 0.071 mmol, 1 eq) using method 1, to afford compound 5 (0.013 g, 47% yield) as a white solid. MS: 386.1 [M+H]⁺. ¹H NMR (400 MHz, DMSO-d6) δ 8.66 (d, J=8.6 Hz, 2H), 8.33 (d, J=2.3 Hz, 1H), 8.14 (d, J=8.6 Hz, 2H), 7.50 (dd, J=5.1, 1.2 Hz, 1H), 7.27 (d, J=3.6 Hz, 1H), 7.16 (s, 1H), 7.07 (d, J=2.3 Hz, 1H), 7.02 (dd, J=5.1, 3.4 Hz, 1H), 5.71 (s, 2H), 3.30 (s, 3H).

### Compound 6

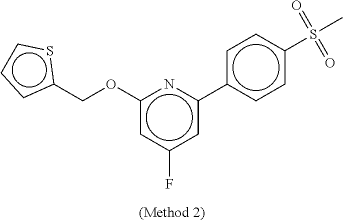

In a flame-dried vial under nitrogen were dissolved 2-bromo-4-fluoro-6-(4-methanesulfonylphenyl)pyridine (20 mg, 0.06057 mmol, 1.0 eq), (tert-butoxy)sodium (6.98 mg, 0.07268 mmol, 1.2 eq), tBuBrettPhos Pd G3 (2.58 mg, 0.003028 mmol, 0.05 eq) and (thiophen-2-yl)methanol (11.4 μL, 0.1211 mmol, 2.0 eq) in Dioxane (302 μL). The mixture was stirred at room temperature overnight. The solvent was evaporated and the crude material was purified by normal phase chromatography (hexane/EtOAc, 20-30%), then by preparative TLC (hexane/EtOAc, 40%, 2 elutions) to afford compound 6 (3.3 mg, 15.0% yield) as a white solid. MS: 364.27. ¹H NMR (400 MHz, Chloroform-d) δ 8.24 (d, J=8.4 Hz, 2H), 8.06 (d, J=8.4 Hz, 2H), 7.31 (dd, J=5.1, 1.1 Hz, 1H), 7.20 (dd, J=9.3, 2.2 Hz, 2H), 7.01 (dd, J=5.1, 3.5 Hz, 1H), 6.51 (dd, J=9.4, 1.9 Hz, 1H), 5.69 (s, 2H), 3.11 (s, 3H). 19F NMR (376 MHz, Chloroform-d) 6-103.73 (t, J=9.2 Hz).

### Compound 7

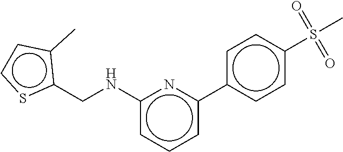

Obtained from 2-bromo-6-(4-methanesulfonylphenyl)pyridine (100 mg, 0.3203 mmol, 1 eq), and 1-(3-methylthiophen-2-yl)methanamine (48.8 mg, 0.3843 mmol, 1.2 eq), using method 2, to afford compound 7 (85 mg, 74.5% yield) as a white solid. MS: 359.25 [M+H]⁺. ¹H NMR (400 MHz, Chloroform-d) δ 8.20 (dt, J=12.8, 2.0 Hz, 2H), 7.99 (dt, J=12.2, 1.8 Hz, 2H), 7.52 (t, J=7.9 Hz, 1H), 7.12 (dd, J=9.0, 6.3 Hz, 2H), 6.83 (d, J=5.1 Hz, 1H), 6.46 (d, J=8.3 Hz, 1H), 4.86 (t, J=5.2 Hz, 1H), 4.72 (d, J=5.5 Hz, 2H), 3.07 (s, 3H), 2.28 (s, 3H).

### Compound 8

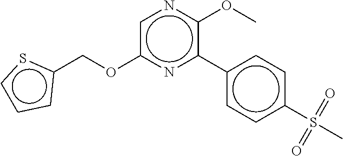

A stirred suspension of 5-chloro-3-(4-methanesulfonylphenyl)-2-methoxypyrazine (0.15 g, 0.5021 mmol, 1 eq), (thiophen-2-yl)methanol (68.7 mg, 0.6025 mmol, 1.2 eq) and caesium carbonate (325 mg, 1.00 mmol, 2.0 eq) in Toluene (4 mL) was degassed with nitrogen gas for 15 min. After 15 min, tBuBrettPhos Pd G3 (21.4 mg, 0.02510 mmol, 0.05 eq) was added to it and heated at 90° C. for 12 h. After 12 h, the reaction mixture was diluted with ethyl acetate (40 mL) and washed with water (2×30 mL). The organic layer was dried over Na2SO4 and evaporated. The residue was purified via Biotage (2:1 Hex/EtOAc; 12S column) to provide compound 8 (0.055 g, 28.5% yield) as yellow semi-solid. MS(ESI): 377.0[M+H]⁺. ¹H NMR (400 MHz, DMSO-d6) δ 8.45-8.35 (m, 2H), 8.05 (d, J=8.7 Hz, 3H), 7.54 (dd, J=5.1, 1.2 Hz, 1H), 7.30-7.22 (m, 1H), 7.03 (dd, J=5.1, 3.4 Hz, 1H), 5.63 (s, 2H), 3.98 (s, 3H), 3.28 (s, 3H).

### Compound 9

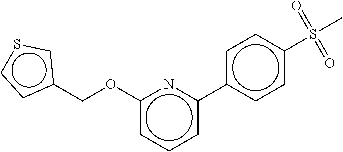

Was prepared using intermediate-4 (100 mg, 0.3203 mmol, 1 eq) and (thiophen-3-yl)methanol (36.1 μL, 0.3843 mmol, 1.2 eq) using method 2. The crude material was purified by flash chromatography (hexane/EtOAc, 10-40%) and the resulting solid was washed with little amount of Et2O to afford compound 9 (23.3 mg, 21.1% yield) as a white solid. MS: [M+H]⁺ 346.15. ¹H NMR (400 MHz, Chloroform-d) δ 8.22 (d, J=8.6 Hz, 2H), 8.03 (d, J=8.5 Hz, 2H), 7.71 (t, J=7.8 Hz, 1H), 7.43 (d, J=7.4 Hz, 1H), 7.38 (d, J=2.9 Hz, 1H), 7.34 (dd, J=5.0, 3.0 Hz, 1H), 7.21 (dd, J=5.0, 1.2 Hz, 1H), 6.83 (d, J=8.2 Hz, 1H), 5.53 (s, 2H), 3.10 (s, 3H).

### Compound 10

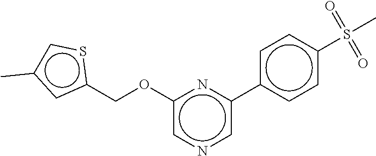

Was prepared using 4-methylthiopen alcohol (46.6 μL, 0.372 mmol, 2 eq) and intermediate-3 (50 mg, 0.1860 mmol, 1 eq) using method 1. The product was purified using flash column chromatography (SiO4, 0-100% EtOAc/hexanes, eluted 10) to afford compound 10 (29 mg, 43.2% yield) as a white solid. MS: 361.32 [M+H]⁺. ¹H NMR (300 MHz, Chloroform-d) δ 8.69 (s, 1H), 8.31-8.24 (m, 3H), 8.09 (dt, J=12.0, 1.7 Hz, 2H), 7.01 (s, 1H), 6.90 (t, J=2.6, 1.1 Hz, 1H), 5.62 (s, 2H), 3.12 (s, 3H), 2.25 (d, 3H).

### Compound 11

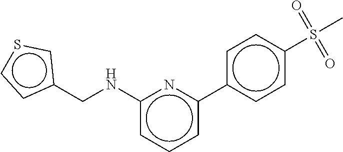

Was prepared using intermediate-4 (100 mg, 0.3203 mmol, 1 eq) and 1-(thiophen-3-yl)methanamine (38.4 μL, 0.3843 mmol, 1.2 eq) using method 2. The crude material was purified by normal phase chromatography (Hexane/EtOAc, 10-80%) to afford compound 11 (79.8 mg, 72.5% yield) as a white solid. MS: [M+H]⁺ 345.26. ¹H NMR (400 MHz, Chloroform-d) δ 8.17 (d, J=8.5 Hz, 2H), 7.99 (d, J=8.3 Hz, 2H), 7.54 (t, J=7.9 Hz, 1H), 7.32 (dd, J=5.0, 3.0 Hz, 1H), 7.22 (dd, J=3.0, 1.4 Hz, 1H), 7.16-7.08 (m, 2H), 6.46 (d, J=8.3 Hz, 1H), 5.01 (s, 1H), 4.63 (d, J=5.7 Hz, 2H), 3.08 (s, 3H).

### Compound 12

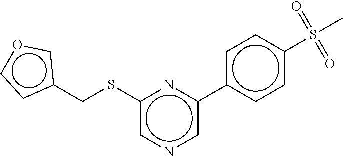

A solution of 6-(4-methanesulfonylphenyl)pyrazine-2-thiol (0.15 g, 0.5632 mmol, 1 eq), 3-(bromomethyl)furan (136 mg, 0.8448 mmol, 1.5 eq) in DMF (3 mL), was added sodium hydride (33.6 mg, 0.8448 mmol, 1.5 eq) at 0° C. and stirred for 1 h. After completion of reaction, the reaction mixture was quenched in cooled water (100 mL) and extracted with ethyl acetate (3×50 mL). Organics were dried over Na2SO4 and evaporated. The residue was purified via Biotage (2:1 Hex/EtOAc; 12S column) to provide compound 12 (0.019 g, 9.8% yield) as a off white solid. MS(ESI): 347.0[M+H]⁺. ¹H NMR (400 MHz, DMSO-d6) δ 9.07 (s, 1H), 8.65 (s, 1H), 8.43 (d, J=8.3 Hz, 2H), 8.09 (d, J=8.3 Hz, 2H), 7.69 (s, 1H), 7.60 (d, J=1.8 Hz, 1H), 6.50 (d, J=1.7 Hz, 1H), 4.42 (s, 2H), 3.29 (s, 3H).

### Compound 13

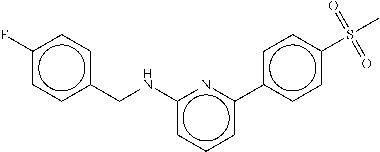

Was prepared using intermediate-4 (100 mg, 0.3203 mmol, 1 eq) and 1-(4-fluorophenyl)methanamine (43.8 μL, 0.3843 mmol, 1.2 eq) using method 2. The crude material was purified by normal phase chromatography (Hexane/EtOAc, 10-60%) to afford compound 13 (87.0 mg, 76.3% yield) as a white solid. MS: [M+H]⁺ 357.08. ¹H NMR (400 MHz, Chloroform-d) δ 8.15 (d, J=8.5 Hz, 2H), 7.99 (d, J=8.6 Hz, 2H), 7.54 (t, J=7.9 Hz, 1H), 7.37 (dd, J=8.5, 5.5 Hz, 2H), 7.13 (d, J=7.5 Hz, 1H), 7.04 (t, J=8.7 Hz, 2H), 6.43 (d, J=8.3 Hz, 1H), 5.10 (s, 1H), 4.60 (d, J=5.7 Hz, 2H), 3.07 (s, 3H). 19F NMR (376 MHz, Chloroform-d) 6-118.56.

### Compound 14

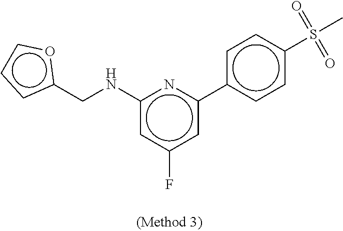

To a stirred solution of 2-bromo-4-fluoro-6-(4-methanesulfonylphenyl)pyridine (0.09 g, 0.2725 mmol, 1 eq), were added furfurylamine (31.7 mg, 0.3269 mmol, 1.2 eq) and caesium carbonate (177 mg, 0.545 mmol, 2 eq) at room temperature and reaction mixture was degassed with argon for 20 min followed by DavePhos-Pd-G3 (20.7 mg, 0.02725 mmol, 0.1 eq) and tris(dibenzylideneacetone) dipalladium (24.9 mg, 0.02725 mmol, 0.1 eq) were added at room temperature and reaction mixture was heated at 120° C. for 16 h. After completion of reaction, the reaction mixture was poured in to water (50 mL) and extracted with EtOAc (3×50 mL). The organic layer was washed with brine solution (2×10 mL), dried over Na2SO₄ and evaporated. The product was purified by normal phase chromatography and was eluted with 15% EtOAc in Hexane to provide compound 14 (0.021 g, 22.2% yield) as a white solid. ¹H NMR (400 MHz, DMSO-d6) δ 8.31 (d, J=8.2 Hz, 2H), 7.99 (d, J=8.2 Hz, 2H), 7.59 (s, 1H), 7.48 (t, J=5.8 Hz, 1H), 7.20 (dd, J=10.1, 2.0 Hz, 1H), 6.43-6.30 (m, 3H), 4.59 (d, J=5.7 Hz, 2H), 3.26 (s, 3H).

### Compound 15

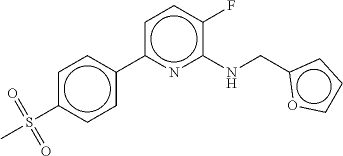

Obtained from 2-bromo-3-fluoro-6-(4-methanesulfonylphenyl)pyridine (150 mg, 0.4543 mmol, 1 eq), and 1-(furan-2-yl)methanamine (48.5 mg, 0.4997 mmol, 1.1 eq) using method 3. The residue was purified via Biotage (1:1 Hex/EtOAc; 12M column) to provide compound 15 (40.58 mg, 25.7% yield) as a brown solid. ¹H NMR (400 MHz, DMSO-d6) δ 8.24 (d, J=8.3 Hz, 2H), 7.97 (d, J=8.2 Hz, 2H), 7.56 (d, J=1.7 Hz, 1H), 7.50 (dd, J=11.1, 8.1 Hz, 1H), 7.42 (t, J=6.0 Hz, 1H), 7.27 (dd, J=8.1, 2.9 Hz, 1H), 6.37 (s, 1H), 6.30 (d, J=3.2 Hz, 1H), 4.64 (d, J=5.8 Hz, 2H), 3.24 (s, 3H). 19F NMR (377 MHz, DMSO-d6) δ-140.10.

### Compound 16

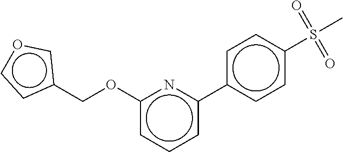

Was prepared using 3-furanmethanol (34.4 μL, 0.3735 mmol, 1 eq) and intermediate-3 (100 mg, 0.3735 mmol, 1 eq) using method 1. The product was purified using flash column chromatography (SiO4, 0-100% EtOAc/hexanes) to afford compound 16 (25 mg, 20.3% yield) as a white solid. MS: 329.65 [M+H]⁺. ¹H NMR (300 MHz, Chloroform-d) δ 8.23 (dt, J=12.5, 2.1 Hz, 2H), 8.03 (dt, J=12.4, 2.0 Hz, 2H), 7.70 (dd, J=8.2, 7.5 Hz, 1H), 7.55 (dd, J=1.5, 0.8 Hz, 1H), 7.45-7.40 (m, 2H), 6.81 (dd, J=8.2, 0.5 Hz, 1H), 6.53 (dd, J=2.6, 1.0 Hz, 1H), 5.39 (s, 2H), 3.09 (s, 3H).

### Compound 17

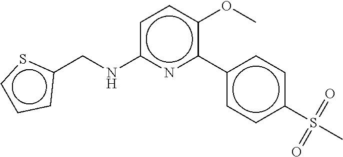

Obtained from 6-bromo-5-methoxy-N-[(thiophen-2-yl)methyl]pyridin-2-amine (0.12 g, 0.4010 mmol, 1 eq), and (4-methanesulfonylphenyl)boronic acid (96.2 mg, 0.4812 mmol, 1.2 eq) using method A. The residue was purified via Biotage (2:1 Hex/EtOAc; 12S column) to provide compound 17 (0.1 g, 66.6% yield) as a yellow solid. MS(ESI): 375.0[M+H]⁺. ¹H NMR (400 MHz, DMSO-d6) δ 8.19 (d, J=8.4 Hz, 2H), 7.99-7.90 (m, 2H), 7.46 (d, J=9.0 Hz, 1H), 7.32 (dd, J=5.2, 1.3 Hz, 1H), 7.03 (d, J=3.4 Hz, 1H), 6.99-6.89 (m, 2H), 6.62 (d, J=8.9 Hz, 1H), 4.66 (d, J=5.8 Hz, 2H), 3.75 (s, 3H), 3.24 (s, 3H).

### Compound 18

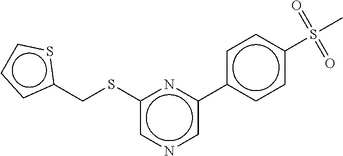

To a stirred suspension of 2-chloro-6-(4-methanesulfonylphenyl)pyrazine (250 mg, 0.9303 mmol) and (thiophen-2-yl)methanethiol (121 mg, 0.9303 mmol) in DMF (4 mL) was added Potassium carbonate (385 mg, 2.79 mmol) and heated at 100° C. for 16 h. After completion of the reaction, the reaction mixture was poured into water (30 mL) and extracted with ethyl acetate (3×25 mL). Combined organics were dried over Na2SO4 and evaporated. The residue was purified via Biotage (2:1 Hex/EtOAc; 12M column) to provide compound 18 (135 mg, 39.7% yield) as a yellow solid. MS(ESI): 363.0 [M+H]⁺. ¹H NMR (400 MHz, Chloroform-d) δ 8.75 (s, 1H), 8.47 (s, 1H), 8.30-8.20 (m, 2H), 8.15-8.04 (m, 2H), 7.17 (dd, J=5.1, 1.2 Hz, 1H), 7.06 (d, J=3.4 Hz, 1H), 6.92 (dd, J=5.2, 3.5 Hz, 1H), 4.74 (s, 2H), 3.12 (s, 3H).

### Compound 19

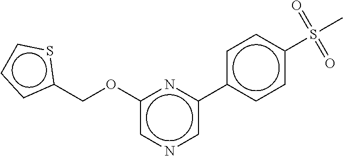

Obtained from 2-chloro-6-(4-(methylsulfonyl)phenyl)pyrazine (100 mg, 0.3721 mmol) and thiophen-2-ylmethanol (46.7 mg, 0.4093 mmol) using method 1, to provide compound 19 (75 mg, 58.5% yield) as a yellow solid. MS(ESI): 347.0 [M+H]⁺. ¹H NMR (400 MHz, Chloroform-d) δ 8.70 (s, 1H), 8.28 (d, J=7.8 Hz, 3H), 8.16-8.03 (m, 2H), 7.34 (dd, J=5.1, 1.2 Hz, 1H), 7.22 (d, J=3.5 Hz, 1H), 7.02 (dd, J=5.1, 3.5 Hz, 1H), 5.70 (s, 2H), 3.12 (s, 3H).

### Compound 20

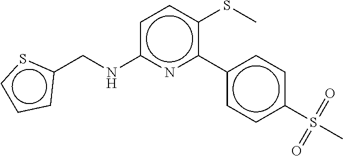

To a stirred solution of 6-(4-methanesulfonylphenyl)-5-(methylsulfanyl)pyridin-2-amine hydrochloride (200 mg, 0.6045 mmol) and (thiophen-2-yl)methanol (69.0 mg, 0.6045 mmol) in Toluene (4 mL) was added potassium hydroxide (101 mg, 1.81 mmol) and bis(lambda2-ruthenium(2+)) bis(cymene) tetrachloride (37.0 mg, 0.06045 mmol). The reaction mixture was heated in microwave at 150° C. for 2 h. After completion of the reaction; the reaction mixture was filtered through celite and filtrate was evaporated. The residue was purified via Biotage (1:1 Hex/EtOAc; 12M column) to provide compound 20 (50 mg, 20.5%) as a off white solid. MS(ESI): 391.4 [M+H]⁺. ¹H NMR (400 MHz, DMSO-d6) δ 7.97 (d, J=8.1 Hz, 2H), 7.84 (d, J=8.0 Hz, 2H), 7.64 (d, J=8.7 Hz, 1H), 7.40 (t, J=6.0 Hz, 1H), 7.35 (d, J=5.2 Hz, 1H), 7.05-6.88 (m, 2H), 6.62 (d, J=8.7 Hz, 1H), 4.64 (d, J=5.9 Hz, 2H), 3.27 (s, 3H), 2.25 (s, 3H).

### Compound 21

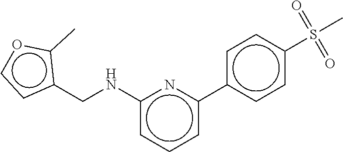

Was prepare using intermediate-4 (0.2 g, 0.6406 mmol, 1 eq) and 1-(2-methylfurana-3-yl)methanamine (85.4 mg, 0.7687 mmol, 1.2 eq) using method 2. The residue was purified via Biotage (2:1 Hex/EtOAc; 12S column) to provide impure product, which was further purified by prep HPLC using (10-60% ACN in 0.1% formic acid in water as modifier) as mobile phase to provide compound 21 (0.12 g, 52.9% yield) as an off white solid. MS(ESI):343.0[M+H]⁺. ¹H NMR (400 MHz, DMSO-d6) δ 8.28 (d, J=8.1 Hz, 2H), 7.98 (d, J=8.1 Hz, 2H), 7.50 (t, J=7.8 Hz, 1H), 7.40 (s, 1H), 7.20 (d, J=7.4 Hz, 1H), 6.96 (t, J=5.7 Hz, 1H), 6.55 (d, J=8.2 Hz, 1H), 6.39 (s, 1H), 4.33 (d, J=5.5 Hz, 2H), 3.25 (s, 3H), 2.28 (s, 3H).

### Compound 22

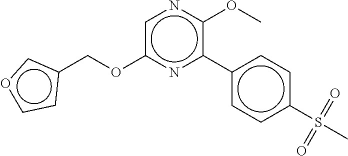

A stirred suspension of 5-chloro-3-(4-methanesulfonylphenyl)-2-methoxypyrazine (0.15 g, 0.5021 mmol, 1 eq), (furan-3-yl)methanol (73.8 mg, 0.7531 mmol, 1.5 eq) and caesium carbonate (407 mg, 1.25 mmol, 2.5 eq) in dioxane (4 mL) was degassed with nitrogen gas for 15 min. After 15 min, tBuBrettPhos Pd G3 (21.4 mg, 0.02510 mmol, 0.05 eq) was added to it and heated at 100° C. for 12 h. After 12 h, the reaction mixture was diluted with water (80 mL) and extracted with ethyl acetate (3×40 mL). The organic layers was dried over Na2SO4 and evaporated. The residue was purified via Biotage (5:1 Hex/EtOAc; 12S column) to provide impure product, which was further purified by prep HPLC using (25-70% ACN in water containing 5 mM ammonium carbonate and 0.1% ammonia in water as modifier) as mobile phase to provide compound 22 (0.008 g, 4.5% yield) as a off white solid. MS(ESI): 361.0[M+H]⁺. ¹H NMR (400 MHz, DMSO-d6) δ 8.42-8.32 (m, 2H), 8.12-7.97 (m, 3H), 7.82 (s, 1H), 7.67 (t, J=1.8 Hz, 1H), 6.60 (d, J=1.8 Hz, 1H), 5.30 (s, 2H), 3.98 (s, 3H), 3.27 (s, 3H).

Compound 23:

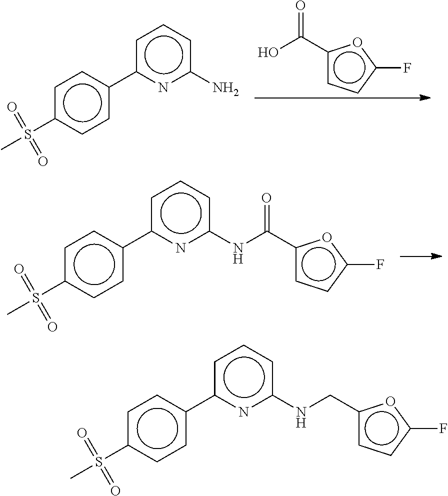

To a stirred solution of 6-(4-methanesulfonylphenyl)pyridin-2-amine (0.15 g, 0.6041 mmol, 1 eq) were added 5-fluorofuran-2-carboxylic acid (0.094 g, 0.7226 mmol, 1.196 eq) and pyridine (0.572 g, 7.21 mmol, 11.935 eq) in DCM (15 mL) at room temperature and reaction mixture was cooled at 0° C., phosphorus oxychloride (0.926 g, 6.03 mmol, 9.982 eq) was added drop wise. Reaction mixture was allow to attain room temperature. The reaction mixture was stirred at room temperature for 16 h. After completion of reaction, the reaction mixture was poured in to water (50 mL) and extracted with DCM (3×50 mL). The organic layer was washed with brine solution (2×10 mL), dried over Na2SO4 and evaporated. The crude product was purify by combiflash using [0-20% EtOAc/Hexanes] to provide 5-fluoro-N-(6-(4-(methylsulfonyl)phenyl)pyridin-2-yl)furan-2-carboxamide (0.121 g, 55.7% yield) as a yellow solid. MS(ESI): 360.36[M+H]⁺.

To a stirred solution of 5-fluoro-N-(6-(4-(methylsulfonyl)phenyl)pyridin-2-yl)furan-2-carboxamide (0.121 g, 0.3357 mmol, 1 eq) in THF (4 mL) at room temperature and reaction mixture was cooled to 0° C., borane dimethylsulfide (126 mg, 1.65 mmol, 4.915 eq) was added and reaction mixture was heated at 50° C. for 2 h. After completion of reaction, the reaction mixture was poured in to water (25 mL) and extracted with EtOAc (3×25 mL). The organic layer was washed with brine solution (2×10 mL), dried over Na2SO₄ and evaporated. The product was added to a Prep HPLC column and was eluted with 20-80% ACN in 5 mM ABC+0.1% NH3 in water as a gradient to provide compound 23 (0.016 g, 13.7% yield) as a white solid. ¹H NMR (400 MHz, DMSO-d6) 8.29 (d, J=8.3 Hz, 2H), 7.99 (d, J=8.2 Hz, 2H), 7.54 (t, J=7.8 Hz, 1H), 7.27-7.17 (m, 2H), 6.60 (d, J=8.2 Hz, 1H), 6.32 (t, J=3.3 Hz, 1H), 5.63 (dd, J=7.0, 3.3 Hz, 1H), 4.53-4.46 (m, 2H), 3.25 (s, 3H).

### Compound 24

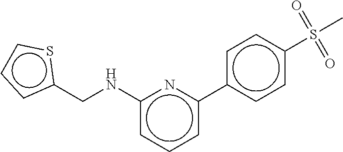

Was prepared using intermediate-3 (100 mg, 0.3735 mmol, 1 eq) and 1-(thiophen-2-yl)methanamine (42.0 μL, 0.4108 mmol, 1.1 eq) using method 2. The crude was purified by prep HPLC (water/acetonitrile, 0-100%, TFA 0.1%) to give compound 24 (4.7 mg, 3.7% yield) as a colorless oil. MS: [M+H]⁺ 345.81. ¹H NMR (300 MHz, Chloroform-d) δ 8.11 (d, J=8.6 Hz, 2H), 7.94 (d, J=8.5 Hz, 2H), 7.88 (d, J=16.5 Hz, 1H), 7.29-7.27 (m, 1H), 7.09 (dd, J=3.5, 1.0 Hz, 1H), 7.02-6.96 (m, 2H), 6.93-6.88 (m, 1H), 4.79 (s, 2H), 3.12 (s, 3H). 19F NMR (376 MHz, CDCl3) δ-79.06, −164.90.

### Compound 25

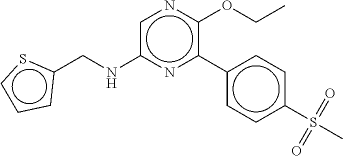

Was prepared using 5-bromo-2-ethoxy-3-(4-(methylsulfonyl)phenyl)pyrazine (36 mg, 0.1007 mmol, 1 eq) and 1-(thiophen-2-yl)methanamine (10.2 μL, 0.1007 mmol, 1 eq) using method 2. The crude was purified by normal phase flash chromatography (Hexanes/EtOAc, 20-100%) to give compound 25 (14.7 mg, 37.4% yield) as a yellow oil. MS: [M+H]⁺ 390.14. ¹H NMR (400 MHz, Chloroform-d) δ 8.42-8.37 (m, 2H), 8.01-7.97 (m, 2H), 7.55 (s, 1H), 7.21 (dd, J=5.1, 1.2 Hz, 1H), 7.05-7.01 (m, 1H), 6.97 (dd, J=5.1, 3.5 Hz, 1H), 4.77 (d, J=5.8 Hz, 2H), 4.73-4.68 (m, 1H), 4.41 (q, J=7.0 Hz, 2H), 3.08 (s, 3H), 1.43 (t, J=7.0 Hz, 3H).

### Compound 26

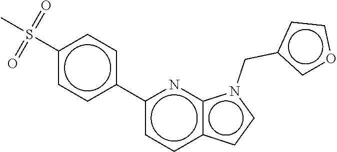

To a stirred solution of 6-(4-methanesulfonylphenyl)-1H-pyrrolo[2,3-b]pyridine (100 mg, 0.3672 mmol) in DMF (2 mL) was added 60% sodium hydride (29.3 mg, 0.7344 mmol) and stirred at same temperature for 30 min. After 30 min, 3-(bromomethyl)furan (177 mg, 1.10 mmol) was drop wise added to it and allowed to stir it at RT for 6 h. After completion of the reaction, the reaction mixture was poured into cold water (20 mL) and obtained solid was filtered through Buchner funnel. The residue was purified via Biotage (1:1 Hex/EtOAc; 12M column) to provide compound 26 (110 mg, 82.1% yield) as a off white solid. MS(ESI): 353.0 [M+H]⁺. ¹H NMR (400 MHz, DMSO-d6) δ 8.46 (d, J=8.2 Hz, 2H), 8.07 (dd, J=29.7, 8.2 Hz, 3H), 7.84 (d, J=8.2 Hz, 1H), 7.78-7.67 (m, 2H), 7.59 (s, 1H), 6.65-6.41 (m, 2H), 5.40 (s, 2H), 3.27 (s, 3H).

### Compound 27

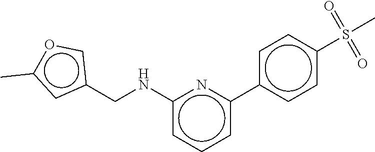

Was prepared using intermediate-4 (0.2 g, 0.6406 mmol, 1 eq) and 1-(5-methylfuran-3-yl)methanamine (85.4 mg, 0.7687 mmol, 1.2 eq) using method 2. The residue was purified via Biotage (2:1 Hex/EtOAc; 12S column) to provide impure product, which was further purified by prep HPLC using (55-25% ACN in 0.1% formic acid in water as modifier) as mobile phase to provide compound 27 (0.052 g, 23.6% yield) as an off white solid. MS(ESI):343.0[M+H]⁺. ¹H NMR (400 MHz, DMSO-d6) δ 8.34-8.20 (m, 2H), 8.04-7.91 (m, 2H), 7.51 (t, J=7.8 Hz, 1H), 7.44 (s, 1H), 7.21 (d, J=7.4 Hz, 1H), 6.97 (t, J=5.6 Hz, 1H), 6.56 (d, J=8.3 Hz, 1H), 6.09 (s, 1H), 4.34 (d, J=5.5 Hz, 2H), 3.24 (s, 3H), 2.20 (s, 3H).

### Compound 28

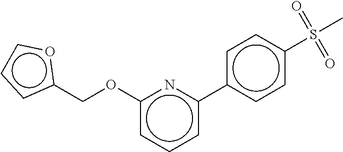

Was prepared using intermediate-3 (50 mg, 0.1867 mmol, 1 eq) and 2-furylmethanol (16.1 μL, 0.1867 mmol, 1 eq) using method 2. The residue was purified using flash column chromatography (SiO4, 0-20% hexanes/EtOAc) to afford compound 28 (34 mg, 55.3% yield). MS: 330.19 [M+H]⁺. ¹H NMR (300 MHz, Chloroform-d) δ 8.24 (dt, J=13.0, 1.8 Hz, 2H), 8.03 (dt, J=11.8, 1.6 Hz, 2H), 7.70 (dd, J=16.1, 0.5 Hz, 1H), 7.51-7.38 (m, 2H), 6.82 (d, J=8.2 Hz, 1H), 6.48 (d, J=3.1 Hz, 1H), 6.39 (dd, J=3.1, 1.9 Hz, 1H), 5.47 (s, 2H), 3.10 (s, 3H).

### Compound 29

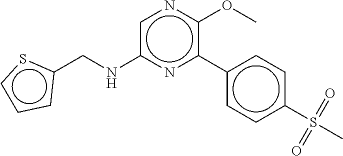

Obtained from 5-chloro-3-(4-methanesulfonylphenyl)-2-methoxypyrazine (0.35 g, 1.16 mmol, 1 eq) and 1-(thiophen-2-yl)methanamine (157 mg, 1.39 mmol, 1.2 eq) using method 3. The residue was purified via Biotage (2:1 Hex/EtOAc; 12S column) to provide compound 29 (0.055 g, 12.6% yield) as yellow solid. MS(ESI): 376.0 [M+H]⁺. ¹H NMR (400 MHz, DMSO-d6) δ 8.32 (d, J=8.3 Hz, 2H), 8.01 (d, J=8.4 Hz, 2H), 7.71 (s, 1H), 7.36 (d, J=4.9 Hz, 1H), 7.30 (t, J=6.0 Hz, 1H), 7.09 (d, J=3.3 Hz, 1H), 6.98 (dd, J=5.1, 3.5 Hz, 1H), 4.71 (d J=_5.9 Hz, 2H), 3.92 (s, 3H), 3.27 s 3H).

Compound 30:

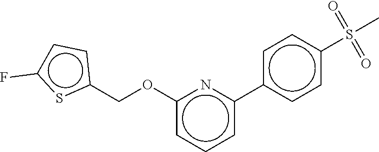

To a stirred solution of 2-chloro-6-(4-methanesulfonylphenyl)pyridine (0.150 g, 0.5602 mmol, 1 eq) were added (5-fluorothiophen-2-yl)methanol (111 mg, 0.8403 mmol, 1.5 eq) and caesium carbonate (364 mg, 1.12 mmol, 2 eq) in Toluene (5 mL) at room temperature and reaction mixture was degassed with argon for 20 min followed by tBuBrettPhos Pd G3 (23.9 mg, 0.02801 mmol, 0.05 eq) was added at room temperature and reaction mixture was heated at 100° C. for 16 h. After completion of reaction, the reaction mixture was poured in to water (25 mL) and extracted with EtOAc (3×25 mL). The organic layer was washed with brine solution (2×10 mL), dried over Na2SO4 and evaporated. The product was added to a Prep HPLC column and was eluted with 55-60% ACN in 5 mM ABC+0.1% NH3 in water as a gradient to provide compound 30 (66.40 mg, 32.7% yield) as a white solid. ¹H NMR (400 MHz, DMSO-d6) δ 8.42 (d, J=8.1 Hz, 2H), 8.05 (d, J=8.2 Hz, 2H), 7.89 (t, J=7.8 Hz, 1H), 7.77 (d, J=7.5 Hz, 1H), 7.00 (t, J=3.8 Hz, 1H), 6.91 (d, J=8.2 Hz, 1H), 6.63 (s, 1H), 5.59 (s, 2H), 3.28 (s, 3H).

### Compound 31

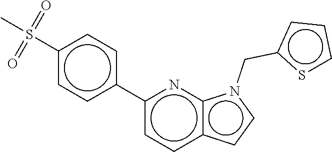

Was prepared using 6-chloro-1-[(thiophen-2-yl)methyl]-1H-pyrrolo[2,3-b]pyridine (0.2 g, 0.8040 mmol, 1 eq) and (4-methanesulfonylphenyl)boronic acid (192 mg, 0.9648 mmol, 1.2 eq) using method A. The residue was purified via Biotage (2:1 Hex/EtOAc; 12S column) and further purified by prep HPLC using (25-85% ACN in water containing 0.1% formic acid) as mobile phase to provide compound 31 (0.035 g, 11.8% yield) as a white solid. MS(ESI):369.0[M+H]⁺. ¹H NMR (400 MHz, DMSO-d6) δ 8.50 (d, J=8.5 Hz, 2H), 8.14 (d, J=8.1 Hz, 1H), 8.06 (d, J=8.4 Hz, 2H), 7.88 (d, J=8.2 Hz, 1H), 7.76 (d, J=3.5 Hz, 1H), 7.42 (dd, J=5.1, 1.3 Hz, 1H), 7.22 (d, J=3.4 Hz, 1H), 6.99 (dd, J=5.1, 3.5 Hz, 1H), 6.58 (d, J 3.4 Hz, 1H), 5.76 (s, 2H), 3.29 (s, 3H).

### Compound 33

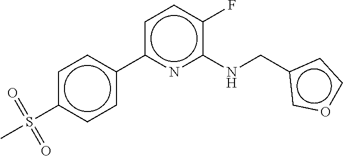

Was prepared from 2-bromo-3-fluoro-6-(4-methanesulfonylphenyl)pyridine (50 mg, 0.1514 mmol, 1.0 eq) and 1-(furan-3-yl)methanamine (15.2 μL, 0.1665 mmol, 1.1 eq) using method 2. The crude material was purified by normal phase chromatography (hexane/EtOAc, 0-50%) and the resulting solid was washed several times with little amounts of Et2O to afford compound 33 (20.6 mg, 39.3% yield) as a white solid. MS: [M+H]⁺ 346.73. ¹H NMR (400 MHz, Chloroform-d) δ 8.16 (d, J=8.3 Hz, 2H), 7.99 (d, J=8.3 Hz, 2H), 7.47 (s, 1H), 7.40 (s, 1H), 7.25 (dd, J=10.4, 8.2 Hz, 1H), 7.09 (dd, J=8.1, 3.0 Hz, 1H), 6.45 (d, J=1.7 Hz, 1H), 4.89 (s, 1H), 4.64 (d, J=5.5 Hz, 2H), 3.08 (s, 3H). 19F NMR (376 MHz, Chloroform-d) δ-145.10.

### Compound 34

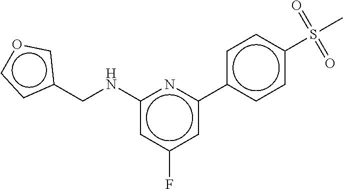

Was prepared from 2-bromo-4-fluoro-6-(4-methanesulfonylphenyl)pyridine (30 mg, 0.09086 mmol, 1 eq) and 1-(furan-3-yl)methanamine (9.97 μL, 0.1090 mmol, 1.2 eq) using method 2. The solvent was evaporated and the crude material was purified by normal phase chromatography to afford compound 34 (23.9 mg, 76.1% yield) as a pale yellow solid. MS: [M+H]⁺ 347.10. ¹H NMR (400 MHz, Chloroform-d) δ 8.15 (d, J=8.3 Hz, 2H), 8.01 (d, J=8.3 Hz, 2H), 7.44 (s, 1H), 7.41 (s, 1H), 6.88 (dd, J=9.4, 1.8 Hz, 1H), 6.43 (d, J=1.7 Hz, 1H), 6.14 (dd, J=10.6, 1.8 Hz, 1H), 4.94 (t, J=5.1 Hz, 2H), 4.44 (d, J=5.5 Hz, 2H), 3.08 (s, 3H).

19F NMR (376 MHz, Chloroform-d) 6-105.89 (t, J=10.0 Hz).

### Compound 35

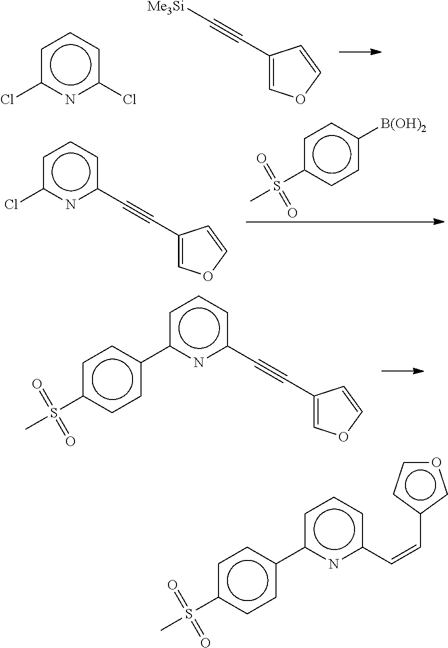

In a flask under nitrogen flow to 2,6-dichloropyridine (500 mg, 3.37 mmol, 2 eq), [2-(furan-3-yl)ethynyl]trimethylsilane (275 mg, 1.68 mmol, 1 eq), Pd(PPh3)2Cl2 (59.1 mg, 0.08425 mmol, 0.05 eq), triphenylphosphine (22.0 mg, 0.08425 mmol, 0.05 eq), CuI (64.1 mg, 0.3370 mmol, 0.2 eq) and triethylamine (469 μL, 3.37 mmol, 2 eq) were added. The mixture was degassed for 10 minutes and then it was diluted with DMF (3.36 mL). The mixture was stirred at room temperature for 5 minutes, then TBAF (1.0 M in THF, 1.68 mL, 1.68 mmol, 1.0 eq) was added dropwise. The reaction mixture was hit up at 50° C. and stirred over night. The mixture was cooled at room temperature. Solvent was evaporated and the aqueous layer was extracted with EtOAc (3×10 mL). The combined organics were dried over MgSO4, filtered through Celite and evaporated. 2-chloro-6-[2-(furan-3-yl)ethynyl]pyridine (260 mg, 76.0% yield) was purified using flash column chromatography (SiO2, 0-100% Hexanes\EtOAc). MS: 204.13 [M+H]⁺. ¹H NMR (400 MHz, Chloroform-d) δ 7.76 (dd, J=1.5, 0.8 Hz, 1H), 7.63 (t, J=7.8 Hz, 1H), 7.43-7.38 (m, 2H), 7.29 (s, 1H), 6.56 (dd, J=1.8, 0.8 Hz, 1H).

In a flask under nitrogen flow to 2-chloro-6-[2-(furan-3-yl)ethynyl]pyridine (250 mg, 1.22 mmol, 1 eq), boronic acid (244 mg, 1.22 mmol, 1 eq), potassium carbonate (505 mg, 3.66 mmol, 3 eq) and Pd(PPh3)4 (70.4 mg, 0.061 mmol, 0.05 eq) were added. The mixture was degassed for 10 minutes and then it was diluted with water (3 mL) and 1,4-dioxane (12 mL). The dark mixture was stirred at 80° C. over night. Then it was cooled at r. t. and solvent was evaporated. The aqueous layer was extracted with EtOAc (3×10 mL), the combined organics were dried over MgSO4, filtered through Celite and evaporated. 2-[2-(furan-3-yl)ethynyl]-6-(4-methanesulfonylphenyl)pyridin (100 mg, 25.3% yield) was purified using flash column chromatography (SiO2, 0-100% Hexanes\EtOAc). MS: 324.43 [M+H]⁺. ¹H NMR (400 MHz, Chloroform-d) δ 8.22 (dt, J=12.7, 1.9 Hz, 2H), 8.04 (dt, J=12.7, 1.9 Hz, 2H), 7.84-7.77 (m, 2H), 7.72 (dd, J=8.0, 1.0 Hz, 1H), 7.52 (d, J=7.6 Hz, 1H), 7.43 (t, J=1.6 Hz, 1H), 6.61-6.58 (m, 1H), 3.09 (s, 3H).

In a flask under nitrogen flow to 2-[2-(furan-3-yl)ethynyl]-6-(4-methanesulfonylphenyl)pyridin (100 mg, 0.3092 mmol, 1 eq) was added Lindlar catalyst (7 mg). The mixture was degassed for 10 minutes and then it was diluted with ethanol (3 mL). The mixture was stirred at r.t. under hydrogenated atmospheric pressure. After 1 h, 23 mg of Lindlar was added and the reaction mixture was stirred over night at r.t. under hydrogenated atmospheric pressure. At last 40 mg of Lindlar was added and the reaction mixture was stirred at r.t. under hydrogenated atmospheric pressure for 24 h. The solvent was evaporated and the crude was extracted with EtOAc and the combined organics were dried over MgSO4, filtered through Celite and DCM mixture and evaporated. The product was purified using flash column chromatography (SiO2, 0-100% EtOAc\Hexanes), and further purified using preparative HPLC (C18 3×150 5 μm, ACN/water+0.1% Formic Acid), to provide 2-[(Z)-2-(furan-3-yl)ethenyl]-6-(4-methanesulfonylphenyl)pyr (11 mg, 10.1% yield). MS: 326.56 [M+H]⁺. ¹H NMR (400 MHz, Chloroform-d) δ 8.27 (d, J=8.5 Hz, 2H), 8.06 (d, J=8.4 Hz, 2H), 7.77 (t, J=7.8 Hz, 1H), 7.70-7.60 (m, 3H), 7.45 (s, 1H), 7.34 (d, J=7.7 Hz, 1H), 6.95 (d, J=15.9 Hz, 1H), 6.72 (d, J=1.5 Hz, 1H), 3.09 (s, 3H).

### Compound 36

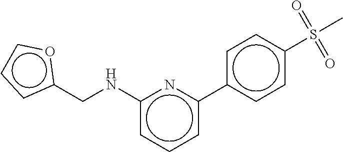

Was prepared using intermediate-3 (100 mg, 0.3735 mmol, 1 eq) and 2-furanmethanamine (43.5 mg, 0.4482 mmol, 1.2 eq) using method 2. The residue was purified using flash column chromatography (SiO4, 0-100% EtOAc/hexanes) to afford compound 36 (67 mg, 54.9% yield). MS: 328.49 [M+H]⁺. ¹H NMR (300 MHz, Chloroform-d) δ 8.18 (dt, J=12.3, 1.9 Hz, 2H), 7.99 (dt, J=12.2, 1.9 Hz, 2H), 7.52 (dd, J=8.2, 7.5 Hz, 1H), 7.37 (dd, J=1.8, 0.8 Hz, 1H), 7.13 (dd, J=7.5, 0.5 Hz, 1H), 6.48 (dd, J=8.9, 7.7 Hz, 1H), 6.33 (dd, J=3.2, 1.9 Hz, 1H), 6.27 (dq, J=3.2, 0.8 Hz, 1H), 4.93 (t, J=5.5 Hz, 1H), 4.63 (d, J=5.7 Hz, 2H), 3.07 (s, 3H).

### Compound 37

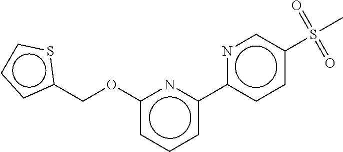

A stirred suspension of 6′-chloro-5-(methylsulfanyl)-2,2′-bipyridine (0.120 g, 0.5069 mmol, 1 eq), (thiophen-2-yl)methanol (86.7 mg, 0.7603 mmol, 1.5 eq) and caesium carbonate (329 mg, 1.01 mmol, 2.0 eq) in 1,4-dioxane (5 mL) was degassed with nitrogen gas for 15 min. After 15 min, tBuBrettPhos Pd G3 (21.6 mg, 0.02534 mmol, 0.05 eq) was added to it and heated at 100° C. for 12 h. After 12 h, the reaction mixture was diluted with water (60 mL) and extracted with ethyl acetate (3×40 mL). The organic layers was dried over Na2SO4 and evaporated. The residue was purified via Biotage (5:1 Hex/EtOAc; 12S column) to provide 5-(methylsulfanyl)-6′-[(thiophen-2-yl)methoxy]-2,2′-bipyridine (56 mg, 35.2% yield) as a off white semi solid.

To a stirred solution of 5-(methylsulfanyl)-6′-[(thiophen-2-yl)methoxy]-2,2′-bipyridine (0.056 g, 0.1781 mmol, 1 eq) in THF (4 mL) was added solution of oxone (59.8 mg, 0.3562 mmol, 2.0 eq) in Water (1 mL) and allowed to stir at RT for 7 h. After completion of the reaction; the reaction mixture was poured into water (10 mL) and extracted with ethyl acetate (3×10 mL). the combined organics were dried over Na2SO4 and evaporated. The residue was purified via Biotage (1:1 Hex/EtOAc; 12M column) to provide compound 37 as an off white solid. MS(ESI): 347.0 [M+H]⁺.

Compounds 38 and 119:

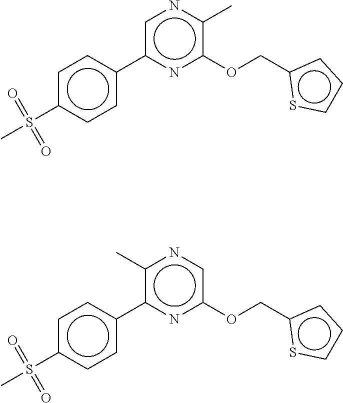

Was prepared from (thiophen-2-yl)methanol (66.5 mg, 0.5828 mmol) and a mixture of 3-chloro-5-(4-methanesulfonylphenyl)-2-methylpyrazine (200 mg, 0.2914 mmol) and 5-chloro-3-(4-methanesulfonylphenyl)-2-methylpyrazine (200 mg, 0.3713 mmol) using method 1. The residue was purified via Biotage (2:1 Hex/EtOAc; 12M column) to provide mixture of two compounds which was further purified by prep HPLC using [50-57% ACN in water containing 0.1% formic acid as a modifier] as a mobile phase to provide compound 38 (60 mg, 57.1% yield) as a yellow semi-solid and compound 119 (50 mg, 46.9% yield) as a off white solid. Compound 38: MS(ESI): 361.0 [M+H]⁺. ¹H NMR (400 MHz, DMSO-d6) δ 8.86 (s, 1H), 8.46 (d, J=8.3 Hz, 2H), 8.07 (d, J=8.2 Hz, 2H), 7.55 (d, J=5.0 Hz, 1H), 7.33 (d, J=3.3 Hz, 1H), 7.05 (dd, J=5.1, 3.5 Hz, 1H), 5.77 (s, 2H), 3.30 (s, 3H), 2.46 (s, 3H). Compound 119: MS(ESI): 361.0 [M+H]⁺. ¹H NMR (400 MHz, DMSO-d6) δ 8.86 (s, 1H), 8.46 (d, J=8.3 Hz, 2H), 8.07 (d, J=8.3 Hz, 2H), 7.55 (d, J=5.1 Hz, 1H), 7.33 (d, J=3.4 Hz, 1H), 7.05 (dd, J=5.1, 3.5 Hz, 1H), 5.77 (s, 2H), 3.30 (s, 3H), 2.46 (s, 3H).

### Compound 39

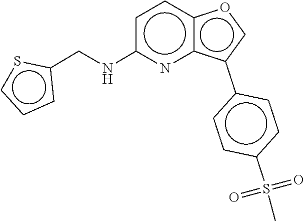

Was prepared from 5-chloro-3-(4-methylsulfonylphenyl)furo[3,2-b]pyridine (0.30 g, 0.920 mmol, 1.00 eq) and 2-thiophenemethylamine (0.11 g, 1.01 mmol, 1.10 eq) using method 3. The residue was purified via Biotage (2:1 Hex/EtOAc; 12M column) to provide compound 39 (240 mg, 69% yield) as an off white solid. MS(ESI): 385.0 [M+H]⁺. ¹H NMR (400 MHz, DMSO-d6) δ 8.75 (s, 1H), 8.52 (d, J=8.2 Hz, 2H), 7.97 (d, J=8.2 Hz, 2H), 7.77 (d, J=9.1 Hz, 1H), 7.34 (dd, J=8.7, 5.2 Hz, 2H), 7.10 (d, J=3.4 Hz, 1H), 7.01-6.91 (m, 1H), 6.60 (d, J=9.1 Hz, 1H), 4.76 (d, J=5.8 Hz, 2H), 3.24 (s, 3H).

### Compound 40

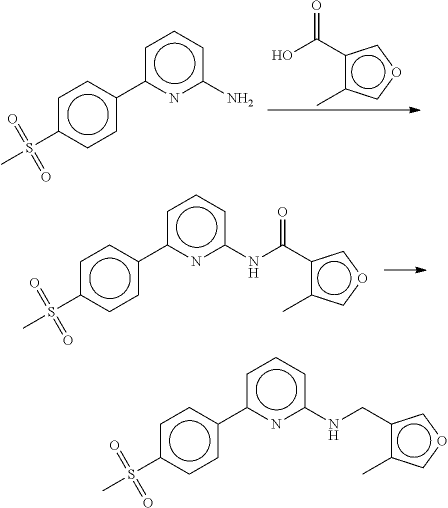

To a stirred solution of 6-(4-methanesulfonylphenyl)pyridin-2-amine (200 mg, 0.8054 mmol) and 4-methylfuran-3-carboxylic acid (101 mg, 0.8054 mmol) in DCM (4 mL) was drop wise added pyridine (779 μL, 9.66 mmol, 12.0 eq) and phosphorus oxychloride (747 μL, 8.05 mmol, 10.0 eq) and allowed it to stir at RT for 2 h. After completion of the reaction, the reaction mixture was poured into saturated NaHCO₃ solution (30 mL) and extracted with DCM (3×20 mL). The combined organics were washed with 10% citric acid solution (50 mL), dried over Na2SO4 and evaporated. The residue was purified via Biotage (1:1 Hex/EtOAc; 12M column) to provide N-[6-(4-methanesulfonylphenyl)pyridin-2-yl]-4-methylfuran-3-carboxamide (100 mg, 30.5%) as a white solid. MS(ESI): 355.0 [M−H]⁻, 357.0 [M+H]⁺.

To a stirred solution of N-[6-(4-methanesulfonylphenyl)pyridin-2-yl]-4-methylfuran-3-carboxamide (100 mg, 0.2805 mmol) in THF (3 mL) was added 2M borane dimethylsulfide complex in THF (700 μL, 1.40 mmol, 5.0 eq) and heated it at 60° C. for 1 h. After completion of the reaction, the reaction mixture was poured into water (15 mL) and extracted with ethyl acetate (3×10 mL). The combined organics were dried over Na2SO4 and evaporated. The residue was purified via Biotage (1:1 Hex/EtOAc; 12S column) to provide impure compound, which was further purified by prep HPLC purification using (15-70% ACN in 5 mM ammonium bicarbonate solution containing 0.1% ammonia as a modifier) as a mobile phase to provide compound 40 (20 mg, 20.1% yield) as a yellow semi-solid. MS(ESI): 343.0 [M+H]⁺. ¹H NMR (400 MHz, DMSO-d6) δ 8.29 (d, J=8.4 Hz, 2H), 7.98 (d, J=8.2 Hz, 2H), 7.52 (dd, J=16.0, 8.2 Hz, 2H), 7.39 (s, 1H), 7.21 (d, J=7.3 Hz, 1H), 6.94 (t, J=5.4 Hz, 1H), 6.58 (d, J=8.3 Hz, 1H), 4.37 (d, J=5.3 Hz, 2H), 3.25 (s, 3H), 1.98 (s, 3H).

### Compound 41

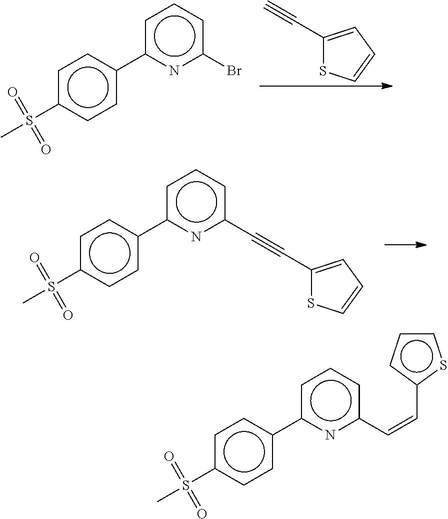

In a flask under nitrogen flow to 2-bromo-6-(4-methanesulfonylphenyl)pyridine (100 mg, 0.3203 mmol, 1 eq), 2-ethynylthiophene (30.4 μL, 0.3203 mmol, 1 eq), Pd(PPh3)2Cl2 (11.2 mg, 0.01601 mmol, 0.05 eq), CuI (6.09 mg, 0.03202 mmol, 0.1 eq), triethylamine (111 μL, 0.8007 mmol, 2.5 eq) and triphenylphosphine (4.19 mg, 0.01601 mmol, 0.05 eq) were added. The mixture was degassed for 10 minutes and then it was diluted with Acetonitrile (2.5 mL), previously degassed. The mixture was stirred at 80° C. for 6 h, then it was cooled at r.t. and solvent was evaporated. The aqueous layer was extracted with EtOAc (3×10 mL) and the combined organics were dried over MgSO4, filtered through Celite and evaporated. 2-(4-methanesulfonylphenyl)-6-[2-(thiophen-2-yl)ethynyl]pyridine (40 mg, 37.0% yield) was purified using flash column chromatography (SiO4, 0-100% hexanes/EtOAc) and further purified using reverse phase chromatography (0-100% water/ACN with 0.1% formic acid). MS: 340.17 [M+H]⁺. ¹H NMR (400 MHz, Chloroform-d) δ 8.24 (dt, J=12.6, 2.0 Hz, 2H), 8.06 (dt, J=12.2, 2.0, 1.4 Hz, 2H), 7.84 (t, J=7.8 Hz, 1H), 7.76-7.64 (m, 2H), 7.56 (dd, J=7.7, 0.8 Hz, 1H), 7.45 (dd, J=5.9, 3.7 Hz, 1H), 7.39 (dd, J=5.1, 1.1 Hz, 1H), 7.06 (dd, J=5.1, 3.7 Hz, 1H), 3.09 (s, 3H).

In a flask under nitrogen flow to 2-(4-methanesulfonylphenyl)-6-[2-(thiophen-2-yl)ethynyl]pyridine (100 mg, 0.2946 mmol, 1 eq) was added Lindlar catalyst (20 mg). The mixture was degassed and then it was diluted with ethanol (3 mL). The mixture was stirred at r.t. under hydrogenated atmospheric pressure. After 2 h 30 mg of Lindlar was added to the reaction mixture. After 1 H, 50 mg of Lindlar was added. The mixture was stirred at r.t. under hydrogenated atmospheric pressure over night. The 344 peak in the LCMS reveals that part of the product was over reduced in the final alkane. The solvent was evaporated and the crude was extracted with EtOAc and the combined organics were dried over MgSO4, filtered through Celite and DCM mixture and evaporated. compound 41 (45 mg, 44.9% yield) was purified using flash column chromatography (SiO2, 0-100% EtOAc\Hexanes). MS: 342.43 [M+H]⁺. ¹H NMR (400 MHz, Chloroform-d) δ 8.25 (dt, J=12.4, 2.0 Hz, 2H), 8.03 (dt, J=12.3, 1.7 Hz, 2H), 7.78 (t, J=7.8 Hz, 1H), 7.67 (d, J=7.6 Hz, 1H), 7.38 (d, J=7.7 Hz, 1H), 7.20 (dd, J=11.9, 4.4 Hz, 2H), 6.99-6.90 (m, 2H), 6.60 (d, J=12.4 Hz, 1H), 3.10 (s, 3H).

### Compound 43

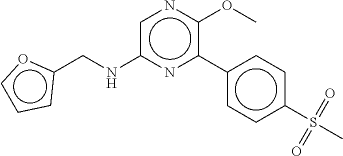

Obtained from 5-chloro-3-(4-methanesulfonylphenyl)-2-methoxypyrazine (0.1 g, 0.3347 mmol, 1 eq) and 1-(furan-2-yl)methanamine (39.0 mg, 0.4016 mmol, 1.2 eq) using method 3. The residue was purified via Biotage (2:1 Hex/EtOAc; 12S column) to provide compound 43 (0.035 g, 29.1% yield) as yellow solid. MS(ESI): 360.0[M+H]⁺. ¹H NMR (400 MHz, DMSO-d6) δ 8.32-8.23 (m, 2H), 8.03-7.95 (m, 2H), 7.71 (s, 1H), 7.58 (d, J=1.9 Hz, 1H), 7.13 (t, J=5.8 Hz, 1H), 6.40-6.27 (m, 2H), 4.49 (d, J=5.8 Hz, 2H), 3.89 (s, 3H), 3.25 (s, 3H).

### Compound 44

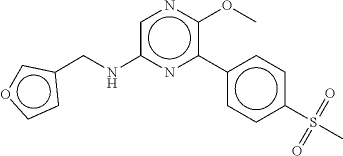

Obtained from 5-chloro-3-(4-methanesulfonylphenyl)-2-methoxypyrazine (0.1 g, 0.3347 mmol, 1 eq) and 1-(furan-3-yl)methanamine (39.0 mg, 0.4016 mmol, 1.2 eq) using method 3. The residue was purified via Biotage (2:1 Hex/EtOAc; 12S column) to provide compound 44 (0.021 g, 17.5% yield) as yellow solid. MS(ESI): 360.0 [M+H]⁺. ¹H NMR (400 MHz, DMSO-d6) δ 8.28 (d, J=8.5 Hz, 2H), 7.99 (d, J=8.5 Hz, 2H), 7.75-7.51 (m, 3H), 6.97 (t, J=5.8 Hz, 1H), 6.49 (d, J=1.7 Hz, 1H), 4.33 (d, J=5.7 Hz, 2H), 3.89 (s, 3H).

### Compound 45

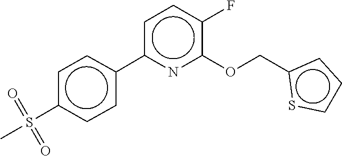

Was prepared from 2-bromo-3-fluoro-6-(4-methanesulfonylphenyl)pyridine (50 mg, 0.1514 mmol, 1.0 eq) and (thiophen-2-yl)methanol (28.6 μL, 0.3028 mmol, 2.0 eq) using method 2. The solvent was evaporated and the crude material was purified by normal phase chromatography (hexane/EtOAc, 30%), then by preparative TLC (hexane/EtOAc, 40%, Rf=0.33). The resulting colorless oil was precipitated in hexane to afford compound 45 (3.5 mg, 6.5% yield) as a white solid. MS: [M+H]⁺ 364.38. ¹H NMR (400 MHz, Chloroform-d) δ 8.20 (d, J=8.2 Hz, 2H), 8.04 (d, J=8.3 Hz, 2H), 7.49-7.39 (m, 2H), 7.31 (d, J=5.1 Hz, 1H), 7.22 (d, J=3.4 Hz, 1H), 7.00 (dd, J=5.1, 3.5 Hz, 1H), 5.77 (s, 2H), 3.10 (s, 3H). 19F NMR (376 MHz, Chloroform-d) 6-141.86 (dd, J=8.9, 3.3 Hz).

### Compounds 46 and 229:

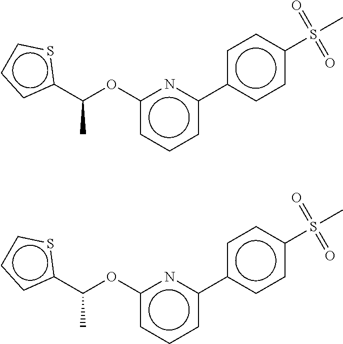

Was prepared using intermediate-3 (500 mg, 1.86 mmol) and 1-(thiophen-2-yl)ethan-1-ol (357 mg, 2.79 mmol) using method 1. The residue was purified by flash chromatography and further purified by prep HPLC purification using (40-65% ACN in 5 mM ammonium bicarbonate containing 0.1% ammonia as a modifier) as a mobile phase to provide 70 mg of racemic compound. The racemic mixture was purified by chiral SFC purification (CHIRALCEL OJ-H (250 mm*4.6 mm, 5 μm)) using CO2 gas and 0.1% diethylamine in methanol as a mobile phase to provide compound 46 (eluting first, 30 mg, 4.38%) as a yellow semi-solid and compound 229 (eluting second, 22 mg, 3.26%) as a yellow semi-solid. Compound 46: ¹H NMR (400 MHz, DMSO-d6) δ 8.36 (d, J=8.2 Hz, 2H), 8.03 (d, J=8.2 Hz, 2H), 7.85 (t, J=7.9 Hz, 1H), 7.70 (d, J=7.5 Hz, 1H), 7.44 (d, J=5.1 Hz, 1H), 7.23 (d, J=3.5 Hz, 1H), 6.99 (t, J=4.2 Hz, 1H), 6.87 (d, J=8.2 Hz, 1H), 6.67 (q, J=6.6 Hz, 1H), 3.27 (s, 3H), 1.74 (d, J=6.6 Hz, 3H). Compound 229: ¹H NMR (400 MHz, DMSO-d6) δ 8.36 (d, J=8.1 Hz, 2H), 8.03 (d, J=8.1 Hz, 2H), 7.85 (t, J=7.9 Hz, 1H), 7.70 (d, J=7.4 Hz, 1H), 7.44 (d, J=5.0 Hz, 1H), 7.23 (d, J=3.4 Hz, 1H), 6.99 (t, J=4.3 Hz, 1H), 6.87 (d, J=8.2 Hz, 1H), 6.67 (q, J=6.5 Hz, 1H), 3.27 (s, 3H), 1.74 (d, J=6.5 Hz, 3H). The absolute stereochemistries of Compounds 46 and 229 were arbitrarily assigned.

### Compound 47: N-(furan-3-ylmethyl)-6-(4-(methylsulfonyl)phenyl)pyridin-2-amine

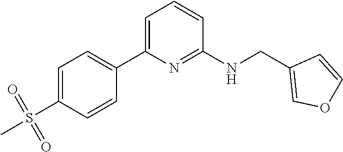 [SMILES: CS(=O)(=O)c1ccc(-c2cccc(NCc3ccoc3)n2)cc1]

Was prepared using intermediate-4 (300 mg, 0.9609 mmol, 1 eq) and 1-(furan-3-yl)methanamine (132 μL, 1.44 mmol, 1.5 eq) using method 2. The residue was purified by reverse phase chromatography (water/acetonitrile+0.1% formic acid, 30-80%) to afford the desired product. It was then washed a few times with little amounts of diethyl ether to remove remaining traces of ligand and afford compound 47 (193.3 mg, 61.2% yield) as a white solid. MS: [M+H]⁺ 328.76. ¹H NMR (400 MHz, Chloroform-d) δ 8.19 (d, J=8.7 Hz, 2H), 8.00 (d, J=8.7 Hz, 2H), 7.54 (t, J=7.9 Hz, 1H), 7.44 (s, 1H), 7.40 (t, J=1.6 Hz, 1H), 7.13 (d, J=7.5 Hz, 1H), 6.46 (d, J=8.2 Hz, 1H), 6.44 (s, 1H), 4.78 (s, 1H), 4.47 (d, J=4.5 Hz, 2H), 3.08 (s, 2H).

### Compound 48

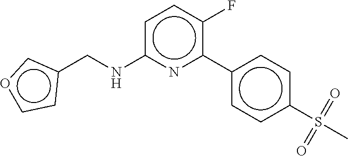

Was prepared from 6-bromo-3-fluoro-2-(4-methanesulfonylphenyl)pyridine (50 mg, 0.1514 mmol, 1.0 eq) and 1-(furan-3-yl)methanamine (15.2 μL, 0.1665 mmol, 1.1 eq) using method 2. The solvent was evaporated and the crude material was purified by normal phase chromatography (hexane/EtOAc, 0-50%) to afford compound 48 (46.5 mg, 88.7% yield) as a pale yellow oil. MS: [M+H]⁺ 346.47. ¹H NMR (400 MHz, Chloroform-d) δ 8.21 (d, J=8.8 Hz, 1H), 8.01 (d, J=8.6 Hz, 2H), 7.43 (p, J=0.8 Hz, 1H), 7.40 (t, J=1.7 Hz, 1H), 7.32 (dd, J=10.5, 8.9 Hz, 1H), 6.45 (dd, J=8.9, 2.6 Hz, 1H), 6.42 (dd, J=1.8, 0.9 Hz, 1H), 4.69 (t, J=4.9 Hz, 1H), 4.42 (d, J=5.6 Hz, 2H), 3.08 (s, 3H). 19F NMR (376 MHz, Chloroform-d) 6-141.19.

### Compound 49

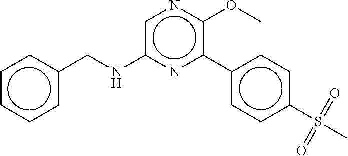

Was prepared from 5-chloro-3-(4-methanesulfonylphenyl)-2-methoxypyrazine (80 mg, 0.2677 mmol, 1 eq) and benzylamine (29.1 μL, 0.2677 mmol, 1 eq) using method 2. The crude was purified by normal phase flash chromatography (Hexane/EtOAc, 0-100%), and again by normal phase flash chromatography to give compound 49 (15.6 mg, 15.7% yield) as a yellow solid. MS: [M+H]⁺ 370.55. ¹H NMR (300 MHz, Chloroform-d) δ 8.30 (d, J=8.6 Hz, 2H), 7.98 (d, J=8.6 Hz, 2H), 7.54 (s, 1H), 7.41-7.28 (m, 5H), 4.57 (s, 2H), 3.98 (s, 3H), 3.07 (s, 3H).

### Compound 51

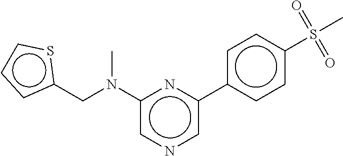

Was prepared using intermediate-7 (150 mg, 0.5582 mmol) and N-methyl-1-(thiophen-2-yl)methanamine (71.0 mg, 0.5582 mmol) using method 2. The residue was purified via Biotage (50:1 CH2Cl2/MeOH; 12M column) to provide impure product, which was further purified by prep HPLC using (40-53% ACN in water containing 0.1% formic acid as modifier) as mobile phase to provide compound 51 (90 mg, 44.9% yield) as a off white solid. MS(ESI): 360.2 [M+H]⁺. ¹H NMR (400 MHz, Chloroform-d) δ 8.40 (s, 1H), 8.29-8.20 (m, 2H), 8.09 (s, 1H), 8.07-7.99 (m, 2H), 7.20 (dd, J=5.1, 1.3 Hz, 1H), 7.02 (d, J=3.4 Hz, 1H), 6.96 (dd, J=5.1, 3.5 Hz 1H) 5.04 (s, 2H), 3.21 (s, 3H), 3.10 (s, 3H).

### Compound 52

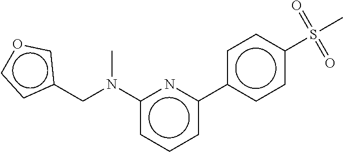

Was prepared using intermediate-4 (0.2 g, 0.6406 mmol, 1 eq) and [(furan-3-yl)methyl](methyl)amine (85.4 mg, 0.7687 mmol, 1.2 eq) using method 2. The residue was purified via Biotage (2:1 Hex/EtOAc; 12S column) to provide impure product, which was further purified by prep HPLC using (55-25% ACN in 0.1% formic acid in water as modifier) as mobile phase to provide compound 52 (0.106 g, 48.4% yield) as an off white solid. ¹H NMR (400 MHz, DMSO-d6) δ 8.31 (d, J=8.2 Hz, 2H), 7.99 (d, J=8.2 Hz, 2H), 7.70-7.59 (m, 2H), 7.57 (t, J=1.8 Hz, 1H), 7.30 (d, J=7.4 Hz, 1H), 6.76 (d, J=8.5 Hz, 1H), 6.39 (d, J=1.8 Hz, 1H), 4.68 (s, 2H), 3.25 (s, 3H), 3.07 (s, 3H).

### Compounds 53 and 249

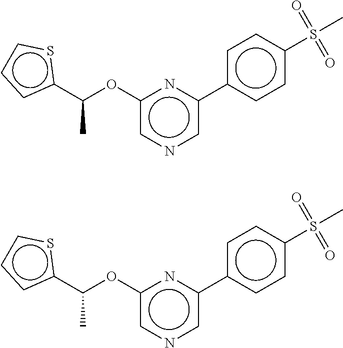

Was prepared using intermediate-7 (300 mg, 1.11 mmol) and 1-(thiophen-2-yl)ethan-1-ol (212 mg, 1.66 mmol) using method 1. The residue was purified via Biotage (2:1 Hex/EtOAc; 40+S column) to provide the racemic mixture which was further purified by chiral SFC (CHIRALCEL OJ-H (250 mm*4.6 mm, 5 μm) to provide compound 53 (eluting first, 70 mg, 17.4% yield) and compound 249 (eluting second, 90 mg, 22.3% yield) as a yellow semi-solid. Compound 53: MS(ESI): 361.0 [M+H]⁺. ¹H NMR (400 MHz, DMSO-d6) δ 8.96 (s, 1H), 8.47-8.39 (m, 2H), 8.35 (s, 1H), 8.13-8.03 (m, 2H), 7.49 (dd, J=5.1, 1.3 Hz, 1H), 7.28 (d, J=3.5 Hz, 1H), 7.01 (dd, J=5.1, 3.5 Hz, 1H), 6.66 (q, J=6.5 Hz, 1H), 3.30 (s, 3H), 1.78 (d, J=6.5 Hz, 3H). Compound 249: MS(ESI): 361.0 [M+H]⁺. ¹H NMR (400 MHz, DMSO-d6) δ 8.96 (s, 1H), 8.48-8.39 (m, 2H), 8.35 (s, 1H), 8.08 (d, J=8.4 Hz, 2H), 7.49 (dd, J=5.0, 1.3 Hz, 1H), 7.28 (d, J=3.4 Hz, 1H), 7.01 (dd, J=5.1, 3.5 Hz, 1H), 6.66 (q, J=6.4 Hz, 1H), 3.30 (s, 3H), 1.78 (d, J=6.5 Hz, 3H). The absolute stereochemistries of Compounds 53 and 249 were arbitrarily assigned.

### Compound 55

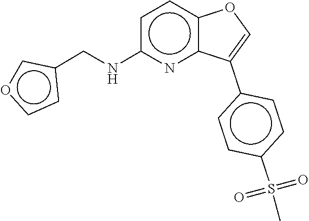

Was prepared using 5-chloro-3-(4-methylsulfonylphenyl)furo[3,2-b]pyridine (0.30 g, 0.988 mmol, 1.00 eq), furan-3-ylmethanamine (0.19 g, 1.95 mmol, 1.97 eq) using method 2. The residue was purified via Biotage (5:1 Hex/EtOAc; 12S column) and further purified by prep HPLC using (25-70% ACN in water containing 5 mM ammonium carbonate and 0.1% ammonia in water) as mobile phase to provide compound 55 (0.01672 g, 4% yield) as a white solid. MS(ESI): 369.5 [M+H]⁺. ¹H NMR (400 MHz, DMSO-d6) δ 8.76 (s, 1H), 8.52 (d, J=8.2 Hz, 2H), 7.99 (d, J=8.2 Hz, 2H), 7.76 (d, J=9.0 Hz, 1H), 7.67 (s, 1H), 7.60 (s, 1H), 7.04 (s, 1H), 6.60 (d, J=9.2 Hz, 1H), 6.53 (s, 1H), 4.41 (d, J=5.6 Hz, 2H), 3.25 (s, 3H).

### Compound 56

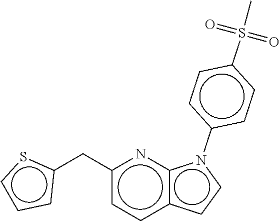

A stirred suspension of [1-(4-methylsulfonylphenyl)pyrrolo[2,3-b]pyridin-6-yl]-(2-thienyl)methanone (0.040 g, 0.105 mmol, 1.00 eq) in Ethylene glycol (1 mL) and Hydrazine hydrate 99% (1 mL) Sodium hydroxide (0.0084 g, 0.209 mmol, 2.00 eq) was added to it and heated at 90° C. for 16 h. The reaction mixture was diluted with water (20 mL) and extracted with ethyl acetate (3×20 mL). The organic layers was dried over Na2SO4 and evaporated. The residue was purified via Biotage (5:1 Hex/EtOAc; 12S column) and further purified by prep HPLC using (25-70% ACN in water containing 5 mM ammonium carbonate and 0.1% ammonia in water) as mobile phase to provide compound 56 (5.0 mg, 13% yield) as an off white solid. MS(ESI): 369.2 [M+H]⁺. ¹H NMR (400 MHz, DMSO-d6) δ 8.39 (d, J=8.7 Hz, 2H), 8.16-7.98 (m, 4H), 7.36 (d, J=4.9 Hz, 1H), 7.24 (d, J=8.0 Hz, 1H), 6.97 (dd, J=10.0, 5.1 Hz, 2H), 6.79 (d, J=3.8 Hz, 1H), 4.39 (s, 2H), 3.29 (s, 3H).

### Compound 57

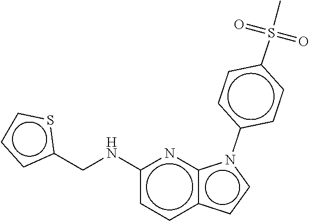

Obtained from 6-bromo-1-(4-methanesulfonylphenyl)-1H-pyrrolo[2,3-b]pyridine (0.066 g, 0.1879 mmol, 1 eq) and 1-(thiophen-2-yl)methanamine (0.025 g, 0.2208 mmol, 1.175 eq) using method 3. The product was added to a Prep HPLC column and was eluted with 30-75% ACN in 0.1% formic acid in water as a gradient to provide compound 57 (0.007 g, 9.7% yield) as a Brown solid. MS: 384.3 [M+H]⁺. ¹H NMR (400 MHz, DMSO-d6) δ8.32 (d, J=8.5 Hz, 2H), 7.98 (d, J=8.6 Hz, 2H), 7.70 (d, J=8.5 Hz, 1H), 7.64 (d, J=3.8 Hz, 1H), 7.39-7.29 (m, 2H), 7.06 (d, J=3.3 Hz, 1H), 6.97 (dd, J=5.1, 3.4 Hz, 1H), 6.55 (d, J=3.8 Hz, 1H), 6.50 (d, J=8.5 Hz, 1H), 4.69 (d, J=5.8 Hz, 2H), 3.26 (s, 3H).

### Compound 58

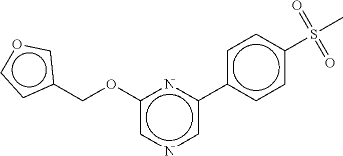

Was prepared using intermediate-7 (40 mg, 0.1488 mmol, 1 eq) and (furan-3-yl)methanol (25.5 μL, 0.2976 mmol, 2 eq) using method 1. The crude was purified by flash chromatography (Hexanes/EtOAc, 0-100%, eluted 60%) to give compound 58 (36.2 mg, 74% yield) as a white solid. MS: 331.15 [M+H]⁺. ¹H NMR (300 MHz, Chloroform-d) δ 8.68 (s, 1H), 8.28-8.21 (m, 3H), 8.10-8.06 (m, 2H), 7.58 (dd, J=1.5, 0.8 Hz, 1H), 7.44 (t, J=1.7 Hz, 1H), 6.55-6.50 (m, 1H), 5.41 (s, 2H), 3.11 (s, 3H).

### Compound 59

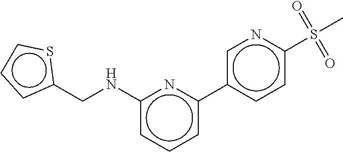

Obtained from 6-bromo-6′-methanesulfonyl-2,3′-bipyridine (200 mg, 0.6386 mmol) and 1-(thiophen-2-yl)methanamine (72.2 mg, 0.6386 mmol) using method 3. The residue was purified via Biotage (2:1 Hex/EtOAc; 12M column) to provide compound 59 (50 mg, 22.7%) as an off white solid. MS(ESI): 346.2 [M+H]⁺. ¹H NMR (400 MHz, DMSO-d6) δ 9.42 (d, J=2.2 Hz, 1H), 8.73 (dd, J=8.2, 2.3 Hz, 1H), 8.11 (d, J=8.2 Hz, 1H), 7.57 (t, J=7.8 Hz, 1H), 7.47 (t, J=6.0 Hz, 1H), 7.38-7.26 (m, 2H), 7.07 (d, J=3.7 Hz, 1H), 6.95 (dd, J=5.1, 3.4 Hz, 1H), 6.62 (d, J=8.3 Hz, 1H), 4.76 (d, J=6.0 Hz, 2H), 3.32 (s, 3H).

### Compound 60

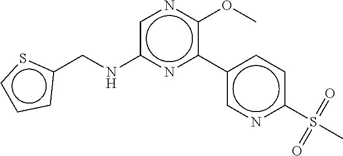

Obtained from 5-chloro-3-(6-methanesulfonylpyridin-3-yl)-2-methoxypyrazine (0.08 g, 0.2669 mmol, 1 eq) and 1-(thiophen-2-yl)methanamine (45.3 mg, 0.4003 mmol, 1.5 eq) using method 3. The residue was purified via Biotage (2:1 Hex/EtOAc; 12S column) to provide impure product, which was further purified by prep HPLC using (35-60% ACN in 0.1% formic acid in water as modifier) as mobile phase to provide compound 60 (0.03 g, 30.0% yield) as a off white solid. MS(ESI):377.0[M+H]⁺. ¹H NMR (400 MHz, DMSO-d6) δ 9.39 (d, J=2.1 Hz, 1H), 8.74 (dd, J=8.3, 2.2 Hz, 1H), 8.14 (d, J=8.3 Hz, 1H), 7.75 (s, 1H), 7.41 (t, J=6.0 Hz, 1H), 7.35 (d, J=5.0 Hz, 1H), 7.07 (d, J=3.4 Hz, 1H), 6.96 (dd, J=5.1, 3.4 Hz, 1H), 4.70 (d, J=5.9 Hz, 2H), 3.93 (s, 3H), 3.32 (s, 3H).

### Compound 61

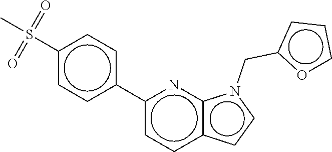

To a stirred solution of 6-(4-methanesulfonylphenyl)-1H-pyrrolo[2,3-b]pyridine (100 mg, 0.3672 mmol) in DMF (2 mL) was added 60% sodium hydride (29.3 mg, 0.7344 mmol) and stirred at same temperature for 30 min. After 30 min, 2-(bromomethyl)furan (177 mg, 1.10 mmol) was drop wise added to it and allowed to stir it at RT for 6 h. After completion of the reaction, the reaction mixture was poured into cold water (20 mL) and obtained solid was filtered through Buchner funnel. The residue was purified via Biotage (1:1 Hex/EtOAc; 12M column) to provide slight impure product which was purified by prep HPLC purification using (10-35% ACN in water containing 0.1% formic acid as modifier) as a mobile phase to provide compound 61 (50 mg, 38.7% yield) as a off white solid. MS(ESI): 353.0 [M+H]⁺. ¹H NMR (400 MHz, DMSO-d6) δ 8.54-8.37 (m, 2H), 8.12 (d, J=8.1 Hz, 1H), 8.08-7.95 (m, 2H), 7.86 (d, J=8.2 Hz, 1H), 7.62 (dd, J=26.8, 2.6 Hz, 2H), 6.57 (d, J=3.5 Hz, 1H), 6.52-6.33 (m, 2H), 5.58 (s, 2H), 3.27 (s, 3H).

### Compound 62

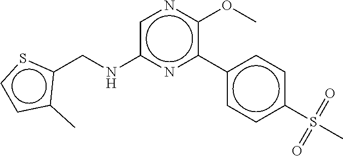

Obtained from 5-bromo-3-(4-methanesulfonylphenyl)-2-methoxypyrazine (125 mg, 0.3642 mmol, 1 eq) and 1-(3-methylthiophen-2-yl)methanamine (55.5 mg, 0.4370 mmol, 1.2 eq) using method 2. The residue was purified using flash column chromatography (SiO2, 0-100% Hexanes\EtOAc) and further purified using reverse phase chromatography (0-100% Water\ACN, 0.1% Formic Acid) to afford compound 62 (10 mg, 6.7% yield). MS: 390.21 [M+H]⁺. ¹H NMR (400 MHz, Chloroform-d) δ 8.35 (dt, J=12.2, 1.8 Hz, 2H), 7.99 (dt, J=12.5, 2.0, 1.6 Hz, 2H), 7.55 (s, 1H), 7.12 (d, J=5.1 Hz, 1H), 6.83 (d, J=5.1 Hz, 1H), 4.66 (s, 2H), 3.99 (s, 3H), 3.08 (s, 3H), 2.27 (s, 3H).

### Compound 63

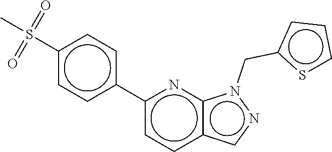

In a flask to 6-chloro-1-[(thiophen-2-yl)methyl]-1H-pyrazolo[3,4-b]pyridin (50 mg, 0.2002 mmol, 1 eq), boronic acid (40.0 mg, 0.2002 mmol, 1 eq), potassium carbonate (82.9 mg, 0.6005 mmol, 3 eq) and XPhos Pd G2 (8.47 mg, 0.01001 mmol, 0.05 eq) were added. The mixture was degassed for 10 minutes and then it was diluted with water (400 μL) and 1,4-dioxane (1.6 mL). The dark mixture was stirred at 80° C. for 44 h. A complete starting material conversion was observed. The solvent was evaporated and the aqueous layer was extracted with EtOAc (3×10 mL), the combined organics were dried over MgSO₄, filtered through Celite and DCM mixture and evaporated. compound 63 (50 mg, 67.6% yield) was purified using flash column chromatography (SiO2, 0-100% Hexanes\EtOAc). MS: 370.22 [M+H]⁺. ¹H NMR (400 MHz, Chloroform-d) δ 8.37 (dt, J=12.2, 1.8 Hz, 2H), 8.16 (d, J=8.3 Hz, 1H), 8.13-8.05 (m, 3H), 7.67 (d, J=8.4 Hz, 1H), 7.21 (dd, J=5.1, 1.1 Hz, 1H), 7.18 (dd, J=3.5, 1.1 Hz, 1H), 6.95 (dd, J=5.1, 3.5 Hz, 1H), 5.95 (s, 2H), 3.12 (s, 3H).

### Compound 65

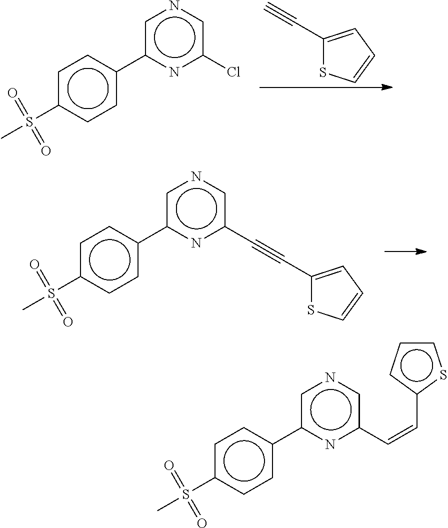

In a flask under nitrogen flow to 2-chloropyrazine (50 mg, 0.1860 mmol, 1 eq) were added 2-ethynylthiophene (30.1 mg, 0.279 mmol, 1.5 eq), Pd(PPh3)Cl2 (6.52 mg, 0.009300 mmol, 0.05 eq), CuI (3.54 mg, 0.01860 mmol, 0.1 eq), triphenylphosphine (2.43 mg, 0.009300 mmol, 0.05 eq) and triethylamine (51.7 μL, 0.372 mmol, 2.0 eq). The mixture was degassed for 15 minutes, diluted with Acetonitrile (1.5 mL) previously degassed and stirred at 90° C. for 1 h. The aqueous layer was extracted with EtOAc (3×10 mL) and the combined organics were dried over MgSO4, filtered through Celite and evaporated. 2-(4-(methylsulfonyl)phenyl)-6-(thiophen-2-ylethynyl)pyrazine (20 mg, 31.5% yield) was purified using reverse chromatography (0-100%, MeCN/H2O). MS: 341.05 [M+H]⁺. ¹H NMR (300 MHz, Chloroform-d) δ 8.97 (s, 1H), 8.75 (s, 1H), 8.27 (dt, J=12.2, 1.9 Hz, 2H), 8.10 (dt, J=11.9, 1.8 Hz, 2H), 7.50-7.42 (m, 2H), 7.09 (dd, J=5.1, 3.7 Hz, 1H), 3.11 (s, 3H).

To the 2-(4-(methylsulfonyl)phenyl)-6-(thiophen-2-ylethynyl)pyrazine (20 mg, 0.05875 mmol, 1 eq) was added Lindlar catalyst (20 mg). The mixture was degassed under nitrogen flow, diluted with ethanol (1 mL) and stirred at r.t. and hydrogenated under atmospheric pressure. The crude was filtered through Celite and evaporated. compound 65 (8 mg, 39.8% yield) was purified using flash column chromatography (SiO4, 0-100% EtOAc/hexanes). MS: 343.14 [M+H]⁺. ¹H NMR (300 MHz, Chloroform-d) δ 8.90 (s, 1H), 8.60 (s, 1H), 8.29 (dt, J=24.2, 3.5, 1.8 Hz, 2H), 8.09 (dt, J=20.0, 2.1, 1.6 Hz, 2H), 7.32-7.26 (m, 2H), 7.15-7.04 (m, 1H), 7.02 (dd, J=8.4, 1.2 Hz, 1H), 6.54 (d, J=12.6 Hz, 1H), 3.13 (s, 3H).

### Compound 66

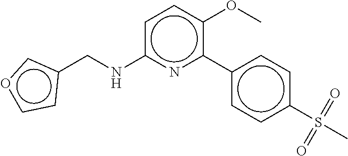

Obtained from 6-bromo-N-[(furan-3-yl)methyl]-5-methoxypyridin-2-amine (0.055 g, 0.1942 mmol, 1 eq) and (4-methanesulfonylphenyl)boronic acid (46.6 mg, 0.2330 mmol, 1.2 eq) using method A. The residue was purified via Biotage (5:1 Hex/EtOAc; 12S column) to provide compound 66 (0.01 g, 14.3% yield) as a yellow solid. MS(ESI):359.0 [M+H]⁺. ¹H NMR (400 MHz, DMSO-d6) δ 8.17 (d, J=8.2 Hz, 2H), 7.94 (d, J=8.4 Hz, 2H), 7.64-7.55 (m, 2H), 7.45 (d, J=8.9 Hz, 1H), 6.62 (d, J=8.3 Hz, 2H), 6.47 (d, J=1.7 Hz, 1H), 4.30 (d, J=5.7 Hz, 2H), 3.74 (s, 3H), 3.24 (s, 3H).

### Compound 67

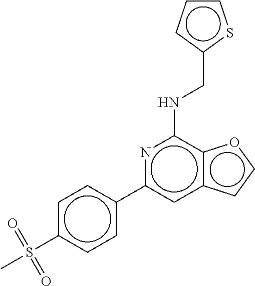

To a stirred solution of 7-bromo-5-(4-methanesulfonylphenyl)furo[2,3-c]pyridine (0.091 g, 0.2583 mmol, 1 eq), were added 1-(thiophen-2-yl)methanamine (35.0 mg, 0.3099 mmol, 1.2 eq) and caesium carbonate (168 mg, 0.5166 mmol, 2 eq) in Dioxane (4 mL) reaction mixture was degassed with argon for 20 min and tBuBrettPhos Pd G3 (22.1 mg, 0.02583 mmol, 0.1 eq) was added and reaction mixture was stirred at 100° C. for 12 h. After completion of reaction, the reaction mixture was poured in to Water (20 mL) and extracted with EtOAc (3×20 mL). The organic layer was washed with brine solution (2×10 mL), dried over Na2SO4 and evaporated. The crude product was purify by combiflash using [0-30% EtOAc/Hexanes] to provide compound 67 (0.0011 g, 1.1% yield) as a white solid. ¹H NMR (400 MHz, DMSO-d6) δ 8.35 (d, J=8.3 Hz, 2H), 8.14 (d, J=2.1 Hz, 1H), 7.97 (d, J=8.3 Hz, 2H), 7.82 (t, J=6.1 Hz, 1H), 7.63 (s, 1H), 7.30 (d, J=4.9 Hz, 1H), 7.10 (d, J=3.4 Hz, 1H), 6.99 (d, J=2.1 Hz, 1H), 6.94 (dd, J=5.1, 3.4 Hz, 1H), 4.91 (d, J=6.1 Hz, 2H), 3.25 (s, 3H).

### Compound 68

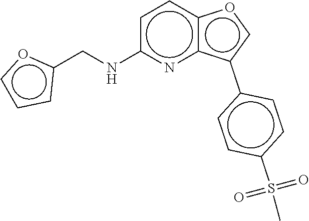

Was prepared using 5-chloro-3-(4-methylsulfonylphenyl)furo[3,2-b]pyridine (0.470 g, 1.53 mmol, 1.00 eq) and furfurylamine (0.16 g, 1.69 mmol, 1.10 eq) using method 3. The residue was purified via Biotage (5:1 Hex/EtOAc; 12S column) and further purified by prep HPLC using (25-70% ACN in water containing 5 mM ammonium carbonate and 0.1% ammonia in water) as mobile phase to provide compound 68 (0.157 g, 27.6% yield) as yellowish solid. MS(ESI):369.0 [M+H]⁺. ¹H NMR (400 MHz, DMSO-d6) δ 8.76 (s, 1H), 8.51 (d, J=8.1 Hz, 2H), 8.00 (d, J=8.1 Hz, 2H), 7.77 (d, J=9.0 Hz, 1H), 7.59 (s, 1H), 7.23 (s, 1H), 6.62 (d, J=9.1 Hz, 1H), 6.47-6.30 (m, 2H), 4.57 (s, 2H), 3.26 (s, 3H).

### Compound 69

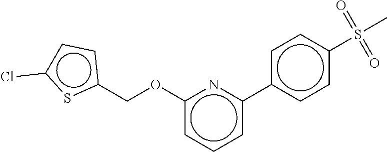

Was prepared using intermediate-3 (0.1 g, 0.3735 mmol, 1 eq) and (5-chlorothiophen-2-yl)methanol (83.2 mg, 0.5602 mmol, 1.5 eq) using method 2. The residue was purified via Biotage (5:1 Hex/EtOAc; 12S column) to provide impure product, which was further purified by prep HPLC using (25-70% ACN in water containing 5 mM ammonium carbonate and 0.1% ammonia in water as modifier) as mobile phase to provide compound 69 (30.0 mg, 21.2% yield) as a off white solid. ¹H NMR (400 MHz, DMSO-d6) δ 8.66 (d, J=8.6 Hz, 2H), 8.33 (d, J=2.3 Hz, 1H), 8.14 (d, J=8.6 Hz, 2H), 7.50 (dd, J=5.1, 1.2 Hz, 1H), 7.27 (d, J=3.6 Hz, 1H), 7.16 (s, 1H), 7.07 (d, J=2.3 Hz, 1H), 7.02 (dd, J=5.1, 3.4 Hz, 1H), 5.71 (s, 2H), 3.30 (s, 3H).

### Compound 70

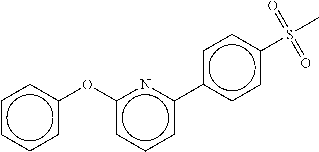

Was prepared using intermediate-4 (100 mg, 0.3203 mmol, 1 eq) and phenol (36.1 mg, 0.3843 mmol, 1.2 eq) using method 2. The product still contained traces of tBuBrettPhos, so it was washed twice with little amount of Et2O to afford compound 70 (39.2 mg, 37.6% yield) as a white powder. MS: [M+H]⁺ 327.15. ¹H NMR (400 MHz, Chloroform-d) δ 8.09 (d, J=8.6 Hz, 2H), 7.96 (d, J=8.6 Hz, 2H), 7.80 (dd, J=8.2, 7.5 Hz, 1H), 7.53 (dd, J=7.5, 0.7 Hz, 1H), 7.43 (dd, J=8.5, 7.4 Hz, 2H), 7.25-7.19 (m, 3H), 6.90 (dd, J=8.2, 0.6 Hz, 1H), 3.05 (s, 3H).

### Compound 71

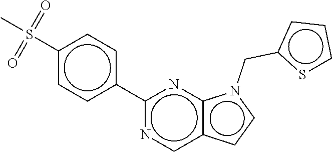

Obtained from 2-chloro-7-[(thiophen-2-yl)methyl]-7H-pyrrolo[2,3-d]pyrimidine (0.3 g, 1.20 mmol, 1 eq) and (4-methanesulfonylphenyl)boronic acid (288 mg, 1.44 mmol, 1.2 eq) using method A. The residue was purified via Biotage (2:1 Hex/EtOAc; 12S column) to provide impure product, which was further purified by prep HPLC using (26-65% ACN in water containing ammonium bicarbonate and 0.1% ammonia in water as modifier) as mobile phase to provide compound 71 (0.04 g, 9.0% yield) as a white solid. MS(ESI):370.0[M+H]⁺.

¹H NMR (400 MHz, DMSO-d6) δ 9.17 (s, 1H), 8.76 (d, J=8.2 Hz, 2H), 8.09 (d, J=8.3 Hz, 2H), 7.82 (d, J=3.6 Hz, 1H), 7.43 (d, J=5.1 Hz, 1H), 7.24 (d, J=3.4 Hz, 1H), 6.98 (t, J=4.3 Hz, 1H), 6.72 (d, J=3.5 Hz, 1H), 5.77 (s, 2H), 3.28 (s, 3H).

### Compound 72

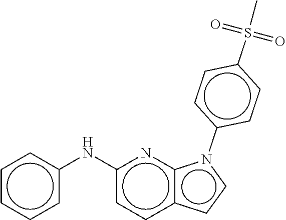

In a vial under nitrogen were dissolved 6-bromo-1-(4-methanesulfonylphenyl)-1H-pyrrolo[2,3-b]pyridine (30 mg, 0.08541 mmol, 1 eq), (tert-butoxy)sodium (16.4 mg, 0.1708 mmol, 2.0 eq), BrettPhos Pd G4 (3.93 mg, 0.004270 mmol, 0.05 eq), BrettPhos (2.29 mg, 0.004270 mmol, 0.05 eq) and aniline (10.0 μL, 0.1110 mmol, 1.3 eq) in degassed dioxane (0.4 mL). The mixture was stirred at 60° C. for 2.5 h and the solvent was evaporated. The crude material was purified by normal phase chromatography (hexane/EtOAc, 0-40%) and the resulting yellow solid was washed a few times with Et2O to afford compound 72 (16.7 mg, 53.8% yield) as a white solid. MS: [M+H]⁺ 364.14. ¹H NMR (400 MHz, Chloroform-d) δ 8.17 (d, J=8.8 Hz, 2H), 8.06 (d, J=8.8 Hz, 2H), 7.80 (d, J=8.4 Hz, 1H), 7.51-7.45 (m, 2H), 7.37-7.31 (m, 3H), 7.04 (t, J=7.4 Hz, 1H), 6.74 (d, J=8.5 Hz, 1H), 6.59 (d, J=3.7 Hz, 1H), 6.49 (s, 1H), 3.11 (s, 3H).

### Compound 73

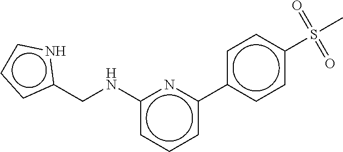

Was prepared using intermediate-4 (250 mg, 0.8008 mmol) and 1-(1H-pyrrol-2-yl)methanamine (76.9 mg, 0.8008 mmol) using method 2. The residue was purified via Biotage (1:1 Hex/EtOAc; 12M column) to provide 80% pure product, which was purified by prep HPLC purification using (30-70% ACN in water containing 10 mM ammonium acetate as modifier) as mobile phase to provide compound 73 (20 mg, 7.6%) as an off white solid. MS(ESI): 328.1 [M+H]⁺. ¹H NMR (400 MHz, DMSO-d6) δ 10.69 (s, 1H), 8.30 (d, J=8.5 Hz, 2H), 7.98 (d, J=8.5 Hz, 2H), 7.51 (t, J=7.8 Hz, 1H), 7.21 (d, J=7.3 Hz, 1H), 6.93 (t, J=5.3 Hz, 1H), 6.68-6.56 (m, 2H), 6.01-5.88 (m, 2H), 4.50 (d, J=5.2 Hz, 2H), 3.25 (s, 3H).

### Compound 74

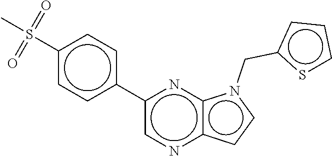

Obtained from 3-chloro-5-[(thiophen-2-yl)methyl]-5H-pyrrolo[2,3-b]pyrazine (0.1 g, 0.4004 mmol, 1 eq) and (4-methanesulfonylphenyl)boronic acid (64.0 mg, 0.3203 mmol, 0.8 eq) using method A. The residue was purified via Biotage (5:1 Hex/EtOAc; 12S column) to provide compound 74 (0.051 g, 32.9% yield) as a white solid. MS(ESI): 370.0[M+H]⁺.

### Compound 75

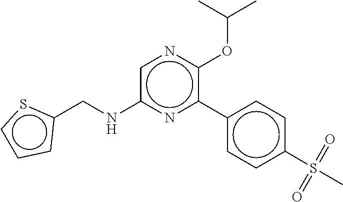

In a flask under argon were added 5-bromo-3-(4-methanesulfonylphenyl)-2-(propan-2-yloxy)pyrazine (67.8 mg, 0.1826 mmol, 1 eq), 1-(thiophen-2-yl)methanamine (18.6 μL, 0.1826 mmol, 1 eq), Pd(OAC)2 (8.19 mg, 0.03652 mmol, 0.2 eq), BrettPhos (39.2 mg, 0.07304 mmol, 0.4 eq) and caesium carbonate (119 mg, 0.3652 mmol, 2 eq) in dioxane (3 mL). The mixture was heated at 90° C. for 3 h and then heated at 100° C. for 20 h. The mixture was cooled down to room temperature and the solvent was evaporated. The crude was purified by normal phase flash chromatography (Hexanes/EtOAc, 0-100%). The product was not pure, it seems that the impurity was BrettPhos. The crude was washed with hexanes and filtered. compound 75 (21.5 mg, 29.2% yield) was obtained as a yellow solid. MS: [M+H]⁺ 404.20. ¹H NMR (400 MHz, Chloroform-d) δ 8.39 (d, J=8.5 Hz, 2H), 7.98 (d, J=8.5 Hz, 2H), 7.55 (s, 1H), 7.21 (dd, J=5.1, 1.1 Hz, 1H), 7.04 (d, J=3.1 Hz, 1H), 6.97 (dd, J=5.0, 3.5 Hz, 1H), 5.27 (hept, J=6.2 Hz, 1H), 4.76 (s, 2H), 4.69 (s, 1H), 3.08 (s, 3H), 1.38 (d, J=6.2 Hz, 6H).

### Compound 76

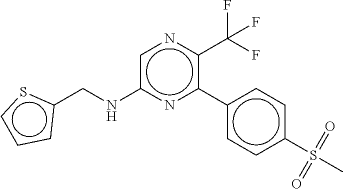

Obtained from 6-chloro-N-[(thiophen-2-yl)methyl]-5-(trifluoromethyl)pyrazin-2-amine (70 mg, 0.2383 mmol) and (4-methanesulfonylphenyl)boronic acid (47.6 mg, 0.2383 mmol) using method A. The residue was purified via Biotage (2:1 Hex/EtOAc; 12M column) to provide compound 76 (40 mg, 40.6% yield) as a white solid. MS(ESI): 412.0 [M−H]⁻, 414.0 [M+H]⁺. ¹H NMR (400 MHz, DMSO-d6) δ 8.65 (t, J=5.9 Hz, 1H), 8.16-7.96 (m, 3H), 7.76 (d, J=8.0 Hz, 2H), 7.41 (d, J=5.1 Hz, 1H), 7.07 (d, J=3.4 Hz, 1H), 6.98 (dd, J=5.0, 3.4 Hz, 1H), 4.72 (d, J=5.8 Hz, 2H), 3.30 (s, 3H).

### Compound 77

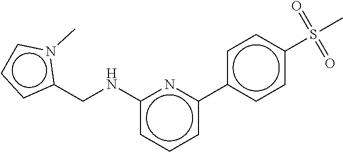

Was prepared using intermediate-3 (200 mg, 0.7470 mmol) and 1-(1-methyl-1H-pyrrol-2-yl)methanamine (82.2 mg, 0.747 mmol) using method 2. The residue was purified via Biotage (1:1 Hex/EtOAc; 12M column) to provide 70% pure product, which was purified by prep HPLC purification using (30-50% ACN in water containing 5 mM ammonium bicarbonate+ammonia as a modifier) as mobile phase to provide compound 77 (25 mg, 9.6%) as an yellow solid. MS(ESI): 342.0 [M+H]⁺, 340.0 [M−H]⁻. ¹H NMR (400 MHz, DMSO-d6) δ 8.37-8.27 (m, 2H), 8.04-7.92 (m, 2H), 7.51 (t, J=7.8 Hz, 1H), 7.21 (d, J=7.3 Hz, 1H), 7.00 (t, J=5.3 Hz, 1H), 6.67 (t, J=2.3 Hz, 1H), 6.60 (d, J=8.3 Hz, 1H), 6.03 (dd, J=3.4, 1.8 Hz, 1H), 5.89 (t, J=3.1 Hz, 1H), 4.53 (d, J=5.2 Hz, 2H), 3.59 (s, 3H), 3.25 (s, 3H).

### Compound 78

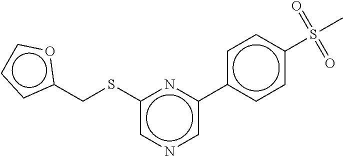

To a mixture of intermediate-7 (40 mg, 0.1488 mmol, 1 eq) and Potassium Carbonate (41.1 mg, 0.2976 mmol, 2 eq) in DMF (1.48 mL) was added 2-furanmethanethiol (22.0 mg, 0.1934 mmol, 1.3 eq). The resulting solution was stirred at 60° C. over night. The aqueous layer was extracted with EtOAc (3×10 mL) and the combined organics were dried over MgSO4, filtered through Celite and DCM mixture and evaporated. Compound 78 (33 mg, 61.7% yield) was purified using flash column chromatography (SiO4, 0-100% EtOAc/hexanes, eluted 70%). MS: 348.59 [M+H]⁺. ¹H NMR (300 MHz, Chloroform-d) δ 8.73 (s, 1H), 8.46 (s, 1H), 8.24 (dt, J=12.0, 1.7 Hz, 2H), 8.08 (dt, J=12.1, 2.0 Hz, 2H), 7.35 (d, J=1.0 Hz, 1H), 6.31-6.24 (m, 2H), 4.56 (s, 2H), 3.11 (s, 3H).

### Compound 80

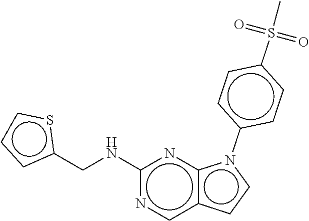

Obtained from 2-chloro-7-(4-methanesulfonylphenyl)-7H-pyrrolo[2,3-d]pyrimidine (0.055 g, 0.1787 mmol, 1 eq) and 1-(thiophen-2-yl)methanamine (24.2 mg, 0.2144 mmol, 1.2 eq) using method 3. The residue was purified via Biotage (2:1 Hex/EtOAc; 12S column) to provide impure product, which was further purified by prep HPLC using (55-25% ACN in 0.1% formic acid in water as modifier) as mobile phase to provide compound 80 (0.008 g, 12% yield) as a yellowish white solid. ¹H NMR (400 MHz, DMSO-d6) δ 8.67 (s, 1H), 8.27 (d, J=8.6 Hz, 2H), 8.03 (d, J=8.5 Hz, 2H), 7.78 (s, 1H), 7.72 (d, J=3.9 Hz, 1H), 7.32 (d, J=5.0 Hz, 1H), 7.04 (d, J=3.5 Hz, 1H), 6.99-6.91 (m, 1H), 6.67 (d, J=3.9 Hz, 1H), 4.68 (d, J=6.2 Hz, 2H), 3.28 (s, 3H).

### Compound 81

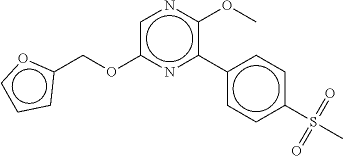

A stirred suspension of 5-chloro-3-(4-methanesulfonylphenyl)-2-methoxypyrazine (0.15 g, 0.5021 mmol, 1 eq), (furan-2-yl)methanol (73.8 mg, 0.7531 mmol, 1.5 eq) and tBuBrettPhos Pd G3 (429 mg, 0.5021 mmol, 1.0 eq) in dioxane (4 mL) was degassed with nitrogen gas for 15 min. After 15 min, caesium carbonate (407 mg, 1.25 mmol, 2.5 eq) was added to it and heated at 100° C. for 12 h. After 12 h, the reaction mixture was diluted with water (80 mL) and extracted with ethyl acetate (3×40 mL). The organic layer was dried over Na2SO₄ and evaporated. The residue was purified via Biotage (5:1 Hex/EtOAc; 12S column) to provide impure product, which was further purified by prep HPLC using (25-75% ACN in water containing 5 mM ammonium carbonate and 0.1% ammonia in water) as mobile phase to provide compound 81 (0.016 g, 8.9% yield) as a off white solid. MS(ESI): 361.0[M+H]⁺. ¹H NMR (400 MHz, DMSO-d6) δ 8.47-8.28 (m, 2H), 8.17-7.96 (m, 3H), 7.71 (d, J=1.8 Hz, 1H), 6.61 (d, J=3.2 Hz, 1H), 6.48 (dd, J=3.3, 1.8 Hz, 1H), 5.40 (s, 2H), 3.98 (s, 3H), 3.28 (s, 3H).

### Compound 82

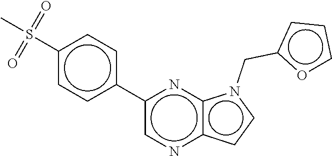

Obtained from 3-chloro-5-[(furan-2-yl)methyl]-5H-pyrrolo[2,3-b]pyrazine (0.075 g, 0.3209 mmol, 1 eq) and (4-methanesulfonylphenyl)boronic acid (77.0 mg, 0.3850 mmol, 1.2 eq) using method A. The residue was purified via Biotage (2:1 Hex/EtOAc; 12S column) to provide impure product, which was further purified by prep HPLC using (25-55% ACN in water containing 0.1% ammonia and ammonium bicarbonate as modifier) as mobile phase to provide compound 82 (0.012 g, 10.6% yield) as a white solid. MS(ESI):354.0[M+H]⁺. ¹H NMR (400 MHz, DMSO-d6) δ 9.20 (s, 1H), 8.53-8.43 (m, 2H), 8.11-8.01 (m, 3H), 7.60 (d, J=1.7 Hz, 1H), 6.77 (d, J=3.7 Hz, 1H), 6.52 (d, J=3.2 Hz, 1H), 6.43 (dd, J=3.3, 1.9 Hz, 1H), 5.60 (s, 2H).

### Compound 83

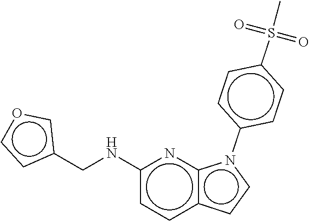

In a vial under nitrogen were dissolved (tert-butoxy)sodium (9.84 mg, 0.1024 mmol, 1.2 eq), BrettPhos Pd G4 (3.93 mg, 0.004270 mmol, 0.05 eq), BrettPhos (2.29 mg, 0.004270 mmol, 0.05 eq), 6-bromo-1-(4-(methylsulfonyl)phenyl)-1H-pyrrolo[2,3-b]pyridine (30 mg, 0.08541 mmol, 1 eq) and 1-(furan-3-yl)methanamine (11.7 μL, 0.1281 mmol, 1.5 eq) in dioxane (0.4 mL). The mixture was stirred at 100° C. for 12 h. The solvent was evaporated and the crude material was purified by normal phase chromatography (hexane/EtOAc, 0-30%) and the resulting oil was washed with hexane to afford compound 83 (3.0 mg, 9.3% yield) as a white solid. MS: [M+H]⁺ 368.06. ¹H NMR (400 MHz, Chloroform-d) δ 8.15 (d, J=8.7 Hz, 2H), 8.02 (d, J=8.9 Hz, 2H), 7.70 (d, J=8.5 Hz, 1H), 7.42 (s, 1H), 7.40 (s, 1H), 7.27 (d, J=4.0 Hz, 1H), 6.54 (d, J=3.7 Hz, 1H), 6.43 (d, J=1.8 Hz, 1H), 6.40 (d, J=8.5 Hz, 1H), 4.64 (d, J=5.8 Hz, 1H), 4.44 (d, J=5.3 Hz, 2H), 3.09 (s, 3H).

### Compound 85

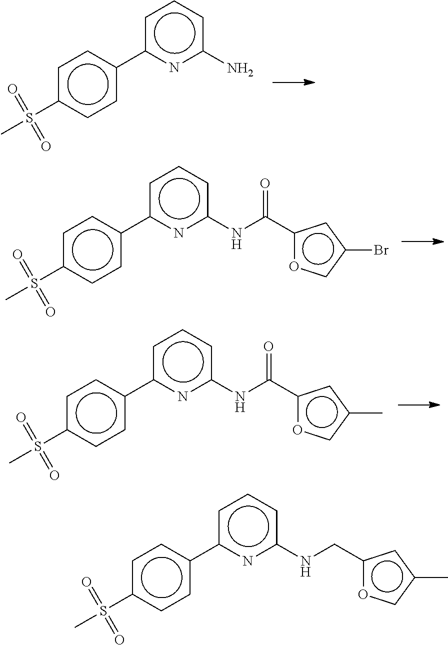

To a stirred solution of 6-(4-methanesulfonylphenyl)pyridin-2-amine (0.5 g, 2.01 mmol, 1 eq) and 4-bromofuran-2-carboxylic acid (383 mg, 2.01 mmol, 1 eq) in DCM (10 mL) was added dropwise pyridine (1.90 g, 24.1 mmol, 12.0 eq) at 0° C. The mixture was added dropwise to a solution of phosphorus oxychloride (3.06 g, 20.0 mmol, 10.0 eq) at 0° C. during a period of 45 min. The resulting mixture was stirred at 0-RT for 2 hour. The mixture was poured in to water (15 mL) and extracted with EtOAc (3×20 mL). The combined extracts were washed with NaHCO₃ (2×15 mL). The organics were dried over Na2SO4 and evaporated to provide 4-bromo-N-(6-(4-(methylsulfonyl)phenyl)pyridin-2-yl)furan-2-carboxamide. MS: [M+H]⁺ 423.0

A stirred suspension of 4-bromo-N-(6-(4-(methylsulfonyl)phenyl)pyridin-2-yl)furan-2-carboxamide (0.250 g, 0.5934 mmol, 1 eq), methylboronic acid (35.5 mg, 0.5934 mmol, 1.0 eq) and caesium carbonate (482 mg, 1.48 mmol, 2.5 eq) in 1,4-Dioxane (5 mL) was degassed with nitrogen gas for 15 min. After 15 min, palladium(2+) bis(cyclopenta-1,3-dien-1-yldiphenylphosphane) (24.2 mg, 0.02967 mmol, 0.05 eq) and Water(2 mL) was added to it and heated at 100° C. for 5 h. After 5 h, the reaction mixture was diluted with ethyl acetate (70 mL) and washed with water (3×40 mL). The organic layers was dried over Na2SO4 and evaporated. The residue was purified via Biotage (5:1 Hex/EtOAc; 12S column) to provide 4-methyl-N-(6-(4-(methylsulfonyl)phenyl)pyridin-2-yl)furan-2-carboxamide (0.093 g, 44.0% yield) as a white solid. MS: [M+H]⁺ 357.2

To a stirred a solution of 4-methyl-N-(6-(4-(methylsulfonyl)phenyl)pyridin-2-yl)furan-2-carboxamide (0.090 g, 0.2525 mmol, 1 eq) in THF (3 mL) at 0° C. and was drop wise added borane dimethylsulfide (95.7 mg, 1.26 mmol, 5.0 eq) and stirred for 1 h at 70° C. The reaction mixture was diluted with water (10 mL) extracted with EtOAc (3×30 mL), and the combined organics were dried over Na2SO4 and evaporated. The residue was purified via Biotage (20:1 CH2Cl2/MeOH; 12M column) to provide impure product, which was further purified by prep HPLC using (15-45% ACN in water containing 10 mM ammonium acetate as modifier) as mobile phase to provide compound 85 (3.0 mg, 3.5% yield) as a brown semi solid. MS: [M+H]⁺ 343.0. ¹H NMR (400 MHz, DMSO-d6) δ 8.28 (d, J=8.3 Hz, 2H), 7.98 (d, J=8.2 Hz, 2H), 7.53 (t, J=7.8 Hz, 1H), 7.31 (s, 1H), 7.22 (d, J=7.3 Hz, 1H), 7.16 (t, J=5.8 Hz, 1H), 6.59 (d, J=8.3 Hz, 1H), 6.17 (s, 1H), 4.51 (d, J=5.6 Hz, 2H), 3.24 (s, 3H), 1.92 (s, 3H).

### Compound 86

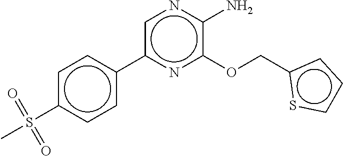

Obtained from 5-bromo-3-[(thiophen-2-yl)methoxy]pyrazin-2-amine (0.55 g, 1.92 mmol, 1.0 eq) and (4-methanesulfonylphenyl)boronic acid (460 mg, 2.30 mmol, 1.2 eq) using method A. The residue was purified via Biotage (50:1 CH2Cl2/MeOH; 12S column) to provide impure product, which was further purified by prep HPLC using (25-52% ACN in water containing 0.1% ammonia as modifier) as mobile phase to provide compound 86 (0.2 g, 28.8% yield) as a white solid. MS(ESI):362.0[M+H]⁺. ¹H NMR (400 MHz, DMSO-d6) δ 8.34 (s, 1H), 8.28-8.26 (m, 2H), 7.97-7.95 (m, 2H), 7.55 (dd, J=4.0 Hz, 1H), 7.34 (d, J=4.0 Hz, 1H), 7.05 (dd, J=4.0 Hz, 1H), 6.75 (s, 1H), 5.75 (s, 2H), 3.25 (s, 3H).

### Compound 87

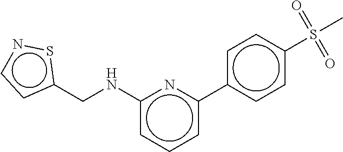

Was prepared using intermediate-3 (0.2 g, 0.7470 mmol, 1 eq) and 1-(1,2-thiazol-5-yl)methanamine hydrochloride (135 mg, 0.8964 mmol, 1.2 eq) using method 2. The residue was purified via Biotage (2:1 Hex/EtOAc; 12S column) to provide impure product, which was further purified by prep HPLC using (35-70% ACN in 0.1% formic acid in water as modifier) as mobile phase to provide compound 87 (0.09 g, 33.7% yield) as a white solid. MS(ESI):346.0[M+H]⁺. ¹H NMR (400 MHz, DMSO-d6) δ 8.42 (d, J=1.7 Hz, 1H), 8.30 (d, J=8.2 Hz, 2H), 7.97 (d, J=8.1 Hz, 2H), 7.58 (t, J=7.7 Hz, 2H), 7.39-7.34 (m, 1H), 7.31 (d, J=7.4 Hz, 1H), 6.61 (d, J=8.2 Hz, 1H), 4.89 (d, J=6.0 Hz, 2H), 3.25 (s, 3H).

### Compounds 89 and 274

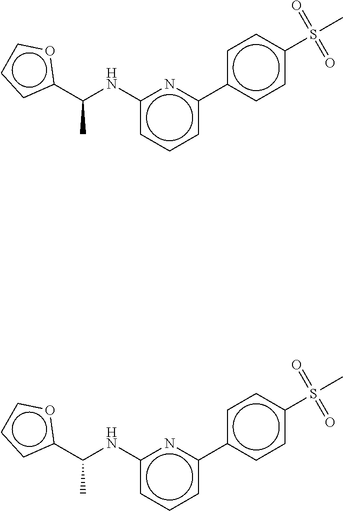

Was prepared using intermediate-3 (0.250 g, 0.9337 mmol, 1 eq) and 1-(furan-2-yl)ethan-1-amine hydrochloride (165 mg, 1.12 mmol, 1.2 eq) using method 2. The residue was purified via Biotage (2:1 Hex/EtOAc; 12S column) to provide impure product, which was further purified by prep HPLC using (55-25% ACN in 0.1% formic acid in water as modifier) as mobile phase to afford the product (0.125 g, 39.1% yield). The racemic mixture was purified by chiral SFC purification by CO2 and 0.1% Diethyl amine in methanol as a co-solvent using Chiralcel OJ-H (250 mm*4.6 mm*5 m) to provide compound 89 (eluting first, 22.2 mg, 24.6% yield) and compound 274 (eluting second, 30.7 mg, 34.1% yield) as white solids. Compound 89: ¹H NMR (400 MHz, DMSO-d6) δ 8.25 (d, J=8.1 Hz, 2H), 7.97 (d, J=8.1 Hz, 2H), 7.63-7.44 (m, 2H), 7.21 (d, J=7.4 Hz, 1H), 7.10 (d, J=8.0 Hz, 1H), 6.58 (d, J=8.3 Hz, 1H), 6.36 (s, 1H), 6.26 (d, J=3.2 Hz, 1H), 5.37 (t, J=7.4 Hz, 1H), 3.24 (s, 3H), 1.50 (d, J=6.9 Hz, 3H). Compound 274: ¹H NMR (400 MHz, DMSO-d6) δ 8.25 (d, J=8.1 Hz, 2H), 7.97 (d, J=8.1 Hz, 2H), 7.64-7.44 (m, 2H), 7.20 (d, J=7.3 Hz, 1H), 7.10 (d, J=8.0 Hz, 1H), 6.58 (d, J=8.3 Hz, 1H), 6.36 (t, J=2.4 Hz, 1H), 6.26 (d, J=3.2 Hz, 1H), 5.45-5.25 (m, 1H), 3.24 (s, 3H), 1.50 (d, J=6.9 Hz, 3H). The absolute stereochemistries of Compounds 89 and 274 were arbitrarily assigned.

### Compound 90

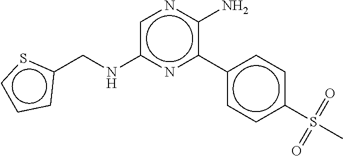

Was prepared using 5-chloro-3-(4-methanesulfonylphenyl)pyrazin-2-amine (638 mg, 2.24 mmol, 1 eq) and 1-(thiophen-2-yl)methanamine (229 μL, 2.24 mmol, 1 eq) using method 2. The crude was purified by normal phase flash chromatography (DCM/Methanol, 0-10%) to give compound 90 (348 mg, 24%) as a yellow solid. MS: [M+H]⁺ 360.87; [M−H]⁻358.98. ¹H NMR (300 MHz, DMSO-d6) δ 8.11-8.05 (m, 2H), 8.00-7.94 (m, 2H), 7.60 (s, 1H), 7.33 (dd, J=5.1, 1.3 Hz, 1H), 7.03 (dd, J=3.4, 1.2 Hz, 1H), 6.95 (dd, J=5.1, 3.4 Hz, 1H), 6.81 (t, J=6.1 Hz, 1H), 5.28 (s, 2H), 4.60 (d, J=6.1 Hz, 2H), 3.24 (s, 3H).

### Compound 91

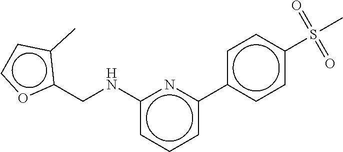

To a stirred solution of 6-(4-methanesulfonylphenyl)pyridin-2-amine (250 mg, 1.00 mmol) and (3-methylfuran-2-yl)methanol (112 mg, 1 mmol) in Toluene (5 mL) was added potassium hydroxide (168 mg, 3.00 mmol) and heated it in microwave at 150° C. for 2 h. After 2 h, the reaction mixture was filtered through celite and filtrate was evaporated. The residue was purified via Biotage (1:1 Hex/EtOAc; 12M column) to provide impure compound which was further purified by prep HPLC using (25-65% ACN in 5 mM ammonium bicarbonate solution contains 0.1% ammonia as a modifier) as a mobile phase to provide compound 91 (10 mg, 2.9%) as an off white solid. MS(ESI): 343.0 [M+H]⁺. ¹H NMR (400 MHz, DMSO-d6) δ 8.28 (d, J=8.1 Hz, 2H), 7.98 (d, J=8.1 Hz, 2H), 7.50 (dd, J=14.9, 7.0 Hz, 2H), 7.20 (d, J=7.4 Hz, 1H), 7.12 (t, J=5.6 Hz, 1H), 6.57 (d, J=8.3 Hz, 1H), 6.27 (s, 1H), 4.53 (d, J=5.5 Hz, 2H), 3.25 (s, 3H), 2.02 (s, 3H).

### Compound 92

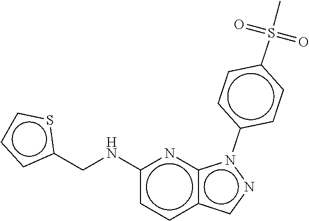

Was prepared using 6-bromo-1-(4-methylsulfonylphenyl)pyrazolo[3,4-b]pyridine (0.13 g, 0.369 mmol, 1.00 eq) and 2-Thiophenemethylamine (0.08 mL, 0.738 mmol, 2.00 eq) using method 2. The residue was purified via Biotage (5:1 Hex/EtOAc; 12S column) and further purified by prep HPLC using (25-70% ACN in water containing 5 mM ammonium carbonate and 0.1% ammonia in water) as mobile phase to provide compound 92 (16.0 mg, 12% yield) as an off white solid. MS(ESI): 385.0 [M+H]⁺. ¹H NMR (400 MHz, DMSO-d6) δ 8.68-8.58 (m, 2H), 8.13 (d, J=3.8 Hz, 2H), 8.03 (d, J=8.9 Hz, 2H), 7.88 (d, J=8.8 Hz, 1H), 7.36 (d, J=5.0 Hz, 1H), 7.14 (d, J=3.4 Hz, 1H), 6.98 (dd, J=5.1, 3.4 Hz, 1H), 6.60 (d, J=8.8 Hz, 1H), 4.80 (d, J=5.7 Hz, 2H), 3.25 (s, 3H).

### Compound 93

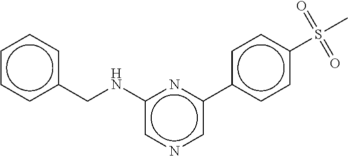

Was prepared using intermediate-7 (100 mg, 0.3721 mmol) and phenylmethanamine (39.8 mg, 0.3721 mmol) using method 2. The residue was purified via Biotage (50:1 CH2Cl2/MeOH; 12M column) to provide impure product; which was further purified by prep HPLC purification by using (30-57% ACN in water containing 0.1% formic acid as a modifier) as a mobile phase to provide compound 93 (15 mg, 11.9% yield) as off white solid. MS(ESI): 340.2 [M+H]⁺. ¹H NMR (400 MHz, Chloroform-d) δ 8.36 (s, 1H), 8.16 (d, J=8.5 Hz, 2H), 8.02 (d, J=8.4 Hz, 2H), 7.94 (s, 1H), 7.43-7.28 (m, 5H), 5.11 (s, 1H), 4.68 (d, J=5.3 Hz, 2H), 3.09 (s, 3H).

### Compound 95

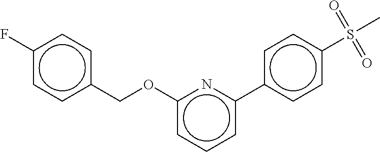

Was prepared using intermediate-4 (100 mg, 0.3203 mmol, 1 eq) and (4-fluorophenyl)methanol (41.6 μL, 0.3843 mmol, 1.2 eq) using method 2. The crude material was purified by normal phase chromatography (Hexane/EtOAc, 5-50%) to afford compound 95 (50.2 mg, 42.7% yield) as a yellow solid. MS: [M+H]⁺ 358.24. ¹H NMR (400 MHz, Chloroform-d) δ 8.24-8.17 (m, 2H), 8.07-8.00 (m, 2H), 7.71 (dd, J=8.2, 7.4 Hz, 1H), 7.51-7.44 (m, 2H), 7.43 (d, J=7.3 Hz, 1H), 7.07 (t, J=8.7 Hz, 2H), 6.84 (d, J=8.2 Hz, 1H), 5.47 (s, 2H), 3.10 (s, 3H). 19F NMR (376 MHz, Chloroform-d) 6-117.48.

### Compound 96

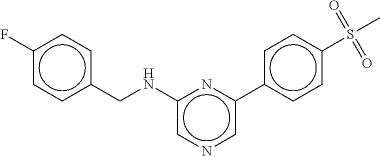

Was prepared using intermediate-7 (150 mg, 0.5582 mmol) and (4-fluorophenyl)methanamine (69.8 mg, 0.5582 mmol) using method 2. The residue was purified via Biotage (50:1 CH2Cl2/MeOH; 12M column) to provide impure product, which was further purified by prep HPLC using (45-50% ACN in water containing 0.1% formic acid as modifier) as mobile phase to provide compound 96 (30 mg, 15.0% yield) as a light yellow solid. MS(ESI): 358.3 [M+H]⁺. ¹H NMR (400 MHz, Chloroform-d) δ 8.39 (s, 1H), 8.17 (d, J=8.3 Hz, 2H), 8.05 (d, 2H), 7.96 (s, 1H), 7.39 (dd, J=8.4, 5.3 Hz, 2H), 7.07 (t, J=8.7 Hz, 2H), 5.10 (t, 1H), 4.67 (d, J=5.5 Hz, 2H), 3.11 (s, 3H).

### Compound 97

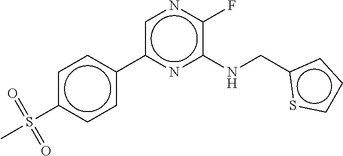

To stirred a solution of 6-(4-(methylsulfonyl)phenyl)-N₂-(thiophen-2-ylmethyl)pyrazine-2,3-diamine (0.1 g, 0.2774 mmol, 1 eq) in fluoboric acid (45% in water, 0.5 mL, 2.77 mmol, 10 eq), was portion wise added sodium nitrite (24.8 mg, 0.3606 mmol, 1.3 eq) at −10° C., while maintaining the temperature below 0° C. The reaction mixture was stirred at 0° C. for 30 min, then at 50° C. for 2 h. After cooling to room temperature, the reaction mixture was basic (pH 9-10) with a saturated aqueous NaHCO3 solution (25 mL) and extracted with ethyl acetate(3×30 mL). The combined organics were dried over Na2SO4 and evaporated. The residue was purified via Biotage (2:1 Hex/EtOAc; 12S column) to provide impure product, which was further purified by prep HPLC using (45-65% ACN in water containing 0.1% formic acid as modifier) as mobile phase to provide compound 97 (0.003 g, 3.0% yield) as a light yellow solid. MS(ESI): 364.0 [M+H]⁺. ¹H NMR (400 MHz, DMSO-d6) δ 8.43-8.20 (m, 3H), 8.16-7.93 (m, 3H), 7.35 (d, J=4.9 Hz, 1H), 7.11 (d, J=3.4 Hz, 1H), 6.96 (dd, J=5.1, 3.4 Hz, 1H), 4.83 (d, J=5.9 Hz, 2H), 3.26 (s, 3H).

### Compound 98

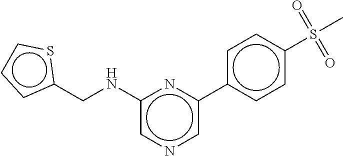

In a flask under nitrogen flow to intermediate-7 (247 mg, 1.09 mmol, 1 eq) were added boronic acid (326 mg, 1.63 mmol, 1.5 eq), potassium carbonate (451 mg, 3.27 mmol, 3 eq) and XPhosPd G2 (42.8 mg, 0.0545 mmol, 0.05 eq). The mixture was diluted with 1,4 dioxane/water (4:1, 5 mL) and stirred at 60° C. for 2 h. The dark mixture was cooled at r.t. and the solvent was evaporated. When 1,4-dioxane was completely evaporated, aqueous layer was extracted with EtOAc (3×10 mL) and the result organic layer was dried over MgSO4, filtered through Celite and DCM mixture and evaporated. The residue was purified using flash column chromatography (SiO2, 0-100% EtOAc/hexanes, eluted 80%) to afford compound 98 (286 mg, 76.0% yield) as a white solid. MS: 346 [M+H]⁺/344 [M−H]⁻. ¹H NMR (300 MHz, Chloroform-d) δ 8.39 (s, 1H), 8.22 (dt, J=8.7, 1.8 Hz, 2H), 8.04 (dt, J=8.8, 1.9 Hz, 2H), 7.95 (d, J=0.2 Hz, 1H), 7.24 (dd, J=5.1, 1.3 Hz, 1H), 7.07 (dq, J=3.5, 0.9 Hz, 1H), 6.98 (dd, J=5.1, 3.5 Hz, 1H), 5.10 (t, J=5.6 Hz, 1H), 4.88 (dd, J=5.9, 0.5 Hz, 2H), 3.10 (s, 3H).

### Compound 99

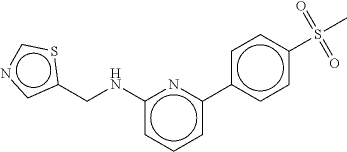

Was prepared using intermediate-3 (0.2 g, 0.7470 mmol, 1 eq) and 1-(1,3-thiazol-5-yl)methanamine hydrochloride (135 mg, 0.8964 mmol, 1.2 eq) using method 2. The residue was purified via Biotage (2:1 Hex/EtOAc; 12S column) to provide impure product, which was further purified by prep HPLC using (25-50% ACN in 0.1% formic acid in water as modifier) as mobile phase to provide compound 99 (0.013 g, 5.0% yield) as a off white solid. MS(ESI):346.0[M+H]⁺. ¹H NMR (400 MHz, DMSO-d6) δ 8.89 (s, 1H), 8.32 (d, J=8.2 Hz, 2H), 7.98 (d, J=8.3 Hz, 2H), 7.87 (s, 1H), 7.55 (t, J=7.8 Hz, 1H), 7.43 (t, J=6.0 Hz, 1H), 7.28 (d, J=7.4 Hz, 1H), 6.57 (d, J=8.3 Hz, 1H), 4.81 (d, J=5.9 Hz, 2H), 3.25 (s, 3H).

### Compounds 100 and 189

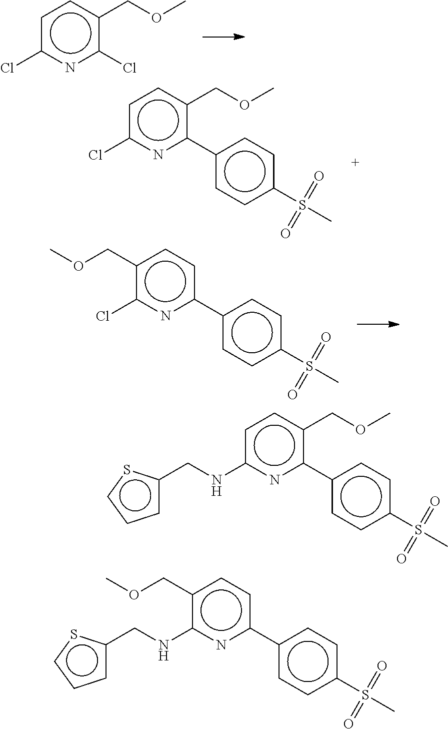

Step 1: Obtained from 2,6-dichloro-3-(methoxymethyl)pyridine (1 g, 5.20 mmol, 1 eq), and (4-methanesulfonylphenyl)boronic acid (1.04 g, 5.20 mmol, 1 eq) using method A. The residue was purified via Biotage (2:1 Hex/EtOAc; 12S column) to provide a mixture of regioisomers (0.5 g, 30.8% yield, ratio of isomer 1:2) as a brown solid. MS(ESI):314.0 [M+H]⁺.

Step 2: Obtained from 1-(thiophen-2-yl)methanamine (217 mg, 1.92 mmol, 1.2 eq) and the mixture of regioisomers (0.5 g, 1.60 mmol, 1 eq) using method 3. The residue was purified via Biotage (2:1 Hex/EtOAc; 12S column) to provide impure product, which was further purified by prep HPLC using (25-55% ACN in 0.1% formic acid in water as modifier) as mobile phase to provide compound 100 (0.08 g, 12.5% yield) as an off white solid and compound 189 (0.14 g, 22.5% yield) as an off white solid. Compound 100: MS(ESI):389.0[M+H]⁺. ¹H NMR (400 MHz, DMSO-d6) δ 8.00 (d, J=8.1 Hz, 2H), 7.87 (d, J=8.1 Hz, 2H), 7.53 (d, J=8.5 Hz, 1H), 7.38 (t, J=6.0 Hz, 1H), 7.34 (d, J=5.1 Hz, 1H), 7.02 (d, J=3.4 Hz, 1H), 6.58 (d, J=8.4 Hz, 1H), 4.68 (d, J=5.9 Hz, 2H), 4.17 (s, 2H), 3.27 (s, 6H). Compound 189: MS(ESI):389.0[M+H]⁺. ¹H NMR (400 MHz, DMSO-d6) δ 8.39-8.29 (m, 2H), 8.01-7.93 (m, 2H), 7.54 (d, J=7.5 Hz, 1H), 7.30 (t, J=5.7 Hz, 2H), 7.07 (d, J=3.4 Hz, 1H), 6.93 (dd, J=5.2, 3.1 Hz, 1H), 6.77 (t, J=6.0 Hz, 1H), 4.86 (d, J=5.8 Hz, 2H), 4.38 (s, 2H), 3.31 (d, J=1.7 Hz, 3H).

### Compound 101

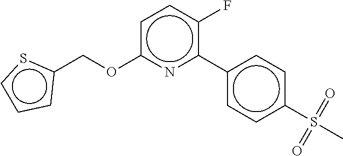

Was prepared using 6-bromo-3-fluoro-2-(4-methanesulfonylphenyl)pyridine (50 mg, 0.1514 mmol, 1.0 eq) and (thiophen-2-yl)methanol (28.6 μL, 0.3028 mmol, 2.0 eq) using method 2. The crude was purified by normal phase chromatography (hexane/EtOAc, 0-100%) to afford compound 101 (15.5 mg, 28.1% yield) as a white powder. MS: [M+H]⁺ 364.09. ¹H NMR (400 MHz, Chloroform-d) δ 8.29 (d, J=8.3 Hz, 2H), 8.05 (d, J=8.3 Hz, 2H), 7.48 (t, J=9.5 Hz, 1H), 7.31 (dd, J=5.1, 1.1 Hz, 1H), 7.17 (d, J=3.4 Hz, 1H), 7.01 (dd, J=5.1, 3.5 Hz, 1H), 6.81 (dd, J=8.9, 2.7 Hz, 1H), 5.63 (s, 2H), 3.11 (s, 3H). 19F NMR (376 MHz, Chloroform-d) 6-136.27 (dd, J=9.9, 3.1 Hz).

### Compound 102

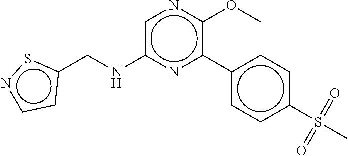

Was prepared using 5-chloro-3-(4-methanesulfonylphenyl)-2-methoxypyrazine (200 mg, 0.6694 mmol) and 1-(1,2-thiazol-5-yl)methanamine (76.4 mg, 0.6694 mmol) using method 2. The residue was purified via Biotage (20:1 CH2Cl2/MeOH; 12M column) and further purified by prep HPLC using 15-45% ACN in water containing 0.1% formic acid as mobile phase, to provide compound 102 (45 mg, 17.9%) as a yellow solid. MS(ESI): 375.0 [M−H]⁻, 377.0 [M+H]⁺. ¹H NMR (400 MHz, DMSO-d6) δ 8.43 (d, J=1.7 Hz, 1H), 8.27 (d, J=8.4 Hz, 2H), 7.98 (d, J=8.4 Hz, 2H), 7.74 (s, 1H), 7.50 (t, J=6.0 Hz, 1H), 7.35 (d, J=1.6 Hz, 1H), 4.84 (d, J=5.9 Hz, 2H), 3.91 (s, 3H), 3.25 (s, 3H).

### Compounds 103 and 145

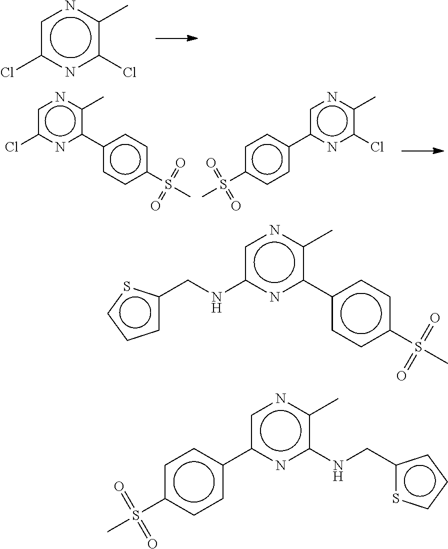

Prepared using 3,5-dichloro-2-methylpyrazine (700 mg, 4.29 mmol) and (4-(methylsulfonyl)phenyl)boronic acid (858 mg, 4.29 mmol) using method A. The residue was purified via Biotage (2:1 Hex/EtOAc; 40+S column) to provide a mixture of unseparable regioisomers (300 mg, 24% yield ratio 2:1) as a off white solid. MS(ESI): 283.2 [M+H]⁺. This mixture (300 mg, 0.6507 mmol) was reacted with thiophen-2-ylmethanamine (110 mg, 0.9760 mmol) using method 3. The residue was purified via Biotage (50:1 CH2Cl2/MeOH; 12M column) and further purified by prep HPLC using (20-55% ACN in water containing 0.1% formic acid) as mobile phase to provide compound 103 (70 mg, 29.4% yield) as a off white solid and compound 145 (35 mg, 14.8% yield) as a off white solid.

Compound 103: MS(ESI): 360.2 [M+H]⁺. ¹H NMR (400 MHz, Chloroform-d) δ 8.06 (d, J=8.4 Hz, 2H), 7.94 (s, 1H), 7.83 (d, J=8.4 Hz, 2H), 7.27-7.20 (m, 1H), 7.04 (d, J=3.4 Hz, 1H), 6.99 (dd, J=5.1, 3.5 Hz, 1H), 5.00 (s, 1H), 4.80 (d, J=4.9 Hz, 2H), 3.13 (s, 3H), 2.52 (s, 3H).

Compound 145: MS(ESI): 360.2 [M+H]⁺. ¹H NMR (400 MHz, Chloroform-d) δ 8.36 (s, 1H), 8.28 (d, J=8.4 Hz, 2H), 8.08 (t, J=7.1 Hz, 2H), 7.27 (d, J=1.3 Hz, 1H), 7.13 (d, J=3.4 Hz, 1H), 7.03 (dd, J=5.1, 3.5 Hz, 1H), 5.05-4.90 (m, 3H), 3.14 (s, 3H), 2.49 (s, 3H).

### Compound 104

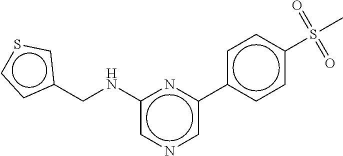

In a flask under nitrogen were added 2-chloro-6-(4-methanesulfonylphenyl)pyrazine (50 mg, 0.1860 mmol, 1 eq), potassium carbonate (52.1 mg, 0.372 mmol, 2 eq) and 3-thiophenemethylamine (42.0 mg, 0.37 mmol, 2 eq) in DMF (2 mL). The mixture was stirred at 100° C. for 24 h. The solvent was evaporated, EtOAc and water were added and the layers were separated. The aqueous layer was extracted with EtOAc. The combined organics were washed with lithium chloride 5% aqueous, dried over MgSO4, filtered with a fritted funnel and the solvent was evaporated. The crude was purified by prep HPLC (water/acetonitrile, TFA 0.01%) to give compound 104 (5.2 mg, 8.1% yield) as a yellow solid. MS: [M+H]⁺ 346.14; [M−H]⁻344.20. ¹H NMR (300 MHz, Chloroform-d) δ 8.35 (s, 1H), 8.18 (dt, 2H), 8.05 (dt, J=8.6 Hz, 2H), 7.96 (s, 1H), 7.35 (dd, J=4.9, 3.0 Hz, 1H), 7.11 (dd, J=5.0, 1.3 Hz, 1H), 4.71 (s, 2H), 3.10 (s, 3H).

### Compound 105

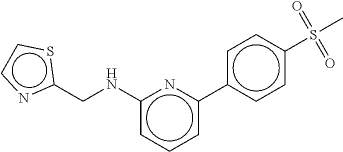

Was prepared using intermediate-3 (0.2 g, 0.7470 mmol, 1 eq) and 1-(1,3-thiazol-2-yl)methanamine (102 mg, 0.8964 mmol, 1.2 eq) using method 2. The residue was purified via Biotage (2:1 Hex/EtOAc; 12S column) to provide compound 105 (0.045 g, 17.4% yield) as a white solid. MS(ESI): 346.0 [M+H]⁺. ¹H NMR (400 MHz, DMSO-d6) δ 8.22 (d, J=8.3 Hz, 2H), 7.94 (d, J=8.3 Hz, 2H), 7.73 (d, J=3.2 Hz, 1H), 7.68 (t, J=6.0 Hz, 1H), 7.63-7.49 (m, 2H), 7.29 (d, J=7.4 Hz, 1H), 6.64 (d, J=8.2 Hz, 1H), 4.88 (d, J=6.0 Hz, 2H), 3.24 (s, 3H).

### Compound 107

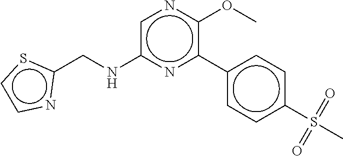

Was prepared using 5-chloro-3-(4-methanesulfonylphenyl)-2-methoxypyrazine (0.2 g, 0.6694 mmol) and 1-(1,3-thiazol-2-yl)methanamine (91.7 mg, 0.8032 mmol) using method 2. The residue was purified via Biotage (1:1 Hex/EtOAc; 12S column) and further purified by prep HPLC using (30-65% ACN in water containing 5M ammonium bicarbonate and 0.1% ammonia) as mobile phase to provide compound 107 (0.06 g, 23.9%) as a yellow solid. MS(ESI): 377.0 [M+H]⁺. ¹H NMR (400 MHz, DMSO-d6) δ 8.21 (d, J=8.6 Hz, 2H), 7.96 (d, J=8.6 Hz, 2H), 7.76 (s, 1H), 7.74 (d, J=3.2 Hz, 1H), 7.61 (t, J=6.1 Hz, 1H), 7.56 (d, J=3.3 Hz, 1H), 4.81 (d, J=6.0 Hz, 2H), 3.91 (s, 3H), 3.24 (s, 3H).

### Compound 108

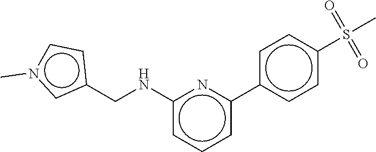

Was prepared using intermediate-3 (200 mg, 0.7470 mmol) and 1-(1-methyl-1H-pyrrol-3-yl)methanamine (82.2 mg, 0.747 mmol) using method 2. The residue was purified via Biotage (1:1 Hex/EtOAc; 12M column) to provide 70% pure product, which was purified by prep HPLC purification using (30-50% ACN in water containing 5 mM ammonium bicarbonate+ammonia as a modifier) as mobile phase to provide compound 108 (13 mg, 5.1%) as an off white solid. MS(ESI): 342.0 [M+H]⁺, 340.0 [M−H]⁻. ¹H NMR (400 MHz, DMSO-d6) δ 8.29 (d, J=8.2 Hz, 2H), 7.98 (d, J=8.1 Hz, 2H), 7.48 (t, J=7.8 Hz, 1H), 7.17 (d, J=7.3 Hz, 1H), 6.80 (t, J=5.4 Hz, 1H), 6.68 (s, 1H), 6.63-6.47 (m, 2H), 6.00 (s, 1H), 4.34 (d, J=5.3 Hz, 2H), 3.55 (s, 3H), 3.24 (s, 3H).

### Compound 109

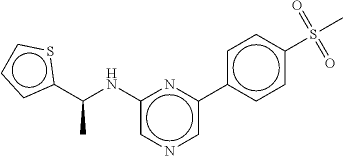

Was prepared using intermediate-7 (150 mg, 0.5582 mmol) and (S)-1-(thiophen-2-yl)ethanamine hydrochloride (91.3 mg, 0.5582 mmol) using method 2. The residue was purified via Biotage (50:1 CH2Cl2/MeOH; 12M column) to provide impure product, which was further purified by prep HPLC using (20-50% ACN in water containing 0.1% formic acid as modifier) as mobile phase to provide compound 109 (70 mg, 35.0% yield) as a off white solid. MS(ESI): 360.2 [M+H]⁺. ¹H NMR (400 MHz, Chloroform-d) δ 8.35 (s, 1H), 8.21-8.13 (m, 2H), 8.06-7.99 (m, 2H), 7.90 (s, 1H), 7.20 (dd, J=5.1, 1.2 Hz, 1H), 7.06 (d, J=3.5 Hz, 1H), 6.97 (dd, J=5.1, 3.5 Hz, 1H), 5.50 (p, J=6.9 Hz, 1H), 5.04 (d, J=7.5 Hz, 1H), 3.09 (s, 3H), 1.74 (d, J=6.7 Hz, 3H).

### Compound 110

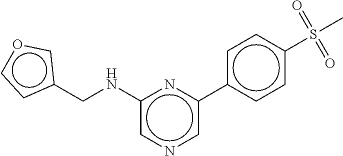

Was prepared using intermediate-7 (0.15 g, 0.5582 mmol, 1 eq) and 1-(furan-3-yl)methanamine (54.2 mg, 0.5582 mmol, 1 eq) using method 2. The residue was purified via Biotage (2:1 Hex/EtOAc; 12S column) to provide impure product, which was further purified by prep HPLC using (7-45% (ACN: MeOH/50:50) in water containing 0.1% ammonia and 5 mm ammonium bicarbonate as modifier) as mobile phase to provide compound 110 (0.030 g, 16.3% yield) as white solid. MS(ESI): 330.0[M+H]⁺. ¹H NMR (400 MHz, DMSO-d6) δ 8.42 (s, 1H), 8.37-8.28 (m, 2H), 8.02 (d, J=7.6 Hz, 3H), 7.68 (s, 1H), 7.66-7.58 (m, 2H), 6.51 (d, J=1.7 Hz, 1H), 4.42 (d, J=5.5 Hz, 2H), 3.26 (s, 3H).

### Compounds 112 and 241

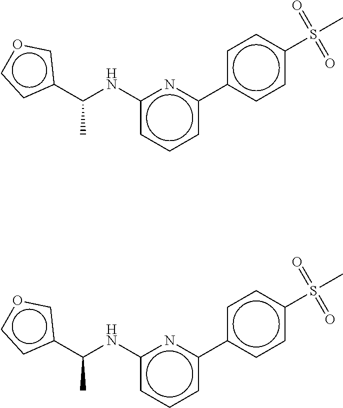

Was prepared using intermediate-3 (0.250 g, 0.9337 mmol, 1 eq) and 1-(furan-3-yl)ethan-1-amine (124 mg, 1.12 mmol, 1.2 eq) using method 2. The residue was purified via Biotage (2:1 Hex/EtOAc; 12S column) to provide impure product, which was further purified by prep HPLC using (55-25% ACN in 0.1% formic acid in water as modifier) as mobile phase to provide the product (0.12 g, 37.6% yield) as an Brown semi-solid. The racemic mixture was purified by chiral SFC purification by CO2 and 0.1% Diethyl amine in methanol as a co-solvent using Chiralcel OJ-H (250 mm*4.6 mm*5 m) to provide compound 112 (eluting second, 19.33 mg, 21.4% yield) and compound 241 (eluting first, 23.0 mg, 25.5% yield) as brown semi-solids. Compound 112: ¹H NMR (400 MHz, DMSO-d6) δ 8.26 (dd, J=8.2, 4.1 Hz, 2H), 7.97 (dd, J=8.3, 4.1 Hz, 2H), 7.68-7.43 (m, 3H), 7.19 (dd, J=7.4, 4.0 Hz, 1H), 6.94 (dd, J=8.4, 4.3 Hz, 1H), 6.66-6.39 (m, 2H), 5.23 (d, J=9.6 Hz, 1H), 3.24 (q, J=3.8, 3.4 Hz, 3H), 1.46 (dd, J=6.9, 3.8 Hz, 3H). Compound 241: ¹H NMR (400 MHz, DMSO-d6) δ 8.34-8.18 (m, 2H), 7.98 (t, J=6.6 Hz, 2H), 7.54 (ddd, J=33.1, 11.6, 4.6 Hz, 3H), 7.20 (t, J=7.8 Hz, 1H), 6.95 (t, J=7.6 Hz, 1H), 6.61-6.45 (m, 2H), 5.22 (d, J=8.8 Hz, 1H), 3.27-3.18 (m, 3H), 1.50-1.42 (m, 3H). The absolute stereochemistries of Compounds 112 and 241 were arbitrarily assigned.

### Compound 113

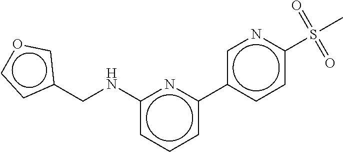

Was prepared using 6-bromo-6′-methanesulfonyl-2,3′-bipyridine (150 mg, 0.4789 mmol) and 1-(furan-3-yl)methanamine (46.5 mg, 0.4789 mmol) using method 2. The residue was purified via Biotage (2:1 Hex/EtOAc; 12M column) and further purified by prep HPLC using (35-55% Acetonitrile in 5 mM ammonium carbonate containing 0.1% ammonia) as a mobile phase to provide compound 113 (9 mg, 5.6%) as an off white solid. MS(ESI): 328.0 [M−H]⁻, 330.0 [M+H]⁺. ¹H NMR (400 MHz, DMSO-d6) δ 9.38 (d, J=2.1 Hz, 1H), 8.71 (dd, J=8.3, 2.2 Hz, 1H), 8.11 (d, J=8.2 Hz, 1H), 7.64 (s, 1H), 7.61-7.49 (m, 2H), 7.31 (d, J=7.3 Hz, 1H), 7.14 (t, J=5.7 Hz, 1H), 6.63 (d, J=8.3 Hz, 1H), 6.50 (d, J=1.8 Hz, 1H), 4.41 (d, J=5.6 Hz, 2H), 3.31 (s, 3H).

### Compound 114

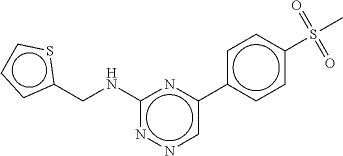

To a stirred solution of 3-methanesulfonyl-5-(4-methanesulfonylphenyl)-1,2,4-triazine (0.3 g, 0.9573 mmol) and 1-(thiophen-2-yl)methanamine (108 mg, 0.9573 mmol) in Acetonitrile (10 mL) was added sodium carbonate (304 mg, 2.87 mmol) and allowed to stir at RT for 16 h. After 16 h, the reaction mixture was poured into water and extracted with ethyl acetate (3×20 mL). The combined organics were dried over Na2SO4 and evaporated to give crude product which was triturated with 2% methanol in DCM to give compound 114 (50 mg, 14.8%) as a yellow solid. MS(ESI): 347.0 [M+H]⁺, 345.2 [M−H]. ¹H NMR (400 MHz, DMSO-d6) δ 9.33 (s, 1H), 8.44 (d, J=8.2 Hz, 2H), 8.34 (s, 1H), 8.11 (d, J=8.5 Hz, 2H), 7.33 (dd, J=5.1, 1.3 Hz, 1H), 7.10 (d, J=3.4 Hz, 1H), 6.96 (dd, J=5.1, 3.4 Hz, 1H), 4.84 (d, J=6.2 Hz, 2H), 3.26 (s, 3H).

### Compounds 115, 177, 314 and 322

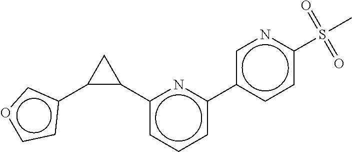

To a stirred solution of N′-(furan-3-ylmethylene)-4-methylbenzenesulfonohydrazide (1 g, 3.78 mmol) in THF (10 mL) was added 60% sodium hydride in mineral oil (108 mg, 4.53 mmol) at 0° c. and allowed to stir at RT for 2 h. After 2 h, resultant solid was filtered through Buchner funnel and obtained solid was added to a solution of 2-ethenyl-6-(4-methanesulfonylphenyl)pyridine (980 mg, 3.78 mmol) and heated at 110° C. for 16 h. After completion, the reaction mixture was filtered through celite and filtrate was evaporated. The residue was purified via Biotage (2:1 Hex/EtOAc; 12M column) and further purified by prep HPLC using (30-73% ACN in water containing 0.1% formic acid as an modifier) as a mobile phase to provide the Syn isomers 117 and 322 (50 mg, 3.90%) as a light brown semi-solid and Anti isomers 115 and 314 (80 mg, 6.3%) as a off white solid. Syn Isomers: MS(ESI): 340.0 [M+H]⁺. ¹H NMR (400 MHz, Chloroform-d) δ 8.11-8.02 (m, 2H), 8.02-7.95 (m, 2H), 7.57 (t, J=7.7 Hz, 1H), 7.48 (dd, J=7.7, 1.0 Hz, 1H), 7.14-7.06 (m, 3H), 6.02 (dd, J=1.7, 0.8 Hz, 1H), 3.08 (s, 3H), 2.61 (td, J=8.6, 6.1 Hz, 1H), 2.38-2.28 (m, 1H), 1.77 (td, J=6.4, 4.8 Hz, 1H), 1.49 (td, J=8.5, 4.8 Hz, 1H). Anti Isomers: MS(ESI): 340.0 [M+H]⁺. ¹H NMR (400 MHz, Chloroform-d) δ 8.25-8.18 (m, 2H), 8.06-7.98 (m, 2H), 7.67 (t, J=7.7 Hz, 1H), 7.58 (dd, J=7.8, 1.0 Hz, 1H), 7.39-7.31 (m, 2H), 7.22 (dd, J=7.7, 1.0 Hz, 1H), 6.25 (dd, J=1.8, 0.9 Hz, 1H), 3.09 (s, 3H), 2.47 (ddd, J=9.0, 6.1, 4.1 Hz, 1H), 2.22 (ddd, J=8.4, 5.3, 4.1 Hz, 1H), 1.77 (ddd, J=9.1, 5.3, 4.2 Hz, 1H), 1.32 (ddd, J=8.4, 6.0, 4.2 Hz, 1H).

2-[2-(furan-3-yl)cyclopropyl]-6-(4-methanesulfonylphenyl)pyridine (50 mg, 0.1473 mmol) was purified by chiral SFC purification by CO2 and 0.1% Diethyl amine in methanol as a co-solvent using Chiralcel OJ-H (250 mm*4.6 mm*5 m) to provide compound 177 (eluting first, 20 mg, 40.0%) and compound 322 (eluting second, 20 mg, 40.0%) as a yellow semi-solid. Compound 177: MS(ESI): 340.0 [M+H]⁺. ¹H NMR (400 MHz, Chloroform-d) δ 8.10-7.97 (m, 4H), 7.58 (t, J=7.7 Hz, 1H), 7.49 (d, J=7.7 Hz, 1H), 7.16-7.06 (m, 3H), 6.02 (d, J=1.7 Hz, 1H), 3.08 (s, 3H), 2.61 (td, J=8.6, 6.1 Hz, 1H), 2.33 (q, J=8.4 Hz, 1H), 1.77 (td, J=6.3, 4.8 Hz, 1H), 1.50 (td, J=8.5, 4.9 Hz, 1H). Compound 322: MS(ESI): 340.0 [M+H]⁺. ¹H NMR (400 MHz, Chloroform-d) δ 8.11-7.97 (m, 4H), 7.58 (t, J=7.7 Hz, 1H), 7.49 (d, J=7.7 Hz, 1H), 7.16-7.07 (m, 3H), 6.02 (d, J=1.8 Hz, 1H), 3.08 (s, 3H), 2.61 (td, J=8.6, 6.1 Hz, 1H), 2.33 (q, J=8.5 Hz, 1H), 1.77 (q, J=6.1 Hz, 1H), 1.50 (td, J=8.5, 4.9 Hz, 1H). The absolute stereochemistries of Compounds 177 and 322 were arbitrarily assigned.

2-[2-(furan-3-yl)cyclopropyl]-6-(4-methanesulfonylphenyl)pyridine (80 mg, 0.2357 mmol) was purified by chiral SFC purification by CO2 and 0.1% Diethyl amine in methanol as a co-solvent using Chiralcel OJ-H (250 mm*4.6 mm*5 m) to provide compound 115 (eluting first, 18 mg, 22.5%) and compound 314 (eluting second, 28 mg, 33.9%) as a yellow semi-solid. Compound 115: MS(ESI): 340.0 [M+H]⁺. ¹H NMR (400 MHz, Chloroform-d) δ 8.30-8.18 (m, 2H), 8.08-7.93 (m, 2H), 7.68 (t, J=7.7 Hz, 1H), 7.58 (d, J=7.7 Hz, 1H), 7.40-7.31 (m, 2H), 7.23 (d, J=7.6 Hz, 1H), 6.25 (s, 1H), 3.09 (s, 3H), 2.47 (ddd, J=9.3, 6.1, 4.1 Hz, 1H), 2.23 (dt, J=8.9, 4.7 Hz, 1H), 1.77 (dt, J=9.2, 4.4 Hz, 1H), 1.33 (ddd, J=8.4, 6.0, 4.1 Hz, 1H). Compound 314: MS(ESI): 340.0 [M+H]⁺. ¹H NMR (400 MHz, Chloroform-d) δ 8.21 (d, J=8.4 Hz, 2H), 8.03 (d, J=8.4 Hz, 2H), 7.68 (t, J=7.7 Hz, 1H), 7.58 (d, J=7.7 Hz, 1H), 7.41-7.31 (m, 2H), 7.23 (d, J=7.7 Hz, 1H), 6.25 (d, J=1.8 Hz, 1H), 3.09 (s, 3H), 2.47 (ddd, J=9.4, 5.9, 4.1 Hz, 1H), 2.23 (dt, J=9.1, 4.9 Hz, 1H), 1.77 (dt, J=9.2, 4.7 Hz, 1H), 1.33 (ddd, J=8.4, 6.1, 4.1 Hz, 1H). The absolute stereochemistries of Compounds 115 and 314 were arbitrarily assigned.

### Compound 116

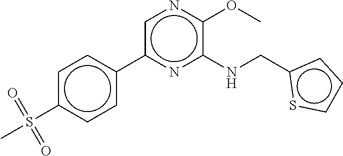

In a flask under nitrogen were added 6-chloro-3-methoxy-N-[(thiophen-2-yl)methyl]pyrazin-2-amine (111.7 mg, 0.4340 mmol, 1 eq), (4-methanesulfonylphenyl)boronic acid (86.8 mg, 0.434 mmol, 1 eq), potassium carbonate (121 mg, 0.868 mmol, 2 eq) and XPhos Pd G2 (17.0 mg, 0.0217 mmol, 0.05 eq) in dioxane/water (5 mL). The mixture was stirred at 80° C. for 2 h. The solvent was evaporated. The crude was purified by normal phase flash chromatography (hexanes/EtOAc, 0-100%, eluted 40%) to give compound 116 (72.6 mg, 44.8% yield) as a yellow solid. MS: [M+H]⁺ 375.93. ¹H NMR (400 MHz, DMSO-d6) δ 8.28 (d, J=7.7 Hz, 2H), 8.06 (s, 1H), 7.98 (d, J=7.6 Hz, 2H), 7.63 (t, J=5.9 Hz, 1H), 7.33 (d, J=5.0 Hz, 1H), 7.09 (d, J=2.6 Hz, 1H), 6.95 (t, J=3.8 Hz, 1H), 4.81 (d, J=6.0 Hz, 2H), 3.99 (s, 3H), 3.26 (s, 3H).

### Compound 117

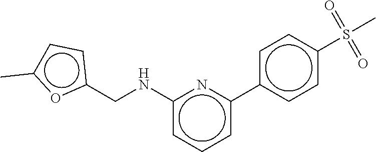

Was prepared using intermediate-4 (200 mg, 0.6406 mmol) and 1-(5-methylfuran-2-yl)methanamine (71.1 mg, 0.6406 mmol) using method 2. The residue was purified via Biotage (2:1 Hex/EtOAc; 12M column) to provide compound 117 (75 mg, 34.2%) as an off white solid. MS(ESI): 341.0 [M−H]⁻, 343.0 [M+H]⁺. ¹H NMR (400 MHz, DMSO-d6) δ 8.29 (d, J=8.2 Hz, 2H), 7.98 (d, J=8.1 Hz, 2H), 7.52 (t, J=7.8 Hz, 1H), 7.22 (d, J=7.3 Hz, 1H), 7.16 (t, J=5.7 Hz, 1H), 6.59 (d, J=8.3 Hz, 1H), 6.18 (d, J=3.0 Hz, 1H), 5.97 (d, J=2.9 Hz, 1H), 4.50 (d, J=5.7 Hz, 2H), 3.25 (s, 3H), 2.23 (s, 3H).

### Compound 118

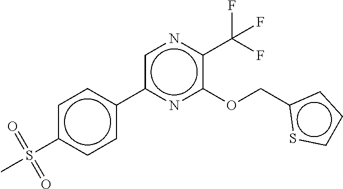

To a stirred solution of 3-chloro-5-(4-methanesulfonylphenyl)-2-(trifluoromethyl)pyrazine (75 mg, 0.2227 mmol) and (thiophen-2-yl)methanol (50.8 mg, 0.4454 mmol) in DMF (1 mL) was added potassium carbonate (92.3 mg, 0.6681 mmol) and heated at 100° C. for 16 h. After completion of the reaction, the reaction mixture was poured into ice-cold water (20 mL) and filtered through Buchner funnel. The residue was purified via Biotage (2:1 Hex/EtOAc; 12S column) to provide compound 118 (30 mg, 32.5% yield) as a white solid. MS(ESI): 415.2 [M+H]⁺. ¹H NMR (400 MHz, Chloroform-d) δ 8.73 (s, 1H), 8.30 (d, J=8.4 Hz, 2H), 8.13 (d, J=8.4 Hz, 2H), 7.33 (dd, J=5.1, 1.3 Hz, 1H), 7.23 (d, J=3.5 Hz, 1H), 7.01 (dd, J=5.1, 3.5 Hz, 1H), 5.83 (s, 2H), 3.13 (s, 3H).

### Compound 120

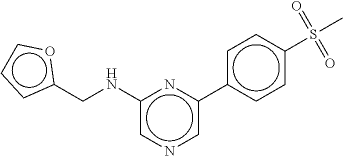

Was prepared using intermediate-7 (150 mg, 0.5582 mmol) and furan-2-ylmethanamine (54.2 mg, 0.5582 mmol) using method 2. The residue was purified via Biotage (50:1 CH2Cl2/MeOH; 12M column) to provide impure product; which was further purified by prep HPLC purification by using (5-60% ACN in water containing 0.1% formic acid as a modifier) as a mobile phase to provide compound 120 (9 mg, 4.9% yield) as off white solid. MS(ESI): 330.2 [M+H]⁺. ¹H NMR (400 MHz, Chloroform-d) δ 8.40 (s, 1H), 8.22 (d, J=8.4 Hz, 2H), 8.06 (d, J=8.4 Hz, 2H), 7.99 (s, 1H), 7.41 (s, 1H), 6.43-6.30 (m, 2H), 5.08 (s, 1H), 4.71 (d, J=5.7 Hz, 2H), 3.12 (s, 3H).

### Compound 121

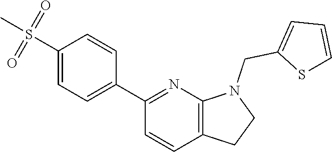 [SMILES: CS(=O)(=O)c1ccc(-c2ccc3c(n2)N(Cc2cccs2)CC3)cc1]

A solution of 6-bromo-2,3-dihydro-1H-pyrrolo[2,3-b]pyridine (0.1 g, 0.5 mmol, 1 eq) and 2-thiophenecarboxaldehyde (0.056 g, 0.5 mmol, 1 eq) in methanol (3 mL) along with catalytic amount of acetic acid followed by addition of sodium cyanoborohydride (0.063 g, 1.0 mmol, 2.00 eq) was stirred at 0° C. and left to warm to RT for 16 h. To the reaction mixture was added ice water (5 mL) and extracted with DCM (3×10 mL). The organics were dried over Na2SO4 and evaporated. The residue was purified via Biotage (5:1 Hex/EtOAc; 12S column) to provide 6-bromo-1-(thiophen-2-ylmethyl)-2,3-dihydro-1H-pyrrolo[2,3-b]pyridine (0.11 g, 74% yield). MS: [M+H]⁺ 297.30.

A stirred suspension of 6-bromo-1-(thiophen-2-ylmethyl)-2,3-dihydro-1H-pyrrolo[2,3-b]pyridine (0.11 g, 0.37 mmol, 1 eq), (4-methylsulfonylphenyl)boronic acid (0.089 g, 0.44 mmol, 1.20 eq), sodium carbonate (0.112 g, 1.11 mmol, 3 eq) in 1,4-dioxane (3 mL) was degassed with nitrogen for 15 min. After 15 min, [1,1′-bis(diphenylphosphino)ferrocene]dichloropalladium(II), complex with dichloromethane (0.006 g, 0.007 mmol, 0.02 eq) and water (1 mL) were added and heated at 100° C. for 6 h. The reaction mixture was diluted with water (10 mL) and extracted with ethyl acetate (3×15 mL). The organic layers was dried over Na2SO4 and evaporated. The residue was purified via Biotage (5:1 Hex/EtOAc; 12S column) and further purified by prep HPLC using (100% ACN and 0.1% formic acid in water) as mobile phase to provide compound 121 (80 mg, 58% yield) as an off white solid. MS: 371.40 [M+H]⁺. ¹H NMR (400 MHz, DMSO-d6) δ 8.32 (d, J=8.3 Hz, 2H), 7.98 (d, J=8.3 Hz, 2H), 7.40 (dd, J=8.9, 6.3 Hz, 2H), 7.23 (d, J=7.4 Hz, 1H), 7.09 (d, J=3.5 Hz, 1H), 6.98 (dd, J=5.2, 3.4 Hz, 1H), 4.81 (s, 2H), 3.47 (t, J=8.3 Hz, 2H), 3.24 (s, 3H), 2.98 (t, J=8.3 Hz, 2H).

### Compound 122

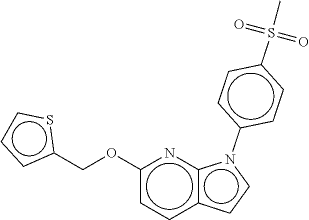

Was prepared using 6-bromo-1-(4-methanesulfonylphenyl)-1H-pyrrolo[2,3-b]pyridine (30 mg, 0.08541 mmol, 1 eq) and (thiophen-2-yl)methanol (12.1 μL, 0.1281 mmol, 1.5 eq) using method 2. The crude residue was purified by normal phase chromatography (hexane/EtOAc, 0-70%), then washed with Et2O to afford compound 122 (8.6 mg, 26.2% yield) as a white solid. MS: [M+H]⁺ 385.09. ¹H NMR (400 MHz, Chloroform-d) δ 8.14 (d, J=8.9 Hz, 2H), 8.08 (d, J=8.9 Hz, 2H), 7.87 (d, J=8.4 Hz, 1H), 7.40 (d, J=3.7 Hz, 1H), 7.29 (dd, J=5.1, 1.2 Hz, 1H), 7.15 (d, J=2.9 Hz, 1H), 7.02-6.98 (m, 1H), 6.73 (d, J=8.5 Hz, 1H), 6.63 (d, J=3.8 Hz, 1H), 5.60 (s, 2H), 3.12 (s, 3H).

### Compound 123

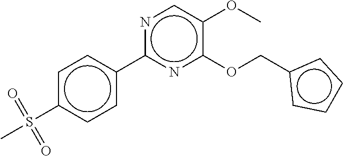

In a flask under argon were added 4-chloro-2-(4-methanesulfonylphenyl)-5-methoxypyrimidine (85 mg, 0.2845 mmol, 1 eq) in THF (2 mL). In another flask under argon were added sodium hydride (26.1 mg, 0.6543 mmol, 2.3 eq) and (thiophen-2-yl)methanol (53.8 μL, 0.569 mmol, 2 eq) in THF (2 mL). The mixture was stirred for 1 h and the solution containing the alcohol was added to the other solution. The mixture was stirred at room temperature for 20 h. The solvent was evaporated. DCM and water were added. The layers were separated. The aqueous layer was extracted with DCM and the combined organics were washed with brine, dried over MgSO4 and the solvent was evaporated. The crude was purified by normal phase flash chromatography (Hexanes/EtOAc, 0-70%) and further purified by prep HPLC under neutral conditions (water/acetonitrile, 0-100%) to give compound 123 (17.3 mg, 16.1% yield) as a colorless oil. [M+H]⁺ 377.46. ¹H NMR (300 MHz, Chloroform-d) δ 8.36 (dd, J=6.6, 2.2 Hz, 3H), 8.06-8.00 (m, 2H), 7.30 (dd, J=5.1, 1.2 Hz, 1H), 7.22-7.16 (m, 1H), 6.99 (dd, J=5.1, 3.5 Hz, 1H), 5.62 (s, 2H), 3.96 (s, 3H), 3.09 (s, 3H).

### Compound 124

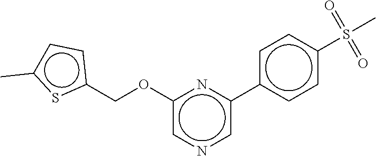

Was prepared using intermediate-7 (50 mg, 0.1860 mmol, 1 eq) and 5-methylthiophenyl alcohol (47.6 mg, 0.372 mmol, 2 eq) using method 1. The crude was purified using flash column chromatography (SiO4, 0-100% EtOAc/hexanes, eluted 50%) to afford compound 124 (45 mg, 67.1% yield) as a white solid. MS: 251.10 [M+H]⁺. ¹H NMR (300 MHz, Chloroform-d) δ 8.69 (s, 1H), 8.28 (dt, J=12.5, 1.9 Hz, 3H), 8.09 (dt, J=12.1, 1.9 Hz, 2H), 7.00 (d, J=3.4 Hz, 1H), 6.68-6.64 (m, 1H), 5.61 (s, 2H), 3.12 (s, 3H), 2.47 (d, J=0.8 Hz, 3H).

### Compound 125

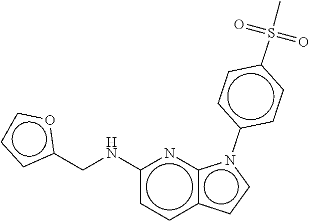

In a vial under nitrogen were dissolved (tert-butoxy)sodium (9.84 mg, 0.1024 mmol, 1.2 eq), BrettPhos Pd G4 (3.93 mg, 0.004270 mmol, 0.05 eq), BrettPhos (2.29 mg, 0.004270 mmol, 0.05 eq), 6-bromo-1-(4-methanesulfonylphenyl)-1H-pyrrolo[2,3-b]pyridine (30 mg, 0.08541 mmol, 1 eq) and 1-(furan-2-yl)methanamine (11.7 μL, 0.1281 mmol, 1.5 eq) in dioxane (0.4 mL). The mixture was stirred at 50° C. for 5 h. Additional 1-(furan-2-yl)methanamine (5 μL, 0.05416 mmol, 0.634 eq) was added and stirring was pursued for 1 h. The solvent was evaporated and the crude material was purified by normal phase chromatography (hexane/EtOAc, 25-50%). The purest fraction was collected and purified again by normal phase chromatography (hexane/EtOAc, 0-30%). The resulting oil was washed with Et2O to afford compound 125 (7.4 mg, 23.6% yield) as a white solid. MS: [M+H]⁺ 368.08. ¹H NMR (400 MHz, Chloroform-d) δ 8.15 (d, J=8.8 Hz, 2H), 8.03 (d, J=8.8 Hz, 2H), 7.70 (d, J=8.5 Hz, 1H), 7.38 (dd, J=1.9, 0.9 Hz, 1H), 7.27 (s, 1H), 6.53 (d, J=3.8 Hz, 1H), 6.42 (d, J=8.5 Hz, 1H), 6.33 (dd, J=3.2, 1.9 Hz, 1H), 6.24 (dd, J=3.1, 0.9 Hz, 1H), 4.79 (s, 1H), 4.60 (d, J=5.8 Hz, 2H), 3.10 (s, 3H).

### Compound 126

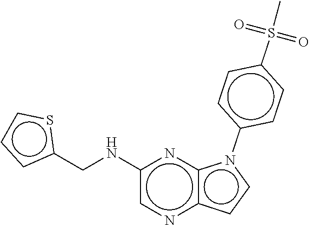

Obtained from 3-chloro-5-(4-methanesulfonylphenyl)-5H-pyrrolo[2,3-b]pyrazine (0.055 g, 0.1787 mmol, 1 eq) and 1-(thiophen-2-yl)methanamine (24.2 mg, 0.2144 mmol, 1.2 eq) using method 3. The residue was purified via Biotage (2:1 Hex/EtOAc; 12S column) to provide impure product, which was further purified by prep HPLC using (55-25% ACN in 0.1% formic acid in water as modifier) as mobile phase to provide compound 126 (15.0 mg, 21.6% yield) as a yellowish solid. ¹H NMR (400 MHz, DMSO-d6) δ 8.28 (d, J=8.5 Hz, 2H), 8.03 (d, J=8.5 Hz, 2H), 7.97 (s, 1H), 7.91 (d, J=3.9 Hz, 1H), 7.86 (d, J=6.0 Hz, 1H), 7.37 (d, J=5.1 Hz, 1H), 7.11 (d, J=3.4 Hz, 1H), 7.02-6.94 (m, 1H), 6.73 (d, J=3.9 Hz, 1H), 4.71 (d, J=5.7 Hz, 2H), 3.28 (s, 3H).

### Compound 127

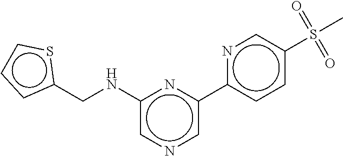

To a stirred solution of 2-chloro-6-(5-methanesulfonylpyridin-2-yl)pyrazine (50 mg, 0.1853 mmol) in DMSO (1 mL) was added 1-(thiophen-2-yl)methanamine (20.9 mg, 0.1853 mmol) and N,N-diisopropylethylamine (71.8 mg, 0.5559 mmol) followed by heating at 1300 for 24 h. After completion of the reaction, the reaction mixture was poured into water (20 mL) and extracted with ethyl acetate (3×10 mL). The combined organics were dried over Na2SO4 and evaporated. The residue was purified via Biotage (2:1 Hex/EtOAc; 12S column) to provide impure product which was further purified by prep HPLC using (0-55% ACN in water containing 0.1% formic acid as modifier) as mobile phase to provide compound 127 (5 mg, 7.8% yield) as a off white solid. MS(ESI): 347.2 [M+H]⁺, 345.2 [M−H]-. ¹H NMR (400 MHz, Chloroform-d) δ 9.23 (d, J=2.3 Hz, 1H), 9.05 (s, 1H), 8.60 (d, J=8.4 Hz, 1H), 8.36 (dd, J=8.4, 2.3 Hz, 1H), 8.09 (s, 1H), 7.28 (dd, J=5.1, 1.2 Hz, 1H), 7.12 (d, J=3.3 Hz, 1H), 7.03 (dd, J=5.2, 3.5 Hz, 1H), 5.18 (s, 1H), 4.93 (d, J=5.7 Hz, 2H), 3.20 (s, 3H).

### Compound 128

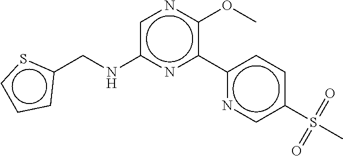

Obtained from 5-chloro-3-(5-methanesulfonylpyridin-2-yl)-2-methoxypyrazine (0.05 g, 0.1668 mmol) and 1-(thiophen-2-yl)methanamine (22.6 mg, 0.2001 mmol) using method 3. The residue was purified via Biotage (1:1 Hex/EtOAc; 12M column) to provide slight impure product which was further purified by prep HPLC purification using (10-65% ACN in water containing 5M ammonium bicarbonate and 0.1% ammonia as modifier) as mobile phase to provide compound 128 (0.004 g, 5.5% yield) as a yellow semi-solid. MS(ESI): 377.3 [M+H]⁺. ¹H NMR (400 MHz, DMSO-d6) δ 9.14 (d, J=2.4 Hz, 1H), 8.40 (dd, J=8.3, 2.5 Hz, 1H), 8.12 (d, J=8.3 Hz, 1H), 7.77 (s, 1H), 7.35 (d, J=5.0 Hz, 1H), 7.29 (t, J=6.1 Hz, 1H), 7.07 (d, J=3.5 Hz, 1H), 6.95 (dd, J=5.1, 3.4 Hz, 1H), 4.65 (d, J=6.0 Hz, 2H), 3.84 (s, 3H), 3.37 (s, 3H).

### Compound 131

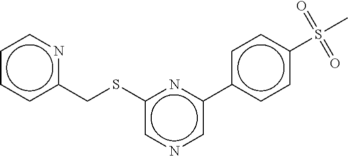

To a mixture of intermediate-7 (40 mg, 0.1488 mmol, 1 eq) and potassium carbonate (41.1 mg, 0.2976 mmol, 2.0 eq) in DMF (1.48 mL) was added 2-pyridylmethanethiol (21.8 μL, 0.1934 mmol, 1.3 eq). The solution was stirred at r.t. for 1 h and at 60° C. over night, then it was cooled at r.t. and solvent was removed. The aqueous layer was extracted with EtOAc (3×10 mL) and the combined organics were dried over MgSO4, filtered through Celite and DCM mixture and evaporated. Compound 131 (20 mg, 36.3% yield) was purified using flash column chromatography (SiO4, 0-100% EtOAc/hexanes, eluted 60%). MS: 358.11 [M+H]⁺. ¹H NMR (300 MHz, Chloroform-d) δ 8.71 (s, 1H), 8.59 (d, J=4.9 Hz, 1H), 8.52 (s, 1H), 8.16 (dt, J=12.0, 1.9 Hz, 2H), 8.05 (dt, J=12.2, 2.0 Hz, 2H), 7.63 (td, J=7.7, 1.8 Hz, 1H), 7.46 (d, J=7.8 Hz, 1H), 7.19 (ddd, J=7.5, 4.9, 1.0 Hz, 1H), 4.66 (s, 2H), 3.11 (s, 3H).

### Compound 132

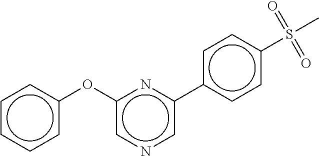

Was prepared using intermediate-7 (40 mg, 0.1488 mmol, 1 eq) and phenol (28.0 mg, 0.2976 mmol, 2 eq) using method 1. The crude was purified using reverse chromatography (0-100%, MeCN/H2O) to afford compound 132 (12 mg, 25% yield). MS: 327.31 [M+H]⁺. ¹H NMR (300 MHz, Chloroform-d) δ 8.80 (s, 1H), 8.41 (s, 1H), 8.07 (dt, J=12.1, 1.9 Hz, 2H), 8.00 (dt, J=12.0, 2.1 Hz, 2H), 7.46 (tt, J=7.8, 2.2 Hz, 2H), 7.31 (dt, J=8.0, 1.4 Hz, 1H), 7.25-7.21 (m, 2H), 3.06 (s, 3H).

### Compound 133

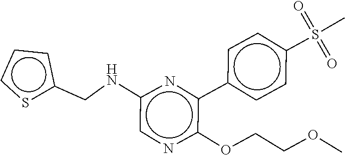

Was prepared using 5-bromo-3-(4-methanesulfonylphenyl)-2-(2-methoxyethoxy)pyrazine (135 mg, 0.3486 mmol, 1 eq) and 1-(thiophen-2-yl)methanamine (35.7 μL, 0.3486 mmol, 1 eq) using method 2. The crude was purified by normal phase flash chromatography (Hexanes/EtOAc, 20%-70%) to give compound 133 (77.6 mg, 53.1% yield) as a yellow solid. ¹H NMR (400 MHz, Chloroform-d) δ 8.43-8.40 (m, 2H), 8.00-7.96 (m, 2H), 7.53 (s, 1H), 7.21 (dd, J=5.1, 1.2 Hz, 1H), 7.05-7.01 (m, 1H), 6.96 (dd, J=5.1, 3.5 Hz, 1H), 4.76 (s, 3H), 4.52-4.47 (m, 2H), 3.79-3.73 (m, 2H), 3.43 (s, 3H), 3.08 (s, 3H).

### Compound 134

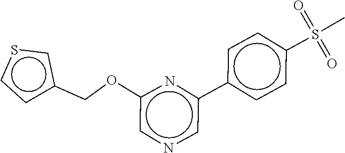

Was prepared using intermediate-7 (50 mg, 0.1860 mmol, 1 eq) and thiophen-3-methanol (35.0 μL, 0.372 mmol, 2 eq) using method 1. The crude was purified using flash column chromatography (SiO4, 0-100% EtOAc/hexanes) to afford compound 134 (51 mg, 78% yield). MS: 347.31 [M+H]⁺. ¹H NMR (300 MHz, Chloroform-d) δ 8.67 (s, 1H), 8.29 (s, 1H), 8.22 (dt, J=12.4, 2.0 Hz, 2H), 8.07 (dt, J=12.3, 2.0 Hz, 2H), 7.43-7.33 (m, 2H), 7.21 (dd, J=5.0, 1.2 Hz, 1H), 5.53 (s, 2H), 3.11 (s, 3H).

### Compound 136

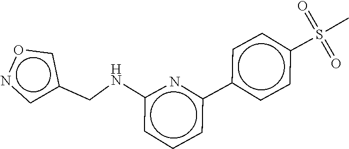

Was prepared using intermediate-4 (100 mg, 0.3203 mmol) and 1-(1,2-oxazol-4-yl)methanamine (37.7 mg, 0.3843 mmol) using method 2. The residue was purified via Biotage (1:1 Hex/EtOAc; 12M column) to provide slightly impure product which was further purified by prep HPLC purification using (20-70% ACN in water containing 0.1% formic acid as modifier) as mobile phase to provide compound 136 (12 mg, 11.4%) as an off white solid. MS(ESI): 330.0 [M+H]⁺, 328.2 [M−H]⁻. ¹H NMR (400 MHz, DMSO-d6) δ 8.88 (s, 1H), 8.58 (s, 1H), 8.28 (d, J=8.2 Hz, 2H), 7.98 (d, J=8.2 Hz, 2H), 7.55 (t, J=7.8 Hz, 1H), 7.26 (d, J=7.4 Hz, 1H), 7.17 (t, J=5.6 Hz, 1H), 6.58 (d, J=8.3 Hz, 1H), 4.45 (d, J=5.6 Hz, 2H), 3.25 (s, 3H).

### Compound 137

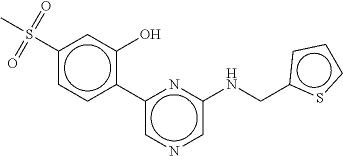

Was prepared using 2-(6-chloropyrazin-2-yl)-5-methanesulfonylphenol (80 mg, 0.2809 mmol) and 1-(thiophen-2-yl)methanamine (31.7 mg, 0.2809 mmol) using method 2. The residue was purified via Biotage (1:1 Hex/EtOAc; 12M column) and further purified by prep HPLC using (35-45% ACN in water containing 0.1% formic acid) as mobile phase to provide compound 137 (10 mg, 10%) as an off white solid. MS(ESI): 362.0 [M+H]⁺, 360.0 [M−H]⁻. ¹H NMR (400 MHz, DMSO-d6) δ 12.42 (s, 1H), 8.58 (s, 1H), 8.22 (d, J=8.4 Hz, 1H), 8.13 (t, J=5.9 Hz, 1H), 8.05 (s, 1H), 7.49-7.34 (m, 3H), 7.09 (d, J=3.3 Hz, 1H), 6.98 (dd, J=5.1, 3.4 Hz, 1H), 4.71 (d, J=5.8 Hz, 2H), 3.23 (s, 3H).

### Compound 138

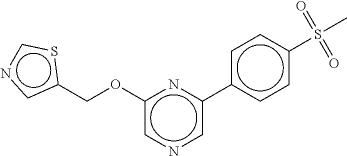

Was prepared using intermediate-7 (50 mg, 0.1860 mmol, 1 eq) and (1,3-thiazol-5-yl)methanol (42.8 mg, 0.372 mmol, 2 eq) using method 1. The crude was purified by normal phase flash chromatography (EtOAc/Hexanes, 0-100%, eluted 72%) to give compound 138 (50.6 mg, 78% yield) as a white solid. MS: [M+H]⁺ 348.10. ¹H NMR (300 MHz, Chloroform-d) δ 8.81 (s, 1H), 8.72 (s, 1H), 8.29 (s, 1H), 8.25 (d, J=8.4 Hz, 2H), 8.09 (d, J=8.4 Hz, 2H), 8.00 (s, 1H), 5.76 (s, 2H), 3.12 (s, 3H).

### Compound 139

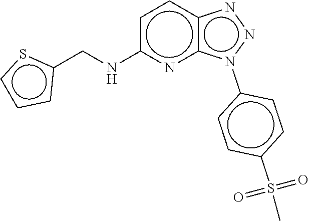

Was prepare using 5-bromo-3-(4-methylsulfonylphenyl)triazolo[4,5-b]pyridine (0.12 g, 0.340 mmol, 1.00 eq) and 2-thienylmethanamine (0.050 g, 0.410 mmol, 1.21 eq) using method 3. The residue was purified via Biotage (4:1 Hex/EtOAc; 12S column) and further purified by prep HPLC using (25-70% ACN in water containing 5 mM ammonium carbonate and 0.1% ammonia in water) as mobile phase to provide compound 139 (8.5 mg, 6% yield) as an off white solid. MS(ESI): 385[M+H]⁺. ¹H NMR (400 MHz, DMSO-d6) δ 8.62 (d, J=8.8 Hz, 2H), 8.47 (d, J=5.9 Hz, 1H), 8.16 (dd, J=8.8, 4.6 Hz, 3H), 7.38 (d, J=5.1 Hz, 1H), 7.14 (d, J=3.4 Hz, 1H), 6.99 (dd, J=5.1, 3.4 Hz, 1H), 6.77 (d, J=9.2 Hz, 1H), 4.81 (d, J=5.7 Hz, 2H), 3.31 (s, 3H), 2.45 (s, 3H).

### Compound 140

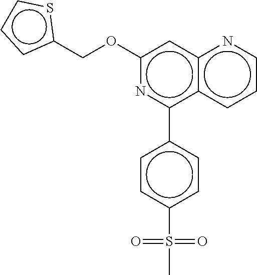

Was prepared using 7-chloro-5-(4-methanesulfonylphenyl)-1,6-naphthyridine (80 mg, 0.2509 mmol, 1 eq) and (thiophen-2-yl)methanol (23.7 μL, 0.2509 mmol, 1 eq) using method 2. The crude material was purified by normal phase chromatography (hexane/EtOAc, 0-100%), then by reverse phase chromatography (water/ACN, 10-100%) to afford compound 140 (2.1 mg, 2.1% yield). MS: [M+H]⁺ 397.26. ¹H NMR (400 MHz, Chloroform-d) δ 9.00 (dd, J=4.4, 1.7 Hz, 1H), 8.26 (d, J=8.4 Hz, 1H), 8.16 (d, J=8.4 Hz, 2H), 7.94 (d, J=8.4 Hz, 2H), 7.43 (d, J=1.0 Hz, 1H), 7.34-7.28 (m, 2H), 7.19 (dd, J=3.5, 1.1 Hz, 1H), 7.00 (dd, J=5.1, 3.5 Hz, 1H), 5.75-5.69 (m, 2H), 3.16 (s, 3H).

### Compound 141

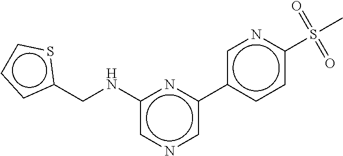

Was prepared using 2-chloro-6-(6-(methylsulfonyl)pyridin-3-yl)pyrazine (150 mg, 0.5561 mmol) and thiophen-2-ylmethanamine (62.9 mg, 0.5561 mmol) using method 3. The residue was purified via Biotage (50:1 CH2Cl2/MeOH; 12M column) and further purified by prep HPLC using (30-60% ACN:Methanol (1:1) in water containing 0.1% formic acid) as a mobile phase to provide compound 141 (50 mg, 26.0% yield) as a off white solid. MS(ESI): 347.2 [M+H]⁺. ¹H NMR (400 MHz, DMSO-d6) δ 9.36 (d, J=2.1 Hz, 1H), 8.58 (dd, J=8.2, 2.2 Hz, 1H), 8.41 (s, 1H), 8.20 (d, J=8.2 Hz, 1H), 8.01 (s, 1H), 7.27-7.21 (m, 1H), 7.08 (d, J=3.4 Hz, 1H), 7.00 (dd, J=5.1, 3.5 Hz, 1H), 5.23 (s, 1H), 4.89 (d, J=5.7 Hz, 2H), 3.30 (s, 3H).

### Compound 142

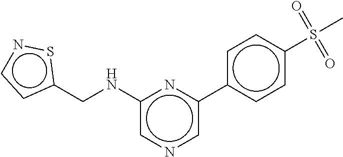

Was prepared using intermediate-7 (150 mg, 0.5582 mmol) and (1,2-thiazol-5-yl)methylamine (84.6 mg, 0.5582 mmol) using method 2. The residue was purified via Biotage (50:1 CH2Cl2/MeOH; 12M column) to provide impure product, which was further purified by prep HPLC using (15-43% ACN in water containing 0.1% formic acid as modifier) as mobile phase to provide compound 142 (25 mg, 12.9% yield) as a off white solid. MS(ESI): 347.2 [M+H]⁺, 345.2 [M−H]⁻. ¹H NMR (400 MHz, Chloroform-d) δ 8.50-8.33 (m, 2H), 8.19 (d, J=8.4 Hz, 2H), 8.12-7.92 (m, 3H), 7.20 (d, J=1.5 Hz, 1H), 5.37 (t, J=6.2 Hz, 1H), 5.01 (d, J=5.9 Hz, 2H), 3.10 (s, 3H).

### Compound 143

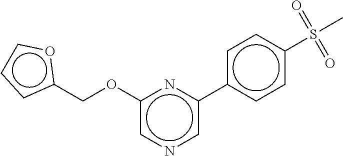

Was prepared using intermediate-7 (0.15 g, 0.5582 mmol, 1 eq) and (furan-2-yl)methanol (54.7 mg, 0.5582 mmol, 1.0 eq) using method 1. The residue was purified via Biotage (50:1 CH2Cl2/MeOH; 12M column) to provide impure product, which was further purified by prep HPLC using (30-55% ACN in water containing 0.1% formic acid as modifier) as mobile phase to provide compound 143 (0.07 g, 38% yield) as a white solid. MS(ESI): 331.0[M+H]⁺. ¹H NMR (400 MHz, DMSO-d6) δ 9.00 (s, 1H), 8.51-8.44 (m, 2H), 8.42 (s, 1H), 8.14-8.06 (m, 2H), 7.75 (d, J=2.2 Hz, 1H), 6.68 (d, J=3.3 Hz, 1H), 6.51 (dd, J=3.3, 1.8 Hz, 1H), 5.54 (s, 2H), 3.31 (s, 3H).

### Compound 144

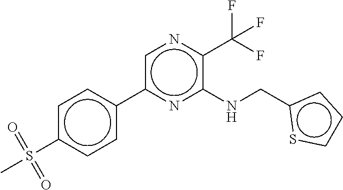

To a stirred solution of 3-chloro-5-(4-methanesulfonylphenyl)-2-(trifluoromethyl)pyrazine (30 mg, 0.08909 mmol) and 1-(thiophen-2-yl)methanamine (10.0 mg, 0.08909 mmol) in DMSO (1 mL) was added N,N-diisopropylethylamine (34.5 mg, 0.2672 mmol) and heated at 120° C. for 16 h. After completion of the reaction, the reaction mixture was evaporated. The residue was purified via Biotage (2:1 Hex/EtOAc; 12S column) to provide compound 144 (18 mg, 48.3% yield) as a off white solid. MS(ESI): 412.0 [M−H]⁻, 414.0 [M+H]⁺. ¹H NMR (400 MHz, Chloroform-d) δ 8.44 (s, 1H), 8.30-8.20 (m, 2H), 8.15-8.05 (m, 2H), 7.24 (dd, J=5.1, 1.2 Hz, 1H), 7.07 (d, J=3.5 Hz, 1H), 6.99 (dd, J=5.1, 3.5 Hz, 1H), 5.64 (s, 1H), 4.99 (d, J=5.5 Hz, 2H), 3.11 (s, 3H).

### Compound 146

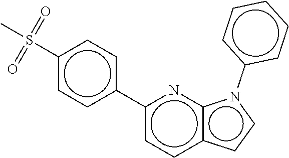

In a vial under argon were dissolved 6-(4-methanesulfonylphenyl)-1H-pyrrolo[2,3-b]pyridine (30 mg, 0.1101 mmol, 1.0 eq), K3PO4 (41 mg, 0.1931 mmol, 1.754 eq), iodocopper (1 mg, 0.005250 mmol, 0.048 eq), N,N′-dimethylethylenediamine (2 μL, 0.01849 mmol, 0.168 eq), iodobenzene (10 μL, 0.08921 mmol, 0.81 eq) in degassed Toluene (1 mL). The mixture was stirred under Argon overnight at 110° C. The reaction was cooled to room temperature and the mixture was diluted with water, extracted with DCM, and the combined organic layers were dried over MgSO4, filtered and evaporated. The crude material was purified by normal phase chromatography (hexane/EtOAc, 0-100%) to afford compound 146 (1.6 mg, 4.2% yield) as a pale yellow oil. MS: [M+H]⁺ 349.11. ¹H NMR (400 MHz, Chloroform-d) δ 8.32 (d, J=8.1 Hz, 2H), 8.10 (d, J=8.2 Hz, 1H), 8.04 (d, J=8.2 Hz, 2H), 7.91 (d, J=7.9 Hz, 2H), 7.74 (d, J=8.2 Hz, 1H), 7.66 (d, J=3.7 Hz, 1H), 7.59 (t, J=7.8 Hz, 2H), 7.40 (t, J=7.4 Hz, 1H), 6.71 (d, J=3.7 Hz, 1H), 3.11 (s, 3H).

### Compound 147

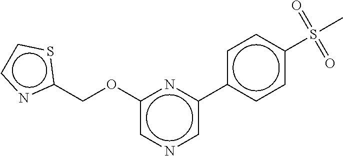

Was prepared using intermediate-7 (0.15 g, 0.5582 mmol, 1 eq) and (1,3-thiazol-2-yl)methanol (64.2 mg, 0.5582 mmol, 1 eq) using method 1. The residue was purified via Biotage (5:1 Hex/EtOAc; 12S column) to provide compound 147 (0.08 g, 41.4% yield) as a off white solid. MS(ESI): 348.0[M+H]⁺. ¹H NMR (400 MHz, DMSO-d6) δ 9.04 (s, 1H), 8.50 (s, 1H), 8.46-8.37 (m, 2H), 8.14-8.02 (m, 2H), 7.86 (d, J=3.3 Hz, 1H), 7.79 (d, J=3.2 Hz, 1H), 5.87 (s, 2H), 3.29 (s, 3H).

### Compound 148

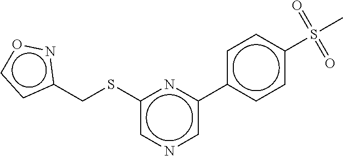

To stirred a mixture of 2-chloro-6-(4-methanesulfonylphenyl)pyrazine (0.15 g, 0.5582 mmol, 1 eq) and sodium hydrate sulfane (167 mg, 2.23 mmol, 4.0 eq) in dimethylformamide (2 mL) was stirred 10 minutes at room temperature. Then, added 3-(bromomethyl)-1,2-oxazole (144 mg, 0.8931 mmol, 1.6 eq) in reaction mixture. The reaction mixture was stirred 12 h at room temperature. After completion of reaction, the reaction mixture was quince in water (80 mL) and extracted with ethyl acetate (3×30 mL). The organics were dried over Na2SO4 and evaporated. The residue was purified via Biotage (2:1 Hex/EtOAc; 12S column) to provide compound 148 (0.025 g, 12.9% yield) as a off white solid. MS(ESI):348.0[M+H]⁺. ¹H NMR (400 MHz, Chloroform-d) δ 8.78 (s, 1H), 8.52 (s, 1H), 8.35 (d, J=1.7 Hz, 1H), 8.25 (d, J=8.4 Hz, 2H), 8.10 (d, J=8.4 Hz, 2H), 6.38 (d, J=1.7 Hz, 1H), 4.59 (s, 2H), 3.12 (s, 3H).

### Compound 149

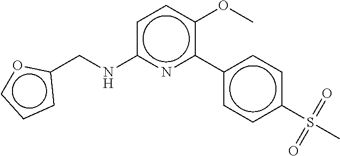

Was prepared using 6-bromo-N-[(furan-2-yl)methyl]-5-methoxypyridin-2-amine (0.1 g, 0.3532 mmol, 1.0 eq) and (4-methanesulfonylphenyl)boronic acid (84.7 mg, 0.4238 mmol, 1.2 eq) using method A. The residue was purified via Biotage (5:1 Hex/EtOAc; 12S column) to provide compound 149 (0.06 g, 47.6% yield) as a yellow solid. MS(ESI):359.0[M+H]⁺.

Compound 150

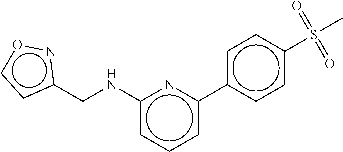

Was prepared using intermediate-3 (0.15 g, 0.5602 mmol, 1.0 eq) and 1-(1,2-oxazol-3-yl)methanamine hydrochloride (90.4 mg, 0.6722 mmol, 1.2 eq) using method 2. The residue was purified via Biotage (2:1 Hex/EtOAc; 12S column) to provide impure product, which was further purified by prep HPLC using (40-55% ACN in 0.1% formic acid in water as modifier) as mobile phase to provide compound 150 (0.03 g, 16.3% yield) as a light yellow solid. MS(ESI):330.0[M+H]⁺. ¹H NMR (400 MHz, DMSO-d6) δ 8.80 (d, J=1.7 Hz, 1H), 8.25 (d, J=8.3 Hz, 2H), 7.96 (d, J=8.2 Hz, 2H), 7.56 (t, J=7.8 Hz, 1H), 7.37 (t, J=5.9 Hz, 1H), 7.27 (d, J=7.4 Hz, 1H), 6.62 (d, J=8.3 Hz, 1H), 6.49 (d, J=1.7 Hz, 1H), 4.66 (d, J=5.9 Hz, 2H), 3.25 (s, 3H).

### Compound 151

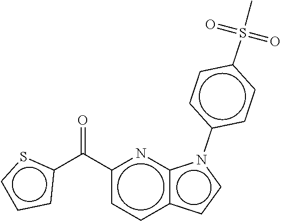

To a solution of 1H-pyrrolo[2,3-b]pyridin-6-yl(2-thienyl)methanone (0.60 g, 2.63 mmol, 1.00 eq) in Dioxane (10 mL), 1-bromo-4-methylsulfonyl-benzene (1.24 g, 5.26 mmol, 2.00 eq), Potassium phosphate tribasic (1.67 g, 7.89 mmol, 3.00 eq), Copper(I) iodide (0.15 g, 0.789 mmol, 0.300 eq) and (±)-trans-1,2-Diaminocyclohexane (0.66 mL, 5.52 mmol, 2.10 eq) were added and reaction mixture was heated at 120° C. for 16 h. After completion, reaction mixture was diluted with water (100 mL) and solid was filter and wash with water and extracted with DCM (100×2). Organic was evaporated and dried with sodium sulfate, Crude product was purified by column chromatography using silicagel (100-200 mesh) and pure product was elute out at 0 to 50% ethyl acetate in hexane to obtain [1-(4-methylsulfonylphenyl)pyrrolo[2,3-b]pyridin-6-yl]-(2-thienyl)methanone (800 mg, 80% yield) as a white solid. MS(ESI): 382.9[M+H]⁺. ¹H NMR (400 MHz, DMSO-d6) δ8.43-8.29 (m, 5H), 8.21-8.11 (m, 3H), 8.07 (d, J=8.5 Hz, 1H), 7.32 (t, J=4.4 Hz, 1H), 7.00 (d, J=3.6 Hz, 1H).

### Compound 152

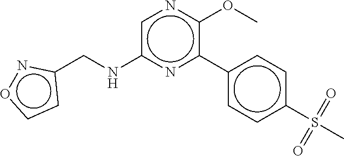

Was prepared using 5-chloro-3-(4-methanesulfonylphenyl)-2-methoxypyrazine (0.2 g, 0.6694 mmol) and 1-(1,2-oxazol-3-yl)methanamine (65.6 mg, 0.6694 mmol) using method 2. The residue was purified via Biotage (2:1 Hex/EtOAc; 12S column) and further purified by prep HPLC using (30-38% ACN in water containing 0.1% ammonia in water and 5 mM ammonium bicarbonate) as mobile phase to provide compound 152 (0.025 g, 10.3%) as a yellow solid. MS(ESI): 361.0 [M+H]⁺. ¹H NMR (400 MHz, DMSO-d6) δ 8.81 (d, J=1.7 Hz, 1H), 8.32-8.14 (m, 2H), 8.10-7.92 (m, 2H), 7.73 (s, 1H), 7.28 (t, J=6.1 Hz, 1H), 6.50 (d, J=1.7 Hz, 1H), 4.60 (d, J=6.0 Hz, 2H), 3.90 (s, 3H), 3.25 (s, 3H).

### Compound 153

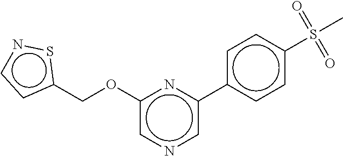

Was prepared using intermediate-7 (50 mg, 0.1860 mmol, 1 eq) and (1,2-thiazol-5-yl)methanol (42.8 mg, 0.372 mmol, 2 eq) using method 1. The crude was purified by normal phase chromatography (EtOAc/Hexanes, 0-100%, eluted 72%) to give compound 153 (51.1 mg, 79.1% yield). MS: [M+H]⁺ 348.14. ¹H NMR (300 MHz, Chloroform-d) δ 8.77-8.72 (m, 1H), 8.45 (d, J=1.7 Hz, 1H), 8.36-8.32 (m, 1H), 8.26-8.20 (m, 2H), 8.11-8.05 (m, 2H), 7.35-7.30 (m, 1H), 5.82 (s, 2H), 3.12 (s, 3H).

### Compounds 154 and 186

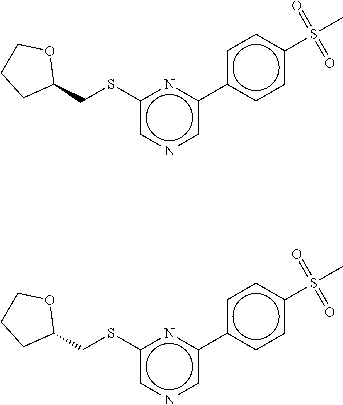

A stirred solution of (oxolan-2-yl)methanethiol (87.9 mg, 0.7442 mmol, 1 eq) in DMF (5 mL) was added, portion wise sodium hydride (26.6 mg, 1.11 mmol, 1.5 eq) at 0° C. and stirred for 1 h. After 1 h, added 2-chloro-6-(4-methanesulfonylphenyl)pyrazine (0.2 g, 0.7442 mmol, 1 eq) at room temperature and stirred for 4 h. After 4 h, the reaction mixture was diluted with ethyl acetate (70 mL) and washed with water (2×100 mL). The organic layer was dried over Na2SO4 and evaporated. The residue was purified via Biotage (2:1 Hex/EtOAc; 12S column) to provide the racemic product (0.163 g, 60.7% yield) as a off white solid. Enantiomers were isolated by chiral SFC (CHIRALCEL OJ-H (250 mm*4.6 mm, 5 μm)) using methanol containing 0.1% diethyl amine as mobile phase to provide two isomer: Compound 154: (eluting first, 0.04 g, 4.5% yield) as a off white solid. MS(ESI): 351.0[M+H]⁺. ¹H NMR (400 MHz, DMSO-d6) δ 9.03 (s, 1H), 8.66 (s, 1H), 8.39 (d, J=8.2 Hz, 2H), 8.09 (d, J=8.2 Hz, 2H), 4.11 (q, J=6.3 Hz, 1H), 3.80 (q, J=7.1 Hz, 1H), 3.67-3.61 (m, 1H), 3.46 (t, J=5.3 Hz, 2H), 3.29 (s, 3H), 2.03 (dt, J=14.9, 6.2 Hz, 1H), 1.92-1.79 (m, 2H), 1.71-1.62 (m, 1H). Compound 186: (eluting second, 0.037 g, 4.0% yield) as a white solid. MS(ESI): 351.0[M+H]⁺. ¹H NMR (400 MHz, DMSO-d6) δ 9.03 (s, 1H), 8.66 (s, 1H), 8.39 (d, J=8.4 Hz, 2H), 8.09 (d, J=8.2 Hz, 2H), 4.12 (p, J=6.3 Hz, 1H), 3.80 (q, J=7.2 Hz, 1H), 3.65 (q, J=7.4 Hz, 1H), 3.46 (t, J=5.2 Hz, 2H), 3.29 (s, 3H), 2.08-2.00 (m, 1H), 1.88 (ddd, J=27.6, 13.1, 6.1 Hz, 2H), 1.71-1.63 (m, 1H). The absolute stereochemistries of Compounds 154 and 186 were arbitrarily assigned.

### Compound 155

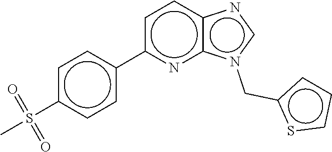

In a flask under nitrogen flow to 5-chloro-3-[(thiophen-2-yl)methyl]-3H-imidazo[4,5-b]pyridine (130 mg, 0.5205 mmol, 1 eq), boronic acid (104 mg, 0.5205 mmol, 1 eq), potassium carbonate (215 mg, 1.56 mmol, 3 eq) and XPhos Pd G2 (22.0 mg, 0.02602 mmol, 0.05 eq) were added. The mixture was degassed for 10 minutes and then it was diluted with water (2 mL) and 1,4-dioxane (8 mL). The dark mixture was stirred at 80° C. for 2 days. LCMS suggests a not complete starting material conversion. The mixture was cooled down at room temperature and solvent was evaporated. The aqueous layer was extracted with EtOAc (3×10 mL) and the combined organics were dried over MgSO4, filtered through Celite and DCM mixture and evaporated. compound 155 (80 mg, 41.6% yield) was purified using flash column chromatography (SiO2, 0-100% Hexanes\EtOAc). MS: 370.23 [M+H]⁺. ¹H NMR (400 MHz, Chloroform-d) δ 8.33 (dt, J=12.5, 2.1 Hz, 2H), 8.16 (d, J=8.6 Hz, 2H), 8.07 (dt, J=12.0, 1.7 Hz, 2H), 7.80 (d, J=8.4 Hz, 1H), 7.29 (dd, J=5.1, 1.2 Hz, 1H), 7.18 (dd, J=3.6, 1.1 Hz, 1H), 7.00 (dd, J=5.1, 3.5 Hz, 1H), 5.71 (s, 2H), 3.11 (s, 3H).

### Compound 159

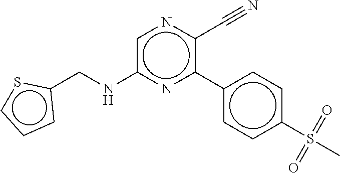

Was prepared using 3-chloro-5-{[(thiophen-2-yl)methyl]amino}pyrazine-2-carbonitrile (0.15 g, 0.5983 mmol, 1 eq) and (4-methanesulfonylphenyl)boronic acid (143 mg, 0.7179 mmol, 1.2 eq) using method A. The residue was purified via Biotage (5:1 Hex/EtOAc; 12S column) to provide compound 159 (0.1 g, 45.2% yield) as a light yellow solid. MS(ESI): 371.3 [M+H]⁺. ¹H NMR (400 MHz, DMSO-d6) δ 8.97 (t, J=5.8 Hz, 1H), 8.11 (dd, J=12.3, 3.7 Hz, 5H), 7.42 (dd, J=5.1, 1.3 Hz, 1H), 7.11 (d, J=3.4 Hz, 1H), 6.99 (dd, J=5.1, 3.4 Hz, 1H), 4.80 (d, J=5.7 Hz, 2H), 3.32 (s, 3H).

### Compound 160

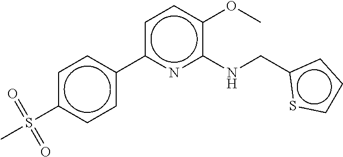

To stirred a solution of 6-(4-methanesulfonylphenyl)-3-methoxypyridin-2-amine (0.1 g, 0.3592 mmol, 1 eq), 2-(bromomethyl)thiophene (95.3 mg, 0.5388 mmol, 1.5 eq) in dimethylformamide (1 mL), was portion wise added sodium hydride (12.9 mg, 0.5388 mmol, 1.5 eq) and stirred at room temperature for 12 hours. After completion of reaction, the reaction mixture was quenched in water (50 mL) and extracted with ethyl acetate (3×30 mL). The organics were dried over Na2SO4 and evaporated. The residue was purified via Biotage (2:1 Hex/EtOAc; 12S column) to provide impure product, which was further purified by prep HPLC using (10-63% ACN in water containing 0.1% formic acid as modifier) as mobile phase to provide compound 160 (0.035 g, 26.1% yield) as a white solid. MS(ESI): 375.0[M+H]⁺. ¹H NMR (400 MHz, DMSO-d6) δ 8.28 (d, J=8.4 Hz, 2H), 7.93 (d, J=8.3 Hz, 2H), 7.36-7.21 (m, 2H), 7.13 (d, J=8.1 Hz, 1H), 7.05 (d, J=3.4 Hz, 1H), 7.01-6.86 (m, 2H), 4.80 (d, J=6.1 Hz, 2H), 3.85 (s, 3H), 3.23 (s, 3H).

### Compounds 162 and 167

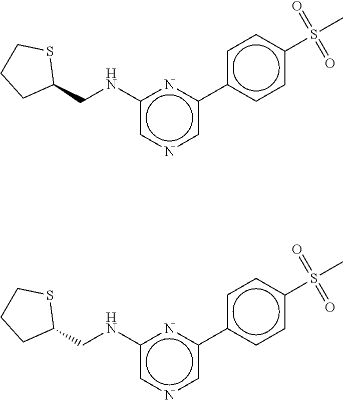

Obtained from 2-chloro-6-(4-methanesulfonylphenyl)pyrazine (0.3 g, 1.11 mmol, 1 eq) and 1-(thiolan-2-yl)methanamine (155 mg, 1.33 mmol, 1.2 eq) using method 3. The residue was purified via Biotage (2:1 Hex/EtOAc; 12S column) to provide impure product, which was further purified by prep HPLC using (20-40% ACN in water containing 0.1% ammonia and 5 mM ammonium bicarbonate as modifier) as mobile phase to provide the product (0.09 g, 23.2% yield) as off white solid. The racemic mixture was separated by chiral SFC (CHIRALPAK IG (250 mm*4.6 mm, 5 μm)) using methanol containing 0.1% diethyl amine in IPA:ACN (50:50) as mobile phase to provide two isomer: Compound 162: (eluting first, 0.035 g, 3.9% yield) as a off white solid. MS(ESI): 350.0[M+H]⁺. ¹H NMR (400 MHz, DMSO-d6) δ 8.38 (s, 1H), 8.28 (d, J=8.2 Hz, 2H), 8.08-7.94 (m, 3H), 7.57 (t, J=5.7 Hz, 1H), 3.64 (q, J=6.2 Hz, 1H), 3.48 (ddt, J=19.7, 13.4, 6.4 Hz, 2H), 3.26 (s, 3H), 2.87 (dt, J=11.7, 6.0 Hz, 1H), 2.77 (dt, J=10.6, 6.1 Hz, 1H), 1.95 (dq, J=18.5, 6.5 Hz, 3H), 1.80 (dd, J=10.7, 4.9 Hz, 1H). Compound 167: (eluting second, 0.033 g, 3.7% yield) as a off white solid. MS(ESI): 350.0[M+H]⁺. ¹H NMR (400 MHz, DMSO-d6) δ 8.38 (s, 1H), 8.28 (d, J=8.4 Hz, 2H), 8.08-7.97 (m, 3H), 7.57 (t, J=5.8 Hz, 1H), 3.64 (q, J=6.2 Hz, 1H), 3.47 (dhept, J=19.8, 6.2 Hz, 2H), 3.26 (s, 3H), 2.87 (dt, J=10.3, 6.0 Hz, 1H), 2.77 (q, J=5.2, 4.3 Hz, 1H), 2.05-1.87 (m, 3H), 1.85-1.75 (m, 1H). The absolute stereochemistries of Compounds 162 and 167 were arbitrarily assigned.

### Compound 163

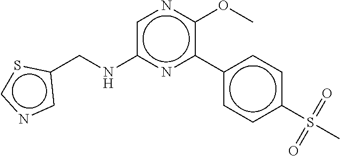

Was prepared using 5-chloro-3-(4-methanesulfonylphenyl)-2-methoxypyrazine (0.2 g, 0.6694 mmol, 1 eq) and 1-(1,3-thiazol-5-yl)methanamine hydrochloride (120 mg, 0.8032 mmol, 1.2 eq) using method 2. The residue was purified via Biotage (2:1 Hex/EtOAc; 12S column) and further purified by prep HPLC using (25-58% ACN in 0.1% formic acid in water) as mobile phase to provide compound 163 (0.004 g, 2% yield) as an off white solid. MS(ESI):377.3[M+H]⁺, 375.3[M+H]⁺. ¹H NMR (400 MHz, DMSO-d6) δ 8.91 (s, 1H), 8.31 (d, J=8.2 Hz, 2H), 8.00 (d, J=8.3 Hz, 2H), 7.87 (s, 1H), 7.70 (s, 1H), 7.35 (s, 1H), 4.74 (d, J=6.0 Hz, 2H), 3.90 (s, 3H), 3.26 (s, 3H).

### Compound 164

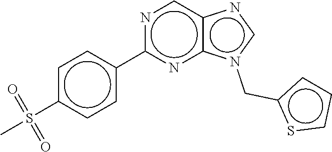

A yellow-orange mixture of 2-chloro-9-(thiophen-2-ylmethyl)-9H-purine (40 mg, 0.1595 mmol, 1 eq), (4-(methylsulfonyl)phenyl)boronic acid (40 mg, 0.1999 mmol, 1.253 eq), potassium carbonate (70 mg, 0.5064 mmol, 3.175 eq), and XPhos Pd G2 (12.5 mg, 0.01588 mmol, 0.1 eq) in 4:1 dioxane/water (1.0 mL) was heated at 60° C. under an argon atmosphere for 2 h. The resulting brown solution was cooled to room temperature and diluted with water and EtOAc. The layers were separated, and the aqueous layer was extracted with EtOAc. The combined organics were dried over MgSO4 and evaporated, and the residue was purified by flash column chromatography (0-10% MeOH/CH2Cl2, eluted ˜5%) to provide compound 164 (45.7 mg, 77.4% yield) as a white solid. MS (ESI) 371.1 [M+H]⁺. ¹H NMR (300 MHz, DMSO-d6) δ 9.32 (s, 1H), 8.81 (s, 1H), 8.74 (d, J=8.5 Hz, 2H), 8.11 (d, J=8.5 Hz, 2H), 7.48 (dd, J=5.1, 1.3 Hz, 1H), 7.31 (dd, J=3.5, 1.3 Hz, 1H), 7.01 (dd, J=5.1, 3.5 Hz, 1H), 5.81 (s, 2H), 3.29 (s, 3H).

### Compound 165

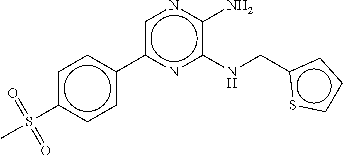

Was prepared using 6-bromo-N₂-[(thiophen-2-yl)methyl]pyrazine-2,3-diamine (0.5 g, 1.75 mmol, 1 eq) and (4-methanesulfonylphenyl)boronic acid (418 mg, 2.09 mmol, 1.2 eq) using method A. The residue was purified via Biotage (20:1 CH2Cl2/MeOH; 12M column) to provide compound 165 (0.5 g, 79.3% yield) as yellow solid.

MS(ESI):361.0[M+H]⁺. ¹H NMR (400 MHz, DMSO-d6) δ 8.21 (dd, J=8.4, 2.7 Hz, 2H), 8.00 (d, J=2.7 Hz, 1H), 7.89 (dt, J=6.1, 3.0 Hz, 2H), 7.35 (s, 1H), 7.11 (d, J=3.1 Hz, 1H), 7.05 (d, J=6.3 Hz, 1H), 6.96 (dt, J=5.4, 3.0 Hz, 1H), 6.45 (s, 2H), 4.84 (t, J=3.7 Hz, 2H), 3.21 (t, J=3.2 Hz, 3H).

### Compound 166

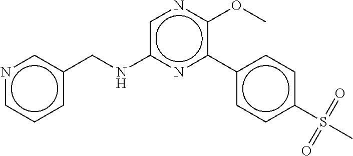

Was prepared using 5-chloro-3-(4-methanesulfonylphenyl)-2-methoxypyrazine (80 mg, 0.2677 mmol, 1 eq) and 1-(pyridin-3-yl)methanamine (58 μL, 0.5733 mmol, 2.142 eq) using method 2. The crude was purified by normal phase flash chromatography (Hexanes/EtOAc, 0-100%) to give compound 166 (45.9 mg, 46.3% yield) as an orange oil. MS: [M+H]⁺ 371.53; [M−H]⁻369.23. ¹H NMR (300 MHz, Chloroform-d) δ 8.67 (d, J=1.8 Hz, 1H), 8.54 (dd, J=4.8, 1.5 Hz, 1H), 8.28-8.22 (m, 2H), 8.00-7.94 (m, 2H), 7.74 (d, J=7.6 Hz, 1H), 7.56 (s, 1H), 7.30 (dd, J=7.6, 5.1 Hz, 1H), 4.81 (t, J=5.7 Hz, 1H), 4.62 (d, J=5.7 Hz, 2H), 3.98 (s, 3H), 3.07 (s, 3H).

### Compounds 168 and 209

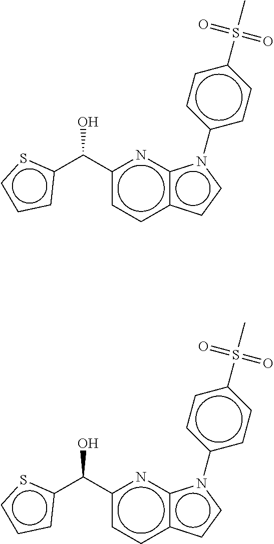

To a solution of [1-(4-methylsulfonylphenyl)pyrrolo[2,3-b]pyridin-6-yl]-(2-thienyl)methanone (0.40 g, 1.05 mmol, 1.00 eq) in THF (5 mL) and Methanol (5 mL), Sodium borohydride (0.079 g, 2.09 mmol, 2.00 eq) was added portionwise at 0° C. and the mixture was stirred for 5 hour at room temperature. The mixture was quenched with water (50 mL) and extracted with EtOAc (2×30 mL). The organics were dried with Na2SO4 and evaporated. The residue was purified viaBiotage (20:1 Hex/EtOAc; 12S column) and further purified by chiral SFC purification by CO2 and 0.1% Diethyl amine in methanol as a co-solvent using Chiralcel OJ-H (250 mm*4.6 mm*5 m) to provide (S)-[1-(4-methylsulfonylphenyl)pyrrolo[2,3-b]pyridin-6-yl]-(2-thienyl)methanol (eluting first, 19.1 mg, 5% yield) and (R)-[1-(4-methylsulfonylphenyl)pyrrolo[2,3-b]pyridin-6-yl]-(2-thienyl)methanol (eluting second, 24.2 mg, 5% yield)) as white solids. MS(ESI): 385.2 [M+H]⁺. ¹H NMR (400 MHz, DMSO-d6) δ 8.37 (d, J=8.5 Hz, 2H), 8.18-8.01 (m, 4H), 7.47 (d, J=8.2 Hz, 1H), 7.39 (d, J=4.9 Hz, 1H), 6.97 (dd, J=19.1, 4.0 Hz, 2H), 6.80 (d, J=3.9 Hz, 1H), 6.45 (d, J=4.7 Hz, 1H), 6.02 (s, 1H), 3.27 (s, 3H). MS(ESI): 385.2 [M+H]⁺. ¹H NMR (400 MHz, DMSO-d6) δ 8.39 (d, J=8.6 Hz, 2H), 8.15-8.04 (m, 4H), 7.48 (d, J=8.1 Hz, 1H), 7.40 (d, J=5.0 Hz, 1H), 6.99 (d, J=3.5 Hz, 1H), 6.94 (t, J=4.3 Hz, 1H), 6.80 (d, J=3.8 Hz, 1H), 6.45 (d, J=4.7 Hz, 1H), 6.03 (d, J=4.6 Hz, 1H), 3.29 (s, 3H). The absolute stereochemistries of Compounds 168 and 209 were arbitrarily assigned.

### Compound 169

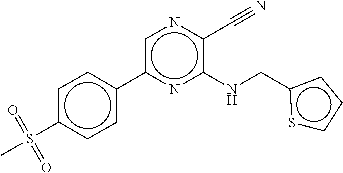

Obtained from 3-chloro-5-(4-(methylsulfonyl)phenyl)pyrazine-2-carbonitrile (0.1 g, 0.34 mmol, 1 eq) and thiophen-2-ylmethanamine (38.5 mg, 0.34 mmol, 1 eq) using method 3. The residue was purified via Biotage (2:1 Hex/EtOAc; 12S column) to provide compound 169 (0.011 g, 8.3% yield) as off white solid. MS(ESI): 360.0 [M+H]⁺. ¹H NMR (400 MHz, Chloroform-d) δ 8.51 (s, 1H), 8.29 (d, J=8.4 Hz, 2H), 8.12 (d, J=8.4 Hz, 2H), 7.26 (s, 1H), 7.11 (d, J=3.5 Hz, 1H), 7.02 (dd, J=5.1, 3.5 Hz, 1H), 5.85 (s, 1H), 5.01 (d, J=5.7 Hz, 2H), 3.13 (s, 3H).

### Compounds 170 and 198

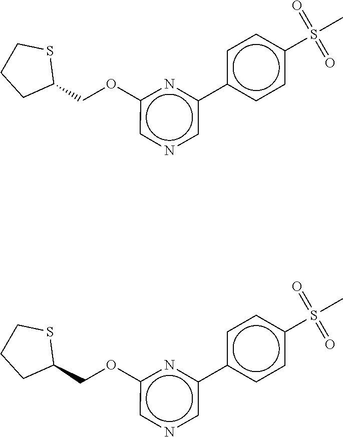

Was prepared using intermediate-7 (0.15 g, 0.5582 mmol, 1 eq) and (thiolan-2-yl)methanol (98.9 mg, 0.8373 mmol, 1.5 eq) using method 1. The residue was purified via Biotage (2:1 Hex/EtOAc; 12S column) to provide the racemic mixture (0.12 g, 61.5% yield) as a off white solid. Enantiomers were isolated by chiral SFC (Chiralcel OJ-H (250 mm*4.6 mm*5 m)) using methanol containing 0.1% diethyl amine in IPA: MeOH (50:50) as mobile phase to provide two isomer: Compound 170: (eluting first, 0.012 g, 2% yield) as an off-white solid. MS(ESI): 351.0[M+H]⁺. ¹H NMR (400 MHz, DMSO-d6) δ 8.95 (s, 1H), 8.37 (d, J=7.3 Hz, 3H), 8.07 (d, J=8.2 Hz, 2H), 4.42 (ddd, J=34.3, 10.6, 7.1 Hz, 2H), 3.79 (p, J=6.0 Hz, 1H), 3.28 (s, 3H), 2.94-2.76 (m, 2H), 2.07-1.85 (m, 4H). Compound 198: (eluting second, 0.01 g, 2% yield) as an off-white solid. MS(ESI): 351.0[M+H]⁺. ¹H NMR (400 MHz, DMSO-d6) δ 8.95 (s, 1H), 8.46-8.31 (m, 3H), 8.07 (d, J=8.3 Hz, 2H), 4.42 (ddd, J=34.3, 10.6, 7.1 Hz, 2H), 3.79 (p, J=6.6 Hz, 1H), 3.28 (s, 3H), 2.84 (ddt, J=31.3, 10.4, 6.0 Hz, 2H), 2.13-1.86 (m, 4H). The absolute stereochemistries of Compounds 170 and 198 were arbitrarily assigned.

### Compound 171

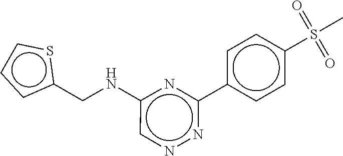

Was prepared using 3-chloro-N-[(thiophen-2-yl)methyl]-1,2,4-triazin-5-amine (60 mg, 0.2646 mmol) and (4-methanesulfonylphenyl)boronic acid (52.9 mg, 0.2646 mmol) using method A. The residue was purified via Biotage (1:1 Hex/EtOAc; 12S column) to provide compound 171 (10 mg, 10.8% yield) as a off white solid. MS(ESI): 347.0 [M+H]⁺, 345.0 [M−H]⁻. ¹H NMR (400 MHz, DMSO-d6) δ 8.95 (t, J=5.8 Hz, 1H), 8.67-8.57 (m, 3H), 8.08 (d, J=8.2 Hz, 2H), 7.42 (d, J=5.1 Hz, 1H), 7.16 (d, J=3.4 Hz, 1H), 6.99 (dd, J=5.1, 3.4 Hz, 1H), 4.87 (d, J=5.8 Hz, 2H), 3.28 (s, 3H).

### Compound 172

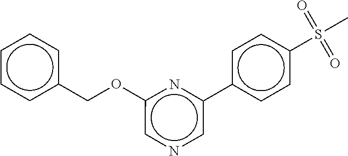

Was prepared using intermediate-7 (0.15 g, 0.5582 mmol, 1 eq) and phenylmethanol (48.2 mg, 0.4465 mmol, 0.8 eq) using method 1. The residue was purified via Biotage (2:1 Hex/EtOAc; 12S column) to provide compound 172 (0.1 g, 52.4% yield) as an off-white solid. MS(ESI): 341.3[M+H]⁺. ¹H NMR (400 MHz, DMSO-d6) δ 8.96 (s, 1H), 8.41 (m, 3H), 8.07 (m, 2H), 7.55 (m, 2H), 7.40 (m, 3H), 5.54 (s, 2H), 3.29 (s, 3H).

### Compound 173

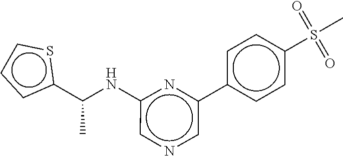

Was prepared using 2-chloro-6-(4-(methylsulfonyl)phenyl)pyrazine (150 mg, 0.5582 mmol) and (R)-1-(thiophen-2-yl)ethanamine (71.0 mg, 0.5582 mmol) using method 3. The residue was purified via Biotage (50:1 CH2Cl2/MeOH; 12M column) and further purified by prep HPLC using (20-50% ACN in water containing 0.1% formic acid) as mobile phase to provide compound 173 (90 mg, 44.9% yield) as a off white solid. MS(ESI): 358.0 [M−H]⁻, 360.2 [M+H]⁺. ¹H NMR (400 MHz, Chloroform-d) δ 8.35 (s, 1H), 8.22-8.11 (m, 2H), 8.06-7.97 (m, 2H), 7.90 (s, 1H), 7.20 (dd, J=5.0, 1.3 Hz, 1H), 7.06 (d, J=3.5 Hz, 1H), 6.97 (dd, J=5.1, 3.5 Hz, 1H), 5.50 (p, J=6.9 Hz, 1H), 5.04 (d, J=7.5 Hz, 1H), 3.09 (s, 3H), 1.74 (d, J=6.8 Hz, 3H).

### Compound 175

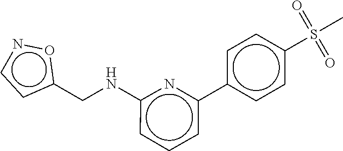

Was prepared using intermediate-4 (150 mg, 0.4804 mmol) and 1-(1,2-oxazol-5-yl)methanamine (47.1 mg, 0.4804 mmol) using method 2. The residue was purified via Biotage (1:1 Hex/EtOAc; 12M column) to provide compound 175 (10 mg, 6.2%) as an off white solid. MS(ESI): 330.3 [M+H]⁺, 328.3 [M−H]⁻. ¹H NMR (400 MHz, DMSO-d6) δ 8.46 (d, J=1.8 Hz, 1H), 8.23 (d, J=8.2 Hz, 2H), 7.96 (d, J=8.2 Hz, 2H), 7.57 (t, J=7.8 Hz, 1H), 7.45 (t, J=5.9 Hz, 1H), 7.27 (d, J=7.4 Hz, 1H), 6.64 (d, J=8.3 Hz, 1H), 6.35 (d, J=1.7 Hz, 1H), 4.75 (d, J=5.8 Hz, 2H), 3.24 (s, 3H).

### Compound 176

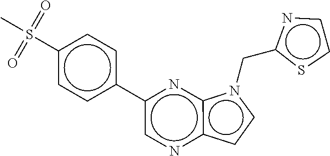

Was prepared using 2-({3-chloro-5H-pyrrolo[2,3-b]pyrazin-5-yl}methyl)-1,3-thiazole (0.1 g, 0.3988 mmol, 1 eq) and (4-methanesulfonylphenyl)boronic acid (95.7 mg, 0.4785 mmol, 1.2 eq) using method A. The residue was purified via Biotage (5:1 Hex/EtOAc; 12S column) and further purified by prep HPLC using (35-40% ACN in water containing 0.1% formic acid) as mobile phase to provide compound 176 (0.01 g, 6.8% yield) as a white solid. MS(ESI): 371.0[M+H]⁺. ¹H NMR (400 MHz, DMSO-d6) δ 9.22 (s, 1H), 8.53-8.38 (m, 2H), 8.19 (d, J=3.7 Hz, 1H), 8.13-7.97 (m, 2H), 7.77 (d, J=3.2 Hz, 1H), 7.67 (d, J=3.2 Hz, 1H), 6.83 (d, J=3.7 Hz, 1H), 5.94 (s, 2H), 3.27 (s, 3H).

### Compound 179

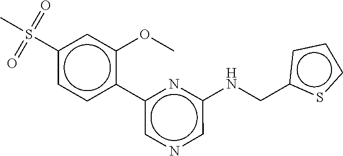

Was prepared using 2-chloro-6-(4-methanesulfonyl-2-methoxyphenyl)pyrazine (200 mg, 0.6694 mmol) and 1-(thiophen-2-yl)methanamine (75.7 mg, 0.6694 mmol) using method 2. The residue was purified via Biotage (1:1 Hex/EtOAc; 12M column) and further purified by prep HPLC purification using (10-60% ACN in water containing 0.1% formic acid) as mobile phase to provide compound 179 (5 mg, 2.0%) as a white solid. MS(ESI): 376.0 [M+H]⁺. ¹H NMR (400 MHz, DMSO-d6) δ 8.32 (s, 1H), 8.07 (d, J=7.9 Hz, 1H), 7.95 (s, 1H), 7.81 (t, J=5.9 Hz, 1H), 7.64-7.56 (m, 2H), 7.37 (dd, J=5.1, 1.3 Hz, 1H), 7.07 (d, J=3.4 Hz, 1H), 6.97 (dd, J=5.1, 3.4 Hz, 1H), 4.71 (d, J=5.9 Hz, 2H), 3.97 (s, 3H).

### Compound 180

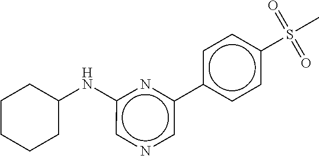

A stirred solution of 2-chloro-6-(4-(methylsulfonyl)phenyl)pyrazine (100 mg, 0.3721 mmol, 1 eq) in cyclohexanamine (1 mL) was heated at 140° C. under microwave irradiation for 1 h. After completion of the reaction, the reaction mixture was filtered through Buchner funnel and washed with water (3×5 mL). Resultant solid was dried under vacuum to provide compound 180 (28 mg, 22.7% yield) as an off white solid. MS(ESI): 332.2 [M+H]⁺. ¹H NMR (400 MHz, DMSO-d6) δ 8.33 (s, 1H), 8.27 (d, J=8.4 Hz, 2H), 8.02 (d, J=8.3 Hz, 2H), 7.95 (s, 1H), 7.20 (d, J=7.4 Hz, 1H), 3.83 (s, 1H), 3.26 (s, 3H), 2.02-1.93 (m, 2H), 1.74 (dd, J=10.9, 6.6 Hz, 2H), 1.62 (d, J=12.7 Hz, 1H), 1.39 (q, J=12.2 Hz, 2H), 1.33-1.23 (m, 3H).

### Example 4

### Materials

Ovine COX-1 (oCOX-1), ovine COX2 (oCOX2), human recombinant COX2 (hCOX2), arachidonic acid, PGE₂, d₄-PGD₂, and d₂-PGE₂ (labeled with deuterium atoms at positions 3 and 4) were purchased from Cayman Chemicals (Ann Arbor, Mich.). The cofactors (−)epinephrine and hematin, and the COX inhibitors indomethacin, resveratrol and diclofenac were purchased from Sigma-Aldrich (St. Louis, Mo.). Celecoxib was purchased from 3B PharmaChem International (Wuhan, China). All organic solvents were HPLC grade or better and were purchased from Thermo Fisher (Hanover Park, Ill.). Formic acid was purchased from EMD Chemicals (San Diego, Calif.). Purified water was prepared by using a Millipore Milli-Q purification system (Millipore, Billerica, Mass.). All other chemicals and solvents were ACS reagent grade, unless stated otherwise.

### COX Inhibition Assay

In an Eppendorf tube, 146 μL of 100 mM Tris.HCl (pH 8.0) buffer, 2 μL of 100 μM hematin (co-factor) and 10 μL of 40 mM L-epinephrine (co-factor) were mixed at room temperature. Next, 20 μL of Tris.HCl (pH 8.0) buffer containing 0.2 μg COX2 or 0.1 μg COX-1 (approximately 1 unit of enzyme; 1 unit COX utilizes 1 nmol 02/mg/min at 37° C.) was added, and the solution was incubated at room temperature for 2 min. A 2 μL aliquot of the COX inhibitor in DMSO was added to the enzyme solution and preincubated at 37° C. for 10 min. Negative controls were identical except that 2 μL aliquots of DMSO without inhibitor were used instead.

Each COX reaction was initiated by adding 20 μL of arachidonic acid in Tris.HCl (pH 8.0) buffer to give a final concentration of 5 μM; and the reaction was terminated after 2 min by adding 20 μL of 2.0 M HCl. The surrogate standards d₄-PGE₂ and d₄-PGD₂ (10 μL aliquot of 50 ng/mL solution in methanol) were added to correct for errors or degradation during sample handling and for variation in injection volume or instrument response during LC-MS-MS. After 30 min, PGE2, PGD2 and their surrogate standards were extracted from each incubation mixture using 800 μL hexane/ethyl acetate (50:50, v/v). The organic phase was removed, evaporated to dryness, and reconstituted in 100 μL methanol/water (50:50, v/v) for analysis using LC-MS-MS.

The concentration of PGE2 in each sample was measured using LC-MS-MS, and the percent of COX inhibition by each test solution was determined by comparing the amount of PGE₂ produced in the experiment with that produced in the negative control incubation. The formation of PGD₂ was measured for quality control purposes, since the levels of PGD₂ should be proportional to those of PGE₂. For IC₅₀ value determination, 12 different concentrations of each inhibitor were assayed three times. The IC₅₀ value of each inhibitor toward COX-1 or COX2 was determined by plotting and analyzing the inhibition curve data using Graph Pad Prism 5 software (Mountain View, Calif.). The selectivity of each inhibitor towards COX2 was calculated as the ratio of the IC₅₀ values (COX2/COX-1). Using 7 concentrations of arachidonic acid from 0 to 32 μM, the initial rates of formation of PGE₂ were determined for ovine COX-1, ovine COX2 and human COX2 using LC-MS-MS. From these data, Michaelis-Menten curves were plotted, and the Kₘ values were determined using SigmaPlot 9 software (Systat Software; San Jose, Calif.).

### LC-MS-MS

Negative ion electrospray tandem mass spectrometric measurement of PGE₂ was carried out using an Applied Biosystems (Foster City, Calif.) API 4000 triple quadrupole mass spectrometer equipped with a Shimadzu (Columbia, Md.) Prominence HPLC system based on the method of Cao et al. A Waters (Milford, Mass.) XTerra MS C₁₈ analytical column (2.1×50 mm, 3.5 m) was used for HPLC separations with an isocratic mobile phase consisting of acetonitrile/aqueous 0.1% formic acid (35:65; v/v) at a flow rate of 200 μL/min. The deprotonated molecules of m/z 351 and m/z 355 corresponding to PGE₂ and the surrogate standard d₄-PGE₂, respectively, were selected for collision-induced dissociation at a collision energy of −23 eV. The abundant product ions of m/z 271 and m/z 275, corresponding to the [M−H−2H₂O—CO₂]— product ions of PGE₂ and d₄-PGE₂, respectively, were measured using selected reaction monitoring.

TABLE 1

| | COX2 2 | | COX2 2 | | COX2 10 | | COX2 30 | | COX1 10 |
|---|---|---|---|---|---|---|---|---|---|
| | minutes | COX2 2 | minutes | COX2 2 | minutes | COX2 10 | minutes | COX2 30 | minutes |
| | pge2/pge2d4 | minutes | pge2 area | minutes | pge2 area | minutes | pge2 area | minutes | pge2/pge2d4 |
| Com- | area ratio | pge2/pge2d4 | (counts) | pge2 area | (counts) | pge2 area | (counts) | pge2 area | area ratio |
| pound | Avg. IC50 | area ratio | Avg. IC50 | (counts) | Avg. IC50 | (counts) | Avg. IC50 | (counts) | Avg. IC50 |
| No. | (microM) | Avg. LLE | (microM) | Avg. LLE | (microM) | Avg. LLE | (microM) | Avg. LLE | (microM) |
| 1 | 0.003274 | 4.86398 | 0.00366194 | 4.80588 | | | | | 11.5227 |
| 2 | 0.005128 | 3.98094 | 0.005451 | 3.97526 | | | | | 52 |
| 3 | 0.005549 | 6.48387 | 0.0053825 | 6.51157 | | | | | 1.09964 |
| 4 | 0.005998 | 4.85475 | 0.014101 | 4.61454 | | | | | |
| 5 | 0.006055 | 3.70756 | 0.0071435 | 3.55257 | | | | | |
| 6 | 0.006256 | 4.36368 | 0.00819625 | 4.24735 | | | | | 2.85688 |
| 7 | 0.006364 | 4.52242 | 0.00711075 | 4.50917 | | | | | 52 |
| 8 | 0.006595 | 5.39905 | 0.00715133 | 5.44577 | | | | | |
| 9 | 0.00803 | 4.29969 | 0.0089062 | 4.27639 | | | | | 48.8761 |
| 10 | 0.008122 | 5.4246 | 0.009536 | 5.34902 | | | | | 5.84612 |
| 11 | 0.008418 | 4.58386 | 0.00813 | 4.6244 | | | | | |
| 12 | 0.008436 | 5.44461 | 0.0097845 | 5.41657 | | | | | 38.5063 |
| 13 | 0.008777 | 4.5476 | 0.00819133 | 4.59002 | | | | | |
| 14 | 0.008811 | 5.17231 | 0.009373 | 5.20287 | | | | | 40.262 |
| 15 | 0.008909 | 5.1257 | 0.009621 | 5.09796 | | | | | 52 |
| 16 | 0.009121 | 4.9079 | 0.00871463 | 4.93216 | | | | | 39.9467 |
| 17 | 0.009622 | 4.65346 | 0.00992075 | 4.64389 | | | | | |
| 18 | 0.009987 | 4.92972 | 0.008216 | 5.01784 | | | | | 3.14496 |
| 19 | 0.010169 | 5.64296 | 0.00901082 | 5.68059 | 0.0015035 | 6.3861 | | | 10.3679 |
| 20 | 0.01082 | 3.9449 | 0.0114642 | 3.94233 | | | | | 52 |
| 21 | 0.01089 | 4.81771 | 0.0138953 | 4.71623 | | | | | 28.7422 |
| 22 | 0.011166 | 5.71891 | 0.0111863 | 5.72913 | | | | | |
| 23 | 0.011446 | 4.59154 | 0.0106787 | 4.61894 | | | | | 0.32338 |
| 24 | 0.011722 | 4.49785 | 0.0102908 | 4.5592 | | | | | 34.6861 |
| 25 | 0.011805 | 5.04051 | 0.0195593 | 4.85705 | | | | | 52 |
| 26 | 0.011958 | 3.77783 | 0.0166104 | 3.65198 | | | | | 11.3679 |
| 27 | 0.013257 | 4.78918 | 0.0108923 | 4.85318 | | | | | 52 |
| 28 | 0.013641 | 4.72197 | 0.0125577 | 4.78012 | | | | | 52 |
| 29 | 0.014893 | 5.39934 | 0.0145044 | 5.47439 | | | | | 52 |
| 30 | 0.015024 | 3.66838 | 0.0122268 | 3.68466 | | | | | 42.8423 |
| 31 | 0.015168 | 3.21807 | 0.0184683 | 3.11628 | | | | | 2.22363 |
| 32 | 0.015492 | 4.43263 | 0.0140263 | 4.49349 | 0.00250333 | 5.33786 | | | |
| 33 | 0.016385 | 4.75691 | 0.015051 | 4.79608 | | | | | 33.2947 |
| 34 | 0.016396 | 4.82571 | 0.0182526 | 4.75243 | | | | | 30.1119 |
| 35 | 0.017242 | 4.23228 | 0.0173682 | 4.21398 | | | | | 22.4193 |
| 36 | 0.01753 | 4.97385 | 0.0182029 | 4.96685 | | | | | 36.3769 |
| 37 | 0.018092 | 5.44017 | 0.014324 | 5.69425 | | | | | 39.2427 |
| 38 | 0.018445 | 5.14243 | 0.020585 | 5.08751 | | | | | 0.698686 |
| 39 | 0.018631 | | 0.0183866 | | | | | | 52 |
| 40 | 0.018834 | 4.50186 | 0.0206597 | 4.48662 | | | | | |
| 41 | 0.019013 | 3.73483 | 0.0216692 | 3.71584 | | | | | 52 |
| 42 | 0.019488 | 3.55144 | 0.0067136 | 3.78019 | | | | | 36.1515 |
| 43 | 0.019513 | 5.82652 | 0.0184922 | 5.85932 | | | | | |
| 44 | 0.019563 | 5.72205 | 0.0201122 | 5.72493 | | | | | 52 |
| 45 | 0.019987 | 3.87706 | 0.0231618 | 3.80482 | | | | | 6.64234 |
| 46 | 0.020224 | 3.46161 | 0.0235353 | 3.43053 | | | | | 50.9153 |
| 47 | 0.021232 | 4.85979 | 0.0155422 | 4.97397 | | | | | 38.7823 |
| 48 | 0.021955 | 4.8978 | 0.0201335 | 4.8663 | | | | | |
| 49 | 0.02219 | 5.23456 | 0.0210365 | 5.22835 | | | | | 52 |
| 50 | 0.02283 | 4.26077 | 0.019993 | 4.34143 | 0.004996 | 4.99587 | 0.000416 | 5.9869 | 32.2654 |
| 51 | 0.023879 | 5.18526 | 0.023185 | 5.21054 | 0.003573 | 6.08789 | | | 2.92533 |
| 52 | 0.023896 | 4.37655 | 0.0132717 | 4.61235 | | | | | 51.8919 |
| 53 | 0.02496 | 4.85169 | 0.0275524 | 4.79907 | | | | | 8.60609 |
| 54 | 0.025496 | 3.72699 | 0.0238152 | 3.7767 | 0.0094315 | 4.18219 | | | 39.1627 |
| 55 | 0.030534 | | 0.026514 | | | | | | |
| 56 | 0.030535 | | 0.032864 | | | | | | |
| 57 | 0.030616 | 3.1281 | 0.0298139 | 3.13165 | | | | | 52 |
| 58 | 0.031004 | 5.69112 | 0.0308701 | 5.69112 | | | | | 34.3875 |
| 59 | 0.032032 | 5.11167 | 0.0426278 | 5.06225 | | | | | 52 |
| 60 | 0.032345 | 5.90211 | 0.0333787 | 5.89117 | | | | | 52 |
| 61 | 0.033287 | 3.44631 | 0.0370088 | 3.40278 | | | | | 52 |
| 62 | 0.034543 | 4.78093 | 0.040473 | 4.65413 | | | | | |
| 63 | 0.035083 | 3.89682 | 0.0270748 | 3.96066 | | | | | 1.99249 |
| 64 | 0.036119 | 5.59485 | 0.0366187 | 5.59132 | | | | | 5.3641 |
| 65 | 0.036972 | 4.70382 | 0.0390095 | 4.68053 | | | | | 52 |
| 66 | 0.037327 | 4.55392 | 0.0420473 | 4.50526 | | | | | 52 |
| 67 | 0.038941 | 3.0886 | 0.0451995 | 3.01624 | | | | | 19.555 |
| 68 | 0.040178 | | 0.040993 | | | | | | |
| 69 | 0.040579 | 2.72168 | 0.0288043 | 2.83378 | | | | | |
| 70 | 0.040602 | 3.79112 | 0.0464507 | 3.7404 | | | | | |
| 71 | 0.041206 | 3.16496 | 0.041636 | 3.16671 | | | | | 49.3416 |
| 72 | 0.041415 | 2.93983 | 0.0342241 | 2.99469 | | | | | 17.6174 |
| 73 | 0.042883 | 4.79951 | 0.0452495 | 4.74043 | | | | | 15.032 |
| 74 | 0.043196 | 3.89925 | 0.0473647 | 3.89402 | 0.000932 | 5.56072 | | | 0.304882 |
| 75 | 0.045444 | 3.98413 | 0.0622888 | 3.88683 | | | | | |
| 76 | 0.048572 | 4.27788 | 0.04849 | 4.29121 | | | | | 31.5673 |
| 77 | 0.048646 | 4.25919 | 0.0538148 | 4.24647 | | | | | |
| 78 | 0.049439 | 4.76806 | 0.0435774 | 4.84605 | | | | | 33.5347 |
| 79 | 0.050579 | 4.19163 | 0.059582 | 4.10911 | | | | | |
| 80 | 0.052023 | 3.32989 | 0.0455175 | 3.38165 | | | | | 37.8265 |
| 81 | 0.05591 | 5.23108 | 0.0409802 | 5.42832 | | | | | |
| 82 | 0.057279 | 4.35343 | 0.0633048 | 4.32768 | | | | | 17.1776 |
| 83 | 0.057923 | 3.24064 | 0.0534613 | 3.27816 | | | | | 52 |
| 84 | 0.058002 | 3.89778 | 0.0524977 | 4.02317 | | | | | |
| 85 | 0.058124 | 4.22991 | 0.115139 | 3.95058 | | | | | 52 |
| 86 | 0.060992 | 5.13384 | 0.0682258 | 5.08663 | | | | | |
| 87 | 0.061907 | 4.85396 | 0.0565143 | 4.88079 | | | | | 42.6246 |
| 88 | 0.063441 | 4.70915 | 0.0693417 | 4.67031 | | | | | 16 |
| 89 | 0.065563 | 3.84044 | 0.0599843 | 3.87485 | | | | | |
| 90 | 0.06713 | 5.44239 | 0.941908 | 4.51005 | | | | | |
| 91 | 0.068593 | 4.03399 | 0.0653387 | 4.0886 | | | | | 38.3012 |
| 92 | 0.068631 | | 0.0583848 | | | | | | |
| 93 | 0.068994 | 5.01725 | 0.0557613 | 5.13899 | 0.00685433 | 5.9924 | | | |
| 94 | 0.071049 | 4.78648 | 0.0699113 | 4.81785 | | | | | 10.1845 |
| 95 | 0.071122 | 3.42956 | 0.090973 | 3.47674 | | | | | |
| 96 | 0.074226 | 4.84994 | 0.0849912 | 4.89661 | 0.0079425 | 5.82996 | | | 34.1608 |
| 97 | 0.075653 | 4.51705 | 0.058054 | 4.62566 | | | | | |
| 98 | 0.075976 | 5.06712 | 0.0615999 | 5.16779 | 0.00556657 | 6.19535 | 0.0027425 | 6.4321 | 9.89231 |
| 99 | 0.077912 | 4.28646 | 0.0869407 | 4.24837 | | | | | 45.9289 |
| 100 | 0.083943 | 3.64489 | 0.105942 | 3.59442 | | | | | |
| 101 | 0.086693 | 3.35697 | 0.0772363 | 3.33221 | | | | | |
| 102 | 0.087472 | 5.64683 | 0.103027 | 5.57186 | | | | | |
| 103 | 0.088846 | 4.75132 | 0.073332 | 4.83808 | | | | | |
| 104 | 0.091146 | 4.90112 | 0.0740952 | 4.97047 | | | | | |
| 105 | 0.091486 | 3.98992 | 0.105218 | 3.91817 | | | | | |
| 106 | 0.09158 | 5.5926 | 0.0664613 | 5.69867 | | | | | |
| 107 | 0.09334 | 4.93161 | 0.0881403 | 4.94772 | | | | | |
| 108 | 0.09448 | 3.84672 | 0.0798657 | 3.92629 | | | | | |
| 109 | 0.09817 | 4.38387 | 0.0793272 | 4.48693 | 0.013217 | 5.29705 | | | |
| 110 | 0.098381 | 5.41104 | 0.0933625 | 5.45105 | | | | | |
| 111 | 0.099129 | 2.29362 | 0.113122 | 2.24221 | | | | | |
| 112 | 0.101697 | 3.61417 | 0.108457 | 3.52465 | | | | | |
| 113 | 0.103332 | 4.95619 | 0.122424 | 4.89658 | | | | | |
| 114 | 0.104267 | 5.0904 | 0.130765 | 5.03447 | | | | | |
| 115 | 0.106969 | 2.86737 | 0.063195 | 2.98166 | | | | | |
| 116 | 0.108279 | 4.50569 | 0.0999338 | 4.56362 | | | | | |
| 117 | 0.110488 | 4.02922 | 0.06148 | 4.18467 | | | | | |
| 118 | 0.114234 | 3.64053 | 0.152745 | 3.56029 | | | | | |
| 119 | 0.116228 | 4.69544 | 0.233449 | 4.21493 | | | | | |
| 120 | 0.118444 | 5.37136 | 0.123839 | 5.43981 | 0.01154 | 6.39748 | | | |
| 121 | 0.121085 | | 0.123989 | | | | | | |
| 122 | 0.127338 | 2.21186 | 0.0949633 | 2.32485 | | | | | |
| 123 | 0.131204 | 3.56939 | 0.120983 | 3.5904 | | | | | |
| 124 | 0.139119 | 4.42528 | 0.1234 | 4.3461 | | | | | |
| 125 | 0.140534 | 3.01745 | 0.173961 | 2.95274 | | | | | |
| 126 | 0.142042 | 3.56849 | 0.121887 | 3.63446 | | | | | |
| 127 | 0.144397 | 6.12073 | 0.168862 | 6.07692 | | | | | |
| 128 | 0.144416 | 5.78441 | 0.138576 | 5.80876 | | | | | |
| 129 | 0.152117 | 5.40513 | 0.141371 | 5.44322 | | | | | |
| 130 | 0.152824 | 2.66062 | 0.210065 | 2.62584 | 0.00942257 | 4.11625 | 0.000242 | 5.42032 | |
| 131 | 0.157003 | 5.06527 | 0.131368 | 5.12938 | | | | | |
| 132 | 0.157813 | 4.45138 | 0.974509 | 3.94205 | | | | | |
| 133 | 0.166521 | 4.55792 | 0.126329 | 4.58156 | | | | | |
| 134 | 0.1831 | 4.3725 | 0.238617 | 4.23533 | | | | | |
| 135 | 0.188985 | 4.82024 | 0.291031 | 4.57924 | | | | | |
| 136 | 0.196435 | 5.1378 | 0.160804 | 5.20639 | | | | | |
| 137 | 0.196657 | 4.97656 | 0.215345 | 4.93646 | | | | | |
| 138 | 0.202156 | 4.95716 | 0.198423 | 4.95676 | | | | | |
| 139 | 0.207429 | | 0.218853 | | | | | | |
| 140 | 0.212115 | 2.8258 | 0.311105 | 2.74976 | | | | | |
| 141 | 0.21277 | 5.49702 | 0.176491 | 5.57897 | 0.022312 | 6.4815 | | | |
| 142 | 0.221463 | 5.555 | 0.211045 | 5.57562 | | | | | |
| 143 | 0.226281 | 4.84226 | 0.216154 | 4.92827 | | | | | |
| 144 | 0.233827 | 3.60452 | 0.22896 | 3.63725 | | | | | |
| 145 | 0.234058 | 4.32105 | 0.217025 | 4.37036 | 0.0387665 | 5.18612 | | | |
| 146 | 0.248025 | 2.10151 | 0.201166 | 2.13229 | | | | | |
| 147 | 0.249551 | 4.54301 | 0.113133 | 4.9085 | | | | | |
| 148 | 0.268025 | 5.48446 | 0.255261 | 5.51838 | | | | | |
| 149 | 0.271767 | 4.05656 | 0.285214 | 4.03261 | | | | | |
| 150 | 0.27636 | 5.05026 | 0.249429 | 5.0953 | | | | | |
| 151 | 0.292933 | | 0.233167 | | | | | | |
| 152 | 0.297286 | 6.03146 | 0.174465 | 6.19654 | | | | | |
| 153 | 0.304185 | 5.15304 | 0.195214 | 5.32264 | | | | | |
| 154 | 0.315007 | 4.68671 | 0.295337 | 4.72617 | | | | | |
| 155 | 0.319974 | 2.32129 | 0.317417 | 2.32397 | | | | | |
| 156 | 0.331684 | 3.20838 | 0.327252 | 3.20364 | 0.0355067 | 4.18893 | | | |
| 157 | 0.35003 | 3.38212 | 0.427944 | 3.28078 | | | | | |
| 158 | 0.358894 | 3.95595 | 0.349083 | 3.97159 | | | | | |
| 159 | 0.36126 | 4.78073 | 0.380963 | 4.75066 | | | | | |
| 160 | 0.385267 | 3.16188 | 0.360522 | 3.17432 | | | | | |
| 161 | 0.388425 | | 0.336742 | | | | | | |
| 162 | 0.400764 | 4.81145 | 0.344839 | 4.88777 | | | | | |
| 163 | 0.409036 | 4.49071 | 0.319356 | 4.59749 | | | | | |
| 164 | 0.411291 | 2.59545 | 0.368497 | 2.63813 | 0.0503105 | 3.53409 | | | |
| 165 | 0.424695 | 4.59242 | 0.390587 | 4.62864 | | | | | |
| 166 | 0.427317 | 5.25904 | 0.37962 | 5.24882 | | | | | |
| 167 | 0.431971 | 4.79239 | 0.432735 | 4.78205 | | | | | |
| 168 | 0.437216 | | 0.42458 | | | | | | |
| 169 | 0.441652 | 4.60086 | 0.294027 | 4.81256 | 0.059128 | 5.52649 | | | |
| 170 | 0.442327 | 4.60978 | 0.931016 | 4.14864 | | | | | |
| 171 | 0.446611 | 4.36407 | 0.452217 | 4.34464 | | | | | |
| 172 | 0.447174 | 4.17424 | 0.52011 | 4.01015 | | | | | |
| 173 | 0.451865 | 3.7158 | 0.325513 | 3.9817 | 0.0383623 | 4.80817 | | | |
| 174 | 0.483537 | | 0.481091 | | | | | | |
| 175 | 0.508472 | 4.7843 | 0.637599 | 4.68545 | | | | | |
| 176 | 0.532466 | 3.19003 | 0.519912 | 3.2105 | | | | | |
| 177 | 0.533607 | 3.06522 | 0.635252 | 2.98151 | | | | | |
| 178 | 0.543507 | 4.69026 | 0.607966 | 4.63642 | 0.119293 | 5.42947 | | | |
| 179 | 0.546129 | 4.28637 | 0.386878 | 4.32243 | | | | | |
| 180 | 0.555389 | 3.95042 | 0.514196 | 3.96102 | | | | | |
| 181 | 0.558571 | 5.19016 | 0.524681 | 5.2357 | | | | | |
| 182 | 0.558884 | 5.04163 | 0.414575 | 5.14672 | | | | | |
| 183 | 0.565997 | 5.67781 | 0.595816 | 5.65489 | | | | | |
| 184 | 0.570024 | | 0.457104 | | | | | | |
| 185 | 0.609973 | 3.62274 | 0.571653 | 3.62296 | | | | | |
| 186 | 0.626912 | 4.31518 | 0.616371 | 4.31559 | | | | | |
| 187 | 0.62915 | | 0.635312 | | | | | | |
| 188 | 0.652237 | 4.27537 | 0.546194 | 4.37264 | | | | | |
| 189 | 0.655722 | 2.76744 | 0.838896 | 2.661 | | | | | |
| 190 | 0.71061 | 3.52848 | 0.773944 | 3.49828 | | | | | |
| 191 | 0.735459 | | 0.88616 | | | | | | |
| 192 | 0.751129 | 4.41429 | 0.602224 | 4.52372 | | | | | |
| 193 | 0.771987 | 4.9351 | 1.08922 | 4.81346 | | | | | |
| 194 | 0.882782 | 2.60664 | 1.04022 | 2.53762 | | | | | |
| 195 | 0.88532 | | 0.969368 | | | | | | |
| 196 | 0.886944 | 2.58752 | 1.01763 | 2.52804 | | | | | |
| 197 | 0.950629 | 3.96926 | 0.832412 | 4.00992 | | | | | |
| 198 | 0.963886 | 4.11637 | 1.22933 | 4.03974 | | | | | |
| 199 | 0.979257 | 4.00296 | 0.984703 | 4.0048 | | | | | |
| 200 | 1.00599 | 2.81889 | 1.37861 | 2.72625 | | | | | |
| 201 | 1.00744 | 4.50992 | 0.83164 | 4.61238 | | | | | |
| 202 | 1.04943 | 5.81503 | 1.19163 | 5.75663 | | | | | |
| 203 | 1.15343 | 4.21569 | 0.804476 | 4.43363 | | | | | |
| 204 | 1.16376 | 2.52242 | 2.05165 | 2.25232 | | | | | |
| 205 | 1.24513 | 3.07031 | 1.05951 | 3.13128 | | | | | |
| 206 | 1.27061 | 2.74086 | 1.59252 | 2.63792 | | | | | |
| 207 | 1.31961 | 3.8683 | 1.06371 | 3.91463 | | | | | |
| 208 | 1.33798 | 4.4967 | 1.31481 | 4.50114 | 0.356655 | 5.15115 | | | |
| 209 | 1.34046 | | 1.43869 | | | | | | |
| 210 | 1.40992 | 2.8514 | 1.16727 | 2.96383 | 0.112421 | 3.93915 | | | |
| 211 | 1.42676 | 3.18017 | 1.23093 | 3.77875 | 1.64065 | 3.30502 | | | |
| 212 | 1.47191 | 2.9422 | 1.66319 | 2.88907 | | | | | |
| 213 | 1.49601 | 4.56666 | 1.3192 | 4.66343 | | | | | |
| 214 | 1.50687 | | 1.19206 | | | | | | |
| 215 | 1.52723 | 4.85857 | 1.58239 | 4.84492 | | | | | |
| 216 | 1.5355 | 3.54401 | 1.21737 | 3.64581 | | | | | |
| 217 | 1.54094 | 3.65969 | 2.24195 | 3.52205 | | | | | |
| 218 | 1.60719 | 3.09226 | 1.72038 | 3.13166 | | | | | |
| 219 | 1.62713 | 4.68633 | 1.69382 | 4.66916 | | | | | |
| 220 | 1.67315 | 3.78442 | 2.09175 | 3.68967 | | | | | |
| 221 | 1.69365 | 4.88656 | 2.6866 | 4.73524 | 1.95917 | 4.83618 | | | |
| 222 | 1.70253 | 3.30645 | 1.70913 | 3.30561 | | | | | |
| 223 | 1.86177 | 2.99171 | 2.23944 | 2.91792 | | | | | |
| 224 | 1.9392 | 4.82832 | 1.83565 | 4.82102 | | | | | |
| 225 | 2.0037 | 3.32843 | 2.40329 | 3.24941 | | | | | |
| 226 | 2.03887 | 3.46953 | 1.79442 | 3.56509 | | | | | |
| 227 | 2.05629 | 4.38424 | 1.59622 | 4.49326 | | | | | |
| 228 | 2.12171 | 4.52793 | 1.79625 | 4.5985 | | | | | |
| 229 | 2.25033 | 1.42853 | 1.99108 | 1.48292 | | | | | |
| 230 | 2.38225 | 4.38301 | 2.47936 | 4.36659 | | | | | |
| 231 | 2.39668 | 3.1763 | 2.7258 | 3.07451 | | | | | |
| 232 | 2.40803 | 2.22935 | 2.3469 | 2.10746 | | | | | |
| 233 | 2.44854 | 2.95754 | 3.06477 | 2.95319 | | | | | |
| 234 | 2.4528 | 3.13717 | 2.4202 | 3.14007 | | | | | |
| 235 | 2.50242 | | 2.14999 | | | | | | |
| 236 | 2.54421 | 4.09173 | 2.50081 | 4.09178 | | | | | |
| 237 | 2.55549 | 4.75256 | 2.22851 | 4.8121 | | | | | |
| 238 | 2.66038 | | 3.28325 | | | | | | |
| 239 | 2.74357 | | 3.09688 | | | | | | |
| 240 | 2.81711 | 2.7806 | 5.2 | 2.394 | 1.31115 | 2.9969 | | | |
| 241 | 2.96324 | 2.08714 | 4.36339 | 1.8776 | | | | | |
| 242 | 3.0136 | | 5.2 | | | | | | |
| 243 | 3.11872 | 3.64181 | 2.31144 | 3.73947 | | | | | |
| 244 | 3.15125 | 1.70276 | | | | | | | |
| 245 | 3.16245 | 4.63199 | 3.33204 | 4.62064 | | | | | |
| 246 | 3.26926 | 4.306 | 3.41197 | 4.2875 | | | | | |
| 247 | 3.35666 | 1.81904 | 5.2 | 1.724 | | | | | |
| 248 | 3.3616 | 3.15873 | 3.2978 | 3.16289 | | | | | |
| 249 | 3.43836 | 2.52635 | 4.08444 | 2.37895 | | | | | |
| 250 | 3.43948 | 3.52921 | 3.47307 | 3.53448 | | | | | |
| 251 | 3.7015 | 3.08179 | 4.84289 | 2.9649 | | | | | |
| 252 | 3.72012 | 4.19944 | 5.2 | 4.054 | | | | | |
| 253 | 3.7575 | 1.5751 | 4.83918 | 1.44961 | | | | | |
| 254 | 3.92735 | 3.07495 | | | | | | | |
| 255 | 4.00567 | 3.11066 | 1.92668 | 3.48519 | | | | | |
| 256 | 4.04486 | | 4.55583 | | | | | | |
| 257 | 4.08946 | 4.28028 | 3.4905 | 4.34711 | | | | | |
| 258 | 4.0986 | 3.44029 | 3.89897 | 3.46189 | | | | | |
| 259 | 4.14983 | 1.91665 | 3.24774 | 2.08842 | | | | | |
| 260 | 4.25734 | 4.22086 | 2.3463 | 4.30681 | | | | | |
| 261 | 4.54825 | 4.06308 | 5.2 | 4.034 | | | | | |
| 262 | 4.57025 | 3.64281 | 4.04349 | 3.69327 | 5.13227 | 3.58969 | | | |
| 263 | 4.60867 | 4.41706 | 4.09133 | 4.46892 | | | | | |
| 264 | 4.62297 | 2.62954 | 4.12507 | 2.70457 | | | | | |
| 265 | 4.81433 | 2.64073 | 5.2 | 2.624 | | | | | |
| 266 | 4.84144 | 2.94951 | 5.2 | 2.934 | | | | | |
| 267 | 5.2 | 1.454 | 2.95258 | 1.6998 | | | | | |
| 268 | 5.2 | 2.624 | 5.2 | 2.624 | | | | | |
| 269 | 5.2 | 3.354 | 5.2 | 3.354 | | | | | |
| 270 | 5.2 | 0.783997 | 5.2 | 0.783997 | | | | | |
| 271 | 5.2 | 2.444 | | | | | | | |
| 272 | 5.2 | 3.674 | 5.2 | 3.674 | | | | | |
| 273 | 5.2 | 3.734 | 5.2 | 3.734 | | | | | |
| 274 | 5.2 | 1.914 | 5.2 | 1.914 | | | | | |
| 275 | 5.2 | 4.054 | 5.2 | 4.054 | | | | | |
| 276 | 5.2 | 2.554 | 4.08328 | 2.65899 | | | | | |
| 277 | 5.2 | | | | | | | | |
| 278 | 5.2 | 4.524 | 3.69708 | 4.67214 | 3.67935 | 4.67423 | | | |
| 279 | 5.2 | 4.334 | 5.2 | 4.334 | | | | | |
| 280 | 5.2 | 3.204 | | | 0.258476 | 4.50758 | | | |
| 281 | 5.2 | 2.264 | | | | | | | |
| 282 | 5.2 | 1.004 | 5.2 | 1.004 | | | | | |
| 283 | 5.2 | | 5.2 | | | | | | |
| 284 | 5.2 | 2.424 | 5.2 | 2.424 | | | | | |
| 285 | 5.2 | 2.514 | 5.2 | 2.514 | | | | | |
| 286 | 5.2 | 5.044 | | | 0.973538 | 5.77165 | | | |
| 287 | 5.2 | 5.044 | 5.2 | 5.044 | 1.53591 | 5.57364 | | | |
| 288 | 5.2 | 2.844 | 5.2 | 2.844 | | | | | |
| 289 | 5.2 | 2.234 | 5.2 | 2.234 | | | | | |
| 290 | 5.2 | | 5.2 | | | | | | |
| 291 | 5.2 | 3.954 | 5.2 | 3.954 | | | | | |
| 292 | 5.2 | 2.424 | | | | | | | |
| 293 | 5.2 | 3.394 | 5.2 | 3.394 | 5.2 | 3.394 | | | |
| 294 | 5.2 | | 5.2 | 2.694 | | | | | |
| 295 | 5.2 | | 5.2 | | | | | | |
| 296 | 5.2 | | 5.2 | | | | | | |
| 297 | 5.2 | | 5.2 | | | | | | |
| 298 | 5.2 | | 5.2 | | | | | | |
| 299 | 5.2 | | 5.2 | | 0.489145 | 5.55056 | | | |
| 300 | 5.2 | | 5.2 | | | | | | |
| 301 | 5.2 | | 5.2 | | | | | | |
| 302 | 5.2 | | 5.2 | | | | | | |
| 303 | 5.2 | | 5.2 | | | | | | |
| 304 | 5.2 | | 5.2 | | | | | | |
| 305 | 5.2 | | 5.2 | | | | | | |
| 306 | 5.2 | | 5.2 | 4.594 | | | | | |
| 307 | 5.2 | | 5.2 | | | | | | |
| 308 | 5.2 | | 5.2 | | | | | | |
| 309 | 5.2 | | 5.2 | 2.514 | | | | | |
| 310 | 5.2 | | 5.2 | | | | | | |
| 311 | 5.2 | | 5.2 | | | | | | |
| 312 | 5.2 | | 5.2 | 2.744 | | | | | |
| 313 | 5.2 | | 5.2 | | | | | | |
| 314 | 5.2 | | 5.2 | 2.024 | | | | | |
| 315 | 5.2 | | 5.2 | | | | | | |
| 316 | 5.2 | | 5.2 | 3.634 | | | | | |
| 317 | 5.2 | | 5.2 | | | | | | |
| 318 | 5.2 | | 5.2 | | | | | | |
| 319 | 5.2 | | 5.2 | | | | | | |
| 320 | 5.2 | | 5.2 | | | | | | |
| 321 | 5.2 | | 5.2 | | | | | | |
| 322 | 5.2 | | 5.2 | | | | | | |
| 323 | 5.2 | | 5.2 | | | | | | |
| 324 | 5.2 | | 5.2 | | | | | | |
| 325 | 5.2 | | 5.2 | | | | | | |
| 326 | 5.2 | | 5.2 | 4.034 | 0.990288 | 4.75424 | | | |
| 327 | 5.2 | | 5.2 | | | | | | |
| 328 | 5.2 | | 5.2 | 4.414 | | | | | |

| | | | | | Human | Human | Mouse | Mouse | Rat | Rat |
|---|---|---|---|---|---|---|---|---|---|---|
| | | COX1 10 | | | Plasma | Plasma | Plasma | Plasma | Plasma | Plasma |
| | COX1 10 | minutes | COX1 10 | PBS | Protein | Protein | Protein | Protein | Protein | Protein |
| | minutes | pge2 area | minutes | Solubility | Binding | Stability | Binding | Stability | Binding | Stability |
| Com- | pge2/pge2d4 | (counts) | pge2 area | (no DMSO) | % | % | % | % | % | % |
| pound | area ratio | Avg. IC50 | (counts) | microM | Bound | Remaining | Bound | Remaining | Bound | Remaining |
| No. | Avg. LLE | (microM) | Avg. LLE | Avg | Avg | Avg | Avg | Avg | Avg | Avg |
| 1 | 1.25134 | 13.768 | 1.13618 | 1.1 | 99.8 | 94 | 99.6 | 119.5 | 99.4 | 100 |
| 2 | −0.0460032 | 49.5592 | −0.0251246 | 0.31 | 99.7 | 99 | 99.6 | 108 | 99.1 | 98 |
| 3 | 4.27957 | 0.9135 | 4.32312 | 8.5 | | | 98.5 | 94.9 | | |
| 4 | | | | | | | | | | |
| 5 | | 52 | −0.356003 | | 99.9 | 102 | | | 99.9 | 99 |
| 6 | 1.69414 | 2.9992 | 1.67301 | | | | | | | |
| 7 | 0.563997 | 52 | 0.563997 | | | | 98.5 | 97.8 | | |
| 8 | | | | | | | 100 | 92.8 | | |
| 9 | 0.46745 | | | 3.7 | 99.7 | 114 | | | 99.7 | 121 |
| 10 | 2.51313 | 5.63841 | 2.52894 | | | | | | | |
| 11 | | 42.8048 | 0.848508 | | | | | | | |
| 12 | 1.78447 | 37.6852 | 1.80123 | 7.4 | | | | | 99.4 | 95 |
| 13 | | | | | | | | | 97.9 | 101 |
| 14 | 1.4251 | 32.6944 | 1.51553 | 6.1 | 98 | 118 | 97.8 | 99 | 97.6 | 104 |
| 15 | 1.314 | 34.4324 | 1.40351 | 4.5 | 97.8 | 108 | | | 98 | 127 |
| 16 | 1.13852 | 46.5472 | 1.07211 | 10 | 95.1 | 121 | 98.9 | 102 | 96.9 | 97.9 |
| 17 | | 52 | 0.883997 | 1.3 | 97.8 | 95 | 96.7 | 102 | 95.3 | 100 |
| 18 | 2.39238 | 4.43212 | 2.30818 | | | | 99.8 | 103.7 | | |
| 19 | 2.58207 | 10.8572 | 2.53937 | 5.8 | 98.5 | 92.4 | 98 | 92.8 | 98.5 | 91.1 |
| 20 | 0.213997 | 52 | 0.213997 | | | | | | | |
| 21 | 1.39148 | 52 | 1.134 | 18 | 97.4 | 99 | | | 95.8 | 100 |
| 22 | | | | | | | | | | |
| 23 | 3.12219 | 0.416998 | 3.08512 | 24 | 97.2 | 97 | | | 95.4 | 104 |
| 24 | 1.01984 | 33.3807 | 1.03895 | 2.1 | 98.1 | 100 | 98.4 | 114.1 | 97.5 | 105.1 |
| 25 | 1.374 | 52 | 1.374 | | | | 98.5 | 93.1 | | |
| 26 | 0.949262 | 16.6684 | 0.643072 | | | | 98.2 | 97.5 | | |
| 27 | 1.134 | 52 | 1.134 | | | | | | 96.3 | 99.7 |
| 28 | 1.104 | 52 | 1.104 | | | | | | 97.5 | 102.8 |
| 29 | 1.794 | 0.048374 | 3.30969 | 2.8 | 98.4 | 93.9 | 97.5 | 93.2 | 98.1 | 114.5 |
| 30 | 0.118127 | 34.2882 | 0.214855 | | | | | | | |
| 31 | 1.01294 | 2.71465 | 0.929045 | | | | | | | |
| 32 | | | | | | | 98.5 | 100.9 | | |
| 33 | 1.41762 | 37.865 | 1.36176 | 0.64 | 97.1 | 100 | | | 97.5 | 128 |
| 34 | 1.4613 | 28.6668 | 1.48407 | 12 | 97.3 | 100 | | | 97.5 | 115 |
| 35 | 1.08938 | 52 | 0.723997 | 37 | 97.6 | 97 | 92.1 | 99.2 | 94.8 | 94.1 |
| 36 | 1.49159 | 29.7055 | 1.69547 | 27 | 90.9 | 100 | 88.2 | 106 | 91.3 | 102.8 |
| 37 | 2.05624 | 33.1147 | 2.12998 | | | | | | | |
| 38 | 3.52572 | 1.23274 | 3.30566 | | | | | | | |
| 39 | | 52 | | | | | | | | |
| 40 | | | | | | | | | | |
| 41 | 0.233997 | 49.6284 | 0.254269 | | | | | | 97.9 | 100 |
| 42 | 0.011874 | 38.677 | −0.0174524 | | | | | | | |
| 43 | | 42.6628 | 2.43995 | 21 | | | 95.4 | 102.6 | 97.2 | 107.3 |
| 44 | 2.274 | 38.0721 | 2.40979 | 28 | | | 95.5 | 105.7 | 95.6 | 104.6 |
| 45 | 1.32768 | 7.05653 | 1.30141 | | | | | | | |
| 46 | 0.0431514 | 34.6613 | 0.210155 | | | | | | | |
| 47 | 1.46137 | | | 88 | 92.9 | 102.5 | 93 | 107.5 | 93.2 | 105.1 |
| 48 | | | | 40 | 98 | 96 | | | 96.9 | 128 |
| 49 | 1.734 | 52 | 1.734 | | | | | | | |
| 50 | 1.09126 | 41.9669 | 0.978519 | | | | 96.9 | 98.9 | | |
| 51 | 3.09382 | 2.23434 | 3.23415 | | | | 98.5 | 109.5 | | |
| 52 | 1.0149 | 52 | 1.014 | | | | | | | |
| 53 | 2.12519 | 11.6894 | 2.0112 | | | | 99.9 | 36.7 | | |
| 54 | 0.517128 | 51.9949 | 0.39404 | | | | | | | |
| 55 | | | | | | | | | | |
| 56 | | | | | | | | | | |
| 57 | −0.196003 | 50.422 | −0.18262 | | 99.6 | 104 | | | 99.3 | 102 |
| 58 | 2.5036 | 52 | 2.324 | | | | 96.2 | 103.1 | | |
| 59 | 1.754 | 52 | 1.754 | 15 | 94.5 | 100 | | | 92.6 | 117 |
| 60 | 2.694 | 52 | 2.694 | | | | | | 95.7 | 100 |
| 61 | 0.213997 | 52 | 0.213997 | | | | | | | |
| 62 | | | | | | | | | | |
| 63 | 2.0906 | 1.82945 | 2.12768 | | | | | | | |
| 64 | 3.4205 | 4.75708 | 3.47266 | | | | | | | |
| 65 | 1.544 | 37.9993 | 1.61211 | | | | | | | |
| 66 | 1.364 | 52 | 1.364 | | | | | | 89.5 | 98.1 |
| 67 | 0.378743 | 21.2307 | 0.343036 | | | | | | | |
| 71 | 0.0467865 | | | | | | | | | |
| 72 | 0.254059 | 30.1897 | 0.0201411 | | | | | | | |
| 73 | 2.18298 | 17.0856 | 2.12737 | | | | 90.7 | 96.1 | 89.9 | 86.9 |
| 74 | 3.04587 | 0.34199 | 2.99599 | 0.72 | 98.2 | 97.5 | 97.1 | 97 | | |
| 75 | | | | | | | | | | |
| 76 | 1.46076 | | | | | | | | | |
| 77 | | 40.8535 | 1.28877 | | | | | | 94.1 | 108 |
| 78 | 1.92451 | 52 | 1.734 | | | | 98.7 | 106.5 | | |
| 79 | | | | | | | 93.1 | 111.8 | | |
| 80 | 0.442204 | | | | | | | | | |
| 81 | | | | | | | | | | |
| 82 | 1.86504 | 17.6406 | 1.85349 | | | | 93.4 | 120 | | |
| 83 | 0.283997 | | | | | | | | | |
| 84 | | 1.7889 | 2.20741 | | | | | | | |
| 85 | 1.134 | 52 | 1.134 | | | | | | | |
| 86 | | | | | | | | | | |
| 87 | 1.97034 | 50.2422 | 1.89893 | | | | | | | |
| 88 | 2.30588 | 16 | 2.30588 | | | | | | | |
| 89 | | | | | | | | | | |
| 90 | | | | | | | | | | |
| 91 | 1.26679 | 52 | 1.134 | | | | | | | |
| 92 | | | | | | | | | | |
| 93 | | 52 | 2.104 | 29 | 94.3 | 106.5 | 94.4 | 120.6 | 94.4 | 109.5 |
| 94 | 2.55206 | 10.6649 | 2.53204 | | | | | | | |
| 95 | | | | | | | | | | |
| 96 | 2.18647 | 39.0415 | 2.12847 | | | | | | | |
| 97 | | | | | | | | | | |
| 98 | 2.94315 | 10.7136 | 2.87927 | 11 | 94.5 | 100.2 | 93 | 87.6 | 92.6 | 94.6 |
| 99 | 1.49791 | | | | | | | | | |
| 106 | | | | | | | 91.2 | 114.2 | | |
| 109 | | | | 30 | 96.3 | 105.7 | 95.1 | 108.8 | 95 | 105.8 |
| 110 | | | | | | | 87.1 | 98.8 | | |
| 114 | | | | | | | 86.8 | 114.1 | 89 | 103.5 |
| 120 | | 52 | 2.724 | 32 | 90.2 | 94.9 | 88.3 | 101.7 | 90.6 | 106.3 |
| 127 | | | | 37 | | | | | | |
| 128 | | | | | | | | | | |
| 129 | | 50.157 | 2.82967 | | | | | | | |
| 130 | | | | 0.84 | 99.2 | 94 | 99.1 | 91.6 | | |
| 141 | | | | 13 | 88.7 | 101.5 | 81.2 | 106.1 | | |
| 142 | | | | | | | | | | |
| 143 | | | | | | | 97.6 | 106.1 | | |
| 156 | | | | | 97.35 | 100.3 | 97.53 | 98.78 | | |
| 232 | | | | | | | 95.6 | 114.8 | | |
| 253 | | 19.3326 | 0.863709 | | | | | | | |

| | Human | Mouse | Rat | | | Mouse | MDR1- | MDR1- | |
|---|---|---|---|---|---|---|---|---|---|
| | Microsome | Microsome | Microsome | | | brain | MCKII | MCKII | |
| | Stability | Stability | Stability | | | protein | Papp | Papp | MDR1- |
| Com- | % | % | % | | | binding | A-B | B-A | MDCKII |
| pound | Remaining | Remaining | Remaining | | | % | (10−6 | (10−6 | Efflux |
| No. | Avg | Avg | Avg | CNS_MPO_6 | CNS_PET_MPO | MEAN | cm/s) | cm/s) | ratio |
| 1 | 38 | | | 4.406 | 2.113704277 | 100 | 33.7 | 29.9 | 0.9 |
| 2 | 27 | | | 3.467700429 | 2 | | | | |
| 3 | | | | 4.486 | 2.770136323 | | | | |
| 4 | | | | 4.519268905 | 2.578333333 | | | | |
| 5 | | | | 3.633166429 | 2 | | | | |
| 6 | | | | 4.277526 | 2 | | | | |
| 7 | | | | 3.9895 | 2 | | | | |
| 8 | | | | 4.179167095 | 2.525 | | | | |
| 9 | 35 | | | 4.484 | 2.178704277 | | | | |
| 10 | | | | 4.637448524 | 2.555 | | | | |
| 11 | | | | 4.399 | 2.346324336 | | | | |
| 12 | | | | 5.0575 | 3.118359694 | | | | |
| 13 | 62 | | | 4.188833333 | 2.819213974 | | | | |
| 14 | 21 | | | 4.863333333 | 2.778320452 | | | | |
| 15 | 45.5 | | | 4.863333333 | 2.778320452 | | | | |
| 16 | 24 | | | 5.138 | 3.366823495 | | | | |
| 17 | | | 0.2 | 3.925925333 | 2.160833333 | 98.8 | | | |
| 18 | | | | 4.076217429 | 2.495 | | | | |
| 19 | | | | 5.050833333 | 3.051011209 | | | | |
| 20 | | | | 2.932671048 | 2 | | | | |
| 21 | 36 | | | 5.001333333 | 2.981016912 | | | | |
| 22 | | | | 5.300254714 | 3.06 | | | | |
| 23 | 11 | | | 4.896333333 | 2.805820452 | | | | |
| 24 | 18 | | 6.9 | 4.321 | 2.281324336 | | | | |
| 25 | | | | 3.652344571 | 2.38 | | | | |
| 26 | 43 | | | 4.962 | 2.940458515 | | | | |
| 27 | 8.8 | | | 5.001333333 | 2.981016912 | | | | |
| 28 | 15 | | | 5.06 | 3.301823495 | | | | |
| 29 | 24 | 4.3 | 2.2 | 4.101534429 | 2.670833333 | | | | |
| 30 | | | | 4.294526 | 2 | | | | |
| 31 | | | | 4.352481857 | 2 | | | | |
| 32 | | | | 3.925925333 | 2.160833333 | | | | |
| 33 | 40 | | | 4.941333333 | 2.843320452 | | | | |
| 34 | 48 | | | 4.941333333 | 2.843320452 | | | | |
| 35 | 19 | | | 4.83 | 3.601459172 | 98.9 | | | |
| 36 | 17 | | 11.6 | 5.068333333 | 3.347172812 | 98.1 | 68.5 | 67.9 | 1 |
| 37 | | | | 4.698333333 | 2.693511209 | | | | |
| 38 | | | | 4.841448524 | 2.725 | | | | |
| 39 | | | | 3.467700429 | 2 | | | | |
| 40 | | | | 4.660333333 | 2.696850245 | | | | |
| 41 | | | | 4.24 | 2.311636898 | | | | |
| 42 | | | | 4.330333333 | 2.304592368 | | | | |
| 43 | | | 11.1 | 5.091833333 | 3.148181818 | | | | |
| 44 | | | 5.3 | 5.130833333 | 3.219090909 | | 39.6 | 47.2 | 1.2 |
| 45 | | | | 4.277526 | 2 | | | | |
| 46 | | | | 4.163 | 2 | | | | |
| 47 | 16 | | 8.2 | 5.146333333 | 3.412172812 | 97.8 | 50.8 | 47.2 | 0.9 |
| 48 | 34 | | | 4.941333333 | 2.843320452 | | | | |
| 49 | | | | 4.810922905 | 2.454166667 | | | | |
| 50 | | | | 3.926425333 | 2.160833333 | | | | |
| 51 | | | | 5.148 | 2.811666667 | | | | |
| 52 | | | | 4.96 | 3.191526373 | | | | |
| 53 | | | | 4.63911519 | 2.556666667 | | | | |
| 54 | | | | 3.815925333 | 2.069166667 | | | | |
| 55 | | | | 5.291388905 | 2.8875 | | | | |
| 56 | | | | 4.231981857 | 2 | | | | |
| 57 | | | | 3.736401095 | 2 | | | | |
| 58 | | | | 5.872166667 | 3.98106605 | | | | |
| 59 | 28 | | | 4.502833333 | 2.768631268 | | | | |
| 60 | | | | 4.42381019 | 3.196363636 | | | | |
| 61 | | | | 4.884 | 2.875458515 | | | | |
| 62 | | | | 3.515344571 | 2.265833333 | | | | |
| 63 | | | | 4.526090952 | 2.396666667 | | | | |
| 64 | | | | 3.861143524 | 2.385454545 | | | | |
| 65 | | | | 4.913 | 2.704256785 | | | | |
| 66 | | | | 5.163333333 | 2.691666667 | | | | |
| 67 | | | | 3.467700429 | 2 | | | | |
| 68 | | | | 4.580788048 | 2.359166667 | | | | |
| 69 | | | | 4.033493143 | 2 | | | | |
| 70 | | | | 4.3745 | 3.49720153 | | | | |
| 71 | | | | 4.831090952 | 2.650833333 | | | | |
| 72 | | | | 4.024312762 | 2.603930131 | | | | |
| 73 | | | | 4.745 | 2.261572347 | | | | |
| 74 | | | | 4.598090952 | 2.456666667 | | 32.2 | 32 | 1 |
| 75 | | | | 3.174154714 | 2.065 | | | | |
| 76 | | | | 3.311139262 | 2 | | | | |
| 77 | | | | 4.872333333 | 3.253818922 | | | | |
| 78 | | | | 5.0185 | 3.051011209 | | | | |
| 79 | | | | 5.216 | 3.087991003 | | | | |
| 80 | | | | 3.870176857 | 2.4125 | | | | |
| 81 | | | | 5.261254714 | 2.99 | | | | |
| 82 | | | | 5.5535 | 2.9225 | | | | |
| 83 | | | | 4.765322048 | 2.434297671 | | | | |
| 84 | | | | 4.600333333 | 3.079902655 | | | | |
| 85 | | | | 4.582333333 | 2.631850245 | | | | |
| 86 | | | | 4.169724286 | 2.145454545 | | | | |
| 87 | | | | 4.541333333 | 2.801131268 | | | | |
| 88 | | | | 4.101534429 | 2.670833333 | | | | |
| 89 | | | | 4.584333333 | 2.633516912 | | | | |
| 90 | | | | 3.930924952 | 2.304545455 | | | | |
| 91 | | | | 4.582333333 | 2.631850245 | | | | |
| 92 | | | | 3.873176857 | 2.415 | | | | |
| 93 | | | | 5.366833333 | 3.390819783 | | | | |
| 94 | | | | 4.712366714 | 2.998333333 | | | | |
| 95 | | | | 4.268 | 2.941484716 | | | | |
| 96 | 54.9 | 30.5 | | 5.195333333 | 2.974665211 | | | | |
| 97 | | | | 4.430835762 | 2.6175 | | | | |
| 98 | 23.8 | 3 | 20.3 | 4.877833333 | 3.227419147 | 98 | 49.7 | 83.8 | 1.7 |
| 99 | | | | 4.905333333 | 3.277419147 | | | | |
| 100 | | | | 4.005735476 | 2.310833333 | | | | |
| 101 | | | | 4.277526 | 2 | | | | |
| 102 | | | | 4.44281019 | 3.230909091 | | | | |
| 103 | | | | 4.736833333 | 2.870833333 | | | | |
| 104 | | | | 4.916833333 | 3.298328238 | | | | |
| 105 | | | | 4.848333333 | 3.173782784 | | | | |
| 106 | | | | 4.166666667 | 2.951013958 | | | | |
| 107 | | | | 4.66381019 | 3.632727273 | | | | |
| 108 | | | | 4.950333333 | 3.318818922 | | | | |
| 109 | | | | 4.557333333 | 2.7025 | | | | |
| 110 | | | | 5.699666667 | 4.161943685 | | | | |
| 111 | | | | 4.464481857 | 2.020833333 | | | | |
| 112 | | | | 4.769333333 | 2.787683579 | | | | |
| 113 | | | | 5.430166667 | 3.671943685 | | | | |
| 114 | | | | 4.478833333 | 3.17283214 | | | | |
| 115 | | | | 4.92 | 3.366136606 | | | | |
| 116 | | | | 4.101534429 | 2.670833333 | | | | |
| 117 | | | | 4.923333333 | 2.916016912 | | | | |
| 118 | | | | 3.476605262 | 2 | | | | |
| 119 | | | | 4.841448524 | 2.725 | | | | |
| 120 | 32.4 | 1.6 | | 5.660666667 | 4.091034594 | | | | |
| 121 | | | | 4.299582714 | 2 | | | | |
| 122 | | | | 3.901867095 | 2 | | | | |
| 123 | | | | 4.522167095 | 2.810833333 | | | | |
| 124 | | | | 4.97461519 | 2.839166667 | | | | |
| 125 | | | | 4.687322048 | 2.369297671 | | | | |
| 126 | | | | 3.946176857 | 2.475833333 | | | | |
| 127 | | | | 4.833333333 | 3.86283214 | | | | |
| 128 | | | | 4.47931019 | 3.297272727 | | | | |
| 129 | | | | 5.133833333 | 3.668660901 | | | | |
| 130 | | | | 3.626013643 | 2.37860262 | | | | |
| 131 | | | | 5.085333333 | 3.063636364 | | | | |
| 132 | | | | 5.399 | 3.661626366 | | | | |
| 133 | | | | 3.844206619 | 2.78 | | | | |
| 134 | | | | 5.089833333 | 3.118359694 | | | | |
| 135 | | | | 5.319333333 | 3.535632422 | | | | |
| 136 | | | | 5.449166667 | 3.706489139 | | | | |
| 137 | | | | 4.288724286 | 2.361818182 | | | | |
| 138 | | | | 5.044666667 | 4.068318142 | | | | |
| 139 | | | | 3.688452619 | 2.4475 | | | | |
| 140 | | | | 3.621743048 | 2 | | | | |
| 141 | 55 | 10 | | 4.802833333 | 3.761923049 | | | | |
| 142 | | | | 4.821833333 | 3.796468504 | | | | |
| 143 | | | | 5.833166667 | 3.910156959 | | | | |
| 144 | | | | 3.311139262 | 2 | | | | |
| 145 | | | | 4.736833333 | 2.870833333 | | | | |
| 146 | | | | 4.2865 | 3.046056637 | | | | |
| 147 | | | | 5.044666667 | 4.017409051 | | | | |
| 148 | | | | 4.844833333 | 3.491045414 | | | | |
| 149 | | | | 5.084833333 | 2.625833333 | | | | |
| 150 | | | | 5.341666667 | 3.511034594 | | | | |
| 151 | | | | 3.684932905 | 2 | | | | |
| 152 | | | | 4.91589781 | 3.614545455 | | | | |
| 153 | | | | 4.945666667 | 3.615590869 | | | | |
| 154 | | | | 5.351833333 | 3.327343282 | | | | |
| 155 | | | | 3.702090952 | 1.590289855 | | | | |
| 156 | | | | 3.596534429 | 2.25 | | | | |
| 157 | | | | 4.598 | 2.547673304 | | | | |
| 158 | | | | 4.101534429 | 2.670833333 | | | | |
| 159 | | | | 4.268700048 | 2.85 | | | | |
| 160 | | | | 3.925925333 | 2.160833333 | | | | |
| 161 | | | | 5.684333333 | 4.191036448 | | | | |
| 162 | | | | 5.085833333 | 3.338220917 | | | | |
| 163 | | | | 4.71581019 | 3.736363636 | | | | |
| 164 | | | | 3.679866714 | 2.030434783 | | | | |
| 165 | | | | 3.930924952 | 2.304545455 | | | | |
| 166 | | | | 5.190198667 | 3.447272727 | | | | |
| 167 | | | | 5.085833333 | 3.338220917 | | | | |
| 168 | | | | 3.768533762 | 2.1975 | | | | |
| 169 | | | | 4.268700048 | 2.85 | | | | |
| 170 | | | | 5.258833333 | 3.158252373 | | | | |
| 171 | | | | 4.633333333 | 3.453741231 | | | | |
| 172 | | | | 5.393 | 3.3463038 | | | | |
| 173 | | | | 4.557333333 | 2.7025 | | | | |
| 174 | | | | 5.723333333 | 4.261945539 | | | | |
| 175 | | | | 5.410166667 | 3.635580048 | | | | |
| 176 | | | | 4.932866714 | 3.399090909 | | | | |
| 177 | | | | 4.92 | 3.366136606 | | | | |
| 178 | | | | 5.562833333 | 4.489678901 | | | | |
| 179 | | | | 4.468534429 | 3.130909091 | | | | |
| 180 | | | | 5.126333333 | 3.339011671 | | | | |
| 181 | | | | 5.821666667 | 3.887943202 | | | | |
| 182 | | | | 5.191698667 | 3.45 | | | | |
| 183 | | | | 4.97789781 | 3.739090909 | | | | |
| 184 | | | | 3.829176857 | 2.5225 | | | | |
| 185 | | | | 4.823 | 2.326572347 | | | | |
| 186 | | | | 5.351833333 | 3.327343282 | | | | |
| 187 | | | | 2.899185476 | 2 | | | | |
| 188 | | | | 5.359333333 | 3.096577091 | | | | |
| 189 | | | | 4.005735476 | 2.310833333 | | | | |
| 190 | | | | 3.668022952 | 2.146666667 | | | | |
| 191 | | | | 3.273328952 | 2 | | | | |
| 192 | | | | 4.694333333 | 3.474545455 | | | | |
| 193 | | | | 4.913333333 | 3.615590869 | | | | |
| 194 | | | | 4.212833333 | 3.396717514 | | | | |
| 195 | | | | 2.899185476 | 2 | | | | |
| 196 | | | | 4.354925333 | 2.518333333 | | | | |
| 197 | | | | 5.427833333 | 3.210743758 | | | | |
| 198 | | | | 5.258833333 | 3.158252373 | | | | |
| 199 | | | | 5.130833333 | 3.219090909 | | | | |
| 200 | | | | 4.882 | 3.126526373 | | | | |
| 201 | | | | 4.833333333 | 4.090104867 | | | | |
| 202 | | | | 5.288 | 4.445538496 | | | | |
| 203 | | | | 4.833333333 | 4.090104867 | | | | |
| 204 | | | | 4.649867095 | 2.658333333 | | | | |
| 205 | | | | 4.401166667 | 2.4425 | | | | |
| 206 | | | | 4.169666667 | 2.0225 | | | | |
| 207 | | | | 4.216577048 | 2.965833333 | | | | |
| 208 | | | | 5.5415 | 3.391818182 | | | | |
| 209 | | | | 3.768533762 | 2.1975 | | | | |
| 210 | | | | 4.145366714 | 2.426086957 | | | | |
| 211 | | | | 5.194 | 3.092774007 | | | | |
| 212 | | | | 5.569356524 | 3.047272727 | | | | |
| 213 | | | | 4.952333333 | 3.68649996 | | | | |
| 214 | | | | 3.273328952 | 2 | | | | |
| 215 | | | | 5.288 | 4.445538496 | | | | |
| 216 | | | | 5.688666667 | 4.141943685 | | | | |
| 217 | | | | 5.012333333 | 4.017409051 | | | | |
| 218 | 15 | | | 4.863333333 | 2.778320452 | | | | |
| 219 | | | | 3.91185281 | 2.633636364 | | | | |
| 220 | | | | 4.805833333 | 3.097797935 | | | | |
| 221 | | | | 5.6245 | 3.542727273 | | | | |
| 222 | | | | 4.874534429 | 4 | | | | |
| 223 | | | | 4.913 | 2.704256785 | | | | |
| 224 | | | | 5.833333333 | 4.370927273 | | | | |
| 225 | | | | 5.2965 | 2.991324336 | | | | |
| 226 | | | | 5.012333333 | 4.068318142 | | | | |
| 227 | | | | 4.97789781 | 4 | | | | |
| 228 | | | | 5.084198667 | 3.254545455 | | | | |
| 229 | | | | 4.163 | 2 | | | | |
| 230 | | | | 5.191698667 | 3.45 | | | | |
| 231 | | | | 5.630666667 | 4.036489139 | | | | |
| 232 | | | | 5.3735 | 2.920370944 | | | | |
| 233 | | | | 4.750833333 | 3.006131268 | | | | |
| 234 | | | | 4.978333333 | 2.931011209 | | | | |
| 235 | | | | 5.468333333 | 3.798309175 | | | | |
| 236 | | | | 4.97789781 | 4 | | | | |
| 237 | | | | 5.561666667 | 4.157002655 | | | | |
| 238 | | | | 4.87925519 | 2.525214702 | | | | |
| 239 | | | | 5.438166667 | 3.686489139 | | | | |
| 240 | | | | 4.673425476 | 2.679665211 | | | | |
| 241 | | | | 4.769333333 | 2.787683579 | | | | |
| 242 | | | | 5.507333333 | 3.869218266 | | | | |
| 243 | | | | 5.553 | 4.249411251 | | | | |
| 244 | | | | 4.723090952 | 2.5625 | | | | |
| 245 | | | | 5.561666667 | 4.068820837 | | | | |
| 246 | | | | 5.288 | 4.445538496 | | | | |
| 247 | | | | 4.83 | 3.601459172 | | | | |
| 248 | | | | 4.216577048 | 2.965833333 | | | | |
| 249 | | | | 4.63911519 | 2.556666667 | | | | |
| 250 | | | | 5.391166667 | 3.601034594 | | | | |
| 251 | | | | 3.933326905 | 2.463333333 | | | | |
| 252 | | | | 6 | 4.190049638 | | | | |
| 253 | | | | 4.637 | 2.912282596 | | | | |
| 254 | | | | 5.620166667 | 4.01739823 | | | | |
| 255 | | | | 5.705666667 | 3.678338777 | | | | |
| 256 | | | | 4.508166667 | 2.973333333 | | | | |
| 257 | | | | 3.91185281 | 2.633636364 | | | | |
| 258 | | | | 5.012333333 | 3.997409051 | | | | |
| 259 | | | | 3.925925333 | 2.160833333 | | | | |
| 260 | | | | 5.084198667 | 3.254545455 | | | | |
| 261 | | | | 4.289652143 | 3 | | | | |
| 262 | | | | 5.69 | 4.535980063 | | | | |
| 263 | | | | 5.833333333 | 4.370927273 | | | | |
| 264 | | | | 4.925 | 2.541912228 | | | | |
| 265 | | | | 3.53760819 | 2.5425 | | | | |
| 266 | | | | 3.933326905 | 2.463333333 | | | | |
| 267 | | | | 4.8345 | 2.471204277 | | | | |
| 268 | | | | 3.53760819 | 2.5425 | | | | |
| 269 | | | | 5.091833333 | 3.148181818 | | | | |
| 270 | | | | 4.461481857 | 2.020833333 | | | | |
| 271 | | | | 4.588333333 | 2.601844543 | | | | |
| 272 | | | | 5.400333333 | 3.709090909 | | | | |
| 273 | | | | 5.828 | 4.793148075 | | | | |
| 274 | | | | 4.584333333 | 2.633516912 | | | | |
| 275 | | | | 6 | 4.190049638 | | | | |
| 276 | | | | 4.721333333 | 2.535833333 | | | | |
| 277 | | | | 5.399166667 | 3.615580048 | | | | |
| 278 | | | | 6 | 5.6374474 | | | | |
| 279 | | | | 4.866333333 | 3.467325221 | | | | |
| 280 | | | | 5.5735 | 4.310541706 | | | | |
| 281 | | | | 4.4235 | 2.7075 | | | | |
| 282 | | | | 4.192833333 | 3.997379913 | | | | |
| 283 | | | | 5.415333333 | 3.704672812 | | | | |
| 284 | | | | 4.401166667 | 2.4425 | | | | |
| 285 | | | | 4.906 | 2.716935953 | | | | |
| 286 | | | | 6 | 4.92564047 | | | | |
| 287 | | | | 6 | 4.898676682 | | | | |
| 288 | | | | 4.645366714 | 2.886666667 | | | | |
| 289 | | | | 5.474333333 | 3.872905149 | | | | |
| 290 | | | | 5.453833333 | 3.771945539 | | | | |
| 291 | | | | 4.97789781 | 4 | | | | |
| 292 | | | | 4.401166667 | 2.4425 | | | | |
| 293 | | | | 5.642 | 4.431072009 | | | | |
| 294 | | | | 5.187 | 2.95110262 | | | | |
| 295 | | | | 4.231833333 | 3.997379913 | | | | |
| 296 | | | | 3.705227762 | 2 | | | | |
| 297 | | | | 5.163333333 | 2.691666667 | | | | |
| 298 | | | | 5.833333333 | 4.204511321 | | | | |
| 299 | | | | 6 | 5.37860262 | | | | |
| 300 | | | | 4.401166667 | 2.4425 | | | | |
| 301 | | | | 4.725090952 | 2.5625 | | | | |
| 302 | | | | 4.146 | 2.475833333 | | | | |
| 303 | | | | 4.720936 | 3.46 | | | | |
| 304 | | | | 5.9795 | 3.767292576 | | | | |
| 305 | | | | 4.289652143 | 3 | | | | |
| 306 | | | | 5.418333333 | 3.495815669 | | | | |
| 307 | | | | 5.418333333 | 3.495815669 | | | | |
| 308 | | | | 4.354090952 | 2.506666667 | | | | |
| 309 | | | | 4.906 | 2.716935953 | | | | |
| 310 | | | | 3.85811519 | 2.020833333 | | | | |
| 311 | | | | 3.39051881 | 2.2025 | | | | |
| 312 | | | | 4.87511519 | 2.753333333 | | | | |
| 313 | | | | 5.384 | 3.440174672 | | | | |
| 314 | | | | 4.92 | 3.366136606 | | | | |
| 315 | | | | 4.945 | 2.562870944 | | | | |
| 316 | | | | 4.127592667 | 3 | | | | |
| 317 | | | | 4.586090952 | 2.448333333 | | | | |
| 318 | | | | 4.604333333 | 2.615177876 | | | | |
| 319 | | | | 4.798882857 | 2.957674672 | | | | |
| 320 | | | | 4.79 | 2.6463559 | | | | |
| 321 | | | | 4.872333333 | 2.838511209 | | | | |
| 322 | | | | 4.92 | 3.366136606 | | | | |
| 323 | | | | 5.761166667 | 3.779247868 | | | | |
| 324 | | | | 5.100080286 | 3.172674672 | | | | |
| 325 | | | | 4.586590952 | 2.448333333 | | | | |
| 326 | | | | 5.918 | 5.218356491 | | | | |
| 327 | | | | 4.481333333 | 3.42131432 | | | | |
| 328 | | | | 5.872 | 3.571838031 | | | | |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of the formula: 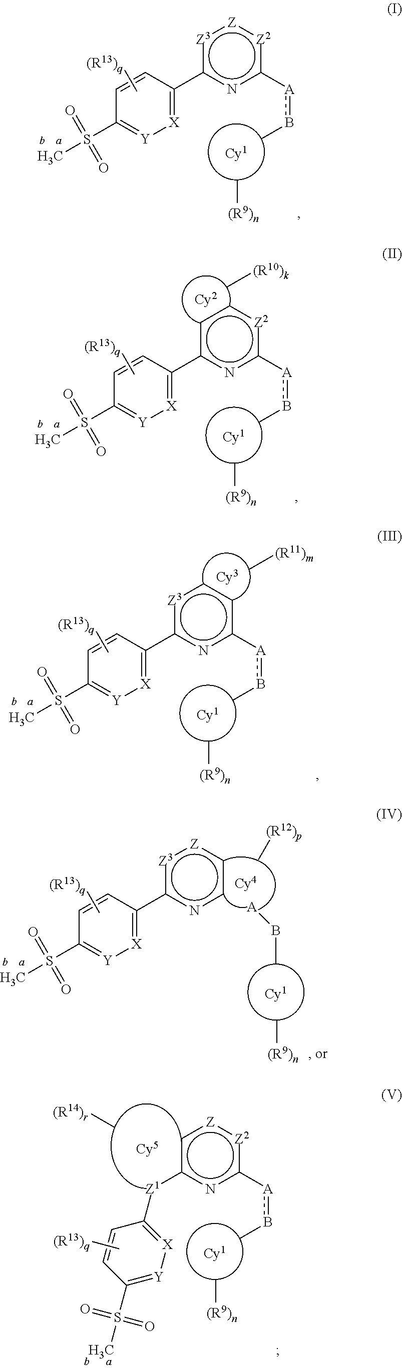
or a salt, solvate, hydrate, polymorph, co-crystal, tautomer, stereoisomer, isotopically labeled derivative, or prodrug thereof, wherein:
Y is CR¹³ or N;
each instance of R¹³ is independently hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
each instance of R^{a} is independently hydrogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form substituted or unsubstituted heterocyclyl or substituted or unsubstituted heteroaryl;
q is 0, 1, or 2;
X is CR¹³ or N;
Z³ is CR¹ or N;
R¹ is hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
Z is CH, CF, or N;
Cy² is monocyclic heteroaryl, monocyclic carbocyclyl, monocyclic heterocyclyl, or phenyl;
each instance of R¹⁰ is independently hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂, or two instances of R¹⁰ on the same carbon atom taken together with the carbon atom form C(═O);
k is 0, 1, 2, 3, 4, 5, 6, 7, or 8, as valency permits;
Z² is CR² or N;
R² is hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
Cy³ is monocyclic heteroaryl, monocyclic carbocyclyl, monocyclic heterocyclyl, or phenyl;
each instance of R¹¹ is independently hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂, or two instances of R¹¹ on the same carbon atom taken together with the carbon atom form C(═O);
m is 0, 1, 2, 3, 4, 5, 6, 7, or 8, as valency permits;
Cy⁴ is monocyclic heteroaryl, monocyclic carbocyclyl, monocyclic heterocyclyl, or phenyl;
each instance of R¹² is independently hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂, or two instances of R¹² on the same carbon atom taken together with the carbon atom form C(═O);
p is 0, 1, 2, 3, 4, 5, or 6, as valency permits;
Z¹ is C, CR¹⁵, or N;
R¹⁵ is hydrogen, halogen, or substituted or unsubstituted alkyl;
Cy⁵ is monocyclic heteroaryl, monocyclic carbocyclyl, monocyclic heterocyclyl, or phenyl;
each instance of R¹⁴ is independently hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂, or two instances of R¹⁴ on the same carbon atom taken together with the carbon atom form C(═O);
r is 0, 1, 2, 3, 4, 5, or 6, as valency permits; [symbol]
is a single bond or double bond;
in any one of Formulae (I) to (III) and (V), A is O, NR¹⁶, S, S(═O), C(═O), or C(R¹⁹)₂, or A and B are joined to form substituted or unsubstituted, monocyclic carbocyclyl, or 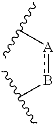
is 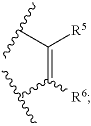
R¹⁶ is hydrogen, substituted or unsubstituted acyl, substituted or unsubstituted alkyl, or a nitrogen protecting group;
each instance of R¹⁹ is independently hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂:
in Formula (IV), A is N, C, or CR¹⁷;
R¹⁷ is hydrogen, halogen, or substituted or unsubstituted alkyl;
B is C(R³)(R⁴), C(═O), NR¹⁸, or a single bond;
R³ is hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
R⁴ is hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
R¹⁸ is hydrogen, substituted or unsubstituted acyl, substituted or unsubstituted alkyl, or a nitrogen protecting group;
R⁵ is hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
R⁶ is hydrogen, halogen, substituted or unsubstituted alkyl, substituted or unsubstituted alkenyl, substituted or unsubstituted alkynyl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂;
Cy¹ is heteroaryl, aryl, carbocyclyl, or heterocyclyl;
each instance of R⁹ is independently hydrogen, halogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted phenyl, substituted or unsubstituted, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —S(═O)R^{a}, —S(═O)OR^{a}, —S(═O)N(R^{a})₂, —S(═O)₂R^{a}, —S(═O)₂OR^{a}, or —S(═O)₂N(R^{a})₂, or two instances of R⁹ on the same carbon atom taken together with the carbon atom form C(═O); and
n is 0, 1, 2, 3, 4, 5, 6, 7, 8, 9, 10, or 11, as valency permits;
provided that:
the compound is not of the formula: 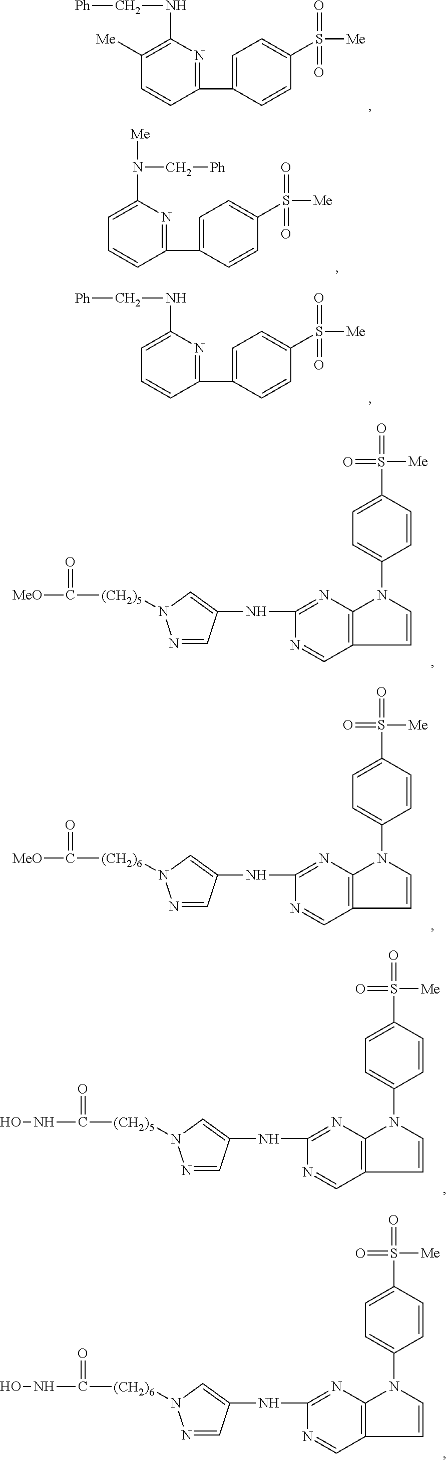 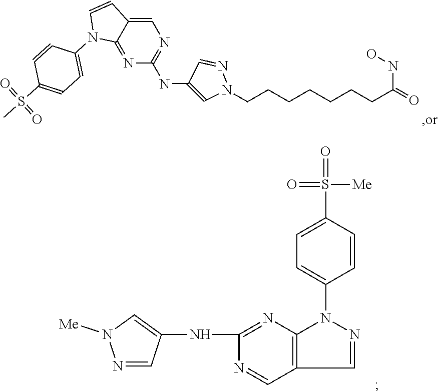 [SMILES: CS(=O)(=O)c1ccc(-n2ccc3cnc(Nc4cnn(CCCCCCCC(=O)NO)c4)nc32)cc1 ; ",or" ; Cn1cc(Nc2ncc3cnn(-c4ccc(S(C)(=O)=O)cc4)c3n2)cn1 ; ";"]
and
the molecular weight of the compound is not more than 500 g/mol.

2-64. (canceled)

65. The compound of claim 1, or a salt, solvate, hydrate, polymorph, co-crystal, tautomer, stereoisomer, isotopically labeled derivative, or prodrug thereof, wherein the compound is of the formula:

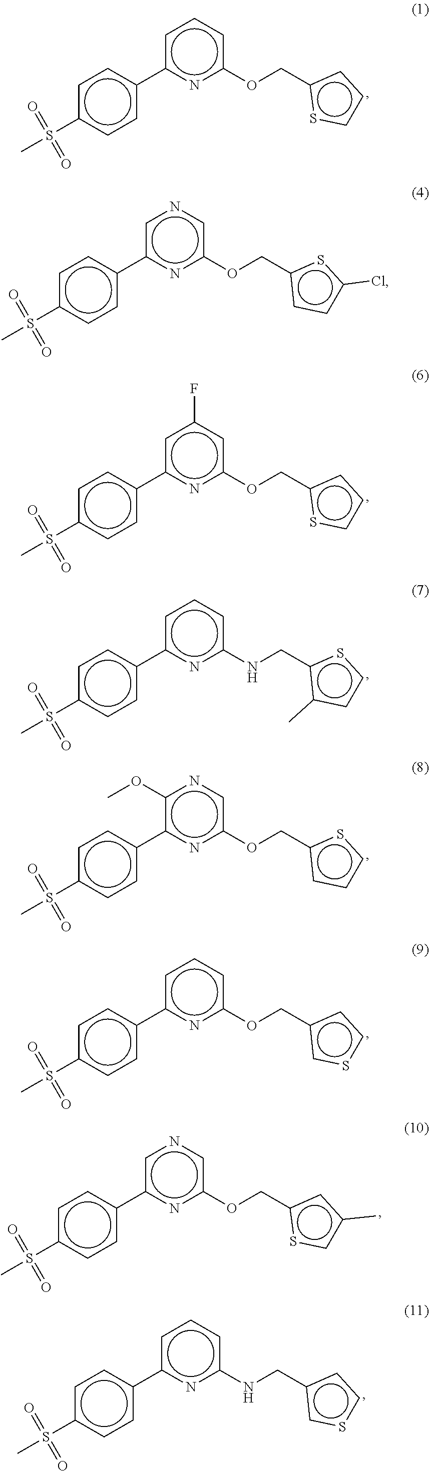

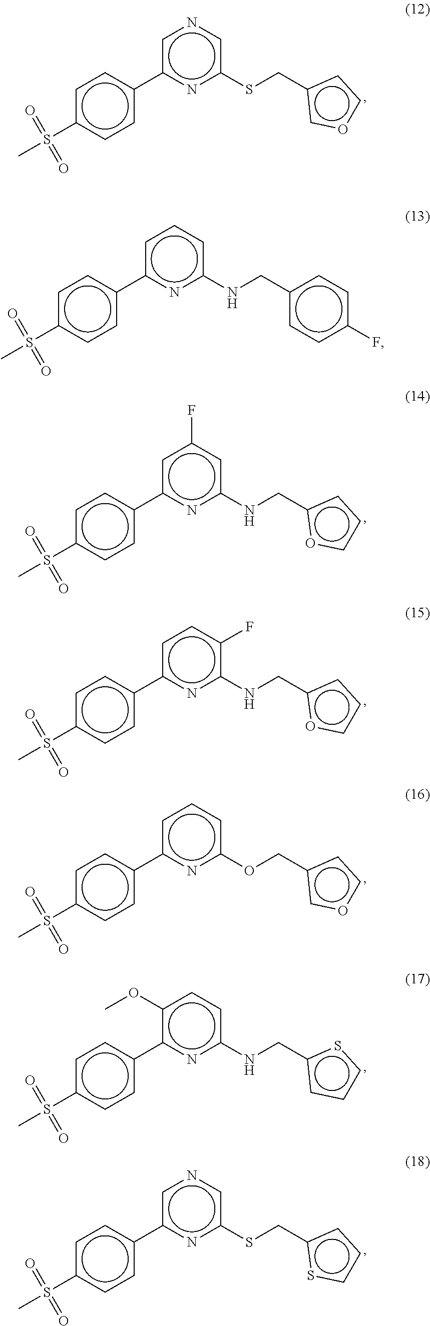

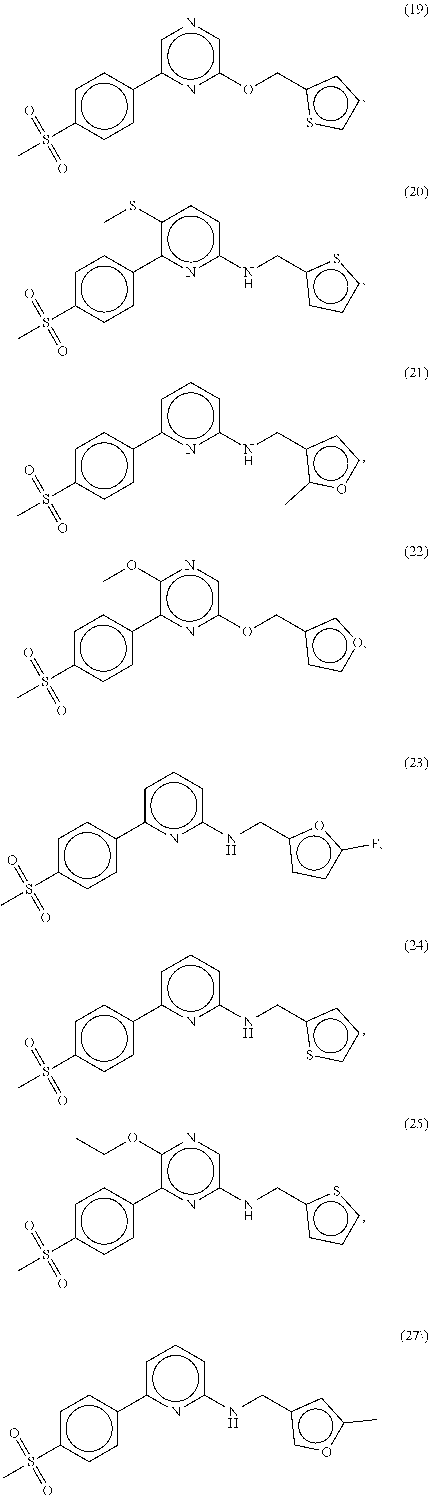

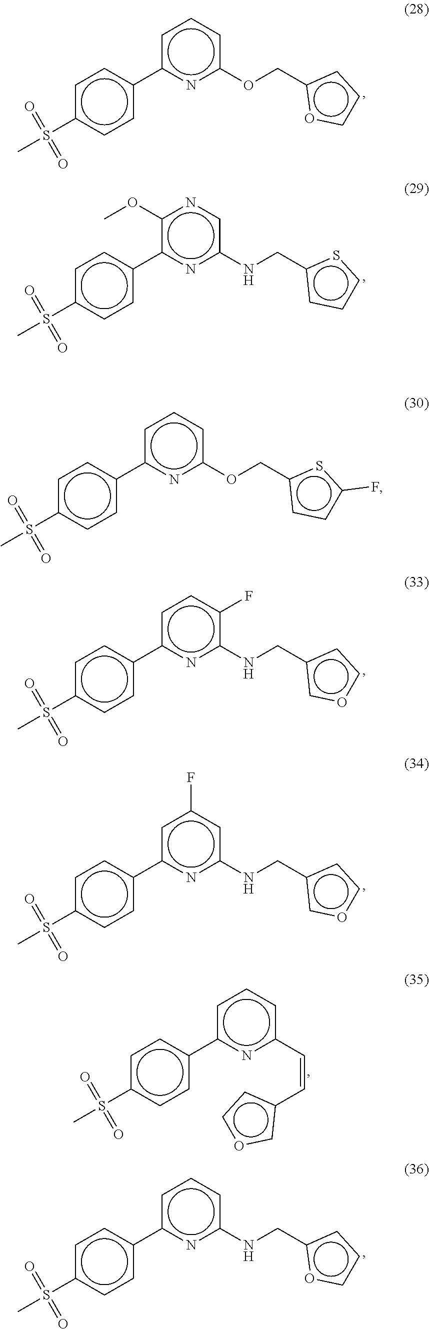

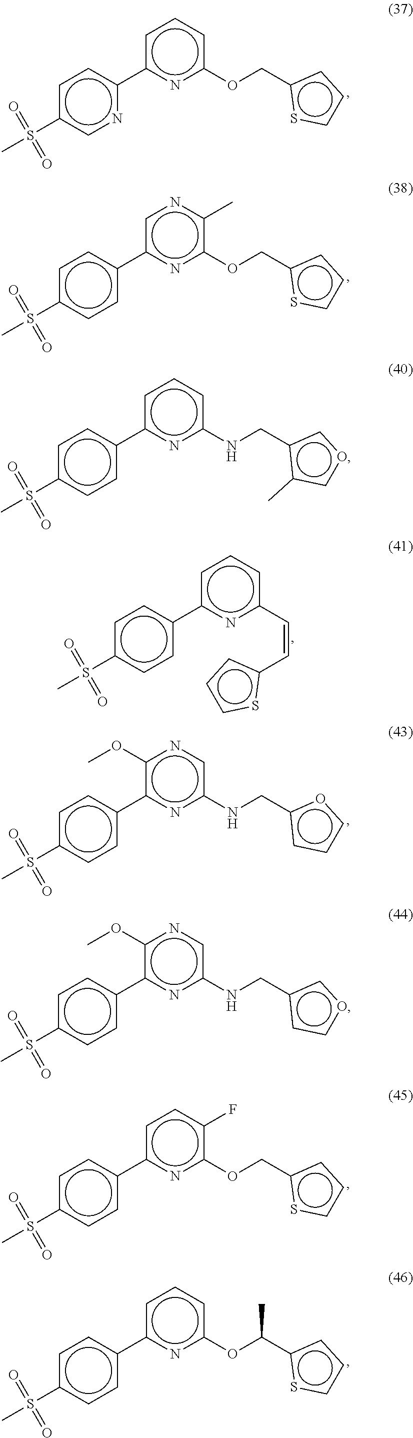

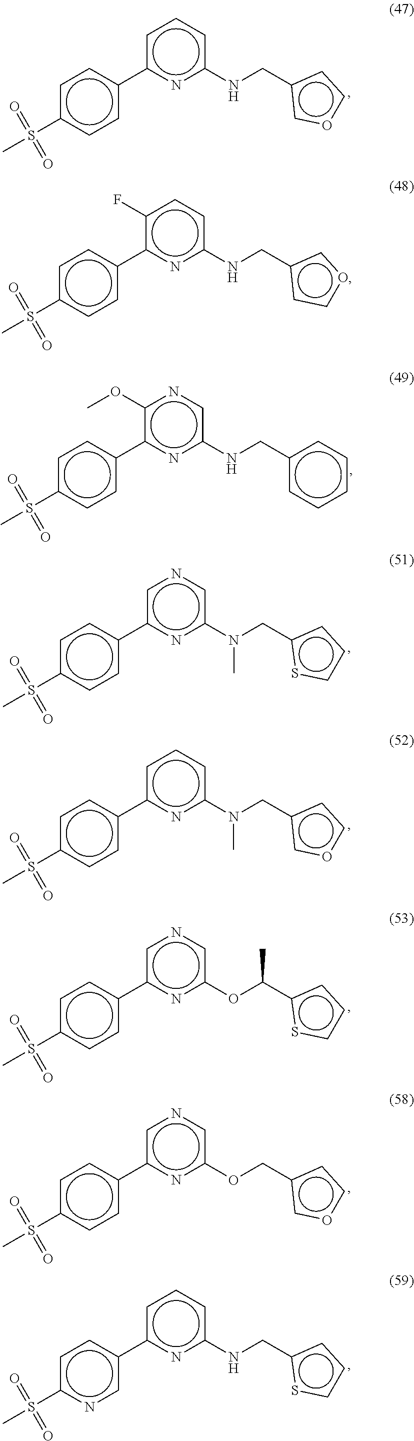

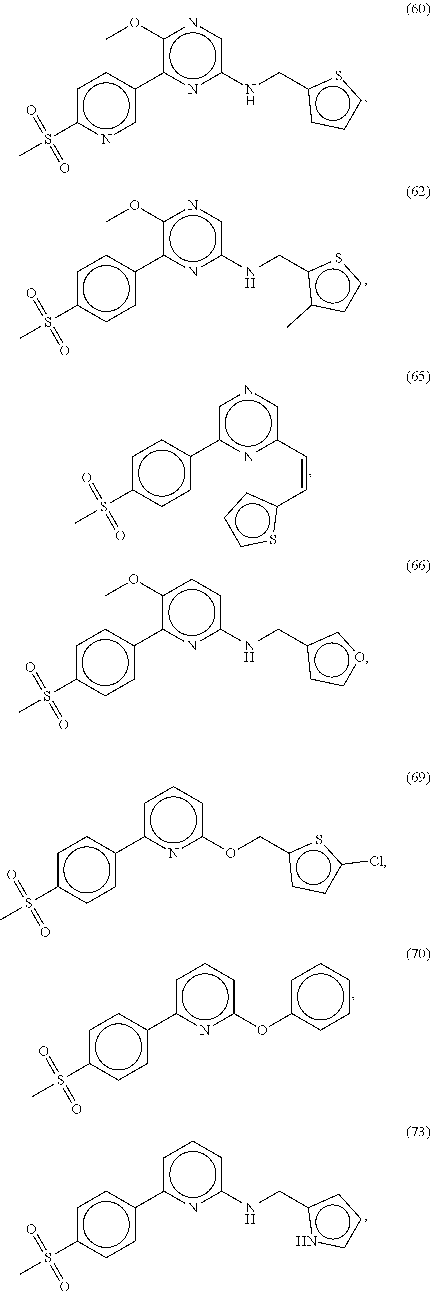

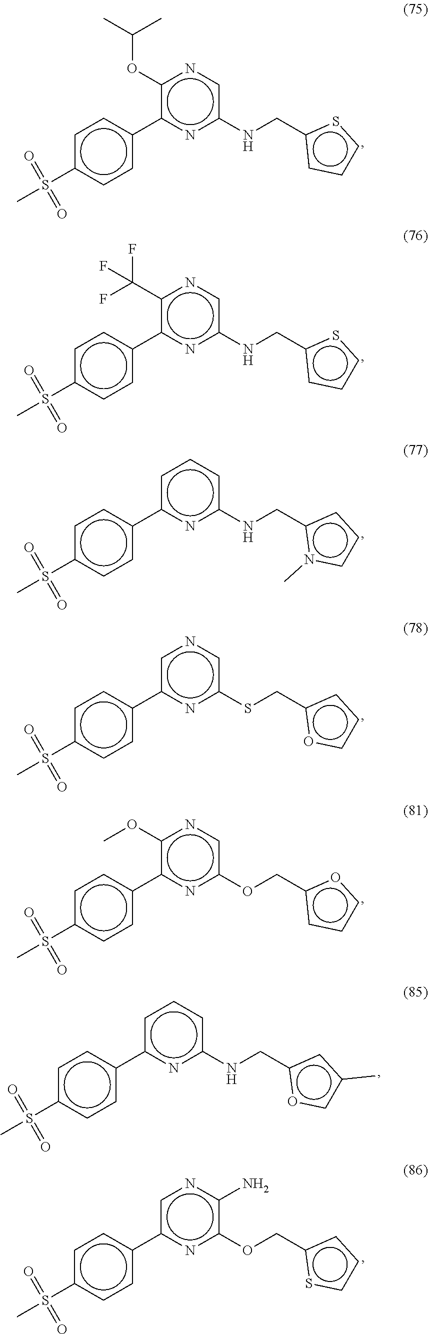

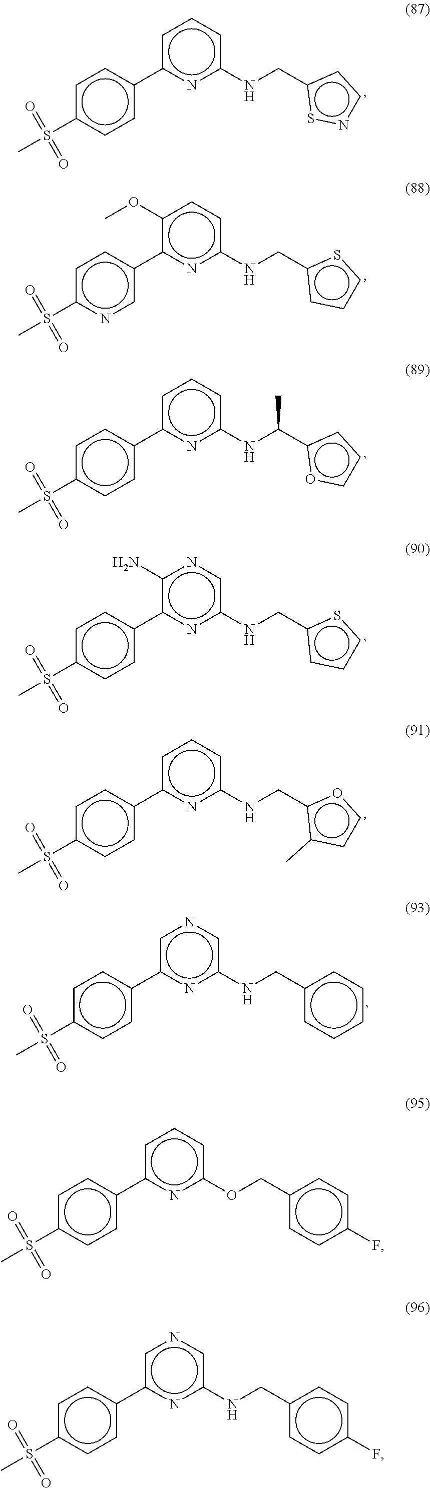

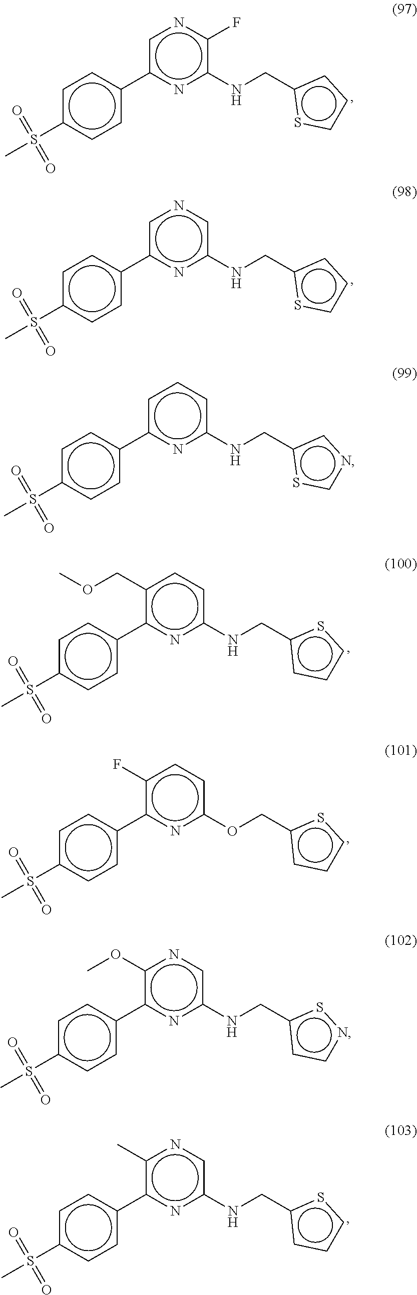

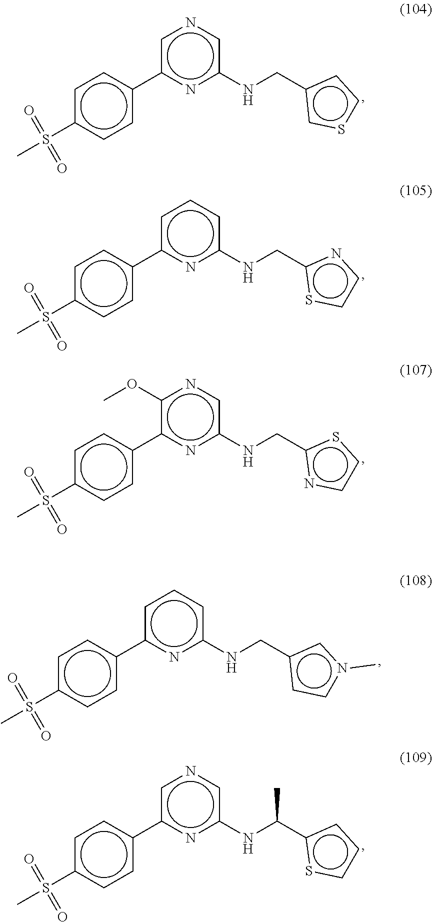

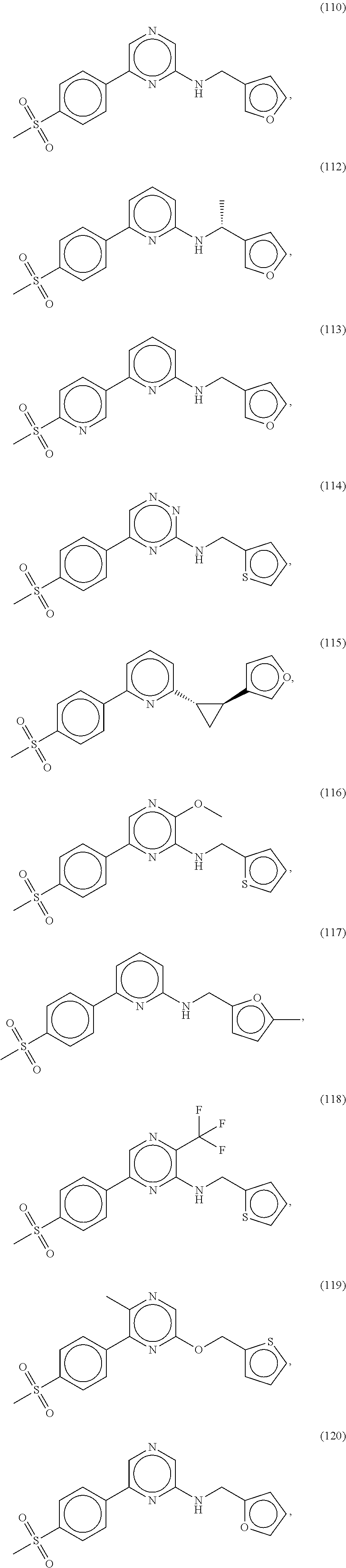

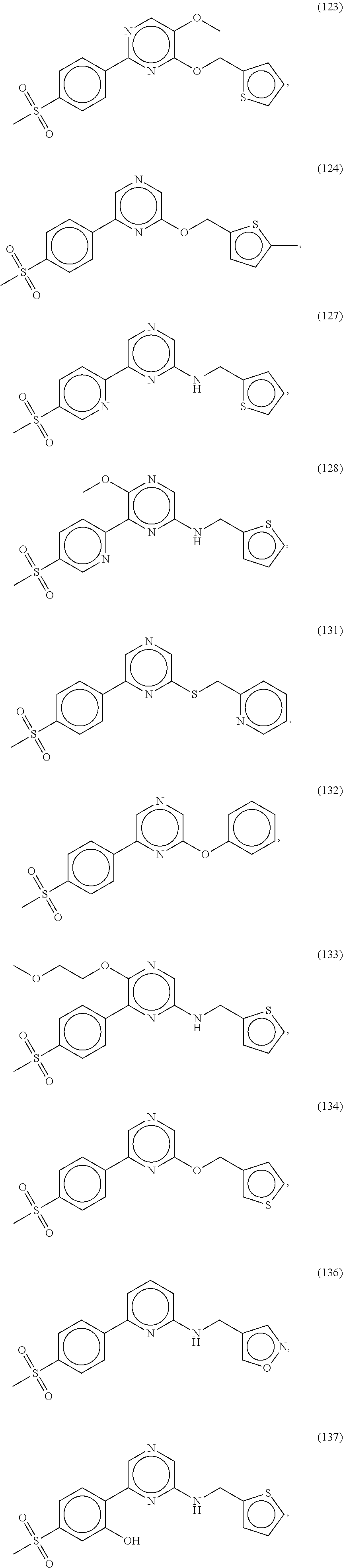

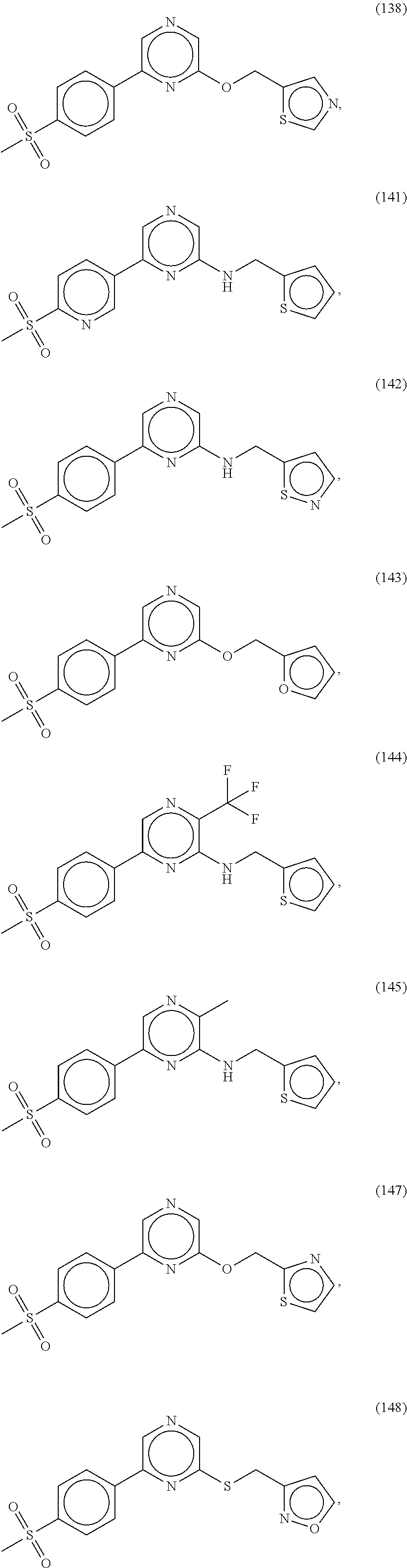

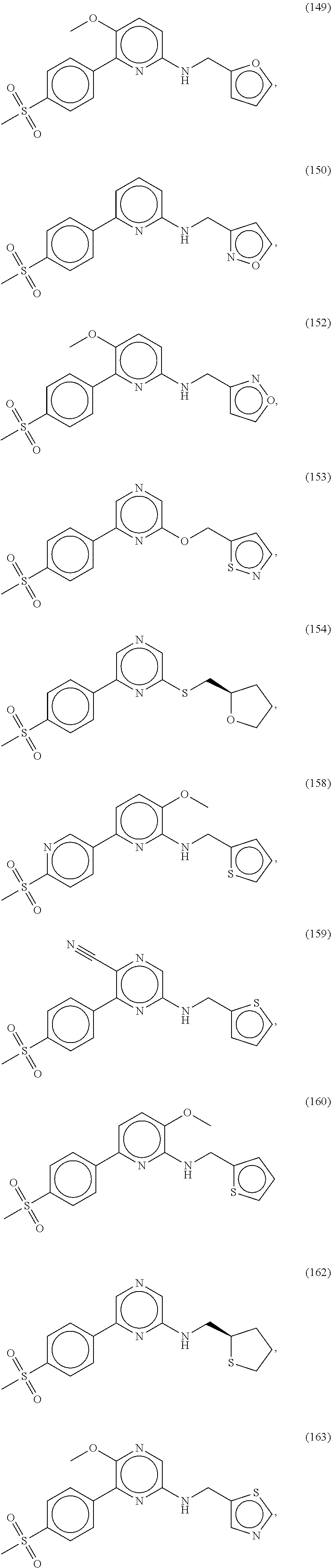

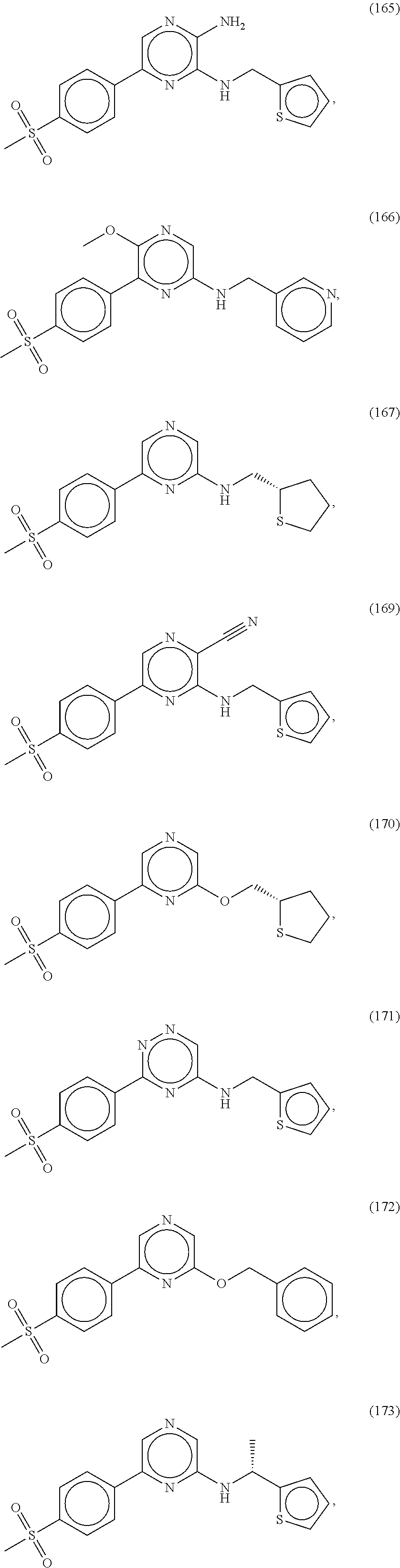

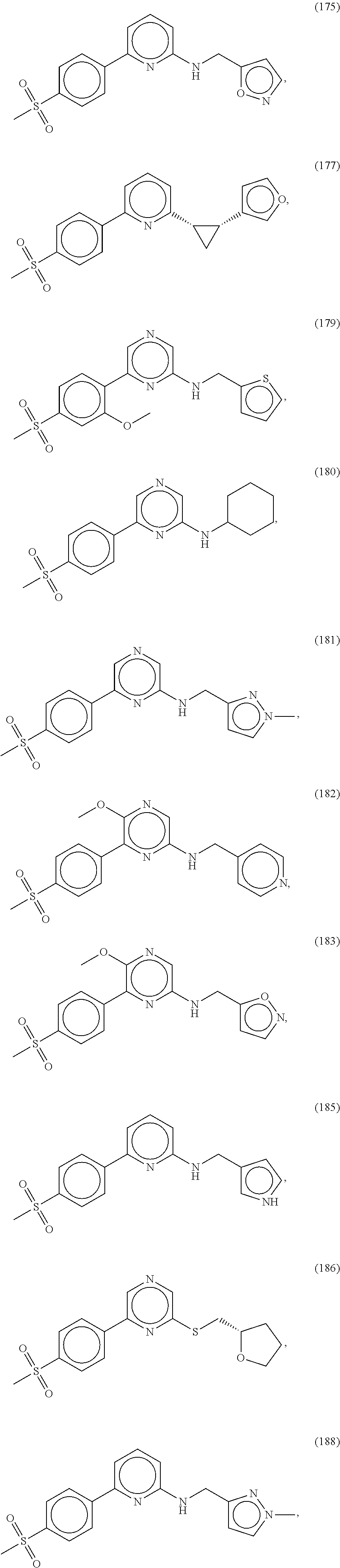

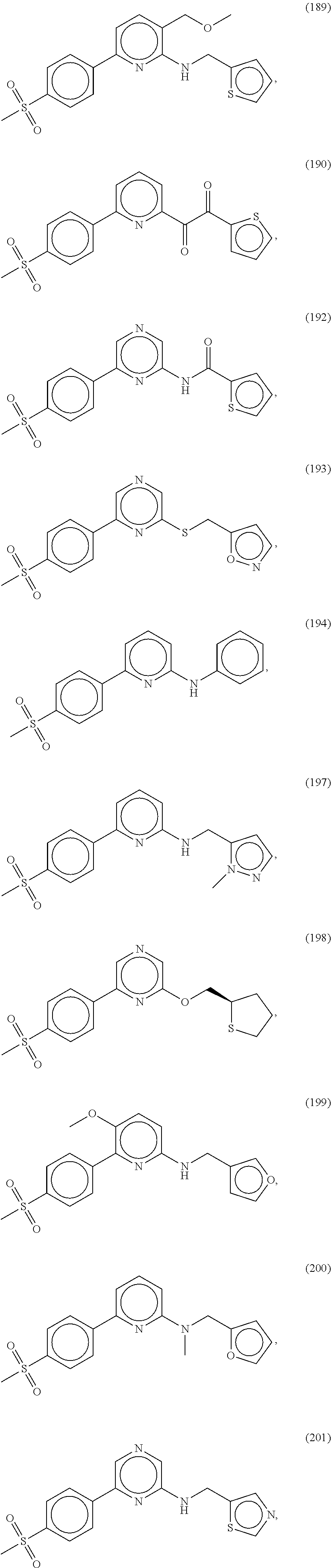

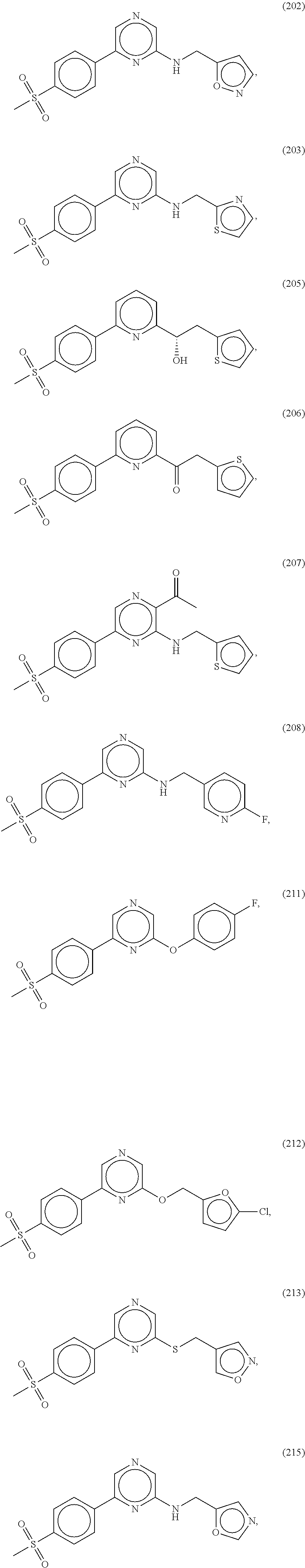

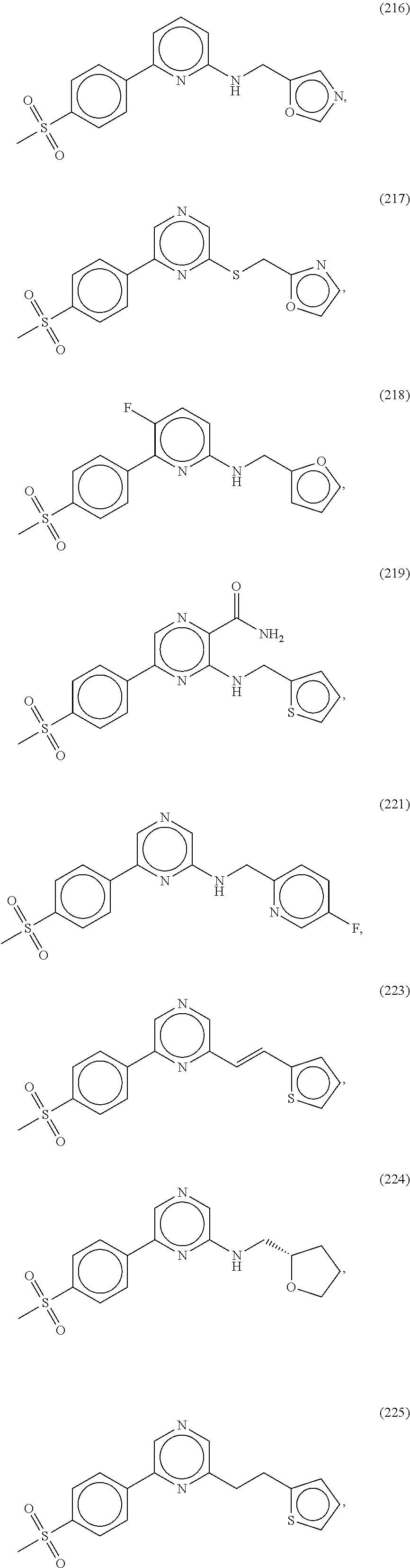

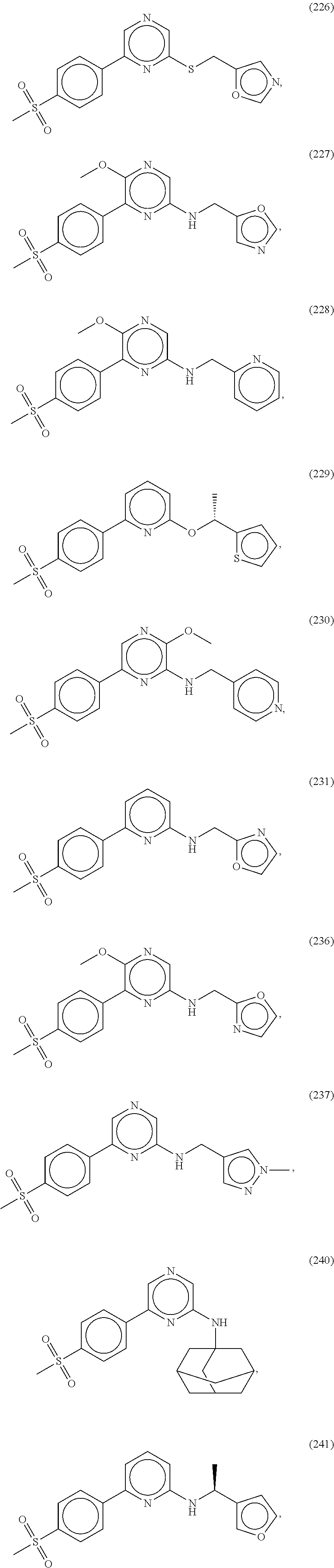

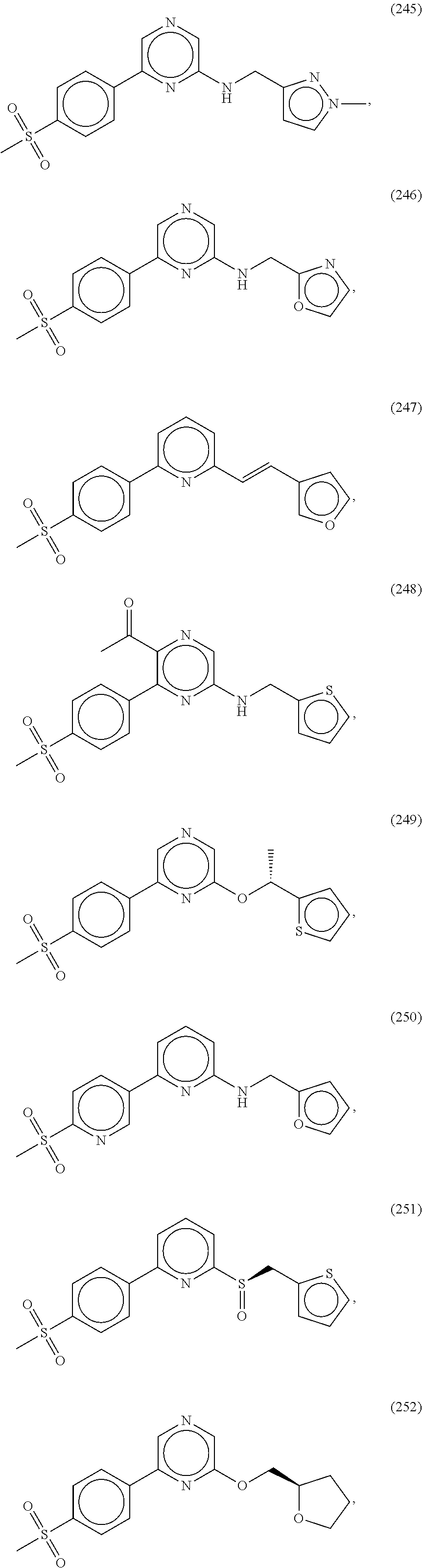

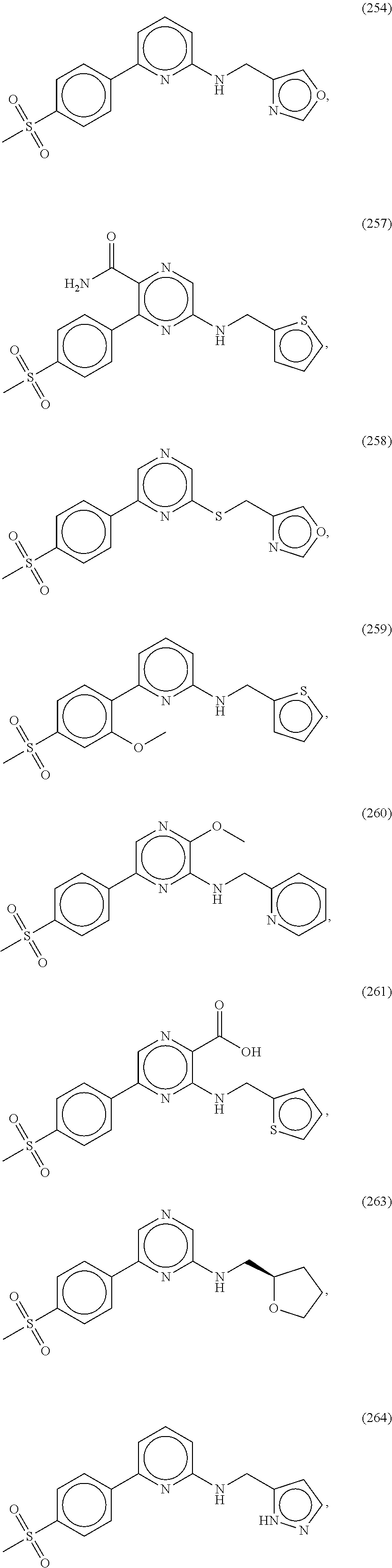

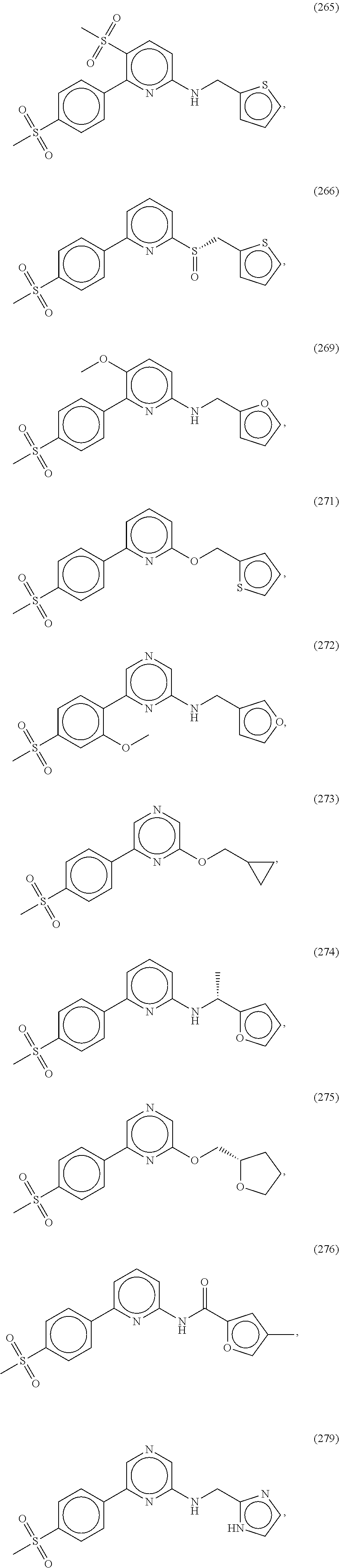

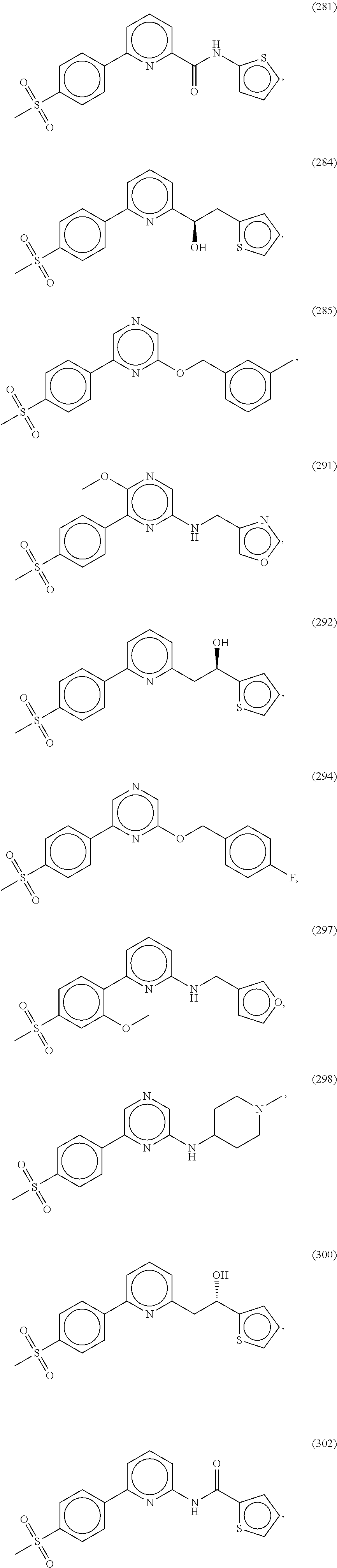

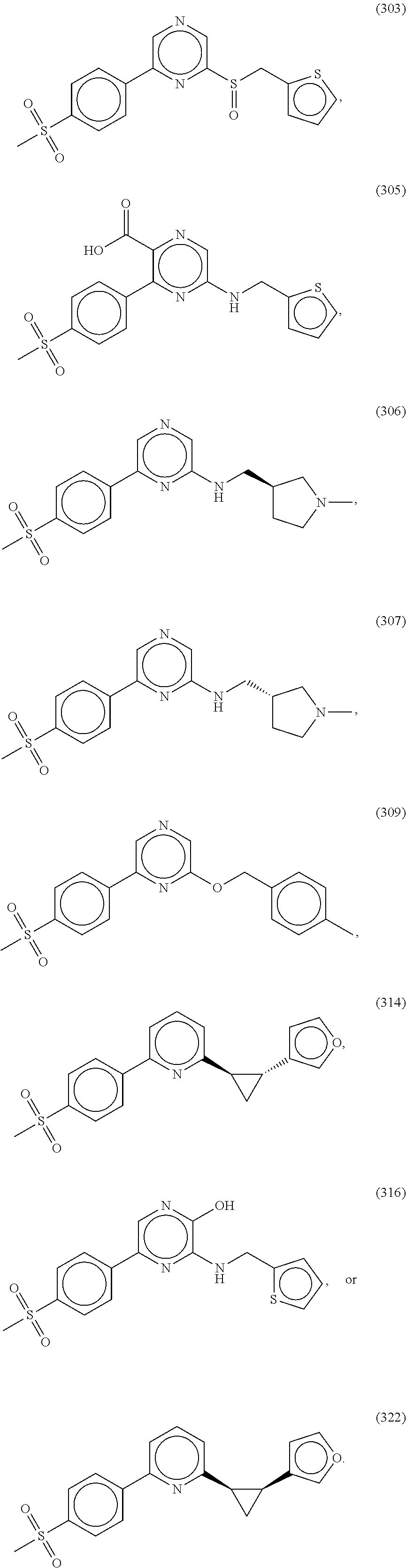

66. The compound of claim 1, or a salt, solvate, hydrate, polymorph, co-crystal, tautomer, stereoisomer, isotopically labeled derivative, or prodrug thereof, wherein the compound is of the formula:

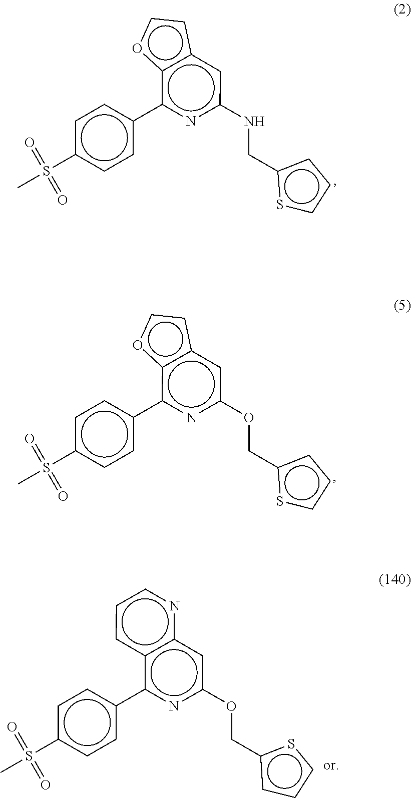

67. The compound of claim 1, or a salt, solvate, hydrate, polymorph, co-crystal, tautomer, stereoisomer, isotopically labeled derivative, or prodrug thereof, wherein the compound is of the formula:

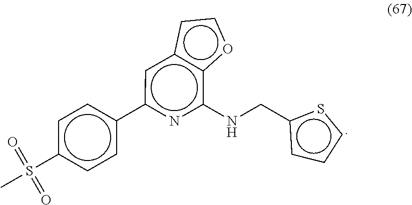

68. The compound of claim 1, or a salt, solvate, hydrate, polymorph, co-crystal, tautomer, stereoisomer, isotopically labeled derivative, or prodrug thereof, wherein the compound is

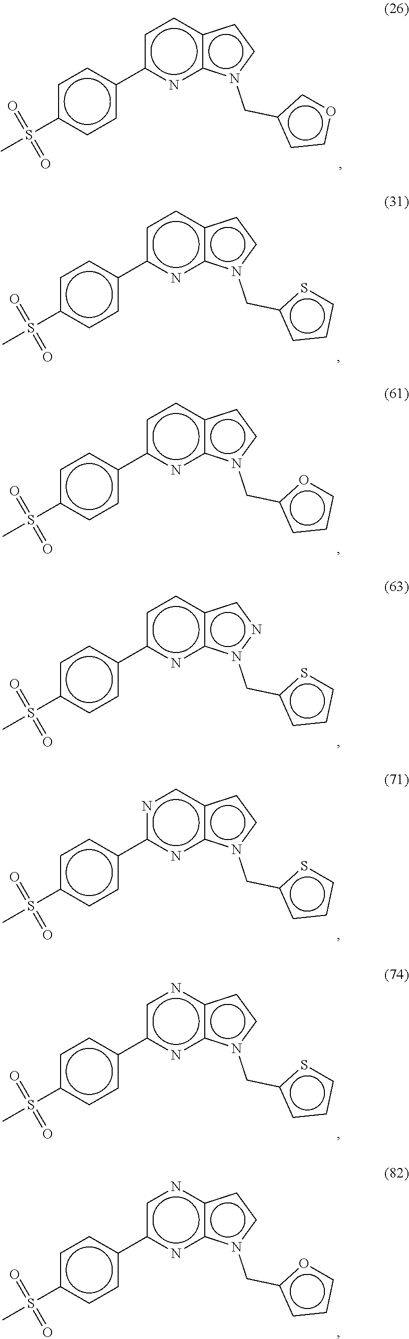

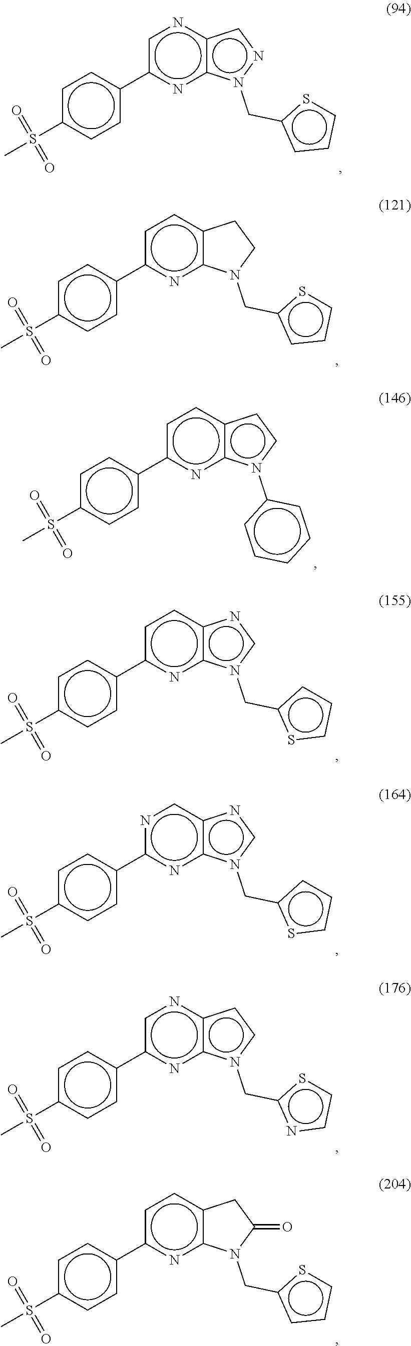

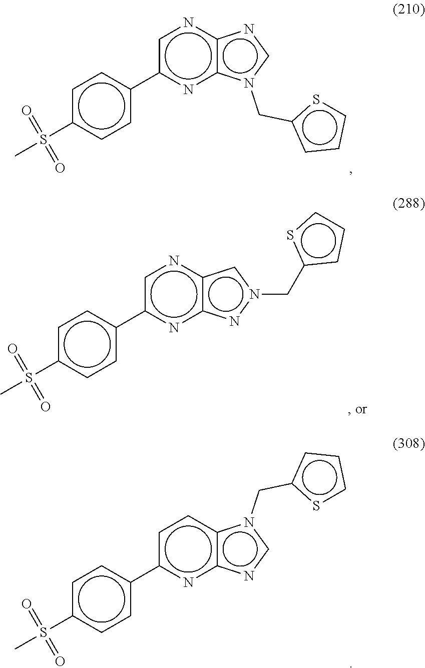

69. The compound of claim 1, or a salt, solvate, hydrate, polymorph, co-crystal, tautomer, stereoisomer, isotopically labeled derivative, or prodrug thereof, wherein the compound is of the formula:

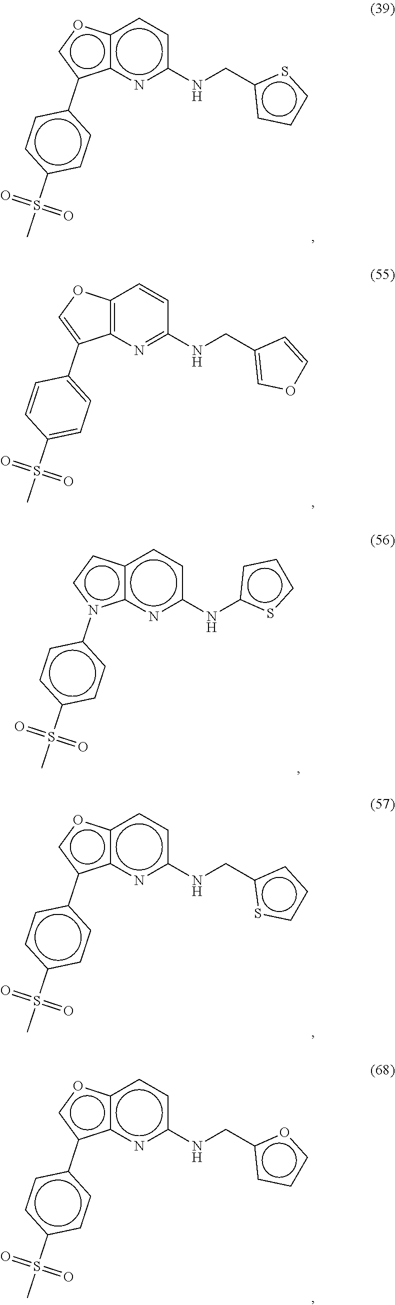

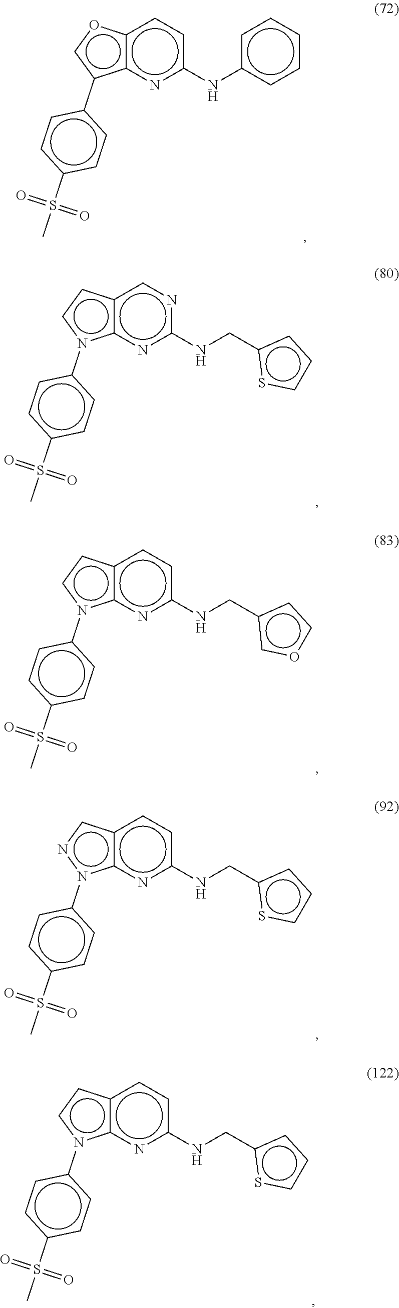

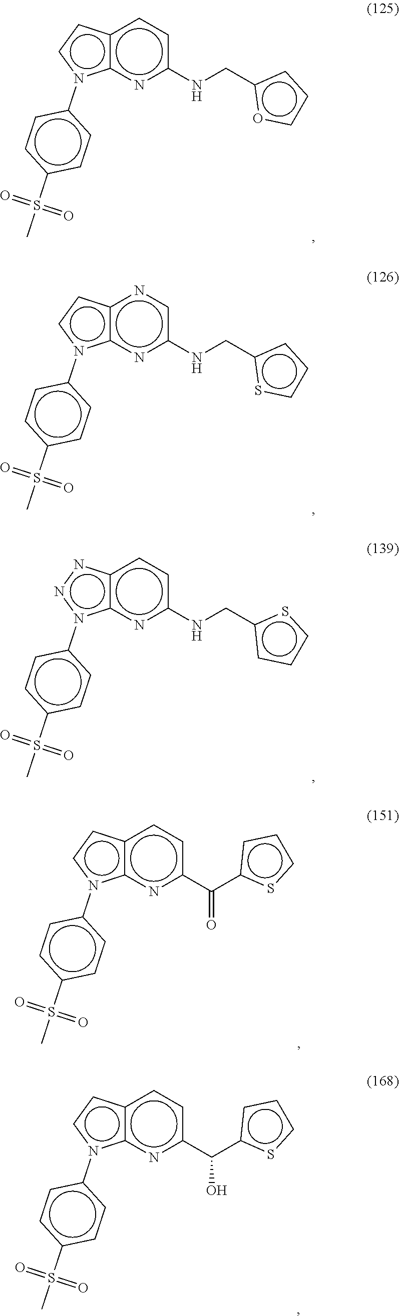

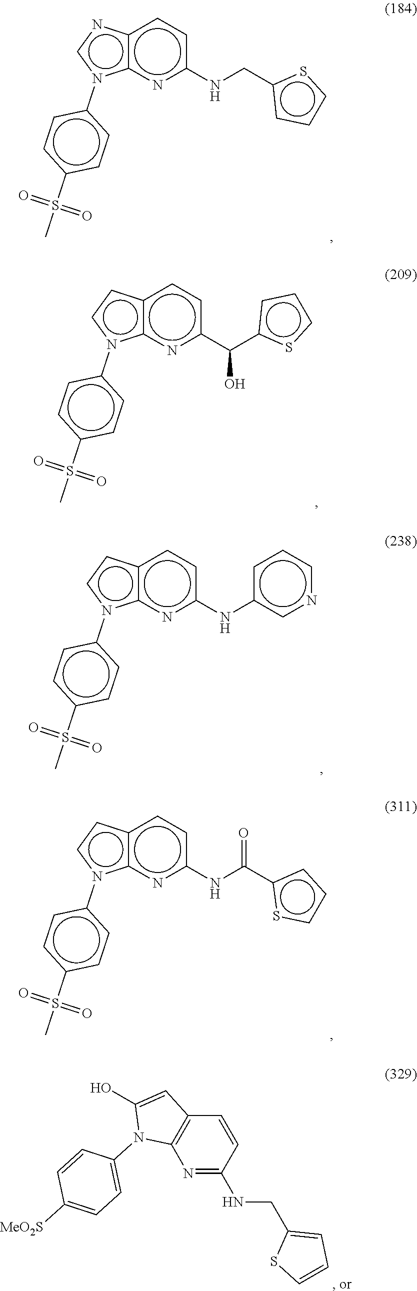

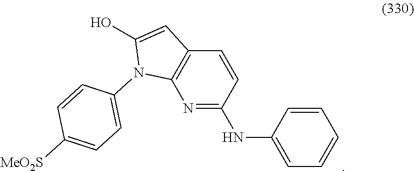 [SMILES: CS(=O)(=O)c1ccc(-n2c(O)cc3ccc(Nc4ccccc4)nc32)cc1 ; "."]

70. (canceled)

71. A compound, or a salt, solvate, hydrate, polymorph, co-crystal, tautomer, stereoisomer, isotopically labeled derivative, or prodrug thereof, wherein the compound is of the formula:

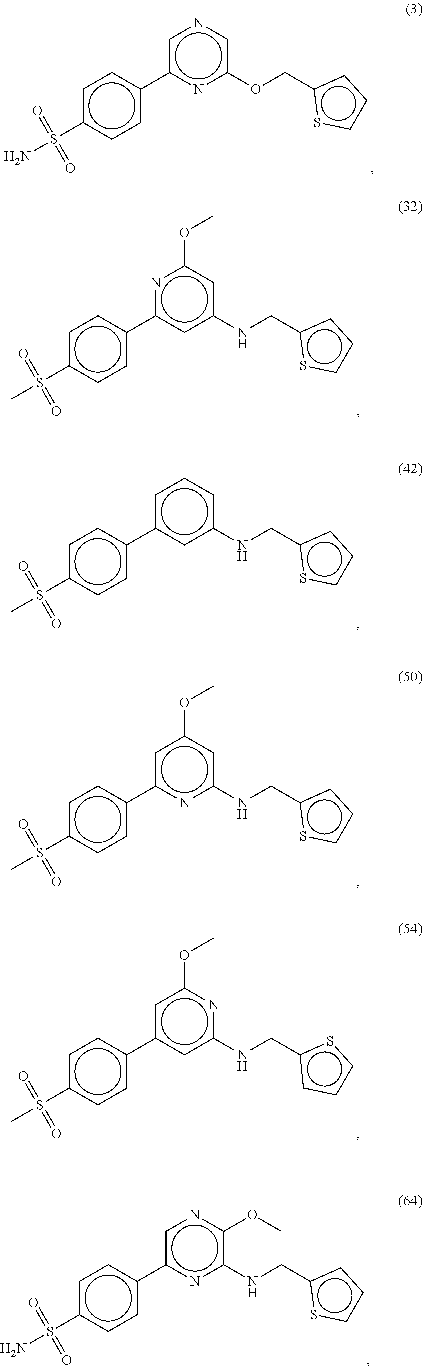

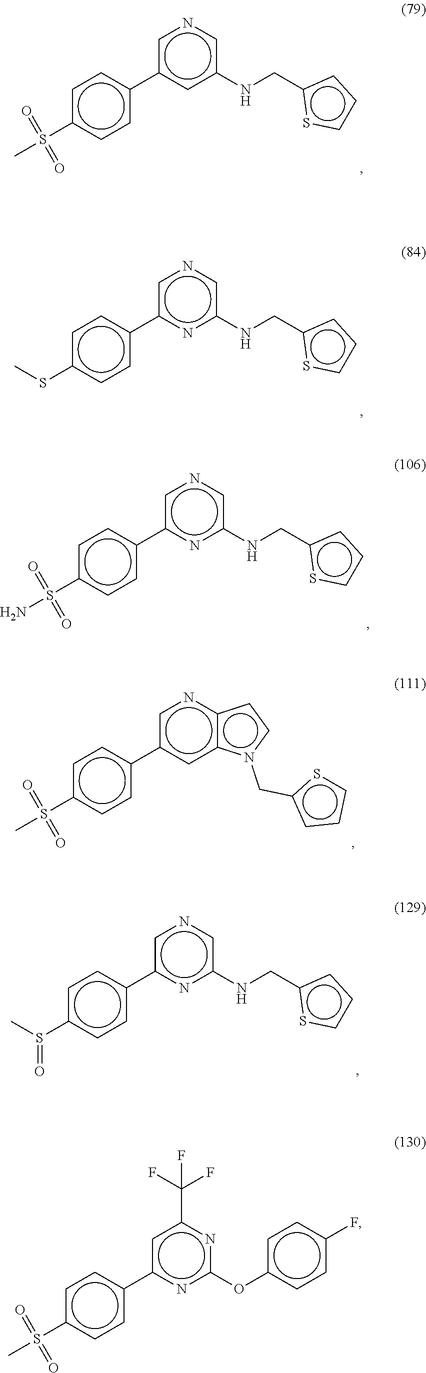

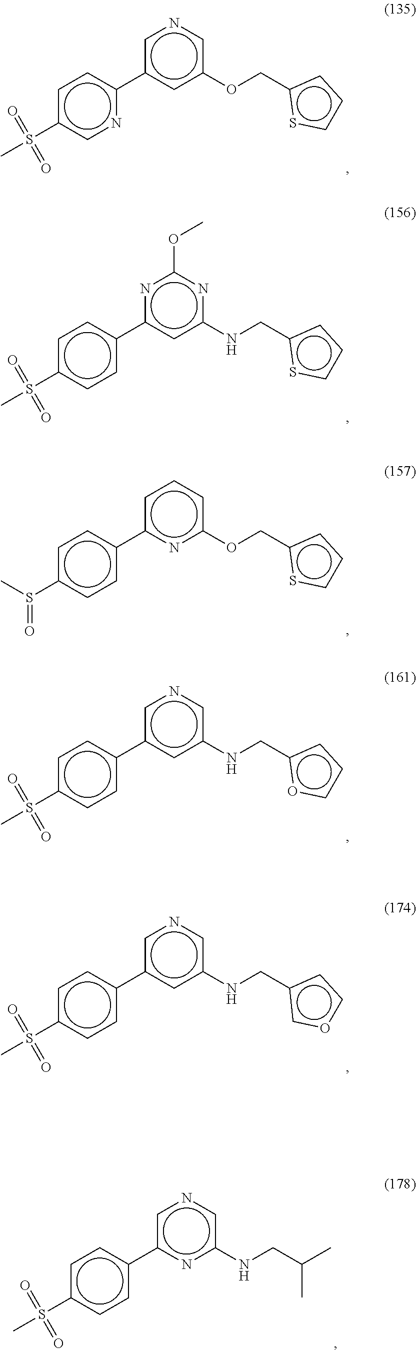

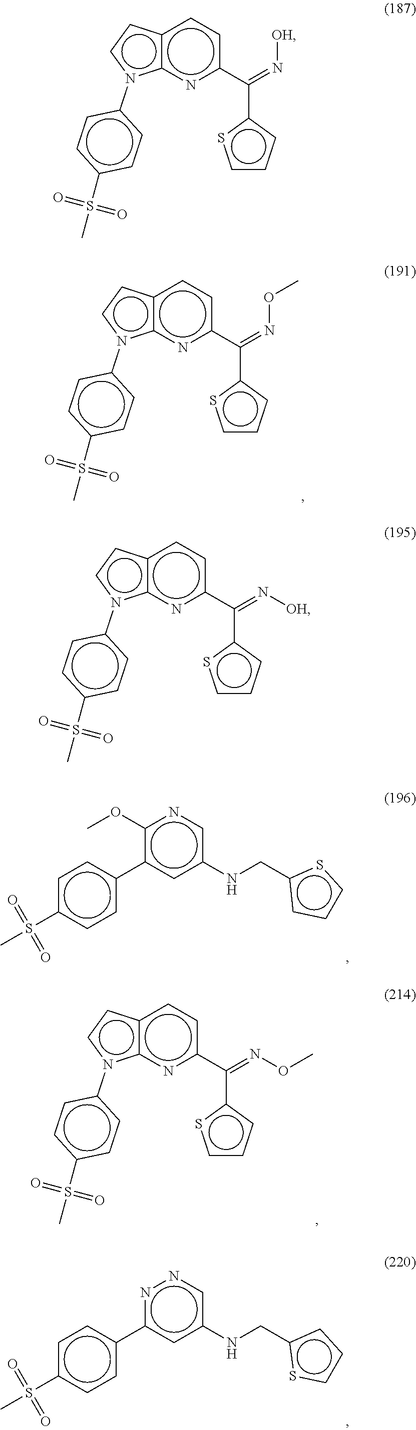

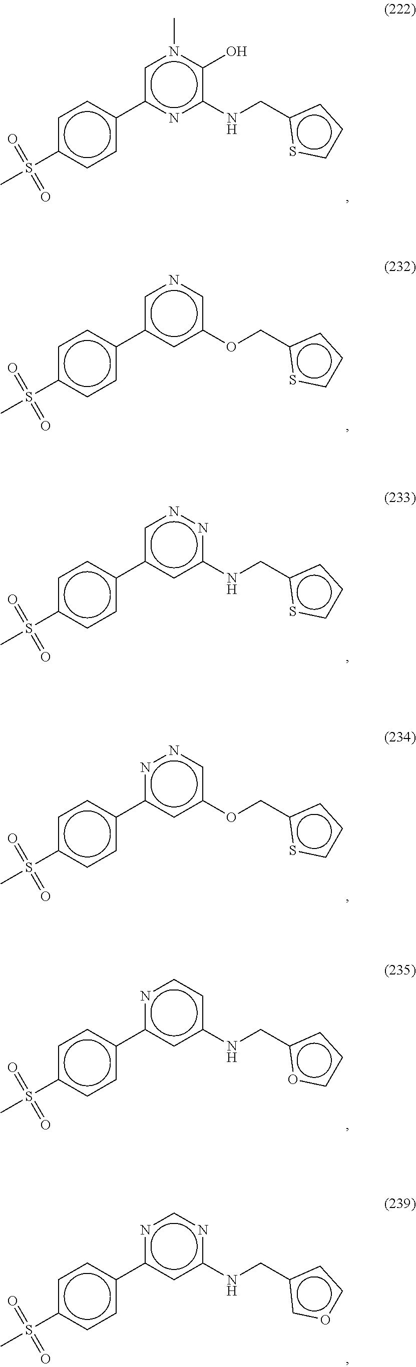

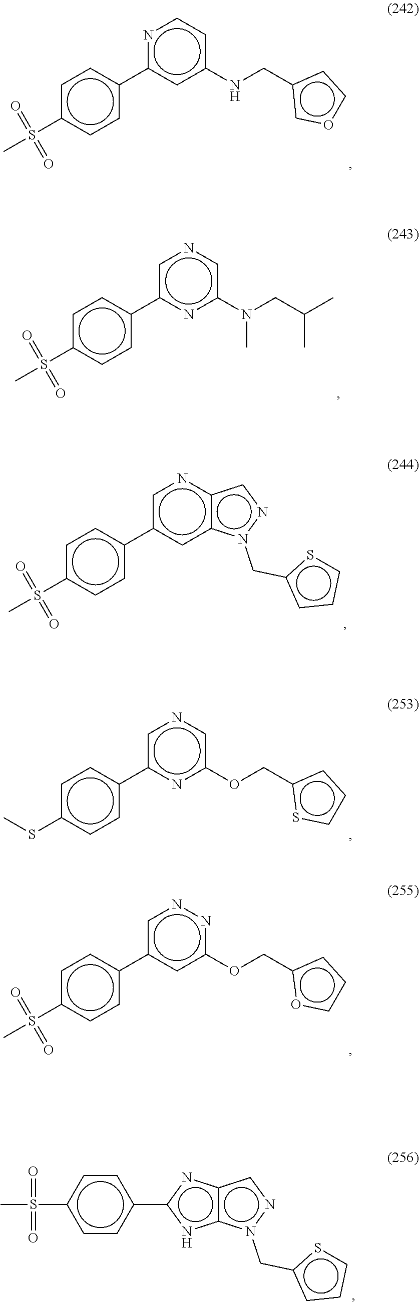

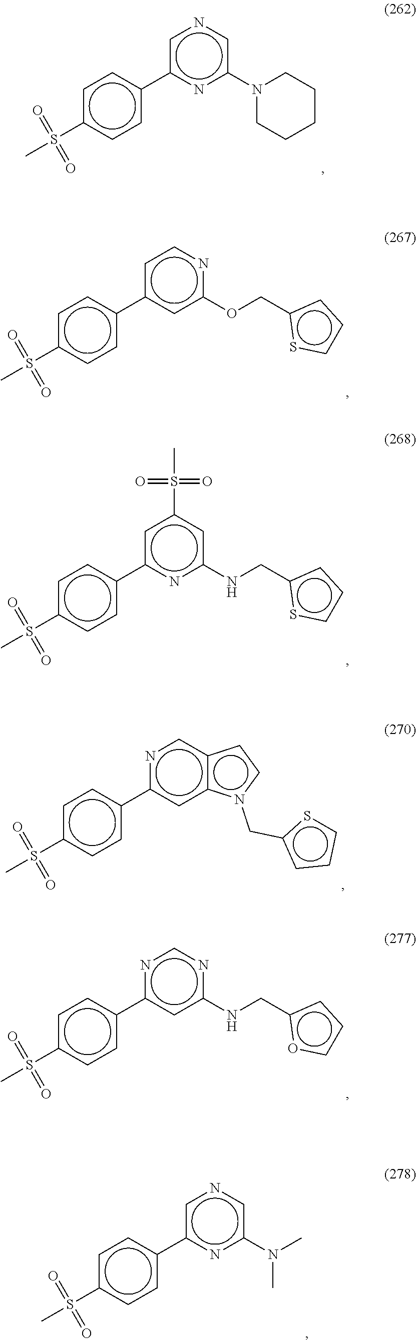

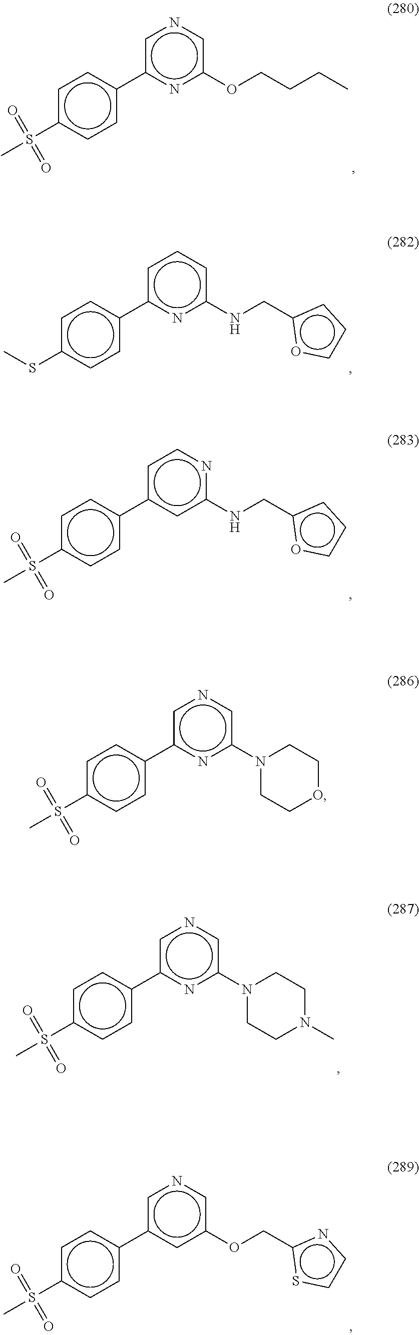

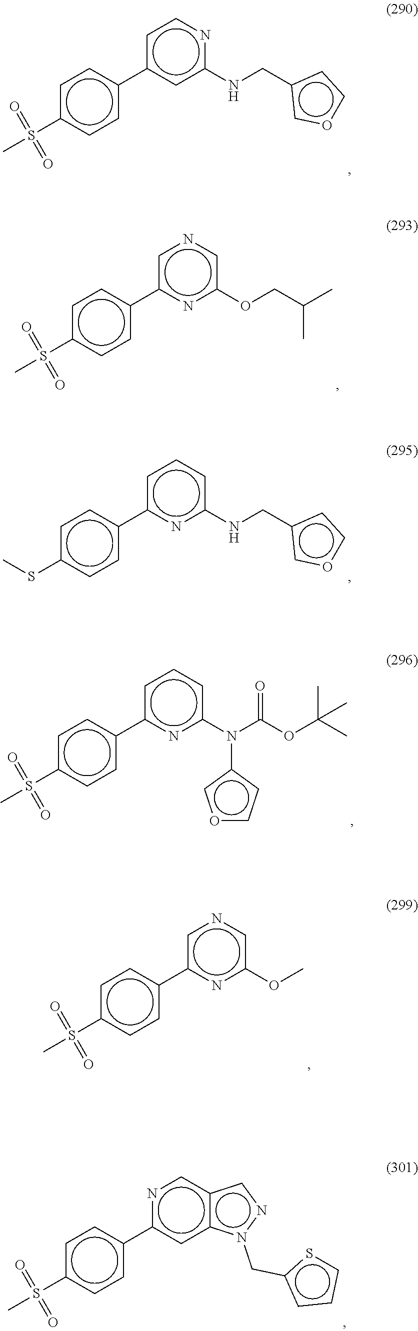

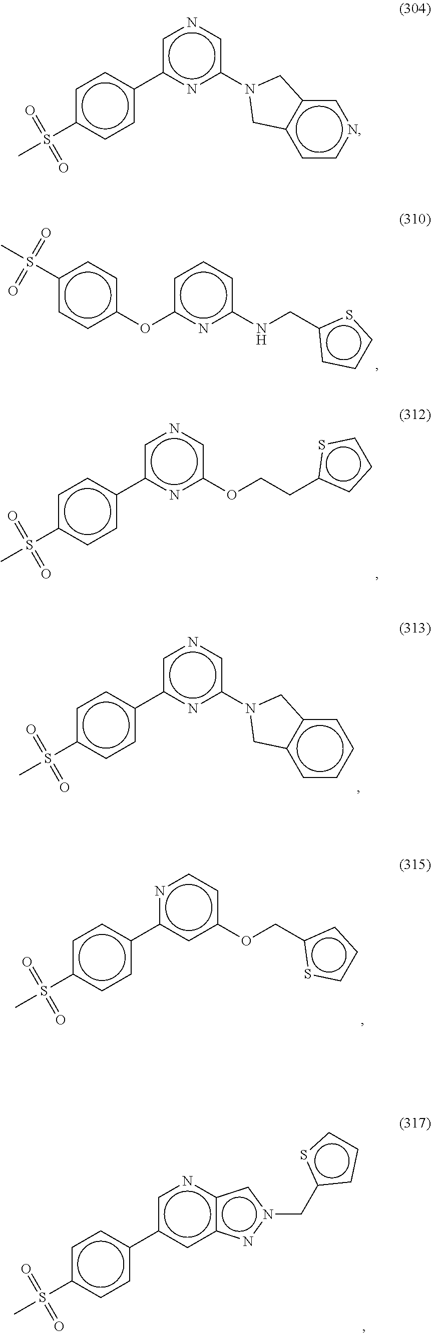

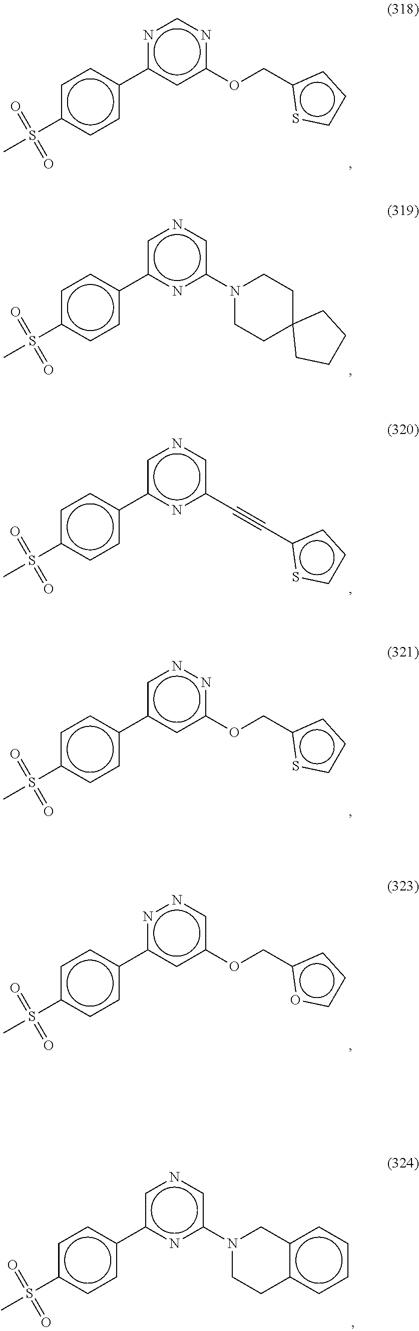

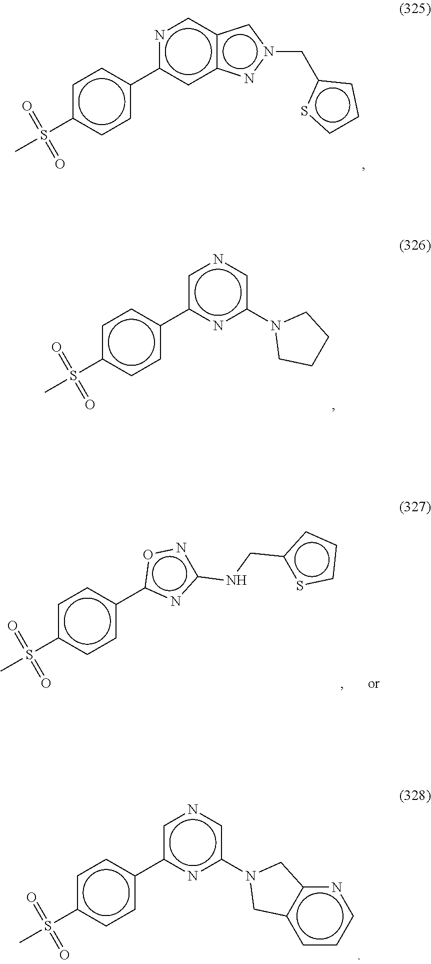

72. A compound, or a salt, solvate, hydrate, polymorph, co-crystal, tautomer, stereoisomer, isotopically labeled derivative, or prodrug thereof, wherein the compound is of the formula:

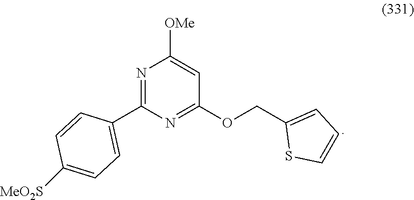 [SMILES: COc1cc(OCc2cccs2)nc(-c2ccc(S(C)(=O)=O)cc2)n1]

73-78. (canceled)

79. A pharmaceutical composition comprising:
a compound of claim 1, or a pharmaceutically acceptable salt, solvate, hydrate, polymorph, co-crystal, tautomer, stereoisomer, isotopically labeled derivative, or prodrug thereof; and
optionally a pharmaceutically acceptable excipient.

80-82. (canceled)

83. A kit comprising:
a compound of claim 1, or a salt, solvate, hydrate, polymorph, co-crystal, tautomer, stereoisomer, isotopically labeled derivative, or prodrug thereof; and
instructions for using the compound, salt, solvate, hydrate, polymorph, co-crystal, tautomer, stereoisomer, isotopically labeled derivative, or prodrug.

84. A method of diagnosing a disease in a subject in need thereof, the method comprising administering to the subject in need thereof an effective amount of a compound of claim 1, or a salt, solvate, hydrate, polymorph, co-crystal, tautomer, stereoisomer, isotopically labeled derivative, or prodrug thereof.

85. (canceled)

86. A method of treating a disease in a subject in need thereof, the method comprising administering to the subject in need thereof an effective amount of a compound of claim 1, or a salt, solvate, hydrate, polymorph, co-crystal, tautomer, stereoisomer, isotopically labeled derivative, or prodrug thereof.

87. A method of preventing a disease in a subject in need thereof, the method comprising administering to the subject in need thereof an effective amount of a compound of claim 1, or a salt, solvate, hydrate, polymorph, co-crystal, tautomer, stereoisomer, isotopically labeled derivative, or prodrug thereof.

88-97. (canceled)

98. A method of inhibiting the activity or decreasing the level of cyclooxygenase 2 (COX2) in a subject in need thereof or in a cell, biological sample, or tissue, the method comprising administering to the subject in need thereof, or contacting the cell, biological sample, or tissue with, an effective amount of a compound of claim 1, or a pharmaceutically acceptable salt, solvate, hydrate, polymorph, co-crystal, tautomer, stereoisomer, isotopically labeled derivative, or prodrug thereof.

99-103. (canceled)

104. A pharmaceutical composition comprising:
a compound of claim 71, or a pharmaceutically acceptable salt, solvate, hydrate, polymorph, co-crystal, tautomer, stereoisomer, isotopically labeled derivative, or prodrug thereof; and
optionally a pharmaceutically acceptable excipient.

105. A kit comprising:
a compound of claim 71, or a salt, solvate, hydrate, polymorph, co-crystal, tautomer, stereoisomer, isotopically labeled derivative, or prodrug thereof; and
instructions for using the compound, salt, solvate, hydrate, polymorph, co-crystal, tautomer, stereoisomer, isotopically labeled derivative, or prodrug.

106. A method of diagnosing a disease in a subject in need thereof, the method comprising administering to the subject in need thereof an effective amount of a compound of claim 71, or a salt, solvate, hydrate, polymorph, co-crystal, tautomer, stereoisomer, isotopically labeled derivative, or prodrug thereof.

107. A method of treating a disease in a subject in need thereof, the method comprising administering to the subject in need thereof an effective amount of a compound of claim 71, or a salt, solvate, hydrate, polymorph, co-crystal, tautomer, stereoisomer, isotopically labeled derivative, or prodrug thereof.

108. A method of preventing a disease in a subject in need thereof, the method comprising administering to the subject in need thereof an effective amount of a compound of claim 71, or a salt, solvate, hydrate, polymorph, co-crystal, tautomer, stereoisomer, isotopically labeled derivative, or prodrug thereof.

109. A method of inhibiting the activity or decreasing the level of cyclooxygenase 2 (COX2) in a subject in need thereof or in a cell, biological sample, or tissue, the method comprising administering to the subject in need thereof, or contacting the cell, biological sample, or tissue with, an effective amount of a compound of claim 71, or a pharmaceutically acceptable salt, solvate, hydrate, polymorph, co-crystal, tautomer, stereoisomer, isotopically labeled derivative, or prodrug thereof.

110. A pharmaceutical composition comprising:
a compound of claim 72, or a pharmaceutically acceptable salt, solvate, hydrate, polymorph, co-crystal, tautomer, stereoisomer, isotopically labeled derivative, or prodrug thereof; and
optionally a pharmaceutically acceptable excipient.

111. A kit comprising:
a compound of claim 72, or a salt, solvate, hydrate, polymorph, co-crystal, tautomer, stereoisomer, isotopically labeled derivative, or prodrug thereof; and
instructions for using the compound, salt, solvate, hydrate, polymorph, co-crystal, tautomer, stereoisomer, isotopically labeled derivative, or prodrug.

112. A method of diagnosing a disease in a subject in need thereof, the method comprising administering to the subject in need thereof an effective amount of a compound of claim 72, or a salt, solvate, hydrate, polymorph, co-crystal, tautomer, stereoisomer, isotopically labeled derivative, or prodrug thereof.

113. A method of treating a disease in a subject in need thereof, the method comprising administering to the subject in need thereof an effective amount of a compound of claim 72, or a salt, solvate, hydrate, polymorph, co-crystal, tautomer, stereoisomer, isotopically labeled derivative, or prodrug thereof.

114. A method of preventing a disease in a subject in need thereof, the method comprising administering to the subject in need thereof an effective amount of a compound of claim 72, or a salt, solvate, hydrate, polymorph, co-crystal, tautomer, stereoisomer, isotopically labeled derivative, or prodrug thereof.

115. A method of inhibiting the activity or decreasing the level of cyclooxygenase 2 (COX2) in a subject in need thereof or in a cell, biological sample, or tissue, the method comprising administering to the subject in need thereof, or contacting the cell, biological sample, or tissue with, an effective amount of a compound of claim 72, or a pharmaceutically acceptable salt, solvate, hydrate, polymorph, co-crystal, tautomer, stereoisomer, isotopically labeled derivative, or prodrug thereof.
